(19) United States
(12) Patent Application Publication
(10) Pub. No.: US 2026/0061003 A1
(43) Pub. Date: Mar. 5, 2026

(54) PLATELET-RICH PLASMA COMPOSITIONS, PREPARATION, AND USES THEREOF

(71) Applicant: VetStem, Inc., Poway, CA (US)

(72) Inventors: Anne S. HALE, Placitas, NM (US); Robert Jerome HARMAN, JR., Ramona, CA (US)

(21) Appl. No.: 19/383,260

(22) Filed: Nov. 7, 2025

Related U.S. Application Data
(63) Continuation-in-part of application No. PCT/US2024/028205, filed on May 7, 2024.
(60) Provisional application No. 63/465,845, filed on May 11, 2023.

(51) Int. Cl. A61K 35/16; A61K 9/00; A61K 9/19; A61K 45/06; A61P 19/02; A61P 27/02; A61P 27/16

(52) U.S. Cl. CPC A61K 35/16; A61K 9/0019; A61K 9/19; A61K 45/06; A61P 19/02; A61P 27/02; A61P 27/16

(57) ABSTRACT

Provided are platelet-rich plasma (PRP) compositions, including freeze-dried (lyophilized) and reconstituted compositions containing primed platelets. The compositions are freeze-dried for long term storage and do not contain cryoprotectants or cryopreservatives.

### RELATED APPLICATIONS

This application is a continuation-in-part of International PCT application No. PCT/US2024/028205, filed on May 7, 2024, and published on Nov. 14, 2024, as International PCT publication No. WO 2024/233577, entitled “PLATELET-RICH PLASMA COMPOSITIONS, PREPARATION, AND USES THEREOF,” to inventors Anne S. Hale and Robert Jerome Harman, Jr., and to applicant VetStem, Inc. This application and International PCT Application No. PCT/US2024/028205 each claim the benefit of priority to U.S. provisional application Ser. No. 63/465,845, entitled “PLATELET-RICH PLASMA COMPOSITIONS, PREPARATION, AND USES THEREOF,” filed May 11, 2023, to inventors Anne S. Hale and Robert Jerome Harman, Jr., and Applicant VetStem, Inc.

The subject matter of each of these applications is incorporated by reference in its entirety.

### FIELD OF THE INVENTION

Provided are platelet-rich plasma (PRP) compositions, including freeze-dried (lyophilized) and reconstituted compositions containing primed platelets. The compositions are freeze-dried for long term storage and do not contain cryoprotectants or cryopreservatives.

### BACKGROUND

Platelet-rich plasma (PRP) is a blood product containing an elevated concentration of platelets compared to whole blood. Platelet-rich plasma (PRP) has a variety of applications, such as a therapeutic, for accelerating healing and for the management of a variety of conditions, including musculoskeletal conditions and injuries. PRP products are known in the art. A problem is that platelets deteriorate quickly and must be used immediately or freeze-dried for storage. Freeze-dried PRP compositions have been prepared. They are prepared in the presence of cryopreservatives; and upon reconstitution a large percentage of the platelets in such compositions are activated, which limits the uses of the platelet compositions. Because of the variety of uses, and the difficulties in preserving and storing platelet compositions, there is a need for compositions with improved properties.

### SUMMARY

Platelets have many therapeutic applications; they can be used to provide or infuse platelets for hemostasis. They also are sources of growth factors and cytokines and other such molecules, and are used, for example, for regenerative therapies for provision of such molecules. Platelets are provided in compositions that contain plasma. Platelets once isolated become activated, and quickly release these regenerative molecules from granules before administration. Methods for preparing platelets result in activated platelets. For storage to attempt to preserve platelets for later use, they are formulated in compositions that contain cryopreservatives, which results in compositions that generally contain about 80% activated platelets. Platelets are provided platelet-rich plasma compositions and are lyophilized (freeze-dried for storage). The cryopreservatives are added prior to lyophilization to protect the platelets from damage during the freezing process.

Provided herein are platelet-rich plasma (PRP) compositions that contain a far higher percentage of platelets that are not fully activated, and methods for preparing such compositions. Platelets in the PRP compositions provided herein are in a primed state such that they have not yet released the regenerative molecules from their granules, but do so upon administration. As detailed herein, primed platelets exhibit some characteristics of activated platelets but are not fully activated; the contents of the platelet granules have not been released. Primed platelets are not quiescent and are not activated, rather they show early markers of activated platelets. Compositions with high percentage (at least about 40%) of platelets in a primed state have heretofore not been produced or provided.

The PRP compositions provided herein, not only have a high percentage of primed platelets, they do not contain cryopreservatives nor do they require cryopreservative for freeze-drying. Methods for preparing PRP compositions that do not include preservatives and that contain primed platelets are provided. The resulting compositions are provided.

Provided are platelet-rich plasma (PRP) compositions that comprise primed platelets, wherein at least 40% of the platelets in the composition are primed, but not fully activated. For example, at least 41%, 45%, 50%, 55%, 60%, 65%, 70%, 75%, 80%, 85%, or more of the platelets are in a primed state. The PRP compositions contain plasma and platelets, and optionally additional agents, such as anti-coagulants, such as those added during collection of the platelets and/or plasma, and can contain additional therapeutic agents. The PRP compositions provided herein do not contain a cryopreservative. In the PRP compositions, plasma comprises at or about between at or about 30% up to 80%, by volume of the composition or can comprise at or about between at or about 20% up to 90%, by volume of the composition.

The PRP compositions, as described herein, are provided in different forms and formulations; in all instances a high percentage of platelets, at least 40% or more are in a primed state. For example, PRP compositions provide herein can be in a liquid, semi-liquid, gel, or solid, such as powder form. In all instances the compositions do not contain a cryopreservative. In accord with the description herein, initially a concentrate of platelets and plasma is produced. Additional plasma can be added. After processing and preparation as described in sections below, the resulting compositions are then freeze-dried to produce a powder for storage. For administration to an animal, the freeze-dried compositions are reconstituted (rehydrated) by adding a liquid, such as saline, or sterile water, or plasma, or other suitable medium for reconstitution to produce a composition for administration. The reconstituted PRP compositions can be in any volume, but generally is a suitable volume for single dosage administration, such as volume of at least or at least about 1 mL, such as 1.5 mL, 2 mL, 2.5 mL, 3 mL, 3.5 mL, 4 mL, 4.5 mL, 5 mL, 5.5 mL, 6 mL, 6.5 mL, 7 mL, 7.5 mL, 8 mL, 8.5 mL, 9 mL, or more.

The PRP compositions can be rehydrated, and after rehydration the PRP composition is for administration within 12 hours, 11.5 hours, 11 hours, 10.5 hours, hours, 9.5 hours, 9 hours, 8.5 hours, 8 hours, 7.5 hours, 7 hours, 6.5 hours, 6 hours, 5.5 hours, 5 hours, 4.5 hours, 4 hours, 3.5 hours, 3 hours, 2.5 hours, 2 hours, 1.5 hours, 1 hour, or less of rehydration, such as 3-6 hours, such as 4 hours for administration within 4 hours of rehydration, or less.

Hence, provided are freeze-dried (lyophilized) PRP compositions and reconstituted (rehydrated) liquid compositions comprising the freeze-dried composition and a liquid, such as saline or sterile water, or plasma, or conditioned medium, or mixtures thereof, or other medium suitable for administration to an animal. The PRP compositions, particularly the freeze-dried compositions, can be stored and provided in a sealed container, such as a vial. Nitrogen can be added to containers prior to sealing.

Provided are containers containing compositions provided herein. Exemplary thereof, the containers can contain 1×10⁹ to 5×10⁹ platelets, or contains at least 1×10⁹, or contains at least 4×10⁹ platelets. In an example, about 1.2 mg freeze-dried powder on the average results from 8 ml of PRP composition, and the compositions and resulting powder contains on the average up to or at least 1×10⁹, 2×10⁹, 3×10⁹, 4×10⁹, or 5×10⁹ platelets in each container, such as a vial. The platelets in the containers or the PRP compositions can be any volume desired, such as composition that is 1-20 mL, or 3-15 mL, or 5-10 mL, or 7-9 mL; or are the platelets in a resulting freeze-dried powder. The amounts of platelets and volumes depend upon the animal to whom the platelets are to be administered. Containers can contain multiple dosages or single dosages. Single dosages can contain, for example, 4×10⁹ platelets, but will vary depending upon the size of the animal. Exemplary formulations and products are described in the Examples.

Provided are freeze-dried platelet-rich plasma (PRP) composition, comprising platelets, wherein at least 40%, 45%, 50%, 55%, 60%, 65%, 70%, 75%, 80%, 85%, or more of the platelets are in a primed state, such that the granules have not released their contents, but do upon administration. The amount of platelets in the compositions is that amount that results from freeze drying a PRP composition that contains platelets (generally in an amount described below that is at least 150,000 platelets/μg up to at most at or about 1,000,000 platelets/μl, such as about 500,000 platelets/μl). As an example, about 1.2 mg of powder is produced from about 8 mL of a PRP composition. As an example, the 1.2 mg of powder contains a total number of platelets that is at or about between 1×10⁹ to 4×10⁹. The powder contains the platelets and plasma, where, for example, at least ⅓ of up to ¾ of the composition is plasma, and the number of platelets is as noted above and below. At least 40% up to 85%, such as 50% or more of the platelets in the PRP compositions are in a primed state.

In some embodiments, provided are platelet-rich plasma (PRP) compositions, comprising platelets and plasma, where: the concentration of platelets is the same as or greater than the concentration of platelets in blood; the amount of plasma in the composition is 30% to 80% by volume; the ratio of platelets to plasma is from about 3:1 to 1:5, such as about ⅓ to ¾ of composition is plasma), and at least 40%, such as at least 45%, 50%, 55%, 60%, 65%, 70%, 75%, 80%, 85%, or more of the platelets in the composition are in a primed state. The PRP compositions provided herein, particularly the reconstituted or rehydrated compositions or the compositions prior to lyophilization, contain at or about 150,000 platelets/μl up to 1,000,000 platelets/μl, or 300,000 platelets/μl to 600,000 platelets/μl, or 300,000 platelets/μl to 500,000 platelets/μl. Provided are freeze dried PRP composition prepared by freeze-drying any of these compositions. Generally, at most 40% of platelets are activated. Provided are freeze-dried platelet-rich (PRP) compositions, where, following reconstitution, upon intralesional administration, the contents of the platelet granules are released. Typically, the contents are released within a few minutes of administration, and generally more quickly than platelets that are not in a primed state and not activated. In the primed state, the platelets are partially activated, such that the regenerative and other molecules in the granules are not released but the granules are such that the contents are released upon administration.

Provided are reconstituted (rehydrated) platelet-rich plasma compositions prepared by adding liquid to a freeze-dried composition. Rehydrated compositions comprise any of the freeze-dried compositions provided herein, and a liquid. In such compositions, the concentration of platelets is sufficient for treatment of a disease, disorder, or condition treated by administration of platelets. The amount of platelets is sufficient for single dosage or multiple dosage administration.

Also provided are freeze-dried (lyophilized) PRP compositions comprising plasma and platelets; wherein the lyophilized PRP composition does not comprise a cryopreservative. Cryopreservatives protect the platelets from cold-induced damage. By virtue of the methods and processing provided herein the platelets can be freeze-dried without adding a cryopreservative(s). Exemplary cryopreservatives that are not in the compositions herein are dimethyl sulfoxide (DMSO), trehalose, glucose, glycerol, maltodextrin, dextran, hydroxyethyl starch (HES), formaldehyde, paraformaldehyde, glutaraldehyde, and permanganate. Prior art PRP compositions contain such cryopreservatives, or, if they are not added, the resulting compositions contain a high level of activated platelets, generally at least 80% of the platelets are activated. In contrast, the PRP compositions provided herein contain at most 40% activated platelets, and, as noted above and demonstrated and described below, at least 40% primed platelets. The concentration of platelets is therapeutically sufficient for regenerative therapy, such as treatments where the locus of therapy is a tendon, joint, or wound. Typically, the concentration of platelets is at least 300,000 platelets/μl of composition, or if freeze dried contains the number of platelets resulting from freeze-drying the composition. As noted above, freeze drying about 8 mL has yielded on the average about 1.2 μg of powder. In some embodiments, about ⅓ to ¾ of the powder comprises plasma, and the rest is platelets or platelets plus any other ingredients or agents, such as anticoagulants, in the liquid prior to freeze-drying. For example, in compositions provided herein the concentration of platelets is 150,00 to 400,000 platelets/μl, or is 150,000 platelets/μl to 500,000 platelets/μl, or is 300,000 platelets/μl to 500,000 platelets/μl, or is 150,000 platelets/μl to 1,000,000 platelets/μl, or is about or is at least 150,000 platelets/μl to 450,000 platelets/μl, inclusive, or is about or is at least 150,000 platelets/μl to 750,000 platelets/μl.

Primed platelets can be identified by phenotypic markers and/or properties. Primed platelets exhibit one or more of the following: externalized phosphatidylserine, intact granules, negative for p-selectin on the platelet surface, retain discoid shape, and do not aggregate upon swirling or light agitation, where the property is lack of aggregation upon swirling or light agitation, a second property of externalized phosphatidylserine, intact granules, negative for p-selectin on the platelet surface, or retention of discoid shape also occurs. In general, the primed platelets can be identified by one of the properties, except and noted, and can be identified by exhibiting at least two of the properties. For example, primed platelets have intact granules and/or have externalized phosphatidylserine, and/or are negative for p-selectin. As noted, primed platelets have intact granules whose content is released immediately upon administration to a lesion.

In the PRP compositions provided herein, at least 40%, 45%, 50%, 55%, 60%, 65%, 70%, 75%, 80%, 85%, or more of the platelet particles in the composition are between 0.5 μm and 5 μm in size (diameter), such as where at least 45%, 50%, 55%, 60%, 65%, 70%, 75%, 80%, 85%, or more of the particles in the composition are between 0.5 μm and 2 μm, or where least 80% of the particles in the composition are between 0.5 μm and 5 μm, or where at least 80% of the particles in the composition are between 0.5 μm and 2 μm. The size indicates that platelets are not activated.

Primed platelets can be identified by the presence of externalized phosphatidylserine. Externalized phosphatidylserine binds to lactadherin, which can be used for detecting primed platelets. In PRP compositions provided herein, at least about or about 40%, 45%, 50%, 55%, 60%, 65%, 70%, 75%, 80%, 85%, 90%, 91%, 92%, 93%, 94%, 95%, 96%, 97%, 98%, 99% or 100% of the platelets in the composition have externalized phosphatidylserine. Primed platelets are not positive for CD62 or other activation marker. In compositions provided herein, the ratio of platelets with externalized phosphatidylserine to platelets without externalized phosphatidylserine in the composition can be at least or at least about 1:1, 1.1:1, 1.2:1, 1.3:1, 1.4:1, 1.5:1, 1.6:1, 1.7:1, 1.8:1, 1.9:1, 2.1:1, 2.2:1, 2.3:1, 2.4:1, 2.5:1, 2.6:1, 2.7:1, 2.8:1, 2.9:1, 3:1, 3.1:1, 3.2:1, 3.3:1, 3.4:1, 3.5:1, 3.6:1, 3.7:1, 3.8:1, 3.9:1, 4:1, 4.1:1, 4.2:1, 4.3:1, 4.4:1, 4.5:1, 4.6:1, 4.7:1, 4.8:1, 4.9:1, 5:1, 5.1:1, 5.2:1, 5.3:1, 5.4:1, 5.5:1, 5.6:1, 5.7:1, 5.8:1, 5.9:1, 6:1, 6.1:1, 6.2:1, 6.3:1, 6.4:1, 6.5:1, 6.6:1, 6.7:1, 6.8:1, 6.9:1, 7:1, 7.1:1, 7.2:1, 7.3:1, 7.4:1, 7.5:1, 7.6:1, 7.7:1, 7.8:1, 7.9:1, 8:1, 8.1:1, 8.2:1, 8.3:1, 8.4:1, 8.5:1, 8.6:1, 8.7:1, 8.8:1, 8.9:1, 9:1, 9.1:1, 9.2:1, 9.3:1, 9.4:1, 9.5:1, 9.6:1, 9.7:1, 9.8:1, 9.9:1, 10:1, 10.1:1, 10.2:1, 10.3:1, 10.4:1, 10.5:1, 10.6:1, 10.7:1, 10.8:1, 10.9:1, 11:1, 11.1:1, 11.2:1, 11.3:1, 11.4:1, 11.5:1, 11.6:1, 11.7:1, 11.8:1, 11.9:1, 12:1, 12.1:1, 12.2:1, 12.3:1, 12.4:1, 12.5:1, 12.6:1, 12.7:1, 12.8:1, 12.9:1, 13:1, 13.1:1, 13.2:1, 13.3:1, 13.4:1, 13.5:1, 13.6:1, 13.7:1, 13.8:1, 13.9:1, 14:1 or more. At least about or about 40%, 45%, 50%, 55%, 60%, 65%, 70%, 75%, 80%, 85%, 90%, 91%, 92%, 93%, 94%, 95%, 96%, 97%, 98%, 99% or 100% of the platelets in the composition are positive for lactadherin binding.

In PRP compositions provided herein, at most up to 40%, 41%, 42%, 43%, 44%, 45%, 46%, 47%, 48%, 49%, or 50%, or 50%, or fewer than 50% of the platelets in the composition are activated platelets. Activation can be detected by detecting CD62; activation is indicated by CD62. In PRP compositions provided herein the ratio of platelets that are lactadherin positive to platelets that are lactadherin negative in the composition can be at least or at least about 1:1, 1.1:1, 1.2:1, 1.3:1, 1.4:1, 1.5:1, 1.6:1, 1.7:1, 1.8:1, 1.9:1, 2.1:1, 2.2:1, 2.3:1, 2.4:1, 2.5:1, 2.6:1, 2.7:1, 2.8:1, 2.9:1, 3:1, 3.1:1, 3.2:1, 3.3:1, 3.4:1, 3.5:1, 3.6:1, 3.7:1, 3.8:1, 3.9:1, 4:1, 4.1:1, 4.2:1, 4.3:1, 4.4:1, 4.5:1, 4.6:1, 4.7:1, 4.8:1, 4.9:1, 5:1, 5.1:1, 5.2:1, 5.3:1, 5.4:1, 5.5:1, 5.6:1, 5.7:1, 5.8:1, 5.9:1, 6:1, 6.1:1, 6.2:1, 6.3:1, 6.4:1, 6.5:1, 6.6:1, 6.7:1, 6.8:1, 6.9:1, 7:1, 7.1:1, 7.2:1, 7.3:1, 7.4:1, 7.5:1, 7.6:1, 7.7:1, 7.8:1, 7.9:1, 8:1, 8.1:1, 8.2:1, 8.3:1, 8.4:1, 8.5:1, 8.6:1, 8.7:1, 8.8:1, 8.9:1, 9:1, 9.1:1, 9.2:1, 9.3:1, 9.4:1, 9.5:1, 9.6:1, 9.7:1, 9.8:1, 9.9:1, 10:1, 10.1:1, 10.2:1, 10.3:1, 10.4:1, 10.5:1, 10.6:1, 10.7:1, 10.8:1, 10.9:1, 11:1, 11.1:1, 11.2:1, 11.3:1, 11.4:1, 11.5:1, 11.6:1, 11.7:1, 11.8:1, 11.9:1, 12:1, 12.1:1, 12.2:1, 12.3:1, 12.4:1, 12.5:1, 12.6:1, 12.7:1, 12.8:1, 12.9:1, 13:1, 13.1:1, 13.2:1, 13.3:1, 13.4:1, 13.5:1, 13.6:1, 13.7:1, 13.8:1, 13.9:1, 14:1, or more. In some embodiments of the PRP compositions provided herein, fewer than about or 20% of the platelets in the composition are inactivated.

The PRP compositions herein can further comprise other agents, such as an anti-coagulant. Anti-coagulants typically, when present, are added during collection and/or processing, and is residual from anti-coagulant added during collection of the platelets and plasma. Anti-coagulants, include, but are not limited to, one or more of acid citrate dextrose solution A (ACD-A; for example ACD-A containing citric acid, dihydrate sodium citrate, monohydrate dextrose), triple citrate, citrate-phosphate-dextrose solution with adenine (CPDA-1), heparin (e.g., sodium, lithium, or ammonium salt of heparin, calcium-titrated heparin), ethylenediaminetetraacetic acid (EDTA) and analogs thereof (e.g., disodium EDTA, tripotassium EDTA, dipotassium EDTA, ethyleneglycoltetraacetic acid (EGTA), ethylendiaminedipropionic acid (EDDP), and hexamethylenediaminetetraacetic acid (HDTA)), citrate (e.g., sodium citrate, trisodium citrate, trisodium citrate dehydrate, trisodium citrate pent hydrate, citrate dextrose, ACD-A, ACD-B), acid citrate dextrose solution B (ACD-B), oxalate (e.g., sodium oxalate, potassium oxalate, ammonium oxalate, lithium oxalic acid), sodium fluoride (e.g., sodium fluoride-potassium oxalate), or other anti-coagulant, such as anti-coagulants used during platelet collection, such as, for example, apheresis. Exemplary of these are ACD-A, triple citrate, heparin, and CPDA-1, or Anticoagulant Citrate Dextrose Solution-Formula A (ACD-A).

The PRP compositions can contain leukocytes which occur in blood and plasma when isolated or can contain added leukocytes, or can be leuko-reduced to reduce or eliminate leukocytes so that the level of leukocytes is lower than level of leukocytes in whole blood. Leukocytes can be identified by detecting CD44.

For example, the resulting PRP composition can contain fewer than 5000, fewer than 4500, fewer than 4000, fewer than 3500, fewer than 3000, fewer than 2500, fewer than 2000, fewer than 1900, fewer than 1800, fewer than 1700, fewer than 1600, fewer than 1500, fewer than 1400, fewer than 1350, fewer than 1300, fewer than 1250, fewer than 1200, fewer than 1050, fewer than 1000, fewer than 950, fewer than 900, fewer than 850, fewer than 800, fewer than 750, fewer than 700, or fewer leukocytes per μL in a liquid PRP composition or the resulting number in a freeze-dried composition produced from a liquid composition. In some embodiments, the compositions do not contain any leukocytes. In other embodiments, the compositions can be leuko-rich, whereby the concentration of leukocytes in the composition is higher than concentration of leukocytes in whole blood. In general, except where the leukocytes are eliminated, up to or at or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10% of the cells in the PRP compositions provided herein are leukocytes.

The PRP compositions provided herein, except any intended for autologous administration, are prepared by pooling platelets and/or plasma are pooled from a plurality of donor animals, generally from same species. For example, the number of donors is at 3, 4, 5, 6, 7, 8, 9, 10, 12, 15, 20, up to 40 or more donor animals. Animals include, for example, but not limited to, horses, dogs, cats, elephants, fish, mammalian water animals, primates, fowl, and ruminants, such as horses, humans, gorillas, monkeys, chickens, goats, cows, deer, sheep, pigs, dolphins, and whales. Exemplary of animals from whom the platelets are obtained and used are equine, such as horses, ponies, and other members of the horse family. The platelets and plasma can be from canine species. The source also can be humans and can be for treatment of humans.

The compositions provided herein, particularly the reconstituted compositions, can be formulated for subcutaneous injection, topical, intra-articular, intralesional, intra-tendonous, oral, intravitreal, periocular, or transdermal administration.

Combinations of the PRP compositions provided herein with one or additional therapeutics are provided. The PRP composition and the additional therapeutic(s) can be in separate combinations, or can be co-formulated. The other therapeutic or therapeutics in the combination, separate compositions, or co-formulations, generally are selected for treatment of the same disease, disorder, or condition, or to treat or prevent an adverse side effect, for the disease, disorder, or condition for which the PRP is administered. Exemplary additional therapeutic agent comprises chemokines, cytokines, antibiotics, anti-inflammatories, immunotherapeutics, growth factors, hormones, steroids, hyaluronic acid, or small molecule(s) drugs. The additional therapeutic can comprise stem cells, and/or ECM- or MSC-derived exosomes and/or mitochondria, and/or bone matrix material, and/or an antibiotic. Other diseases, disorders, and conditions include ocular diseases, disorders, and conditions. Exemplary of ocular diseases, disorders, and conditions is one or more of an ocular lesion, keratitis, corneal lesion, and corneal ulcer, or thyroid eye disease, or keratoconjunctivitis sicca (KCS), or dry eye.

Methods of preparing the PRP compositions provided herein that contain the high concentration of primed platelets, and/or that do not require cryopreservatives for lyophilization or storage. It is shown herein that such compositions can be prepared by refrigerating the platelets following collection. In accord with methods provided herein, the platelets are refrigerated following collection and, for freeze-dried compositions, prior to lyophilization (freeze-drying). The methods can further comprise combining platelets with additional plasma after refrigerating the platelets.

Provided are methods of preparing PRP compositions, comprising: (a) collecting platelets from a subject; (b) incubating the platelets at refrigerated (refrigerator) temperatures; (c) collecting plasma from a subject or subjects; (d) combining the platelets and plasma; and (e) optionally, freeze-drying the composition. Refrigeration is effected at temperatures between about 0° C. and 10° C., inclusive, such as about 4° C. to 10° C., or such as between at or about 4° C. and 8° C., inclusive. The platelets are refrigerated for at or about 4 hours to 14 days, such as up to 13, 12, 11, 10, 9, 8, 7, 6, or 5 inclusive. In accord with methods provided herein the platelets are refrigerated immediately after collection, when mixed with additional plasma, can be refrigerated prior to mixing with any additional plasma. Platelets are refrigerated within at or about 10 minutes, 11 minutes, 12 minutes, 13 minutes, 14 minutes, 15 minutes after collection. By virtue of the methods of preparation provided herein, the PRP compositions provided herein do not include nor require cryopreservatives.

Methods of treatment by administering the PRP compositions and combinations provided herein are provided. Uses of the compositions for treatment are provided, and the compositions for use are provided. Typically, the PRP compositions are reconstituted by adding liquid to the freeze-dried compositions, and optionally any additional therapeutic or other agents, and optionally additional plasma. The compositions can be administered for formulated in other forms, include solids and gels. Generally, the concentration of platelets in the reconstituted PRP compositions are in the ranges recited above, such as 150,000 platelets/μl to no more than 1,000,000 platelets/μl, such as about 300,000-500,000 platelets/μl. The methods include the steps of administering the composition or combination to treat a disease, disorder, or condition that can be treated by a PRP composition. Diseases, disorders, conditions, and injuries that can be treated include, but are not limited to: inflammatory diseases, disorders or conditions; cardiovascular diseases, disorders or conditions; nervous system diseases, disorders or conditions; tumors; demyelinating diseases, disorders or conditions; dermatological system diseases, disorders or conditions; digestive system diseases, disorders or conditions; endocrine system diseases, disorders or conditions; reproductive system diseases, disorders or conditions; hemic and lymphatic diseases; disorders or conditions; immunological diseases, disorders or conditions; mental disorders; musculoskeletal diseases, disorders or conditions; neuromuscular diseases, disorders or conditions; metabolic diseases, disorders or conditions; skin and connective tissue diseases, disorders or conditions or injuries; spinal diseases, disorders or conditions or injuries; disorders or conditions or injuries of tendons, muscles, bone, and joints; and urological diseases, disorders, and conditions.

Exemplary of uses is of PRP compositions is where a disease, disorder, or condition is injured or diseased tissue and/or organ, and the composition is for healing the injured or diseased tissue or organ. This includes, for example, healing of injured or diseased tissues and organs, such as, but not limited to, skin, heart, kidney, liver, uterus, pancreas, and tissues and organs of the digestive system or nervous system or immune system or reproductive system. The PRP compositions can effect healing and/or decrease healing time. In other examples, uses is of PRP compositions or the PRP compositions in combination with other therapeutics can be used for treating osteoarthritis (e.g., stifle joint osteoarthritis (OA)); for orthopedic tissue reconstruction; for spinal fusion; for treating spinal disc defects or injuries; for treating adhesive capsulitis; for treating bone injury or bone dysfunction or bone degradation or lack of bone regrowth or regeneration or a bone disorder; for treating musculoskeletal pathologies; for treating tendon injury or tendon dysfunction or tendon degradation or lack of tendon regrowth or regeneration or a tendon disorder (e.g., tendonitis, tendinosis); for treating ligament injury or ligament dysfunction or ligament degradation or lack of ligament regrowth or regeneration or a ligament disorder; for treating a muscle injury or muscle dysfunction or muscle degradation or lack of muscle regrowth or regeneration or a muscle disorder; for tissue regeneration, such as tendon, bone or ligament regeneration; for wound healing; wound healing; for treating conjunctivitis, for treating corneal ulceration, for reconstruction of alveolar-mandibular defects, for treating hoof injuries including sole abscess in equine subjects, for treating septic inflammatory response syndrome (SIRS), for treating laminitis, for treating inflammatory myopathies, for treating endotheliopathy (traumatic), for treating endotheliopathy (septic), for treating asthma and inflammatory bronchitis; to support the action of MSCs; supporting growth and maturation of ex vivo explants for transfusion or transplant; use as a media additive to replace bovine serum albumin (BSA); for treating age-related macular degeneration, for treating coronary artery disease; for treating peripheral vascular disease; for islet cell transplantation; for fracture and tendon repair, for reconstructive surgery; for tissue engineering; for treating restenosis; for treating cancer, for treating diabetic retinopathy; for treating rheumatoid arthritis; for treating psoriasis, for treating hemangioma/AEDS-related Kaposi's sarcoma; for treating atherosclerotic plaque rupture; for treating uterine injuries; for treating contaminated wounds, tissue lesions or tears; and/or for treating otitis externa.

Since the PRP compositions can be prepared by pooling platelets from a plurality of donors, the platelets in the composition are allogeneic to the subject to whom the composition is administered. In some embodiments, the PRP composition is prepared from the subject to whom the composition is to be administered whereby the platelets in the composition are autologous to the subject to whom the composition is administered.

Provided are methods, where: the PRP composition is reconstituted for administration; is administered intravenously, subcutaneously, intramuscularly, or intralesionally.

As detailed herein, platelets in the compositions are primed, but not activated. Compositions for administration can be prepared from the freeze-dried compositions and are administered to the subject for treatment of conditions in which the primed platelets that release the contents of the granules upon treatment are for therapy, such as for treatment of regenerative diseases. There are embodiments in which the platelets are activated prior to administration; such diseases, disorders, and conditions generally are those in which the compositions are administered to provide platelets, such as for hemostasis or for treatments that do not require the regenerative molecules that are sequestered in the granules.

The PRP compositions can be used for delivery of chemokines and/or cytokines and/or growth factors. The PRP compositions provided herein that contain a high percentage (40% or more) of primed platelets are useful for such delivery. As noted, the PRP compositions used for treatment of diseases, disorders, and conditions generally are prepared from or are the freeze-dried PRP compositions. They can be reconstituted prior to administration, administered as a powder, as a solid, or as a gel, or any other form suitable for treatment of a disease, disorder, or condition. These diseases, disorders, and conditions include, as noted above, inflammatory diseases, disorders or conditions; cardiovascular diseases, disorders or conditions; nervous system diseases, disorders or conditions; tumors; demyelinating diseases, disorders or conditions; digestive system diseases, disorders or conditions; endocrine system diseases, disorders or conditions; reproductive system diseases, disorders or conditions; hemic and lymphatic diseases; disorders or conditions; immunological diseases, disorders or conditions; mental disorders; musculoskeletal diseases, disorders or conditions; neuromuscular diseases, disorders or conditions; metabolic diseases, disorders or conditions; skin and connective tissue diseases, disorders or conditions; and urological diseases, disorders or conditions; or is for treating osteoarthritis, such as, for example, stifle joint osteoarthritis (OA), for treating adhesive capsulitis, for treating bone injury or bone dysfunction or bone degradation or lack of bone regrowth or regeneration or a bone disorder, for tissue regeneration, such as tendon, bone or ligament regeneration; for wound healing; or for treating contaminated wounds, tissue lesions or tears. The PRP compositions are effective for treating otitis externa in canines.

The PRP compositions can be formulated for single dosage or multiple dose administration. For effecting treatment, the PRP compositions can be administered once or a plurality of times; the regimen depends upon the disease, disorder, or condition treated and other parameters as determined by and within the skill of a treating professional. The PRP composition can be administered as a single dose or can be administered in multiple doses. For example, where the PRP composition is administered as multiple doses, the multiple doses can be administered in intervals of at least or at least about 2 hours, 3 hours, 6 hours, 12 hours, one day, two days, three days, four days, five days, six days, one week, two weeks, three weeks, one month, 6 weeks, two months, three months, four months, six months, eight months, ten months, one year, fourteen months, sixteen months, eighteen months, or two years apart. Exemplary of such regimens are those where a PRP composition is administered as multiple doses, where the multiple doses are administered at least 2 days apart. The PRP compositions can be administered at least 2, 3, 4, 5, 6 7, or more times, including regularly for many days, months, or years.

In some embodiments, the platelets in the PRP compositions provided herein can be activated prior to administration. Activation can be effected by adding into the composition or contacting the composition with a thrombin agonist or thrombin or calcium prior to administration.

In some embodiments the PRP compositions or platelets can be cultured with stem cells prior to administration. In some embodiments the PRP composition can be reconstituted with prepared with MSC-conditioned media, MSC and/or targeted macrophages.

The route of administration of the PRP compositions provided herein includes any suitable for treatment of the disease, disorder, or condition. Routes include, but are not limited to, intra-articular, intra-lesional, intra-tendonous, oral, intra-nasal, pulmonary, periocular, parenteral, transdermal, intradermal intravitreal, periocular, subcutaneously, or topical administration.

The PRP compositions can be administered to a joint or are for use for administration to a joint, such as a stifle or radial carpal joint, or can be administered to a tendon or used for administration to a tendon, such as a calcaneal tendon, where the PRP composition is for treating damage to the tendon. The PRP compositions can be used for or administered for treating tendon, ligament or bone diseases, disorders, or conditions, such for osteoarthritis (OA), such as for treating stifle joint osteoarthritis (OA), or for treating stifle joint osteoarthritis (OA) at the time of tibial leveling osteotomy (TPLO). The PRP compositions can be used for or administered for treating a foreign body in a subject. Exemplary of such foreign bodies are pathogens, such as bacteria, parasites, fungi, protozoans, and other pathogens or foreign bodies. The PRP compositions are effective for treating otitis externa in canines.

In embodiments of the methods and uses, where the PRP is combined with another therapeutic, the PRP composition can be administered at the same site or at a different site from the therapeutic. They can be administered serially, intermittently, or according to a regimen. For example, the PRP composition can be administered or is for co-administration with other agents or products, such as, but not limited to, autologous stem cells or allogeneic stem cells or with bone matrix materials, an antibiotic. Exemplary thereof are embodiments in which the PRP composition is administered with or is for administration with mesenchymal stem cells (MSCs). Other examples of combination therapies are those where the PRP composition is for administration with one or more of an antibiotic, antimycotic, or other anti-inflammatory medication, and/or eye lubricant.

### BRIEF DESCRIPTION OF THE DRAWINGS

FIG. 1 depicts a platelet and plasma collection step and subsequent steps in the PRP preparation process.

FIG. 2 depicts a platelet and plasma collection step and subsequent steps in the PRP preparation process.

FIGS. 3A-3B: FIG. 3A depicts a sample plot of the SSC/FSC (side scatter/forward scatter) of equine platelet and leukocyte rich plasma isolated from fresh EDTA blood. FIG. 3B depicts a sample plot of the SSC/FSC (side scatter/forward scatter) of reconstituted platelet-rich plasma product as collected by apheresis and prepared and lyophilized as provided herein.

FIGS. 4A-4F are histograms depicting the particle size distribution of the platelet rich plasma product, with most particles falling within the 0.5 to 2 micron gate. FIGS. 4A-4F depict the flow cytometry results for six vials, vials 1-6, respectively, of reconstituted lyophilized equine PRP, and set forth in Table 3. The flow cytometry plot results from a collection of more than 5,000 events within an FSC/SSC gate.

FIGS. 5A-5F are histograms depicting the cells positive for platelet and white blood cell markers, for samples 1-6, respectively. The results are set forth in Table 5. The leftmost and middle panels in each of FIGS. 5A-5F depict the results of labeling with platelet- and leukocyte-specific antibodies. The leftmost panels depict the results of labeling with an anti-CD44 antibody, which is specific for leukocytes. The middle panels depict the results of labeling with an anti-CD61 antibody, which is specific for platelets. The rightmost panels depict the results of labeling with an anti-CD9 antibody, which is specific for platelets.

FIGS. 6A-6F are histograms depicting the cells positive for lactadherin, which is a marker for outer membrane phosphatidylserine, for samples 1-6, respectively. The results are set forth in Table 5.

FIGS. 7A-7F are histograms depicting the cells positive for platelet markers and platelet activation, for samples 1-6, respectively. The results are set forth in Table 5. The leftmost panels depict the results of labeling with platelet-specific antibodies (CD9). The rightmost panels depict the results of labeling with an anti-CD62P antibody, which is specific for activated platelets (p-selectin).

FIGS. 8A-8C depict the flow cytometry results for three vials, vials 1-3, respectively, of reconstituted lyophilized canine PRP, and set forth in Table 6. The flow cytometry plot results from a collection of more than 5,000 events within an FSC/SSC gate (backgating colors defined by particle size gate).

FIG. 9 shows the anti-microbial properties of PRP; all PRP lots exhibit none or very low bacterial growth; whereas PBS and growth medium each showed significant bacterial growth.

FIG. 10 shows the anti-microbial properties of PRP; all PRP lots exhibit none or very low bacterial growth; whereas PBS and growth medium each showed significant bacterial growth.

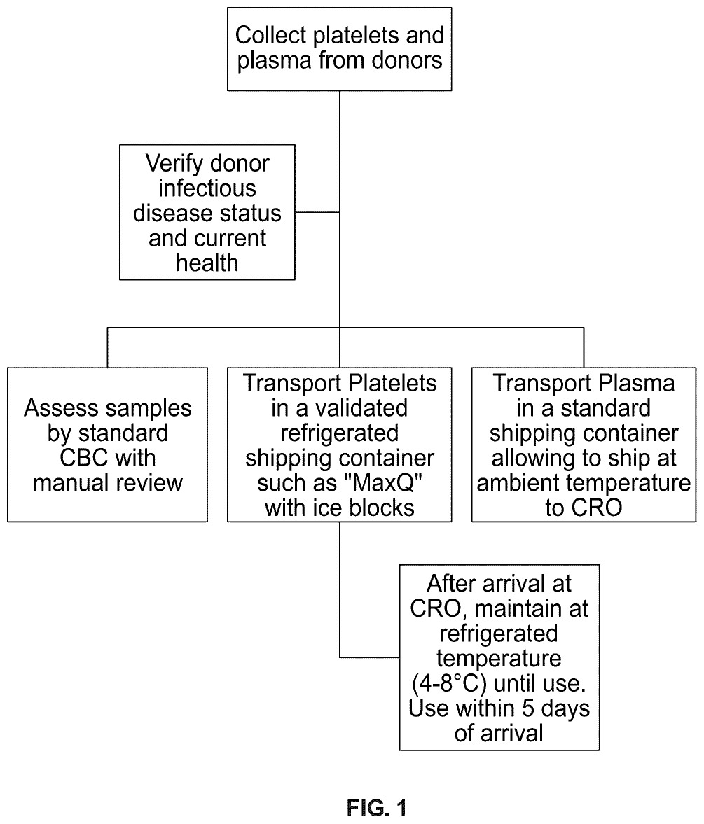

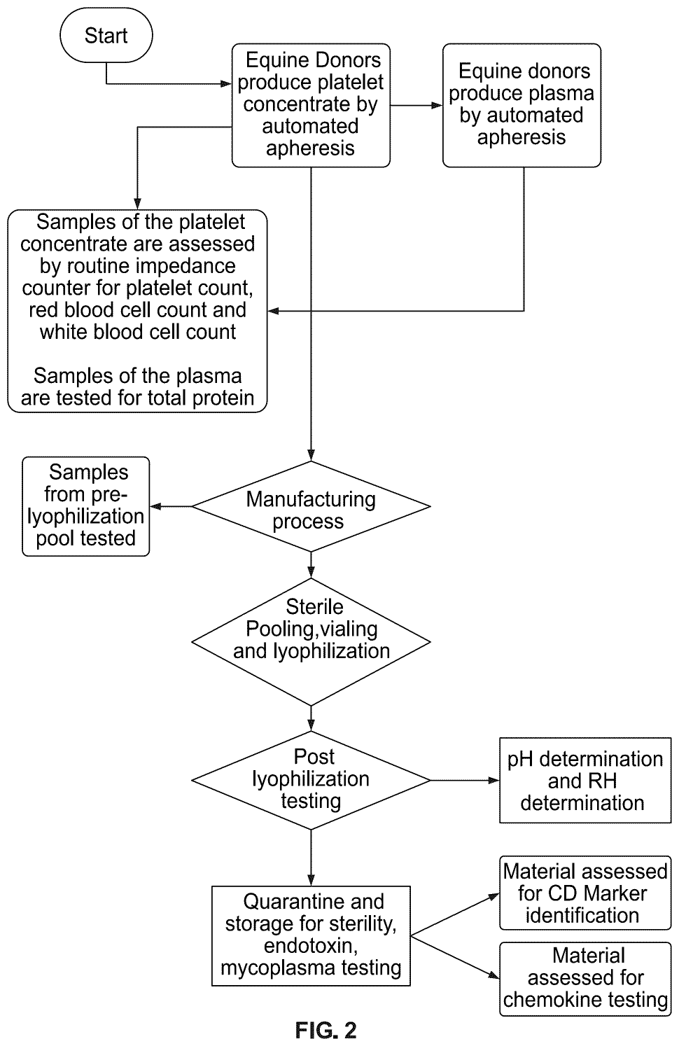

### DETAILED DESCRIPTION

### Outline

A. DEFINITIONS
B. PLATELET-RICH PLASMA AND OVERVIEW
   1. Overview of the Methods for Preparing a Platelet-rich Plasma Composition without Cryopreservative
   2. Preparation of a Platelet-Rich Plasma Composition
   3. Primed Platelets in a Platelet-Rich Plasma Composition
C. PLATELETS AND PLATELET-RICH PLASMA
   1. Platelet Function and Activation
   2. Platelet Storage-Refrigerated Storage
   3. Platelet Storage-Freezing and Freeze-Drying Problems
   4. Commercially Available Platelet Products
   5. Cryopreservatives Used in Prior Art Platelet Preparation and Storage
      Dimethyl sulfoxide
      Trehalose
      Other Cryopreservatives and Combinations
D. PLATELET COLLECTION AND ISOLATION
   1. Donors
      a. Pathogen reduction
      b. Sample Pooling
      c. Other Considerations
   2. Platelet Collection
      Leukoreduced or Leukorich Platelet Rich Plasma Composition
      a. Apheresis
         i. Donor Considerations
         ii. Apheresis Procedures
         iii. Commercially Available Apheresis Devices
         iv. Additional Apheresis Techniques
            Membrane Separation Apheresis
            Selective Adsorption
            Apheresis Procedures for Separating Cellular Components
      b. Whole Blood-Derived (WBD) Platelets
      c. Buffy Coat (BC) Method of Platelet collection
E. COLD INCUBATION OF PLATELETS
F. LYOPHILIZATION
G. RECONSTITUTION
   1. Reconstitution in Water or Saline
   2. Other Solutions for Platelet Reconstitution and Resuspension
H. PRP AND PLATELET CHARACTERIZATION-Characterization of Platelet Activation and/or Activity in a PRP Composition
   1. Assays and Markers for Assessing Platelet Activation State
   2. Antibody Labeling and Flow Cytometry
   3. Platelet Size and Shape
   4. Phosphatidylserine externalization
   5. Swirling
   6. Light transmission aggregometry (LTA)
   7. Granulation
   8. Calcium Levels
   9. Platelet Vesiculation or Microparticle Generation
   10. Receptor Changes
   11. Platelet Aggregation
   12. Other Assays
I. THERAPEUTIC USES AND METHODS OF TREATMENT
   1. Regenerative Therapies
      a. Musculoskeletal Disorders, Tissue, Tendon and Ligament Regeneration
      b. Osteoarthritis
      c. Adhesive capsulitis
      d. Wound Healing
      e. Rheumatoid Arthritis
   2. Other Diseases, Disorders and Conditions
J. COMBINATION THERAPIES
K. FORMS OF PRP COMPOSITIONS
   Liquid PRP Composition
   Lyophilized PRP Composition
   Reconstituted Lyophilized PRP Composition
   Other Blood Cells in the Composition
   Stability
L. ADMINISTRATION
   Administration Route
   Subject for Administration
M. DOSAGES
N. EXAMPLES

### A. DEFINITIONS

Unless defined otherwise, all technical and scientific terms used herein have the same meaning as is commonly understood by one of skill in the art to which the invention(s) belong. All patents, patent applications, published applications and publications, databases, websites, and other published materials referred to throughout the entire disclosure herein, unless noted otherwise, are incorporated by reference in their entirety. If there are a plurality of definitions for terms herein, those in this section prevail. Where reference is made to a URL or other such identifier or address, it is understood that such identifiers can change and information on the internet can come and go, but equivalent information can be found by searching the internet. Reference thereto evidences the availability and public dissemination of such information.

As used herein, platelet or platelets refers a blood component that functions in a variety of process, including, but not limited to, clotting and wound healing. Reference to platelets includes whole platelets, fragmented platelets, and platelet derivatives. Platelets include fresh platelets, such as those collected from whole blood or by apheresis. Platelets also include those that have been subjected to treatment(s) such as freeze-drying or lyophilization. Freeze dried or lyophilized platelets can be reconstituted in a liquid, such as saline or water. Platelets include those of mammals, such as humans and non-human mammals.

As used herein, compositions containing platelets include, but are not limited to, for example, platelets in whole blood, platelets in plasma, platelets in buffer optionally supplemented with plasma or plasma proteins, cold stored platelets, dried platelets, frozen platelets, thawed platelets, rehydrated dried platelets, rehydrated cryopreserved platelets, lyopreserved platelets, thawed lyopreserved platelets, or rehydrated lyopreserved platelets.

As used herein, platelet-rich plasma (PRP) refers to a blood-derived product containing an increased number or concentration of platelets, compared to blood, and containing plasma. PRP is plasma with a platelet count above that of peripheral blood. PRP compositions provided herein are prepared by a method provided herein whereby platelets in the PRP are in a primed state such that the platelets are partially activated. In a partially activated state or primed state, the platelets, which are rich in growth factors, have not released growth factors. Upon, for example, intralesional administration, granule contents of primed platelets are immediately released.

As used herein, plasma refers to the liquid portion of blood that does not include blood cells, such as white blood cells, red blood cells, and platelets. Most of the blood plasma is water, and plasma also contains salt, enzymes, antibodies (immunoglobulin), clotting/coagulation factors, albumin, fibrinogen, and other proteins.

As used herein, a reconstituted lyophilized PRP composition and rehydrated lyophilized PRP composition are used interchangeably and refer to a platelet-rich plasma (PRP) composition that has been lyophilized, and the lyophilized PRP composition has been rehydrated, such as by adding sterile saline or water. In examples, the PRP composition (e.g., PRP composition prepared without cryopreservatives) is lyophilized and then is rehydrated in sterile water for injection or saline (e.g., 0.9% saline) to produce a reconstituted lyophilized PRP composition.

As used herein, a cryopreservative, used interchangeably with cryoprotectant, refers to a substance that is added to a composition containing a biological product, such as a platelet or plasma, to protect the biological product from damage from cold or freezing conditions, such as during freeze drying (lyophilization). Such cryopreservatives include lyoprotectants, which are added to protect cells and preserve the structure of biomolecules in freeze-dried liquid preparations, such as during freeze drying or lyophilization. Cryoprotectants protect proteins from damage during freezing, while lyoprotectants provide protection against the stresses that occur during drying. Cryopreservatives can stabilize biologics against both the freezing and drying stresses during processing. For example, a cryopreservative in a PRP composition containing platelets protects the platelets by protecting the cell membrane from cold-induced cellular stress. For example, the cryopreservative can coat the platelet surface to protect the cell. In other examples a cryopreservative can incorporate into the lipid bilayer to maintain the integrity of the lipid bilayer. Cryopreservatives can decrease the freezing point of the composition to which the cryopreservative is added. For example, the cryopreservative can protect the platelet cell from crystallization. For purposes herein, a cryopreservative is not an excipient and cryopreservatives do not include anti-coagulants, such as anti-coagulants added during blood collection or apheresis, nor do they include other products, such as salts, that lower freezing point. Cryopreservatives do not exert a therapeutic effect. Exemplary cryopreservatives in the art for protecting platelets from cold-induced damage include, but are not limited to, for example: dimethyl sulfoxide (DMSO); carbohydrates and polysaccharides and sugars, such as, for example, trehalose, glucose, glycerol, maltodextrin, dextran, and hydroxyethyl starch (HES); and formaldehydes, such as, for example, formaldehyde, paraformaldehyde, glutaraldehyde, and permanganate. The PRP compositions provided herein do not include such cryopreservatives.

As used herein, pooling of blood products refers to combining the product from multiple donors, such as at least 5, 10, 15, 20, 25, 30, 35, 40, or more donors.

As used herein, anti-coagulant refers to a substance that prevents or inhibits coagulation of blood. In the methods herein, an anticoagulant can be added during platelet or plasma or whole blood collection, for example, to prevent clotting during the collection process. In some examples, an anticoagulant is added during apheresis to prevent clotting of the extracorporeal circuit. In some examples, the anticoagulant is selected from among one or more of acid citrate dextrose solution A (ACD-A; for example ACD-A containing citric acid, dihydrate sodium citrate, monohydrate dextrose), triple citrate, citrate-phosphate-dextrose solution with adenine (CPDA-1), heparin (e.g., sodium, lithium, or ammonium salt of heparin, calcium-titrated heparin), ethylenediaminetetraacetic acid (EDTA) and analogs thereof (e.g., disodium EDTA, tripotassium EDTA, dipotassium EDTA, ethyleneglycoltetraacetic acid (EGTA), ethylendiaminedipropionic acid (EDDP), and hexamethylenediaminetetraacetic acid (HDTA)), citrate (e.g., sodium citrate, trisodium citrate, trisodium citrate dehydrate, trisodium citrate pent hydrate, citrate dextrose, ACD-A, ACD-B), acid citrate dextrose solution B (ACD-B), oxalate (e.g., sodium oxalate, potassium oxalate, ammonium oxalate, lithium oxalic acid), sodium fluoride (e.g., sodium fluoride-potassium oxalate), or other anti-coagulant, such as anti-coagulants used during platelet collection, such as, for example, apheresis. In some examples, a PRP composition provided herein contains an anti-coagulant selected from among ACD-A, triple citrate, heparin, and CPDA-1. For example, the PRP composition comprises an anticoagulant such as Anticoagulant Citrate Dextrose Solution-Formula A (ACD-A).

As used herein, lyophilization, used herein interchangeably with freeze drying, refers to a process where liquid is removed from a composition to generate a solid form of the composition, such as a powder.

As used herein, cold storage refers to storage at refrigerated temperatures or at temperatures that are below ambient temperature but are above freezing temperatures. In examples herein, cold storage refers to storage for greater than or at or about 1, 2, 3, 4, 5, 6, 7, 8, 9, 10, 11, 12, 13, 14, 15 hours or more at refrigerated temperatures.

As used herein, refrigerated temperatures include temperatures between or between about 0° C.-10° C. Platelets can be incubated at refrigerated temperatures prior to pooling and/or prior to pooling with plasma. Platelets incubated at refrigerated temperatures prior to pooling remain in a primed, partially activated state prior to freeze drying and retain the primed, partially activated state after lyophilization and subsequent reconstitution.

As used herein, room temperature (RT) or ambient temperature include temperatures between or between about 15° C.-30° C. Generally, environmentally controlled indoor or ambient temperatures range from 18° C.-24° C. If necessary for precision RT is 20° C. to 22° C., for more precision it is 20° C.

As used herein, immediately and immediate refers to an action that is completed without delay. If there are two steps, the first step is completed and the second step that is initiated immediately is initiated as soon as the first step is completed. The referenced time for im-mediate action or for an action to happen immediately can depend on the referenced action. There are actions that are effected by human intervention, such as serial administration, in which the human, for example administers one agent, and then when the first ad-ministration is complete administers a second agent. In other instances, the occurrence is a naturally-occurring, such as in vivo occurrences following administration of an agent. For example, an immediate release of the contents of platelet granules upon administration refers to occurrences following administration; such release can be within at or about 2 to 5 minutes, and is said to be immediate. In other examples herein, where platelets are placed at refrigerated temperatures immediately after collection, the platelets are placed in re-frigerated temperatures without further processing and within about 10 to 20 minutes, such as at or about 15 minutes of collection, such as within at or about 10 to 20 minutes, such as at or about 15 minutes of an apheresis event. In examples herein, where primed platelets are activated immediately before administration to a subject, such as examples where platelets are activated ex vivo, the activated platelets are administered to a subject within at or about 2 to 5 minutes, such as at or about 5 minutes. In examples where the primed platelets combined with the additional therapeutic or therapeutics immediately prior to administration, the additional therapeutic(s) are added within at or about 1 to 4 minutes, such as within at or about 2 minutes.

As used herein, leukorich refers to a composition contains the same level of leukocytes as whole blood or that is enriched for leukocytes. A leukorich PRP composition can have the same concentration of leukocytes (white blood cells) as whole blood or can have a higher concentration of platelets compared to whole blood. In examples, a leukorich PRP composition has more leukocytes or is more concentrated for leukocytes compared to whole blood. A leukorich composition, such as a leukorich PRP composition, can be prepared by collecting platelets by apheresis without use of a filter to leukoreduce the preparation. In other examples, a leukorich composition, such as a leukorich PRP composition, can be prepared by collecting whole blood and treating the whole blood to prepare a composition containing platelets and leukocytes.

As used herein, leukoreduced refers to a composition that does not contain leukocytes or contains a lower concentration of leukocytes than whole blood. A leukoreduced PRP composition does not contain leukocytes or contains a lower concentration of leukocytes than whole blood. In examples, a leukoreduced PRP composition has fewer leukocytes or is less concentrated for leukocytes compared to whole blood. A leukoreduced composition, such as a leukoreduced PRP composition, can be prepared by collecting platelets by apheresis and subjecting the collected platelet preparation to treatment with a filter to leukoreduce the preparation. In other examples, a leukoreduced composition, such as a leukoreduced PRP composition, can be prepared by collecting whole blood and treating the whole blood to prepare a composition containing platelets that is leukoreduced.

As used herein, allogeneic such as allogeneic cell therapy involves the infusion or transplant of cells that are derived from a donor other than the subject. Autologous cell therapy refers to reinfusion of the cells of a subject.

As used herein, externalized phosphatidylserine (PS) refers to the location of phosphatidylserine on the platelet membrane. Phospholipids such as phosphatidylserine are distributed between the inner and outer leaflets of plasma membranes depending on the state of the platelet cell. In quiescent platelets, anionic phospholipids such as phosphatidylserine are present only in the inner leaflet of the membrane bilayer. An early step in platelet activation includes translocation of phosphatidylserine (PS) from the inner to the outer leaflet of the platelet membrane bilayer. PS is preferentially exposed on the cell surface during physiological processes, such as platelet activation. Externalized PS can be measured by assays known in the art. For example, externalized PS can be measured by lactadherin binding, and assessed by assays known in the art, such as, for example, flow cytometry. In examples herein, platelets in a PRP composition incubated at refrigerated temperatures prior to lyophilization herein have increased externalized PS compared to platelets that are not incubated at refrigerated temperatures prior to lyophilization.

As used herein, inactivate or inactivated with reference to platelets refers to platelets that are in a quiescent state. Inactivated platelets do not have characteristics of primed or activated platelets. For example, inactivated platelets do not have phosphatidylserine externalized to the outer leaflet of the platelet membrane. Inactivated platelets also have intact granules, such as intact alpha granules. Inactivated platelets also are not positive or are positive in low amounts when assessed by immunohistochemistry, such as by staining with antibodies to p-selectin. Inactivated platelets have a different shape from activated platelets. This in contrast to primed platelets provided in the PRP compositions herein.

As used herein, primed platelets, used interchangeably with partially activated platelets, are platelets that exhibit some characteristics of activated platelets but are not fully activated; the contents of the platelet granules have not been released. Primed platelets are not quiescent and are not activated, rather they show early markers of activated platelets. Primed platelets can be characterized phenotypically, such as by the presence of and absence of markers indicative of activation. For phenotypic identification, primed platelets have externalized phosphatidylserine. Primed platelets are not positive for CD62 or other markers that indicate that the platelet granules have emptied their granule contents. To identify PRP compositions that contain primed platelets, platelets in the composition are positive for (or for markers indicative of) externalized phosphatidylserine, but only up to, at most 40%, and generally less than 40% are positive for a marker, such as CD62 or as assessed by another marker of platelet activation such as by mean platelet volume that indicates platelet activation. Hence, for purposes herein, the platelets in the PRP compositions comprise at most 40% activated platelets, such as at most 40%, such as up to at or about 35% or 30% activated platelets; and at least 50% of the platelets are primed platelets, which can be identified by externalized phosphatidylserine, which, for example, can be identified by lactadherin binding.

In the lyophilized PRP compositions provided herein, a majority, at least about or about 50%, 55%, 60%, 65%, 70%, 75%, 80%, 85%, 90%, 91%, 92%, 93%, 94%, 95%, 96%, 97%, 98%, 99% or 100% of the platelets in the PRP composition have externalized phosphatidylserine, such as phosphatidylserine on the outer leaflet of the plasma membrane, indicating that they are primed or partially activated. These primed platelets are not positive for CD62 or other activation marker. In examples herein, the ratio of platelets with externalized phosphatidylserine to platelets without externalized phosphatidylserine in the composition is at least or at least about 1:1, 1.1:1, 1.2:1, 1.3:1, 1.4:1, 1.5:1, 1.6:1, 1.7:1, 1.8:1, 1.9:1, 2.1:1, 2.2:1, 2.3:1, 2.4:1, 2.5:1, 2.6:1, 2.7:1, 2.8:1, 2.9:1, 3:1, 3.1:1, 3.2:1, 3.3:1, 3.4:1, 3.5:1, 3.6:1, 3.7:1, 3.8:1, 3.9:1, 4:1, 4.1:1, 4.2:1, 4.3:1, 4.4:1, 4.5:1, 4.6:1, 4.7:1, 4.8:1, 4.9:1, 5:1, 5.1:1, 5.2:1, 5.3:1, 5.4:1, 5.5:1, 5.6:1, 5.7:1, 5.8:1, 5.9:1, 6:1, 6.1:1, 6.2:1, 6.3:1, 6.4:1, 6.5:1, 6.6:1, 6.7:1, 6.8:1, 6.9:1, 7:1, 7.1:1, 7.2:1, 7.3:1, 7.4:1, 7.5:1, 7.6:1, 7.7:1, 7.8:1, 7.9:1, 8:1, 8.1:1, 8.2:1, 8.3:1, 8.4:1, 8.5:1, 8.6:1, 8.7:1, 8.8:1, 8.9:1, 9:1, 9.1:1, 9.2:1, 9.3:1, 9.4:1, 9.5:1, 9.6:1, 9.7:1, 9.8:1, 9.9:1, 10:1, 10.1:1, 10.2:1, 10.3:1, 10.4:1, 10.5:1, 10.6:1, 10.7:1, 10.8:1, 10.9:1, 11:1, 11.1:1, 11.2:1, 11.3:1, 11.4:1, 11.5:1, 11.6:1, 11.7:1, 11.8:1, 11.9:1, 12:1, 12.1:1, 12.2:1, 12.3:1, 12.4:1, 12.5:1, 12.6:1, 12.7:1, 12.8:1, 12.9:1, 13:1, 13.1:1, 13.2:1, 13.3:1, 13.4:1, 13.5:1, 13.6:1, 13.7:1, 13.8:1, 13.9:1, 14:1 or more. In the reconstituted PRP compositions provided herein up to 100% of the reconstituted lyophilized platelets are in the partially activated state. Similarly, in the reconstituted PRP compositions provided herein, at least about or about 50%, 55%, 60%, 65%, 70%, 75%, 80%, 85%, 90%, 91%, 92%, 93%, 94%, 95%, 96%, 97%, 98%, 99% or 100% of the platelets in the PRP composition have externalized phosphatidylserine, which can be identified by lactadherin binding.

Phosphatidylserine externalization can be assessed by flow cytometry assay and/or immunohistochemical staining. In reconstituted PRP compositions provided herein, the ratio of platelets that are lactadherin positive to platelets that are lactadherin negative in the composition is at least or at least about 1:1, 1.1:1, 1.2:1, 1.3:1, 1.4:1, 1.5:1, 1.6:1, 1.7:1, 1.8:1, 1.9:1, 2.1:1, 2.2:1, 2.3:1, 2.4:1, 2.5:1, 2.6:1, 2.7:1, 2.8:1, 2.9:1, 3:1, 3.1:1, 3.2:1, 3.3:1, 3.4:1, 3.5:1, 3.6:1, 3.7:1, 3.8:1, 3.9:1, 4:1, 4.1:1, 4.2:1, 4.3:1, 4.4:1, 4.5:1, 4.6:1, 4.7:1, 4.8:1, 4.9:1, 5:1, 5.1:1, 5.2:1, 5.3:1, 5.4:1, 5.5:1, 5.6:1, 5.7:1, 5.8:1, 5.9:1, 6:1, 6.1:1, 6.2:1, 6.3:1, 6.4:1, 6.5:1, 6.6:1, 6.7:1, 6.8:1, 6.9:1, 7:1, 7.1:1, 7.2:1, 7.3:1, 7.4:1, 7.5:1, 7.6:1, 7.7:1, 7.8:1, 7.9:1, 8:1, 8.1:1, 8.2:1, 8.3:1, 8.4:1, 8.5:1, 8.6:1, 8.7:1, 8.8:1, 8.9:1, 9:1, 9.1:1, 9.2:1, 9.3:1, 9.4:1, 9.5:1, 9.6:1, 9.7:1, 9.8:1, 9.9:1, 10:1, 10.1:1, 10.2:1, 10.3:1, 10.4:1, 10.5:1, 10.6:1, 10.7:1, 10.8:1, 10.9:1, 11:1, 11.1:1, 11.2:1, 11.3:1, 11.4:1, 11.5:1, 11.6:1, 11.7:1, 11.8:1, 11.9:1, 12:1, 12.1:1, 12.2:1, 12.3:1, 12.4:1, 12.5:1, 12.6:1, 12.7:1, 12.8:1, 12.9:1, 13:1, 13.1:1, 13.2:1, 13.3:1, 13.4:1, 13.5:1, 13.6:1, 13.7:1, 13.8:1, 13.9:1, 14:1 or more.

As used herein, activated platelets refer to platelets in which the granule membrane proteins have incorporated with the platelet membrane and granule contents released into the extracellular environment. Activated platelets are characterized phenotypically and can be identified as activated by assays known in the art, such as flow cytometry and immunostaining. Among the markers for identification of activated platelets is CD62P (P-selectin) positive. For purposes herein, in PRP compositions provided herein, a portion, up to at or about or at most 30%, 31%, 32%, 33%, 34%, 35%, 36%, 37%, 38%, 39%, or 40% of the platelets in the PRP composition provided herein can be activated platelets, such as indicated by being positive for CD62. Activated platelets also are characterized by their shape, such as by using methods such as light scatter, microscopy, or swirling. Mean platelet volume (MPV) can be used to determine platelet shape. In examples, larger MPV indicates activation.

As used herein, activation of primed platelets refers to conversion of primed platelets into activated platelets. For example, primed platelets, such as those that are in the PRP compositions provided herein can be activated prior to therapeutic use for embodiments in which activated platelets are employed. For example, a lyophilized PRP composition containing primed platelets is reconstituted in a liquid, such as sterile water or saline, and the primed platelets are activated prior to administration. In other embodiments, the PRP composition is administered and activates in vivo. Activation can be effected in vitro by changing conditions or adding an agonist. For example, activation can be effected by changing the moisture content, temperature, loading time, the presence or absence of a post-lyophilization heat step, and/or by the addition of agonists to activate the platelets. Platelets in a primed state that are activated prior to administration are for uses in which an activated state is desired, such as for topical administration, such as for wound healing, or other uses where the PRP composition is applied to an injury, such as a wound or post-surgery or during surgery. In some examples, an agonist can be added to the PRP composition containing primed platelets prior to administration of the primed platelets. Agonists for activation include, but are not limited to, one or more of arachidonic acid, collagen, epinephrine, thrombin receptor-activating peptides (TRAP), ristocetin, and/or other such agonists. The addition of an agonist to primed platelets, prior to administration to a subject, activates the primed platelets; the activated platelets are then administered to the subject.

As used herein, “biological sample” or “sample” are used interchangeably and refer to a portion of a body fluid or body tissue obtained from a living source and includes any body fluid or tissue of a subject from which cells, such as platelets, protein, or serum can be obtained. The biological sample can be a sample obtained directly from a biological source. For example, isolated nucleic acids that are amplified constitute a biological sample. Biological samples include, but are not limited to, body fluids, such as whole blood and blood plasma.

As used herein, a “sample previously obtained from a subject” refers to samples obtained from subjects prior to analysis.

As used herein, “patient” or “subject” to be treated includes humans and non-human animals. “Animal” includes any animal, such as, but are not limited to horses; dogs; cats; elephants; fish; mammalian water animals, such as dolphins and whales; primates including humans, gorillas, and monkeys; rodents, such as mice and rats; fowl, such as chickens; ruminants, such as goats, cows, deer, sheep, pigs, and other animals. Non-human animals exclude humans as the contemplated animal.

As used herein, a functional activity with reference to a platelet, such as a platelet in a platelet-rich plasma (PRP) composition refers to a platelet-mediated function including, but not limited to, clot formation, coagulation, thrombosis, maintaining primary hemostasis, maintaining blood flow, responding to vascular insult or injury, platelet recruitment to a thrombus, wound healing, immunity (e.g., against bacteria, virus, or tumors), autophagy, growth factor releases, release particles (e.g., miRNA, mRNA, enzymes, proteins, small molecules). Exemplary of assays for testing platelet activity include blood clot formation and detection of factors released from platelets such as by ELISA or SDS-PAGE.

As used herein, a “control” refers to a sample that is substantially identical to the test sample, except that it is not treated with a test parameter, or, if it is a plasma sample, it can be from a normal volunteer not affected with the condition of interest. A control also can be an internal control.

As used herein, a “composition” refers to any mixture of two or more elements. It can be a solution, suspension, liquid, powder, paste, aqueous, non-aqueous or any combination thereof. In examples herein, a platelet-rich plasma composition (PRP composition) contains pooled platelets from multiple donors and plasma from multiple donors, and does not contain a cryopreservative. In some examples herein a platelet-rich plasma composition (PRP composition) contains pooled platelets from multiple donors, plasma from multiple donors and an anti-coagulant, and does not contain a cryopreservative.

As used herein, a “combination” refers to any association between or among two or more items. The combination can be two or more separate items, such as two compositions or two collections, can be a mixture thereof, such as a single mixture of the two or more items, or any variation thereof. The elements of a combination are generally functionally associated or related. In examples herein, the PRP composition can be provided in a combination with other compounds, such as another therapeutic, such as a drug. For example, the PRP composition can be provided in combination with one or more of stem cells, antibodies, growth factors, bone matrix materials (e.g., for bone healing), ECM or MSC exosomes or mitochondria, anti-inflammatoir other compounds or therapeutics or drugs.

As used herein, “fluid” or “liquid” refers to any composition that can flow. Fluids thus encompass compositions that are in the form of semi-solids, pastes, solutions, aqueous mixtures, gels, lotions, creams, and other such compositions. In examples herein, a lyophilized PRP composition is reconstituted in a liquid, such as sterile saline or water. A reconstituted lyophilized PRP composition is a liquid composition.

As used herein, treatment refers to any effects that ameliorate symptoms of a disease or disorder. Treatment encompasses prophylaxis, therapy and/or cure. Treatment also encompasses any pharmaceutical or therapeutic use of the PRP compositions provided herein. Treatment refers to any effects that ameliorate or prevent or other reduce or eliminate any symptom or manifestation of a disease or disorder.

As used herein an “effective amount” of a compound or composition, such as a PRP composition provided herein, for treating a particular disease or condition is an amount that is sufficient to ameliorate, or in some manner reduce the symptoms associated with the disease or condition, or decrease the time to onset of the disease or condition. Such amount can be administered as a single dosage or can be administered according to a regimen, whereby it is effective. The amount can cure the disease or condition but, typically, is administered to ameliorate the symptoms of the disease or condition. In some examples, repeated administration is required to achieve a desired amelioration of symptoms.

As used herein, a “therapeutically effective amount” or a “therapeutically effective dose” refers to the quantity of an agent, compound, material, or composition containing a compound that is at least sufficient to produce a therapeutic effect following administration to a subject. Hence, it is the quantity necessary for preventing, curing, ameliorating, arresting, or partially arresting a symptom of a disease or disorder or condition. In examples herein, a therapeutically effective amount can refer to the number of platelets required to be administered to produce a therapeutic effect. In some examples herein, a therapeutically effective amount of platelets in a PRP composition is at least about 300,000 platelets per microliter of the PRP composition (i.e., the reconstituted lyophilized PRP composition).

As used herein, a “therapeutic effect” means an effect resulting from treatment of a subject that alters, typically improves or ameliorates, the symptoms of a disease or condition or that cures a disease or condition.

As used herein, a “prophylactically effective amount” or a “prophylactically effective dose” refers to the quantity of an agent, compound, material, or composition containing a compound that when administered to a subject, have the intended prophylactic effect, e.g., preventing or delaying the onset, or reoccurrence, of disease or symptoms, reducing the likelihood of the onset, or reoccurrence, of disease or symptoms, or reducing the incidence of viral or bacterial infection. The full prophylactic effect does not necessarily occur by administration of one dose, and in some examples can occur only after administration of a series of doses. Thus, a prophylactically effective amount can be administered in one or more administrations.

As used herein, the singular forms “a,” “an” and “the” include plural referents unless the context clearly dictates otherwise. Thus, for example, reference to the addition of a nucleic acid probe to a sample can include one or a plurality of probes being added to the sample.

As used herein, ranges and amounts can be expressed as “about” a particular value or range. About also includes the exact amount. Hence “about 5 platelets” means “about 5 platelets” and also “5 platelets.”

As used herein, “optional” or “optionally” means that the subsequently described event or circumstance does or does not occur, and that the description includes instances where said event or circumstance occurs and instances where it does not. For example, an optionally substituted group means that the group is unsubstituted or is substituted.

As used herein, a “kit” refers to a combination of components, such as a combination of the compositions herein and another item for a purpose including, but not limited to, reconstitution, activation, and instruments/devices for delivery, administration, diagnosis, and assessment of a biological activity or property. A kit is a packaged combination. Kits optionally include instructions for use.

For clarity of disclosure, and not by way of limitation, the detailed description is divided into the subsections that follow.

### B. PLATELET-RICH PLASMA AND OVERVIEW

Blood is a mixture of various components, including red blood cells, white blood cells, platelets, and plasma. Plasma also contains a mixture of salts, enzymes, antibodies (immunoglobulin), clotting/coagulation factors, and other proteins; most plasma is water. Blood components can be separated from each other using various methods, including differential centrifugation, which separates blood components based on size and/or density.

All blood cells derive from a common pluripotent hematopoietic stem cell, which differentiates into different cell lines. Each of these cell series contains precursors that can divide and mature. Platelets are a blood component involved in the clotting process, vascular repair and control of inflammation. Platelets, also called thrombocytes, develop from the bone marrow; they are anucleate, discoid cellular elements with different sizes and a density of approximately 2 μm in diameter, the smallest density of all blood cells. The physiological count of platelets circulating in the blood stream ranges from about 150,000 to 400,000 platelets per μL and varies by species, age, gender, and health state. Each platelet contains about 50-80 granules. Platelets contain several secretory granules for platelet function. There are 3 types of granules: dense granules, α-granules, and lysosomes. Platelets are responsible for the aggregation process, and their primary function is to contribute to homeostasis, which is effected through three processes: adhesion, activation, and aggregation. During a vascular injury, platelets are activated, and their granules release factors that promote coagulation. Platelets contain an abundance of growth factors and cytokines that affect inflammation, angiogenesis, stem cell migration, and cell proliferation (see, e.g., Alves et al. (2017) Skin appendage discord 4:18-24). Platelets are used as therapeutics.

Purified platelets and compositions comprising platelets have variety of uses, including, but not limited to, treating subjects with low platelet count (thrombocytopenia) or with abnormal platelet function (thrombasthenia), and also controlling bleeding after injury or during surgery, and other therapeutic purposes described herein and/or known to those of skill in the art. Purified platelets can be combined with plasma for use for therapeutic purposes, such as for regenerative medicine, for healing injured tendons, ligaments, muscles, joints, and for other therapeutic uses as detailed herein and/or known in the art.

Platelets can be provided in platelet-rich plasma compositions, which contain platelets, generally at a higher concentration than in blood, and plasma. Platelet-rich plasma (PRP) compositions are provided herein. The PRP compositions provided herein contain primed platelets, prepared by methods described herein, that result in compositions containing primed platelets. Upon activation of primed platelets in the PRP, in vivo or in vitro or ex vivo, the α-granules are degranulated and release the growth factors (GFs) and cytokines, which in vivo modify the pericellular microenvironment. GFs released by platelets in the PRP compositions provided herein include, but are not limited to, vascular endothelial GF (VEGF), fibroblast GF (FGF), platelet-derived GF BB (PDGF-BB), epidermal GF (EGF), hepatocyte (HGF), insulin-like GF-1 (IGF-1), IGF-2, matrix metalloproteinases 2, 9, and interleukin 8 (IL-8).

Previously prepared platelet compositions and preparations required and contained cryopreservatives to prevent cell damage that can occur during cold storage or freeze drying treatment. Freezing platelets prepared as in the prior art results in altered platelet morphology including fractured platelets unless cryopreservatives are added to the compositions to protect the platelets.

The PRP compositions provided herein contain plasma and platelets and do not contain or require cryopreservatives. Methods are provided herein for preparing platelets and PRP that do not require cryopreservatives. Methods are provided herein that permit preparation of PRP compositions that do not contain nor require cryopreservatives to maintain the integrity of the platelets. Data herein demonstrate that platelets prepared in accord with the methods provided herein are intact and functional. The resulting PRP compositions do not contain or require cryopreservatives. Data herein show that platelets prepared in accord with the methods provided herein do not require cryopreservatives in the compositions to maintain function after freezing, such as freeze drying.

Platelets prepared in accord with the methods provided herein, including post-collection storage at refrigerated temperatures such as 0-10° C., enter a primed state and retain the primed state during storage; they also retain the primed state after lyophilization and subsequent reconstitution in a liquid. The primed platelets in the PRP compositions herein have intact platelet α-granules. The freeze dried platelets in PRP compositions provided herein do not contain nor are they are prepared in cryopreservative(s). The compositions are freeze dried. The resulting freeze dried platelets are primed, in a partially activated state. The freeze dried platelets in the PRP compositions provided herein remain in a primed, partially activated state, following reconstitution of the plasma rich platelet composition in a liquid. The primed platelets as provided herein are therapeutically active upon administration.

### 1. Overview of the Methods for Preparing a Platelet-rich Plasma Composition Without Cryopreservative

Provided herein are methods of preparing platelets and platelet-rich plasma (PRP) compositions. The method includes a step of refrigerating the platelets, which are collected in plasma, prior to adding the platelets in plasma to additional plasma. As a result, the resulting platelet compositions do not require cryopreservatives for freeze drying or for long-term storage as frozen compositions or freeze-dried compositions. Provided are the resulting platelets and PRP compositions, which do not contain a cryopreservative.

The methods include the steps of: collecting platelets in plasma from animal donors, followed by cold storage of platelets at or about 0° C.-10° C., such as at 4° C. Within up to about 14 days of storage, generally at least 18 hours to 36 hours, up to 14 days, such as up to 13, 12, 11, 10, 9, 8, 7, 6, or 5 at about 0° C.-10° C., the cold-stored platelets are pooled with additional plasma to prepare a platelet-rich plasma (PRP) composition containing the platelets, plasma and anticoagulant. As shown and described herein, platelets placed in refrigerated conditions at about 0° C.-10° C. immediately after collection have advantageous properties. Platelets subjected to incubation at refrigerated temperatures, such as at or about 48 hours up to 14 days, enter a primed or partially activated state. The primed platelets harbor characteristics of partially activated platelets, such as externalized phosphatidylserine, indicating that they are not fully activated; they have not emptied their granule contents. Primed platelets have intact platelet granules. The primed platelets described herein with intact granules, such as alpha-granules, can be used to deliver cytokines to a location, as once the platelets reach the desired location they can be activated. Primed platelets in a PRP composition provided herein, upon activation, degranulate and have not degranulated prior to administration or external activation. The primed state is maintained after further treatment, such as freeze drying of the platelets and the primed state is retained after reconstitution of the lyophilized PRP composition.

### 2. Preparation of a Platelet-Rich Plasma Composition

In the methods herein, platelets are collected from multiple donors, such as more than 5, 10, 20, 30, or 40, or more of a species of interest, such as equine, canine, or human. Platelets from multiple donors can be pooled to yield a more uniform functional profile (i.e., growth factor concentration; activation potential) and target platelet count compared to platelets isolated from a single donor. In some examples, the resulting compositions contain at least 200,000, 300,000, 400,000 or 500,000 platelets/μL.

During platelet collection anticoagulant(s) can be added. The PRP compositions can contain an anticoagulant, such as anticoagulants added during platelet or plasma collection (e.g., apheresis). Any anticoagulant can be added that does not impact platelet activity. In some examples, the anticoagulant is selected from among one or more of acid citrate dextrose solution A (ACD-A; contains citric acid, dihydrate sodium citrate, monohydrate dextrose), heparin (e.g., sodium, lithium, or ammonium salt of heparin, calcium-titrated heparin), ethylenediaminetetraacetic acid (EDTA) and analogs thereof (e.g., disodium EDTA, tripotassium EDTA, dipotassium EDTA, ethyleneglycoltetraacetic acid (EGTA), ethylendiaminedipropionic acid (EDDP), and hexamethylenediaminetetraacetic acid (HDTA)), citrate (e.g., sodium citrate, trisodium citrate, trisodium citrate dehydrate, trisodium citrate pent hydrate, citrate dextrose, ACD-A, ACD-B), acid citrate dextrose solution B (ACD-B), citrate-phosphate-dextrose solution with adenine (CPDA-1), oxalate (e.g., sodium oxalate, potassium oxalate, ammonium oxalate, lithium oxalic acid), sodium fluoride (e.g., sodium fluoride-potassium oxalate), or other anti-coagulant, such as anti-coagulants used during platelet collection, such as, for example, apheresis. For example, the PRP composition comprises an anticoagulant such as Anticoagulant Citrate Dextrose Solution-Formula A (ACD-A). No cryopreservative is added.

It is understood that in accord with the methods provided herein, any method for isolating and/or collecting platelets can be used, such as any methods known in the art or methods using commercially available equipment or devices for platelet isolation and/or collection. As exemplified herein, platelets can be collected by apheresis. The platelets can be collected from a donor or multiple donors by apheresis, or can be collected by other methods such as isolating platelets from whole blood, such as by centrifugation and, optionally, additional treatment, such as treatment or filtration for leukoreduction. For example, blood components can be separated by size, such as by filtration, to remove larger white blood cells from the composition.

After collection, the platelets in plasma immediately are incubated as detailed herein, for at least 18 hours, such as 36 or 48 hours, up to 14 days at refrigerated temperatures, at or about 0° C.-10° C., such as at 4° C. to 8° C. It is shown herein that platelets stored at refrigerated temperatures for this time period, such as for 3-5 days, are in a partially activated or primed state, which is shown here to be advantageous for storage and for certain therapeutic uses. In addition to resulting in primed platelets, containing intact granules, benefits of refrigerating platelets immediately after collection include decreased risk of contamination from pathogens, such as bacteria and/or mycoplasma. In accord with the methods provided herein, no cryopreservatives are added to the platelets following collection.

The platelets can be added to additional plasma prior to further treatment or processing, such as freeze-drying. The platelets, which can be collected from multiple donors, can be added to additional plasma that is collected from multiple donors. In some examples, platelets and plasma are collected from multiple donors and the platelets and plasma are combined. The collected plasma from multiple donors can be pooled and frozen and stored. Frozen plasma is thawed prior to combining with platelets. No cryopreservatives are added to the plasma following collection from the donor animals. No preservatives are added to the platelets or to PRP compositions; no cryopreservatives are added to PRP for freeze drying.

Platelets and additional plasma can be combined to produce the PRP at ambient temperatures. In some examples, once platelets and plasma are pooled at ambient temperatures to prepare a platelet-rich plasma (PRP) composition, the PRP composition is handled at ambient temperatures for not more than 4 hours prior to administration or freeze drying. For example, the PRP composition is at ambient temperatures for not more than about 4, 5, 6 hours, such as at least or at least about 4 hours, during preparation prior to freeze drying. For example, the PRP composition is held at ambient temperatures while preparing for vialing and placement in the lyophilizer prior to freeze drying. The PRP composition generally is held at ambient temperatures for up to but no longer than about four hours.

In examples herein, the PRP composition is further processed, such as freeze dried. In some examples, the PRP composition is further processed, such as freeze dried, within four hours of pooling or combining of the platelets and additional plasma. No cryopreservative is added prior to freeze drying. In examples of the PRP compositions provided herein, the PRP composition contains plasma pooled from multiple donors, platelets pooled from multiple donors and an anticoagulant; the PRP composition does not contain additional excipients.

In the methods herein, the cold-treated platelets are combined with additional plasma isolated from animal donors and the combination of platelets and plasma (platelet-rich plasma) can be lyophilized. Lyophilization as detailed herein includes a controlled freezing process and subsequent freeze-drying without addition of any cryopreservative. For example, pooled plasma combined with pooled platelets containing anticoagulant and no other excipients can be freeze dried. An anticoagulant can be added prior to freeze drying, such as during platelet and/or plasma collection. For example, the PRP composition comprises an anticoagulant such as Anticoagulant Citrate Dextrose Solution-Formula A (ACD-A). The PRP composition containing platelets and plasma and optionally the anticoagulant, and no cryoprotectant, can be freeze dried.

In the methods for preparing PRP composition containing primed platelets provided herein, the PRP composition is subjected to a controlled freezing process from ambient temperature to freezing temperatures prior to lyophilization. The controlled process can include a slow temperature drop over several minutes to hours. For example, the PRP composition can be subjected to a controlled freezing process prior to freeze drying of about 1° C./minute for about 60 minutes. The PRP composition can be frozen during the controlled freezing process to sub-freezing temperatures, such as temperatures below −20° C., such as at or about −40° C., prior to freeze drying. Incubation of the collected platelets at below ambient, refrigerated temperatures, at or about 0° C.-10° C., such as at or about 4° C., results in intact platelets that survive the lyophilization process without any cryopreservative. Primed platelets are among the platelets in the PRP composition following lyophilization.

The freeze-dried (lyophilized) PRP composition presents as a solid, such as a powder or a wafer. The powdered PRP composition can be rehydrated prior to use, for administration. The lyophilized PRP composition containing primed platelets can be reconstituted in liquid, such as saline or water or other liquid where the characteristics of the platelets prior to lyophilization are maintained after reconstitution of the lyophilized PRP composition. For example, the powdered PRP can be rehydrated in sterile water for injection or in sterile saline, such as, for example 0.9% saline.

The number of platelets in the composition following lyophilization and reconstitution is similar to the number of platelets in the PRP composition prior to lyophilization. The concentration of platelets in the reconstituted PRP composition is similar to or greater than the platelet concentration in whole blood, where normal platelet count in humans is about or is 150,000 to 450,000 platelets/μL of blood. In examples, the platelet concentration in PRP is at supraphysiologic concentrations. The PRP compositions provided herein, when reconstituted, contain, for example, 300,000 to 500,000 platelets per microliter up to at or about 1,000,000 platelets/μL.

### 3. Primed Platelets in a Platelet-Rich Plasma Composition

As described above and herein, high percentage of platelets in the PRP compositions provided herein are in the primed state. Primed platelets show some characteristics of activated platelets, but are not fully activated. This is achieved by the production method described herein. As shown herein, incubation of the platelets at refrigerated temperatures for a limited time, of at least about 18 hours up to 14 days, immediately post-donation preserves a high number of platelets, maintains the functional integrity of platelets, in the absence of cryopreservative, and results in platelets that are in a primed but not fully activated state. As described and demonstrated herein, refrigeration of platelets prior to lyophilization effects a partially-activated state of the platelets, and results in composition that, when freeze-dried, is a storage-stable lyophilized platelet rich plasma composition, and does not require or contain cryopreservatives. Most of the primed platelets in the PRP composition retain the primed state following lyophilization and following reconstitution of the lyophilized PRP composition. This primed state is advantageous for various applications. As detailed below, platelets in the PRP compositions provided herein are primed and ready to act; upon administered to a joint, the primed platelets are clinically effective compared to quiescent platelets. Primed platelets are activated upon administration, such as intralesional administration, and release cytokines upon activation for an intralesional therapeutic effect. Primed platelets in a PRP composition provided herein do not require the activation stimuli necessary to activate quiescent platelets and can be used for regenerative therapies and wound healing where the platelets are delivered to the region of interest, such as a joint, tendon or for treatment of a wound. Early release of platelet granule contents during storage, prior to therapeutic administration, negates therapeutic efficacy of administered platelets; the granule contents are not available for platelet functions, such as, for example, leukocyte and platelet recruitment, promoting clotting and wound healing, and providing immunity against infection. Also, granule contents released during storage, and present in the storage medium, can cause transfusion reactions upon clinical administration. Full platelet activation during storage is undesirable and can negate the therapeutic benefits of platelets, such as the therapeutic benefits of release of the granule contents at the injury site. Platelets that retain the ability to activate upon removal from storage can be used for therapeutic purposes, such as regenerative therapies and for wound healing. Activation upon administration results in release of granule contents for therapeutic uses.

The primed state can be characterized phenotypically. For example, primed platelets in the compositions herein have externalized phosphatidylserine. In some examples, the majority of platelets in the reconstituted PRP composition have externalized phosphatidylserine (e.g., greater than 50% of the platelets have externalized phosphatidylserine). The majority of platelets in the PRP compositions provided herein are in a primed state of partial activation. For example, the majority of platelets have externalized phosphatidylserine, and the majority of cells are not positive for CD62P. The lyophilized and reconstituted platelet rich plasma compositions provided herein, at least about 40%, and up to 100% of the platelets exhibit externalized phosphatidylserine, up to 100% of the platelets are primed platelets in the partially activated state.

Primed platelets in the PRP compositions provided herein are not fully activated; they are phenotypically distinct from fully activated platelets. Primed platelets have one or more of the following characteristics of primed or inactivated platelets: externalized phosphatidylserine, intact granules, negative for p-selectin on the platelet surface, retain discoid shape, and do not aggregate upon swirling or light agitation. Primed platelets have externalized phosphatidylserine and can be activated in vitro by external stimuli, such as addition of Ca++. The primed platelets are functional following storage at freezing temperatures in the absence of a cryopreservative.

In prior PRP compositions, platelets are in a quiescent state. Platelets in the quiescent state that are delivered for therapeutic purposes generally require external activation for therapeutic activity and do not activate normally upon administration to a joint. Hence, quiescent platelets administered to a joint do not consistently activate and are not as therapeutically useful. Platelets in the PRP compositions provided herein are primed and ready to act; upon administered to a joint, the primed platelets are clinically effective compared to quiescent platelets. Primed platelets are activated upon administration, such as intralesional administration, and release cytokines upon activation for an intralesional therapeutic effect. Primed platelets in a PRP composition provided herein do not require the activation stimuli necessary to activate quiescent platelets and can be used for regenerative therapies and wound healing where the platelets are delivered to the region of interest, such as a joint, tendon or for treatment of a wound.

### C. PLATELETS AND PLATELET-RICH PLASMA

### 1. Platelet Function and Activation

Platelets are anucleate cells produced by the bone marrow that circulate in the bloodstream. Platelets maintain vascular integrity, play a central role in the hemostatic response to vascular injury, and contribute to inflammatory and immune responses. Platelet transfusions are common in critically ill human patients, and are commonly transfused in a perioperative setting as either prophylaxis before surgery or for treatment of bleeding or hemorrhage during or after surgery.

Under normal conditions, in the absence of cellular stress or injury, platelets circulate through the circulatory system in an inactive, non-adhesive state. Inactive platelets have a discoid shape with proteins, nonprotein molecules, and enzymes packaged into various types of platelet granules, including alpha-granules, electron dense granules, and platelet lysosomes. Platelet alpha-granules contain proteins including P-selectin, fibrinogen, coagulation factors, hemostatic factors (e.g., factor V, vWF, fibrinogen), mitogenic factors, angiogenic factors (e.g., angiogenin, VEGF), anti-angiogenic factors (e.g., angiostatin, PF4), growth factors (e.g., PDGF, bFGF, SDF1α), proteases (e.g., MMP9 and MMP2), chemokines and cytokines (e.g., TNFα, TNFβ, RANTES). Nonprotein molecules including serotonin, histamine, ADP, ATP, pyrophosphate, and calcium are packaged into electron dense platelet granules. Enzymes such as glycosidases, acid proteases, and cationic proteins are packaged into platelet lysosomes. Upon activation, platelets can release the contents of their granules.

Phenotypically, inactivated platelets show full granules, non-membrane blebbing, low levels or absent platelet microparticles or platelet membrane vesicles (0.1 to 1.0 μm), and changes in the expression of platelet integrin receptors. In inactivated platelets, intracellular levels of calcium, cytokines, serotonin, histamine, ADP, ATP are absent or remain low; and expression of soluble P-selectin and surface receptors for platelet activation and platelet aggregation, including monocyte-platelet aggregates, neutrophil-platelet aggregates, and platelet microparticles, is low/absent. Inactivated platelets do not translocate the anionic aminophospholipid phosphatidylserine (PS) to the outer leaflet of the platelet plasma membrane.

Circulating platelets are in a state of low activation but high responsiveness. Circulating platelets can be activated by endogenous stimuli. Upon activation, platelets undergo morphological changes, including restructuring of the platelet membrane (Sharda A, Flaumenhaft R. F1000Research (2018) February 28; 7:236. doi: 10.12688/f1000research.13283.1). Activated platelets undergo morphological changes from a discoid shape (discocyte) to a spherical shape (spherocytes) with pseudopods. Upon activation, phosphatidylserine translocates from the inner to outer leaflet of the plasma membrane, where it serves as a scaffold for the activation of various clotting factors. Phosphatidylserine exposure on the outer membrane of activated platelets can function in platelet blood clotting activities through various pathways and/or by various mechanisms. For example, phosphatidylserine on the membrane of activated platelets, can regulate, such as promote thrombin production through various cellular pathways, which can, in turn, activate downstream signaling pathways to signal clot formation to achieve a hemostatic effect. Expression of negatively charged phospholipids such as phosphatidylserine and phosphatidylethanolamine on the outer leaflet of the platelet membrane of activated platelets offers a surface for the prothrombinase complex which, in turn, contributes to procoagulant activity. Exposed phosphatidylserine is a marker of primed platelets; platelets in the partially activated state have phosphatidylserine on the surface.

When platelets become activated, activation triggers the release (e.g., exocytosis) of platelet granular contents and other metabolites into plasma (Sharda and Flaumenhaft (2018)/1000 Research 7:236). CD62 is normally stored in platelet granules and is externalized upon activation and emptying of platelet granules. Platelets can become activated upon storage at ambient or refrigerated temperatures. PS exposure and the release of α-granule contents during ambient temperature storage or prolonged storage follows the loss in mitochondrial membrane potential, cytoskeletal damage, and apoptosis due to depletion of glucose and lactate accumulation that occurs from continuous metabolic activity (Albanyan et al., (2009) Transfusion 49 (1): 108-17). Platelets that have emptied their alpha granules prior to administration for therapeutic purposes only are efficacious for a limited number of uses because the clinical benefits from platelets derive from maintenance of intact alpha granules at the time of administration.

PRP prepared from pooled human blood and stored frozen at −80° C. or freeze-dried and stored at room temperature (RT) was previously shown to retain the ability to be activated after storage; platelet counts remained constant after 8 weeks of storage in frozen and freeze-dried samples (Shiga et al. (2017) Asian Spine J 11 (3): 329-336). Freeze dried platelets were prepared by rotation in an ethanol bath for a preliminary membrane freeze at −60° C., freezing at −30° C., and subsequent freeze drying. Following activation by calcium chloride and thrombin, flow cytometry analysis indicated approximately 80% of the platelets were CD62P positive regardless of storage condition. Almost no growth factors were detected in the RT samples after 8 weeks, while low but statistically significant growth factor expression was detected in the frozen and freeze-dried PRP, although the actual amounts of growth factors was not assessed. Shiga et al. state that flow cytometry results revealed that platelets were activated and activation was maintained in freeze dried PRP. Prior to assessing platelet activity, activator(s) such as thrombin was added to the samples as an activator. Platelet morphology was not assessed.

### 2. Platelet Storage-Refrigerated Storage Problems

More than fifty years ago, it was recognized that platelets stored in refrigerated temperatures were quickly cleared from the circulation and room-temperature stored platelets had significantly longer circulation time. The undesirable affects associated with cold storage, and the desire for clinically effective platelets to be hemostatically functional and survive in circulation for several days resulted in room-temperature storage as the art-recognized storage method.

Platelets for therapeutic administration generally are stored at room temperature. Platelet collections as dictated by the blood-banking community for at least the past 20 years employ a room-temperature only protocol.

Contrary to the results shown herein and the methods provided herein, it had been the understanding in the art that, if the cells were incubated at ambient temperatures and subjected to decreased temperatures prior to and during lyophilization, the platelet cells would rupture. When platelets are prepared for freeze drying, cryopreservatives are added to platelet rich plasma compositions prior to lyophilization to maintain the integrity of the platelet membrane to prevent platelet rupture.

A problem with room temperature (RT) storage is that platelets stored at room temperature have increased bacterial growth compared to refrigerated samples; platelet transfusions carry greater risks of infection, sepsis, and death than any other blood product, primarily due to bacterial contamination. It also was previously shown that RT-stored platelets have a reduced ability to aggregate following ADP, collagen, and thrombin receptor activating peptide (TRAP) stimulation. Because fresh platelets have a shelf-life of only 3 to 5 days at room temperature (RT, 20-24° C.), methods or conditions for extending platelet shelf-life are needed.

Platelet cells previously have been stored for extended time periods at cold temperatures, or treated at cold temperatures and freeze dried for later reconstitution and use (i.e., administration); however, cold storage or components of the storage medium in which the cells are stored have negative effects on the platelets. Previous methods of platelet preparation that employ cold storage and/or cryopreservation have presented a challenge as cold storage, cryopreservation and/or lyopreservation can effect biochemical, structural, and functional changes to the platelet, such as, for example, platelet storage lesion. Cold stored platelets, such as platelets stored at refrigerated temperatures, generally are not used for therapeutics in a general hospital setting.

Refrigeration of platelets could be an alternative to the standard of RT storage. Refrigeration offers advantages over 22° C. storage with respect to metabolic function, contamination, and stability and shelf life. Storage of platelets at refrigerated temperatures, such as at or about 1-6° C., can prolong storage times by reducing the bacterial contamination. Cold storage also can preserve cell function, slow cellular metabolism, and decrease decline in platelet hemostatic function that occurs with room temperature storage. Cold stored platelets also can be used in actively bleeding patients, because cold-stored platelets can support hemostasis at the injury site more effectively than room temperature-stored platelets (Apelseth et. al. (2017) ISBT Sci Ser. 12:488-495).

For example, cryopreservation and/or lyopreservation can cause platelet storage lesion, which is a general term for storage-related platelet deterioration. Platelet storage lesions comprise a series of biochemical, structural, and functional changes that occur after collection and result in deleterious changes in platelet structure and function. Platelet storage lesions are associated with decreased platelet recovery and survival in vivo, and decreased hemostatic activity after transfusion. Platelets that undergo a storage lesion are readily cleared from the circulation by the spleen following transfusion and, thus, are not readily used for systemic administration. Platelet preparation and storage conditions that decrease platelet storage lesion are desired.

Refrigerated storage also can result in problems associated with platelet morphology. Extended storage at cold temperatures results in platelets that undergo morphological changes; platelets undergo a change from discoid shape to a spherical configuration after refrigerated storage; platelet morphology changes from a typical disc-shaped platelet to a spherical-shaped platelet with cryopreservation, or to a filamentous shape with lyophilization. Shape changes can result in biological dysfunction.

Long-term refrigerated storage also can result in platelet adhesion and aggregate formation. For example, platelets can spontaneously aggregate, and can fail to recover functional activity after incubating or storing platelets at refrigerated temperatures. Incubating or storing platelets at refrigerated temperatures also can result in an increase in the mean platelet volume (MPV). Increased MPV can indicate increased platelet activation or increased numbers of large, hyperaggregable platelets. During storage, there also is can be an accumulation of immunoactive proteins such as leukocyte-derive cytokines (tumor necrosis factor α, interleukin (IL) la, IL-6, IL-8) and soluble CD40 ligand which can be involved in transfusion-related injury and nonhemolytic transfusion reactions.

Full platelet activation during storage is undesirable. Platelets can be activated following exposure to foreign surfaces, trauma, low pH, agonists (thrombin, ADP), and shear stress. Cryopreserved and lyophilized platelets also can show characteristics of activated platelets, including loss of their discoid morphology and an increased number of pseudopods, an irregular ruffled cell surface, increased permeability to the cytoplasmic membrane, extracellular vesicle formation, and phosphatidylserine translocation from the inner to the outer leaflet of the plasma membrane. Platelet surface receptors also can be expressed differently after cryopreservation with an increased expression of P-selectin, and/or decrease in GPIba expression compared to platelets stored at room temperature (RT, at or about 22-25° C.).

Prolonged refrigerated storage also can result in platelet activation and release of granule contents and cell lysis. Platelet activation stimulates release of granular contents and expression of sequestered membrane proteins on outer surface. Release of granule contents by platelets at an injury site is necessary for platelet function. Early release of platelet granule contents during storage, prior to therapeutic administration, negates therapeutic efficacy of administered platelets; the granule contents are not available for platelet functions, such as, for example, leukocyte and platelet recruitment, promoting clotting and wound healing, and providing immunity against infection. Also, granule contents released during storage, and present in the storage medium, can cause transfusion reactions upon clinical administration. Full platelet activation during storage is undesirable and can negate the therapeutic benefits of platelets, such as the therapeutic benefits of release of the granule contents at the injury site. Platelets that retain the ability to activate upon removal from storage can be used for therapeutic purposes, such as regenerative therapies and for wound healing. Activation upon administration results in release of granule contents for therapeutic uses.

### 3. Platelet Storage-Freezing and Freeze-Drying Problems

Freezing is a common method of platelet cold storage; however, storage at freezing temperatures can cause cold-shock damage to the cells. In the absence of cryoprotectants and/or lyoprotectants, frozen cells can be destroyed after thawing; adding cryoprotective and/or lyoprotectants substances can maintain cellular integrity. Various methods of cryopreservation and lyophilization, and the inclusion of cryoprotectants are used for cryopreservation and lyophilization to preserve platelets to maintain their quality and effectiveness (i.e., activation state, morphology, and recovery). Previous methods for storing platelets at below freezing temperatures, such as freezing followed by or including lyophilization, are understood in the art to require cryopreservatives for preserving platelet structure and function.

Previous methods for preparing platelets include cryoprotecting the platelets for cold storage (see U.S. Pat. Nos. 5,622,867; 5,656,498; 5,736,313; 5,958,670; 5,800,978, 6,723,497; 6,833,236; 8,486,617). Cryoprotection can involve various methods and/or cryoprotectants. Previously, when platelets are stored at cold temperatures cryoprotectants such as one or more of DMSO, a carbohydrate, saccharide, maltodextrin, dextran, trehalose or glucose, or other polymers or mixtures of polymers or hydroxyethyl starch, are added prior to incubation in the cold (i.e., at or below freezing temperatures). In some examples, a cryoprotectant can be added while the platelets are incubated at ambient temperatures, such as, for example, while the platelets are incubated at ambient temperatures prior to freezing or freeze drying. In some examples, a cryoprotectant can be added while the platelets are incubated at ambient temperatures and then a second cryoprotectant can be added prior to lyophilization. For example, platelets can be incubated in the presence of at least one saccharide at ambient temperatures and then a cryoprotectant can be added, followed by freeze drying. In other examples, any of the cryoprotectants or other cryoprotectants known in the art, can be combined with an amphiphilic agent (i.e., arbutin). In one example, platelets in plasma are treated with saccharide, a biopolymer, an acid, or an acid salt prior to cold treatment and freeze drying (see e.g., U.S. Pat. No. 5,656,498). In other examples, platelets are incubated in a phosphate buffer (e.g., phosphate citrate buffer) which contains a carbohydrate. Previous lyophilized platelet compositions require addition of one or more cryoprotectants to the platelet composition prior to cold storage, such as freeze drying. Cryopreservatives, such as DMSO, can negatively impact cellular viability or other platelet properties, or can have negative consequences upon platelet administration.

The methods and resulting platelets and PRP compositions provided herein solve these problems. The cryopreservative-free compositions provided herein maintain a high number of platelets, maintain the functional integrity of platelets, retain functional activity, and do not cause toxicity when administered in vivo. The results herein show that freeze-dried platelet preparations, which are prepared without cryopreservatives by the methods described herein, retain a high number of platelets after freeze-drying and reconstitution; the platelets do not sustain cold-induced damage and maintain their cellular integrity. The platelets prepared in accord with the methods provided herein are, as shown herein, in a primed state; they are not in the completely activated state following cold treatment and preservation, and do not contain any toxic cryoprotectant that can cause adverse effects upon administration. Platelet preparation and treatment as provided herein avoids negative cellular changes and activation associated with previous methods of cold storage and preparation.

### 4. Commercially Available Platelet Products

Platelet-containing products, including lyophilized platelet products, are commercially available. Commercially available lyophilized platelets are prepared with a cryopreservative, such as DMSO or trehalose. Paraformaldehyde treatment also has been used to treat platelets prior to lyophilization; development of a product using paraformaldehyde treatment was suspended. Exemplary commercially available platelets are stored in an activated state. Commercially available freeze-dried PRP compositions have about 85% activated platelets in the compositions. Previously described, commercially available PRP compositions containing activated platelets generally are useful for administration at or near the site or injury or site for use. For example, commercially available products containing activated platelets are useful for administration, such as topical administration, on or near the site of a wound, for example, for wound healing.

For example, a platelet-derived freeze-dried hemostatic agent for treating humans is commercially available under the product name Thrombosomes® (from Cellphire). Application of the Thrombosomes® platelet product inhibits bleeding by aggregating at the bleeding site and triggering the hemostatic clotting cascade, while recruiting circulating platelets. The Thrombosomes® platelet product provides long-term storage of stable platelets (up to 3 years). The Thrombosomes® platelet product contains the cryopreservative trehalose. The Thrombosomes® platelet product is produced by pooling cells from many donors and stabilizing the platelets with trehalose and polysucrose prior to and during lyophilization, to produce, at scale, a safe and consistent, pathogen-reduced product. Platelets in the Thrombosomes® product are in an activated state when assessed by expression of CD62P, CD41, and phosphatidylserine (see e.g., U.S. Pat. No. 11,529,587). About 95% of the platelets in the Thrombosomes® platelet product have externalized phosphatidylserine based on annexin V staining, and an average of about 80-91% of platelets or platelet derivatives are in the activated states based on CD62 and CD41 positivity.

Due to their activated state, upon administration of the Thrombosomes® product the platelets are stable for up to one passage through the circulation and then are cleared by the body. Because they are in an activated state, the platelets in the Thrombosomes® product are most widely used for injection at a site of injury for immediate action, such as for cytokine release at the injection site, and are not used for therapies that require extended action or duration of activity.

A lyophilized derivative of canine platelets is available under the tradename StablePlate RX™ and sold by BodeVet (see U.S. Pat. Nos. 8,486,617 and 7,811,558). Leukoreduced canine platelets for inclusion in the StablePlate RX™ platelet product are collected by apheresis or whole blood donation, and the final product containing canine platelets is provided in a buffer that contains HEPES, NaCl, KCl, NaHCO₃, dextrose, trehalose, ethanol, and polysucrose. The StablePlate RX™ platelet product contains the cryopreservative trehalose. The StablePlate RX™ product is used to treat acute uncontrolled hemorrhage in bleeding dogs secondary to thrombocytopenia. The lyophilized StablePlate RX™ product is resuspended in water prior to injection into the animals.

The majority of platelets in the lyophilized and reconstituted StablePlate RX™ product are CD62 positive (BD Pharmingen anti-human CD62P antibody); the majority of platelets in the StablePlate RX™ product are in the activated state. Upon visual inspection, the reconstituted freeze-dried platelets showed swirled platelets (U.S. Pat. No. 8,486,617). As detailed herein, swirling can be used as a metric of platelet shape, but does not consistently evaluate activation status. Canine platelets in the StablePlate RX™ product showed activation in all other assays, and likely showed swirling due to the presence of the cryopreservative trehalose/polysaccharide/polysucrose in the platelet composition, and swirling does not indicate inactivated platelets therein. The Platelets in the activated state, such as platelets in the StablePlateRX product, are for use as a hemostat, for promoting clotting or decreasing the time to clot, or for wound healing.

A frozen cryopreserved platelet (CPP) product from Cellphire Therapeutics contains frozen platelets stabilized with DMSO. The CPP product can be stored for up to 5 years. The CPP product is thawed prior to use and administered. The frozen CPP contains the cryopreservative DMSO.

### 5. Cryopreservatives Used in Prior Art Platelet Preparation and Storage

The PRP compositions provided herein do not contain cryopreservatives and/or cryoprotectants. No cryopreservatives/cryoprotectants are added to the PRP compositions provided herein prior to storage and/or freeze drying. Cryoprotectants/cryopreservatives are added to compositions to prevent cold-induced damage to the biological products in the composition. To prevent or decrease the morphological changes and activation previously associated with cold storage, and to protect the integrity of the cells from mechanical injury during the storage procedure, various agents, such as cryopreservatives or cryoprotectants, have been added to previously prepared platelet compositions for storage. Cryoprotectants were previously thought to be necessary in platelet preparations to protect the platelets, for example, by coating the platelet surface to protect the cell, by incorporating into the lipid bilayer to maintain the integrity of the lipid bilayer, by decreasing the freezing point of the platelet composition to which the cryopreservative is added, and/or protect the platelets from crystallization. As used herein a cryopreservative is not an excipient and cryopreservatives do not include anti-coagulants, such as anti-coagulants added during blood collection or apheresis.

Cryoprotectants, lyoprotectants, and combinations of two or more cryo- and/or lyoprotective agents have previously been used in platelet preparation and storage, including, but not limited to, dimethyl sulfoxide (DMSO), carbohydrates and polysaccharides and sugars (e.g., trehalose, glucose, glycerol, maltodextrin, dextran, and hydroxyethyl starch (HES)), and formaldehydes (e.g., formaldehyde, paraformaldehyde, glutaraldehyde, and permanganate) for preserving and/or fixing platelets.

### Dimethyl Sulfoxide

In order to maintain platelet integrity, platelets have previously been treated with a cryopreservatives such as DMSO prior to freeze-drying. Dimethyl sulfoxide (DMSO) is a commonly used cryoprotectant. Previously, DMSO generally is used at about 2% to 10% of the composition, and also can be used in combination with other cryoprotectants such as carbohydrates (e.g., dextrose) and second messenger effectors (e.g., ThromboSol® cryoprotectant cocktail containing DMSO; composol; and epinephrine). ThromboSol® platelet storage medium from LifeCell Corporation (The Woodlands, Tex) contains 2% DMSO (see e.g., U.S. Pat. No. 5,622,867). Platelet loss of up to 50% of DMSO-cryopreserved platelets has previously been reported (Baythoon et al., (1980) J Clin Path 35 (8): 870-874; Valeri et al. (1974) Blood 43:131-136). DMSO also can have adverse effects on the subject after administration; platelets prepared in DMSO generally are washed prior to administration to wash out the DMSO from the preparation, to avoid adverse effects. For example, a combination of DMSO, maltodextrin, dextran, hydroxyethyl starch (HES), and glucose was previously used to preserve and store platelets maintained at low temperature (U.S. Pat. No. 5,622,867); the combination must be washed out prior to use due to its toxic effects and potential to cause adverse events related to the cryo- and lyo-protective agents. Standard protocols for preserving platelets comprise storage in 5-6% DMSO or 2% DMSO with ThromboSol®, and require washing out the cryoprotectant prior to platelet transfusion.

Platelet cryopreservation in the presence of cryopreservatives such as DMSO can have detrimental effects on the subjects to whom the platelets are administered. For example, studies report related adverse events related to the cryoprotectant, DMSO, including nausea, vomiting, local vasospasm, bradycardia, back and/or abdominal pain, headache, hypertension, and chest pain; and also cardiac, neurological, skin, respiratory, renal, reproductive system, or vascular system disorders or reactions, or allergic reactions, or sepsis. In some examples, to avoid adverse effects of DMSO, platelets are washed in water or saline in order to remove the cryopreservative, prior to administration to a subject. For example, DMSO can be washed and then centrifuged to remove the supernatant prior to administration; these additional manipulations are not practical in situations that require a quick supply of platelets.

### Trehalose

Trehalose, a non-reducing disaccharide made of two glucose molecules, was previously used as a cryoprotectant and lyoprotectant to preserve phospholipids, proteins, and cells from damage, such as, by stabilizing platelet membranes by forming hydrogen bonds with polar residues in phospholipids and proteins and preventing solid-liquid transition phase of the platelet membrane and/or by forming an amorphous glassy matrix during cryopreservation. Subjects administered platelets stored in trehalose reportedly have fewer adverse side effects or toxic effects compared to those administered platelets prepared and/or stored in DMSO (Crowe et al., (2001) Cryobiology 43 (2): 89-105). Trehalose was previously used at a concentration of at or about 10 to 300 mM, or at or about 45-150 mM for preparing and/or storing platelets (see U.S. Pat. Nos. 7,169,606 and 6,221,575; Nie et al., (2005) Biotechnology and Bioengineering 92 (1): 79-90), or 5-150 mM (U.S. Pat. No. 6,221,575). Trehalose also has previously been used in combination with one or more additional protective agents. Preservation with trehalose alone requires a high concentration and can be costly. Platelets prepared with trehalose as the lone cryoprotectant are not storage stable for extended periods of time especially in humid temperatures and/or super ambient temperatures, and as a result, can lose their activity in hours or days depending on the humidity and temperature of the storage condition. Therefore, freeze-drying with trehalose has its limitations for extended term storage of biological materials because the material will degrade and not have sufficient activity upon reconstitution. Additionally, trehalose with polymeric gelling agents cannot be used for medical purposes because the polymer gelling agents are not accepted by the human body, so trehalose with polymeric gelling agents are not practical.

### Other Cryopreservatives and Combinations

Other methods of preserving platelets include a combination of cryo- and lyo-protective agents in the preparation and/or storage medium. Extracellular cryoprotectants including hydroxyethyl starch (HES) and dextran were found to be poor cryopreservatives, and resulted in low platelet yields and poor in vitro viability compared to those cryopreserved in DMSO (Taylor (1981) J Clin Pathol. 34 (1): 71-5.) Combinations of glycerol and glucose also were previously used as cryoprotectants (Balduini et al., (2003) Haematologica 78 (2): 101-104; Kotelba-Witkowska and Schiffer (1982) Transfusion 22 (2): 121-124). Use of glycerol-glucose in platelet preparation reduced the ability of platelets to release ATP, aggregated platelets in response to a single agonist, increased cytoplasmic Ca²⁺ in response to thrombin; however, platelets sustained damage and showed defects in platelet function following freezing and thawing (Balduini et al., (2003) Haematologica 78 (2): 101-104; Kotelba-Witkowska and Schiffer (1982) Transfusion 22 (2): 121-124.)

Formaldehydes have also been used to preserve platelets. Examples of formaldehydes for preserving platelets include formaldehydes, paraformaldehyde, glutaraldehyde, and formalin used singly or in combination with other formaldehydes and/or permanganate. Low doses of formaldehyde in humans and goats show activated platelets and increased primary hemostasis (Ali and Abdus (1973) Indian Vet J 50:27-31; Pfueller et al. (1978) Thromb Haemosta 39:546-548). High doses of formaldehyde (0.74%) in healthy horses resulted in adverse effects including muscle fasciculations, tachycardia, tachypnea, serous ocular and nasal discharge, agitation, and restlessness (Taylor et al., (2000) American J Veterinary 61 (10): 1191-1196). Platelets prepared with formaldehydes must be washed prior to administration due to the toxic properties of formaldehydes. Administration of platelets prepared with formaldehydes can be toxic to the recipient if the formaldehydes are not completely removed prior to administration.

### D. PLATELET COLLECTION AND ISOLATION

Platelets and plasma for inclusion in the PRP compositions herein can be collected by any methods known in the art and as described herein. Platelets and plasma generally are collected from multiple donors and pooled. Pooling of blood products, such as platelets and/or plasma, from multiple donors is common in human and veterinary medicine. Individual platelet donors can vary widely in platelet count, the concentration of growth factors, including, PDGF-BB, VEGF, in the platelets (e.g., granules) and the activation potential of the platelets. Pooling platelets can yield a more uniform functional profile. According to the FDA Center for Biologics Evaluation and Research (CBER) whole blood-derived PRP from multiple donors can be combined to provide the high volumes that can be required for therapeutic administration. For example, pooled platelets, pooled plasma, and pooled PRP, such as pooled platelets, plasma and PRP from multiple donors can be used for therapeutic uses.

As provided herein, platelets suspended in a small amount of plasma are collected from multiple donors under sterile conditions to reduce pathogen contamination. Platelets are collected under conditions and using methods to maximize collection of intact platelets while decreasing the number of contaminants (i.e., pathogens) and, in some examples, other cell types (i.e., red blood cells, white blood cells) in the composition. In examples of a PRP composition herein where the platelets and plasma are collected from multiple donors, platelets in the PRP composition are in a primed state, where the platelets are not completely activated, and remain in a primed state upon reconstitution following lyophilization.

### 1. Donors

PRP has been studied in multiple species with varying use profiles. PRP can be used as a topical, intra-articular and/or intralesional therapy in various species. In some examples herein, the final PRP composition is species-specific and pooled from multiple donors. Exemplary PRP compositions, such as equine compositions, are formulated using “universal donors” to minimize incompatibility. “Universal donors” have a compatible blood type to most domestic horses. In some examples, donors are horses that are positive for Aa, Ca and Qa antigens and negative for plasma antibody to know equine erythrocyte antigens. Pooling of product with 5-32 donors allows for uniformity in platelet response and growth factor/chemokine release.

Platelets in a PRP composition herein can be from any animal source. In some examples the platelets are from a non-human animal, such as a horse, dog, pig, cow, sheep, goat, rabbit, rat, mouse, monkey, or cat. In other examples the platelets are isolated from a human. In some examples the platelets are collected from one animal, or the platelets can be collected from multiple individual animals and the platelets from the different individual animals are combined into one sample that contains the platelets from the multiple animals. For example, platelets can be provided as a mixture from two or more sources (i.e., two or more individuals), such as a mixture of two or more units of blood obtained from two or more blood donors to a public blood bank.

In general, the platelets and PRP compositions, which contain pooled platelets from a plurality of animals, such as horses or humans, are for allogeneic use. Platelets and PRP compositions can be for autologous use where the platelets are collected from the intended recipient. In such instances, the platelets and PRP necessarily do not contain pooled platelets. In such examples, where the platelets are intended to be used at a later date for infusion back into the donor, the platelets are autologous platelets for the purposes of the compositions, methods and provided herein. For example, the platelets can be originally obtained from the ultimate recipient of the freeze-dried platelets or reconstituted platelets. For example, the platelets can be collected from a particular donor by a known method in the art, including apheresis, and are incubated at refrigerated temperatures prior to lyophilization, mixed with plasma, generally from the donor, and subjected to lyophilization without adding or including a cryopreservative. Following lyophilization the platelets can be stored, such as placed in long term storage, including for a year up to several years. The lyophilized PRP for autologous use can be reconstituted, such as reconstituted in water or saline, and returned to the donor, for example, for therapeutic uses, including any described herein

a. Pathogen Reduction

Infusion of allogeneic cells, proteins, or blood components can cause immune-mediated reactions. Blood products for administration can contain blood-borne pathogens which can transmit to the recipient of the infusion. Blood products such as platelets and plasma can be treated prior to administration to decrease pathogen transmission to recipients. Donors also can be selected based on selection criteria, such as screening criteria described herein, to decrease pathogen transmission to recipients.

Prior to infusion of allogeneic cells, proteins, or blood components, donors can be screened and/or tested for infectious agents, pathogens, and/or other factors that can cause adverse reactions in the recipient. The presence of any of variety of infectious agents, pathogens, and/or other factors can be assessed (see e.g., Wardrop et al., (2016) J Vet Intern Med 30:15-35). Pathogens can be species-dependent. For example, pathogens can infect one species preferentially. In such examples, screening can be dictated by the donor species and infectious or pathogenic agents or genetic disorders that are known to infect or occur in the donor species. For example, donors can be screened for pathogens if it is known that pathogens are present in the donor species. Any assay known in the art can be used for pathogen screening, such as, for example, PCR or ELISA or molecular or biochemical assays.

Donor screening also can include assessment of physical characteristics, for example, by visual physical examination. In some examples, screening for blood borne pathogens can be supplemented by physical examination. In some examples, the animals are assessed for physical fitness. For example, animals can be assessed for heat by manual detection, joint effusion, flexion pain, general health, and appetite. In some examples, temperature (e.g., body temperature in ° C.), pulse, respiration (i.e., beats/minute), CBC, flexion pain, effusion, lameness, or other assessments, such as those that are particular for the disease, disorder or condition being treated or prevented.

Prior to platelet, plasma and/or whole blood collection for platelet collection, donors can be screened for pathogenic agents, such as infectious agents, to prevent introduction of the pathogenic agents into the PRP composition and subsequent introduction to the recipients of the PRP composition. Donors can be assessed for the presence of diseases or conditions or infectious agents, such as microorganisms. Assessments can be conducted according to methods known to those skilled in the art, such as the skilled veterinarian or physician. The donors can be screened for any disease or condition or infectious agent prior to blood and/or platelet collection to determine if the donor has a disease or condition or the presence of an infectious agent or microorganism. Potential donors that are positive for a pathogenic agent, or disease or condition can be excluded as a donor.

In examples, the presence of a disease or condition or infectious agent will preclude collection from the donor. Donors that are negative by PCR and/or ELISA for the tested infectious agents or diseases are selected for whole blood or plasma or platelet collection. In some examples the donors are screened by PCR and/or ELISA and/or other molecular or biochemical assays for the presence of disease (e.g., infectious disease, or infectious or pathogenic agent). In some examples donors that are screened by PCR and/or ELISA and/or other molecular or biochemical assays for the presence of disease also are screened by physical examination for signs or symptoms of the disease or condition or pathogen or infectious agent. In examples, a positive result from screening for the presence of a disease or condition or infectious agent, such as by PCR and/or ELISA and/or other molecular or biochemical assays or by physical examination will preclude collection from the donor.

In some examples, donor eligibility for donation of animal cells, tissues, and cell- and tissue-based products (ACTPs), such as blood products containing platelets, or isolated platelets, is in compliance with governmental or regulatory guidelines, such as regulations set forth by the U.S. Food and Drug Administration. For example, infectious disease evaluation can be conducted in compliance with governmental or regulatory standards or guidelines. In some examples, infectious disease evaluation can be in compliance with standards set forth by the U.S. Food and Drug Administration (FDA). For example, infectious disease evaluation can be in compliance with FDA Guidance #254 (see, e.g., Government Furnished Information (GFI) #254 Donor Eligibility for Animal Cells, Tissues, and Cell- and Tissue-Based Products, updated October 2022).

ACTPs, such as platelets that are intended for use for the treatment or prevention of disease or for any therapeutic purpose, can be manufactured in accordance with Current Good Manufacturing Practices (CGMPs) to ensure that products meet the safety requirements of the Federal Food, Drug, and Cosmetic Act (FD&C Act) and have the properties they purport to possess. Donors can be evaluated for risk factors for, or clinical or physical factors of, or test results indicating the presence of a disease or pathogen or infection. Donors can be selected if it is demonstrated that the donor is free from risk factors for, and clinical evidence of, infection with relevant disease agents and diseases, and/or tests for a pathogenic agent are negative and/or nonreactive.

In some examples, donors, such as equine donors, can be assessed for the presence of diseases or conditions or infectious agents, such as microorganisms. For example, equine donors can be screened for one or more of: equine infectious anemia (EIA), equine viral arteritis (EVA), Dourine, Brucellosis, Piroplasmosis, Glanders, and Equine Parvovirus-hepatitis Virus (EqPV-H). In examples herein, donor animals can be excluded if there is evidence of for the presence of diseases or conditions or infectious agents, such as microorganisms.

b. Sample Pooling

A concern about platelet effectiveness is associated with a lack of uniformity and standardization (Everts et al., (2022) Biomedicines 10 (11): 2933; Garbin et al., (2021) BMC Vet Res 18 (1): 141). Pooling can address this concern. Sample pooling also can increase the amount or number of platelets and/or plasma that can be combined and, thus, can increase the number of or volume of platelets for administration. Studies in human medicine demonstrate the use of pooled PRP without negative effects on immunogenicity. Commercially available PRP products contain pooled platelets (e.g., StablePlate RX® Canine PRP) and are used without significant adverse events (see e.g., Goggs et al., (2020) J Vet Intern Med 34 (6): 2384-2397). Canine and equine transfusion medicine routinely use multiple units of whole blood, red blood cells, plasma and platelet concentrates collected from multiple donors for treatment of human patients, such as in trauma and surgical settings (see Jutkowitz et al., (2002). J Am Vet Med Assoc. 220 (11): 1664-9). Standardization of the components and production process of PRP can decrease variability among samples. Pooling platelets yields a more uniform function profile, as individual platelet donors vary widely in count, growth factor concentration, and activation potential. (Alhumaidan, 2011 #71).

Pooling of blood products, such as PRP, is common practice in human and veterinary medicine. Pooled samples can offer numerous advantages compared to samples collected from a single donor. Pooling of samples can balance inherent donor variation. For example, pooling of samples can balance inherent donor variation in production of platelet-derived growth factors and cytokines. In examples herein, apheresis-derived platelet concentrate is prepared from multiple donors and pooled. In examples herein, plasma is collected from multiple donors and the plasma is pooled into one or are pooled to control the final product platelet count and provide uniformity in the PRP product. The pooled platelets (i.e., pooled platelet concentrates) are combined with pooled plasma from multiple donors to prepare a pooled PRP composition with increased uniformity compared to platelets and plasma from a single donor.

In examples herein, platelets can be collected from multiple donors. For example, platelets can be collected from 2, 3, 4, 5, 6, 7, 8, 9, 10, 11, 12, 13, 14, 15 or more donors and the platelets from each donor can be pooled with platelets from the other donor(s). In some examples, platelets are collected from at least 6, 7, 8, 9, 10, 11 or 12 donors. The number of donors from which the platelets are pooled can be determined by the skilled artisan, such as the skilled veterinarian. The number of donors can vary depending on various factors, including the number of platelets collected, platelet variability in the donors, desired uniformity in the product, and other factors.

c. Other Considerations

Other donor characteristics also can be considered when selecting platelet and/or plasma donors. For example, the blood type or platelet receptor type of the donor can be considered.

For example, for platelet and/or plasma collection from horses, the blood type of the horse can effect donor eligibility. In some examples of blood product collection from horses, the animals are erythrocyte antigen positive, such as A, C, and Q erythrocyte antigen negative, such as, for example, Aa and Ca erythrocyte antigen positive. They may be negative for plasma antibody to erythrocyte type that they are not. Animals can be selected based on a likelihood, such as a decreased likelihood, to produce a reaction when the platelets and/or plasma, such as a PRP composition, are administered into a recipient animal.

In examples herein, the donor animals (horses) are A, C, and Q erythrocyte antigen negative, such as, for example, Aa and Ca erythrocyte antigen negative. Donors also were negative for plasma antibody to erythrocyte antigen.

Human PRP can be blood type and platelet receptor type specific for the recipient. In examples, donors can be low titer individuals for erythrocyte antibody to antigen that they do not possess. In the case of the human, blood type specific as well platelet receptor type platelets are used.

Donor animals also can be pre-screened by standard complete blood count (CBC) for hematocrit and platelet count and chemistry profile. Donors that show a normal hematocrit and platelet levels for their species are selected. It is known that the hematocrit and platelet count and chemistry profile can differ based on the species of the donors. It also is known that the hematocrit and platelet count and chemistry profile can differ based on the sex of the donors.

For example, for humans, donors with a hematocrit percentage greater than at or about 35%, 36%, 37%, 38%, 39%, 40%, 41%, 42%, 43%, 44%, 45%, 46%, 47%, 48%, 49%, 50%, 51%, 52%, 53%, 54%, or 55% are selected. In other examples, human donors with a platelet count in whole blood greater than at or about 140,000/μL, 150,000/μL, 160,000/μL, 170,000/μL, 180,000/μL, 190,000/μL, 200,000/μL, 210,000/μL, 220,000/μL, 230,000/μL, 240,000/μL, 250,000/μL, 260,000/μL, 270,000/μL, 280,000/μL, 290,000/μL, 300,000/μL, 310,000/μL, 320,000/UL, 330,000/μL, 340,000/μL, 350,000/μL, 360,000/μL, 370,000/μL, 380,000/μL, 390,000/L, 400,000/μL, 410,000/μL, 420,000/μL, 430,000/μL, 440,000/μL, 450,000/μL, 460,000/μL, 470,000/μL, 480,000/μL, 490,000/μL, 500,000/μL or more. Exemplary donors that have a hematocrit percentage greater than at or about 36% and a platelet count greater than at or about 150,000/μL in whole blood are selected for automated plateletpheresis.

For example, for horses, animal donors with a hematocrit percentage greater than at or about 25%, 26%, 27%, 28%, 29%, 30%, 31%, 32%, 33%, 34% or 35% are selected. In other examples, horse donors with a platelet count in whole blood greater than at or about 75,000/μL, 80,000/μL, 85,000/μL, 90,000/μL, 95,000/μL, 100,000/μL, 105,000/μL, 110,000/μL, 115,000/μL, 120,000/μL, 125,000/μL, 130,000/μL, 135,000/μL, 140,000/μL, 145,000/μL, 150,000/μL, or more. Exemplary donors that have a hematocrit percentage greater than at or about 32% and a platelet count greater than at or about 130,000/μL in whole blood are selected for automated plateletpheresis.

### 2. Platelet Collection

Platelets in the PRP compositions provided herein can be collected using any method known in the art, including, for example, those listed and described herein. Platelets and plasma isolated for therapeutic uses generally are whole blood-derived or apheresis-derived. Apheresis is a method of automated collection of platelets and plasma where the red blood cells are returned to the donor. Apheresis has been used successfully in horses and dogs to collect platelet concentrate (Callan et al., (2009) J Vet Emerg Crit Care 19 (5): 401-15; Sutter et al., (2004) Am J Vet Res. 65 (7): 924-30). Most FDA-cleared apheresis devices (e.g., Haemonetics, Fresenius Kabi, Terumo) can be used to collect both platelets and plasma. Multiple FDA-cleared devices have been used in horses and dogs to produce autologous PRP for point of care use by clinicians in the treatment of musculoskeletal injuries and wound management (Brossi et al., (2015) BMC Vet Res. 11:98).

Platelets and plasma can be isolated from whole blood using any method known in the art. Platelets generally are isolated in plasma and then additional plasma is added to form a PRP composition, such as a PRP composition with primed platelets. In some examples, platelets (e.g., platelets in plasma) and plasma can be isolated from whole blood using methods such as, for example, the platelet-rich-plasma (PRP) technique, buffy coat (BC) method, or can be collected by apheresis. Plasma and platelets from donors generally are whole blood-derived (WBD) or apheresis-derived.

Devices used in the apheresis process have been developed to draw blood from the donor, centrifuge the blood for specific blood components of interest, and return the other blood components back to the donor in one course. Unlike the WBD methods of collecting platelets and plasma, collection of apheresis-derived platelets and plasma is automated by the medical device and any remaining components (e.g., red blood cells) are returned to the donor. The method of preparing platelet concentrates (i.e., apheresis, buffy coat, whole blood collection) can impact platelet storage lesions (Bock et al., (2002) Transfus Med. 12 (5): 317-24).

The total number of platelets collected can vary based on several parameters, including the size and type (i.e., species) of the donor, the collection time, collection efficiency, method of collection, and other parameters. The concentration of platelets can depend on the particular donor. The total number of platelets collected also can vary depending on the number of donors and manufacturing batch size.

Platelet concentrates can contain platelets from multiple donors. In examples of the PRP compositions herein, the recovered platelet concentrates containing platelets in plasma collected from a single donor are in volumes of less than about 1 L. For example, recovered platelet concentrates from a single donor are in volumes of less than about 1.0 L, 0.9 L, 0.8 L, 0.75 L, 0.7 L, 0.65 L, 0.6 L, 0.55 L, 0.5 L, 0.45 L, 0.4 L, 0.35 L, 0.3 L, 0.25 L, or 0.2 L or less, such as between at or about 200 mL and 1,000 mL, 200 mL and 800 mL, 200 mL and 600 mL, 200 mL and 500 mL, 200 mL and 300 mL, 200 mL and 400 mL, 400 mL and 1,000 mL, 400 mL and 800 mL, 400 mL and 600 mL, 400 mL and 500 mL, 600 mL and 1,000 mL, 600 mL and 800 mL, and 800 mL and 1,000 mL. In some examples herein the platelet concentrates from individual donors are in volumes of at or about between 200 mL and 500 mL, such as, for example, volumes of at least or at least about 200 mL, 210 mL, 220 mL, 230 mL, 240 mL, 250 mL, 260 mL, 270 mL, 280 mL, 290 mL, 300 mL, 310 mL, 320 mL, 330 mL, 340 mL, 350 mL, 360 mL, 370 mL, 380 mL, 390 mL, 400 mL, 410 mL, 420 mL, 430 mL, 440 mL, 450 mL, 460 mL, 470 mL, 480 mL, 490 mL, 500 mL, 510 mL, 520 mL, 530 mL, 540 mL, 550 mL, 560 mL, 570 mL, 580 mL, 590 mL, or 600 mL. For example, platelet concentrates from an individual donor can be between 200 mL and 500 mL prior to pooling and mixing with additional plasma.

In examples of the PRP compositions herein, the pooled platelet concentrates containing platelets in plasma collected from multiple donors are in volumes of greater than 2 L, such as greater than at or about 3 L, or 4 L. The total volume can vary depending on the number of animals from which the collected platelets in plasma are pooled. For example, platelet concentrates pooled from multiple donors are in volumes of at least or at least about 6 L, 5.75 L, 5.6 L, 5.5 L, 5.4 L, 5.3 L, 5.2 L, 5.1 L, 5 L, 4.75 L, 4.6 L, 4.5 L, 4.4 L, 4.3 L, 4.2 L, 4.1 L, 4 L, 3.75 L, 3.6 L, 3.5 L, 3.4 L, 3.3 L, 3.2 L, 3.1 L, 3 L, 2.75 L, 2.6 L, 2.5 L, 2.4 L, 2.3 L, 2.2 L, 2.1 L, 2 L, 1.75 L, 1.6 L, or 1.5 L, such as between at or about from between or between about 1.5 L and 6 L, 1.5 L and 5.75 L, 1.5 L and 5.5 L, 1.5 L and 5 L, 1.5 L and 4.75 L, 1.5 L and 4.5 L, 1.5 L and 4 L, 1.5 L and 3.75 L, 1.5 L and 3.5 L, 1.5 L and 3 L, 1.5 L and 2.75 L, 1.5 L and 2.5 L, 1.5 L and 2 L, 2.0 L and 6 L, 2.0 L and 5.75 L, 2.0 L and 5.5 L, 2.0 L and 5 L, 2.0 L and 4.75 L, 2.0 L and 4.5 L, 2.0 L and 4 L, 2.0 L and 3.75 L, 2.0 L and 3.5 L, 2.0 L and 3 L, 2.0 L and 2.75 L, 2.0 L and 2.5 L, 2.5 L and 6 L, 2.5 L and 5.75 L, 2.5 L and 5.5 L, 2.5 L and 5 L, 2.5 L and 4.75 L, 2.5 L and 4.5 L, 2.5 L and 4 L, 2.5 L and 3.75 L, 2.5 L and 3.5 L, 2.5 L and 3 L, 2.5 L and 2.75 L, 3.0 L and 6 L, 3.0 L and 5.75 L, 3.0 L and 5.5 L, 3.0 L and 5 L, 3.0 L and 4.75 L, 3.0 L and 4.5 L, 3.0 L and 4 L, 3.0 L and 3.75 L, 3.0 L and 3.5 L, 3.5 L and 6 L, 3.5 L and 5.75 L, 3.5 L and 5.5 L, 3.5 L and 5 L, 3.5 L and 4.75 L, 3.5 L and 4.5 L, 3.5 L and 4 L, 3.5 L and 3.75 L, 4.0 L and 6 L, 4.0 L and 5.75 L, 4.0 L and 5.5 L, 4.0 L and 5 L, 4.0 L and 4.75 L, 4.0 L and 4.5 L, 4.5 L and 6 L, 4.5 L and 5.75 L, 4.5 L and 5.5 L, 4.5 L and 5 L, 4.5 L and 4.75 L, 5.5 L and 6 L, and 5.5 L and 5.75 L. For example, platelet concentrates pooled from multiple donors can be in volumes of 3000 mL to 4600 mL, such as at or about 4600 mL, prior to mixing with additional plasma.

In examples of the PRP compositions herein, the platelet concentrates containing platelets in plasma are added to additional plasma to prepare the liquid PRP compositions. The total volume of the PRP compositions can vary depending on the number of animals from which the collected platelets in plasma are pooled and the concentration of platelets in the platelet concentrates. In some examples additional plasma is added to the platelet concentrates to prepare a PRP composition with a platelet concentration of between 200,000 platelets/μL and 1,000,000 platelets/μL, such as, for example at or about 400,000 platelets/μL=100,000 platelets/μL. In examples herein, PRP compositions herein contain a ratio of plasma volume to platelet volume of 1:1, 1.1:1, 1.2:1, 1.3:1, 1.4:1, 1.5:1, 1.6:1, 1.7:1, 1.8:1, 1.9:1, 2.1:1, 2.2:1, 2.3:1, 2.4:1, 2.5:1, 2.6:1, 2.7:1, 2.8:1, 2.9:1, 3:1, 3.1:1, 3.2:1, 3.3:1, 3.4:1, 3.5:1, 3.6:1, 3.7:1, 3.8:1, 3.9:1, 4:1, 4.1:1, 4.2:1, 4.3:1, 4.4:1, 4.5:1, 4.6:1, 4.7:1, 4.8:1, 4.9:1, 5:1, 5.1:1, 5.2:1, 5.3:1, 5.4:1, 5.5:1, 5.6:1, 5.7:1, 5.8:1, 5.9:1, 6:1, 6.1:1, 6.2:1, 6.3:1, 6.4:1, 6.5:1, 6.6:1, 6.7:1, 6.8:1, 6.9:1, 7:1, 7.1:1, 7.2:1, 7.3:1, 7.4:1, 7.5:1, 7.6:1, 7.7:1, 7.8:1, 7.9:1, 8:1, 8.1:1, 8.2:1, 8.3:1, 8.4:1, 8.5:1, 8.6:1, 8.7:1, 8.8:1, 8.9:1, 9:1, 9.1:1, 9.2:1, 9.3:1, 9.4:1, 9.5:1, 9.6:1, 9.7:1, 9.8:1, 9.9:1, or 10:1. For example, platelet concentrates can be combined with additional plasma at a ratio of plasma volume to platelet volume of 1:1, 1.1:1, 1.2:1, 1.3:1, 1.4:1, 1.5:1, 1.6:1, 1.7:1, 1.8:1, 1.9:1, 2.1:1, 2.2:1, 2.3:1, 2.4:1, 2.5:1, 2.6:1, 2.7:1, 2.8:1, 2.9:1, 3:1, 3.1:1, 3.2:1, 3.3:1, 3.4:1, 3.5:1, 3.6:1, 3.7:1, 3.8:1, 3.9:1, 4:1. In such examples, in a PRP composition containing platelets and plasma, the portion of the PRP composition, by volume, that is plasma is between about 50% and 80%.

In examples, the PRP composition comprising platelet concentrates (platelets in plasma) and additional plasma comprises total plasma in an amount between about 20% and about 90%, between about 20% and about 85%, between about 20% and about 80%, between 20% or about 20% and 75% or about 75%, between 20% or about 20% and 70% or about 70%, between 30% or about 30% and 90% or about 90%, between 30% or about 30% and 85% or about 85%, between 30% or about 30% and 80% or about 80%, between 30% or about 30% and 75% or about 75%, between 30% or about 30% and 70% or about 70%, between 40% or about 40% and 90% or about 90%, between 40% or about 40% and 85% or about 85%, between 40% or about 40% and 80% or about 80%, between 40% or about 40% and 75% or about 75%, between 40% or about 40% and 70% or about 70%, between 45% or about 45% and 90% or about 90%, between 45% or about 45% and 85% or about 85%, between 45% or about 45% and 80% or about 80%, between 45% or about 45% and 75% or about 75%, between 45% or about 45% and 70% or about 70%, between 50% or about 50% and 90% or about 90%, between 50% or about 50% and 85% or about 85%, between 50% or about 50% and 80% or about 80%, between 50% or about 50% and 75% or about 75%, between 50% or about 50% and 70% or about 70%, between 55% or about 55% and 90% or about 90%, between 55% or about 55% and 85% or about 85%, between 55% or about 55% and 80% or about 80%, between 55% or about 55% and 75% or about 75%, between 60% or about 60% and 90% or about 90%, between 60% or about 60% and 85% or about 85%, between 60% or about 60% and 80% or about 80%, between 60% or about 60% and 75% or about 75%, between 65% or about 65% and 90% or about 90%, between 65% or about 65% and 85% or about 85%, between 65% or about 65% and 80% or about 80%, by volume, of the composition. For example, the portion of the PRP composition, by volume, that is plasma is between about 50% and 80%, such as between 50% and 75%, between 60% and 75%, or the amount of plasma in the PRP composition is at least or at least about or about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% plasma by volume of the total volume of the PRP composition. In particular examples the about 75% of the volume is plasma and the remaining about 25% is platelets, with a small volume of cryopreservative in the composition.

### Leukoreduced or Leukorich Platelet Composition

The collected platelets in plasma can be processed prior to combination with the plasma composition. For example, the collected platelets in plasma can be filtered to remove cells or impurities. In some examples, the platelets concentrated containing platelets in plasma can be passed through a filter to remove cells, such as, for example, red blood cells or white blood cells, or impurities, such as, for example, pathogens such as bacteria.

The PRP composition can be formulated as leukoreduced composition. If a leukoreduced PRP composition is desired, the samples can be leukoreduced by any appropriate method, such as centrifugation or filtration following apheresis. For example, following platelet collection, for preparation of leukoreduced platelet concentrates (PCs), platelet compositions, such as pooled platelets from multiple donors (e.g., platelet concentrates) are subject to treatment to eliminate or decrease the number of white blood cells (WBCs; leukocytes) in the composition. The platelet concentrates can be passed through a filter to limit the number of white blood cells (WBCs) in the final product. In some examples, for preparation of leukoreduced platelet concentrates, platelet concentrates are passed through a leukoreduction filter, such as, for example, a leukoreduction filter produced by PALL® Corporation, Port Washington, New York to limit the number of white blood cells (WBCs) in the final product.

The normal number of WBCs in blood in humans is from 4,500 to 11,000 WBCs per microliter (4.5 to 11.0×10⁹/L). In examples of preparing a PRP composition, the platelet composition (e.g., pooled platelet concentrates) can be treated (i.e., passed through a filter) to remove the WBCs from the platelet concentrate composition so that the number of WBCs and/or concentration of WBCs in the platelet concentrates is less than the number of WBCs and/or concentration of WBCs in whole blood or in platelet concentrates that are not subjected to a leukoreduction treatment.

For example, the PRP composition can be prepared to have a concentration of leukocytes that is less than the level of leukocytes in whole blood. For example, the PRP composition can be treated, such as filtered so that there are less than 5000, less than 4500, less than 4000, less than 3500, less than 3000, less than 2500, less than 2000, less than 1900, less than 1800, less than 1700, less than 1600, less than 1500, less than 1400, less than 1350, less than 1300, less than 1250, less than 1200, less than 1050, less than 1000, less than 950, less than 900, less than 850, less than 800, less than 750, less than 700, or less leukocytes per μL of the PRP composition. In some examples, the final platelet concentrate composition is leukoreduced wherein the platelet concentrate composition contains less than 1200 white blood cells per microliter of the platelet concentrate composition (<1200 WBCs/μL).

In other examples, a PRP composition provided herein can be formulated as leukocyte rich composition. In some examples, at the time of apheresis the formulation can be varied to create a leukocyte rich PRP. For example, the PALL filter or other filter for leukoreduction is not used and the platelet-rich plasma does not have fewer white blood cells than whole blood, or has concentrated white blood cells compared to whole blood.

The PRP provided herein can be formulated as leukorich in accord with the desired application (e.g., therapeutic use). For example, the PRP composition can be prepared to have a concentration of leukocytes that is equal to or greater than the level of leukocytes in whole blood. For example, the PRP composition can contain at or greater than at or about 4000, greater than at or about 4500, greater than at or about 5000, greater than at or about 5500, greater than at or about 6000, greater than at or about 6500, greater than at or about at or about 7000, greater than at or about 7500, greater than at or about 8000, greater than at or about 8500, greater than at or about 9000, greater than at or about 9500, greater than at or about 10000, greater than at or about 10500, greater than at or about 11000, greater than at or about 11500, greater than at or about 12000, greater than at or about 12500, greater than at or about 13000, greater than at or about 13500, greater than at or about 14000, greater than at or about 14500, greater than at or about 15000, greater than at or about 15500, or more leukocytes per μL of the PRP composition.

Immediately after the apheresis collection process, platelets are placed in cold storage at 0° C.-8° C. until further assessment and/or treatment and/or pooling with other platelets and/or plasma.

a. Apheresis

Apheresis refers to a method for collecting plasma or plasma and platelets where blood from a donor is passed through a medical device to separate one or more particular components (e.g., platelets, red blood cells, leukocytes), collect the desired components, and return the remaining components to the donor. For example, the components can be separated and collected using centrifugation, membrane filtration, or selective adsorption. Following collection of the plasma or plasma and platelets, the remaining blood components, such as red blood cells, are returned to donor circulation. Platelet recovery by apheresis (plateletpheresis) is well-established and can be conducted using techniques, protocols and devices known in the art. In some examples, a commercially available collection system, such as an automated apheresis device can be used to separate or isolate platelets from the whole blood to recover platelet concentrate. Apheresis is the most common method of platelet isolation in the United States and Europe; 80% of platelets collected in the United States and 50% of the platelets collected in Europe are collected by apheresis methods.

i. Donor Considerations

Apheresis protocols can be developed taking into account characteristics of the donor. For example, a donor's total blood volume (TBV), total plasma volume (TPV), body surface area (BSA), extracorporeal volume (ECV), and collection efficiency (CE) can be assessed and used to prepare the apheresis protocol or procedure (Neyrinck et al., (2015) J Clin Apheresis 30:38-42). It is within the level of skill of the treating medical professional to adopt or design an apheresis protocol particular to the donor or taking into consideration donor characteristics. The skilled medical or scientific professional can use formulae and protocols known in the art, in conjunction with donor characteristics, to develop apheresis protocols.

For example, Nadler's formula can be used to calculate TBV of an adult human being with respect to gender, height, and weight. The formulae for calculating TBV in males and females are (0.3669*Height3)+(0.03219*Weight)+0.6041, and (0.3561*Height3)+(0.03308*Weight)+0.1833, respectively. In other examples, Gilcher's rule of five and the 1977 model by Linderkamp et al. can be used for predicting blood volume in children (Linderkamp et al., (1977) Eur J Pediatr 125:227-234). Other characteristics, such as donor age and body mass index (BMI) also can influence, and therefore be used to calculate blood volume. In humans, the ECV (e.g., volume of blood removed from the body during a procedure) generally does not exceed 15% of the TBV. TPV is related to TBV and is calculated using following formula: TPV=TBV*(1-Hct). Hematocrit (Hct) levels influence the plasma level and/or amount of fluid required to replace the collected plasma and procedure time. Collection efficiency impacts the amount of donor plasma or cells or platelets collected to achieve the desired amount in the final product. Donor hemoglobin and hematocrit levels also can be considered. Hemoglobin levels can be calculated from hematocrit levels using following formula: 3*the Hb (in g/dL), or [(Hb (in mmol/L)*10)/2]-2.

Apheresis procedures have been used successfully in horses and dogs to collect platelet concentrates (Callan (2009) Journal of Veterinary Emergency and Critical Care 19 (5): 401-415; Sutter (2004) American Journal of Veterinary Research 65:7; Sumer et al., (2017) Transfusion 57:1755-1762). Collection parameters and techniques can be modified based on differentiating characteristics of the particular animal, such as size, skin thickness, vein availability, total blood volume (TBV), total plasma volume (TPV), body surface area (BSA), extracorporeal volume (ECV), and collection efficiency. For example, needle size and needle access site in equine and canine animals have previously been modified as compared to humans to collect blood through a single jugular venipuncture.

Anti-coagulants can be added before or during apheresis to prevent clotting of the circuit. Anti-coagulants for use in apheresis procedures include citrate-based anti-coagulants such as Acid-Citrate-Dextrose Formula A (ACD-A) and ACD-B, and sodium citrate; and heparin. In some examples, citrate-based anti-coagulants can be added in an amount ranging from at or about 2% to 4%, including at least or at least about 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, and 4%. In some examples, heparin can be added before or during apheresis or to the composition. For example, heparin can be added to in an amount of at least or at least about 0.5 to 2.0 IU/mL, such as, for example, 0.5 IU/mL, 0.55 IU/mL, 0.6 IU/mL, 0.65 IU/mL, 0.7 IU/mL, 0.75 IU/mL, 0.8 IU/mL, 0.85 IU/mL, 0.9 IU/mL, 0.95 IU/mL, 1.0 IU/mL, 1.1 IU/mL, 1.2 IU/mL, 1.3 IU/mL, 1.4 IU/mL, 1.5 IU/mL, 1.6 IU/mL, 1.7 IU/mL, 1.8 IU/mL, 1.9 IU/mL, or 2.0 IU/mL.

ii. Apheresis Procedures

Apheresis starts with inserting a needle into the donor's arm, leg, or catheter and connecting the needle to medical tubing to transport the donor blood to the chamber of the apheresis device. After blood collection, blood components, such as plasma, platelets, leukocytes, and erythrocytes, or components found in the blood, such as proteins and/or toxins, can be removed from the blood by subsequent centrifugation, membrane filtration, and/or selective adsorption. In CFC, two needles are inserted in the donor.

An intermittent-flow centrifugation (IFC) blood cell separator or continuous flow centrifugation (CFC) can be used in the apheresis procedure. In IFC, the apheresis comprises interrupted cycles that include withdrawal of whole blood, separation of desired blood components (i.e., platelets) by centrifugation and processing for specific components, and subsequent reinfusion of the remaining (undesired) blood components through the primary venous port. Continuous flow centrifugation (CFC) differs from IFC in that it is an uninterrupted or continuous flow that includes withdrawal, separation, and reinfusion; and requires two venipunctures, one for collection and one for reinfusion. CFC takes less time than IFC and is more hemodynamically stable. Each cycle of IFC processes a small volume of blood, and as a result, the procedure is longer than CFC. There also are larger fluctuations in extracorporeal blood volume with IFC compared to CFC.

Centrifugation variables (e.g., speed, rotor size, cycle, and time), membrane material and pore size, and/or column material and pore size can be adjusted to remove the product of interest by utilizing physical, chemical, biochemical, biophysical qualities of the cell.

For example, blood components can be separated using centrifugal-based apheresis by varying centrifugal force, spin duration, and the number of centrifugal cycles. Increasing centrifugal force by centrifugation results in partitioning of blood components from heaviest (i.e., densest) at the bottom to lightest at the top; erythrocytes generally settle at the bottom, followed by leukocytes, platelets, and plasma as the top layer. For example, centrifugation speed, rotor size, cycle, and timing can be adjusted depending on the blood component to be isolated and physical qualities of the blood. For example, centrifugation parameters can be adjusted for isolating platelets from red blood cells, where red blood cells are larger and denser than platelets, and thus, red blood cells will settle at the bottom of the tube first and platelets will remain in the top layer. After selecting and filtering the components of interest, the other components are returned to the donor to reenter the donor circulation via a second needle inserted into the donor. The time for completion of apheresis procedures can vary based on, for example, how quickly blood filters through the apheresis device, and the amount of blood for processing to obtain the product of interest (e.g., platelets) in the amount of interest (e.g., concentrated platelets compared to whole blood).

In some examples, blood components are separated using centrifugal-based apheresis at different centrifugal force, such as, for example varying centrifugal force (e.g., rotational centrifugal force (RCF)) at speeds between at or about 1500 to 2700 rpm. As is known in the art, RCF is measured in multiples of the standard acceleration due to gravity at the Earth's surface (x g) and will vary depending on various factors, such as the size (e.g., diameter) of the rotor and the rotation speed. The spin duration can be at or about 3 minutes to 120 minutes, such as at least 3, 3.5, 4, 4.5, 5, 5.5, 6, 6.5, 7, 7.5, 8, 8.5, 9, 9.5, 10, 10.5, 11, 12, 13, 14, 15, 20, 25, 30, 40, 50, 70, 75, 80, 85, 90, 95, 100, 105, 110, 115, 120, or 125 minutes. In some examples herein the spin duration is at or about 3 to 10 minutes. The number of centrifugal cycles can be any as required to distinguish the different cell types in the sample can be 1 to 10 cycles, such as 1 to 3 cycles.

iii. Commercially Available Apheresis Devices

For preparation of the platelets in a PRP as provided herein, any apheresis device designed or modified for platelet collection can be used. For example, commercially available apheresis devices and FDA-cleared apheresis devices can be used to collect platelets and plasma. Apheresis previously has been used in various animal species, such as horses and dogs, to collect platelet concentrate (Callen et al., J Vet Intern Med. (2009) 23 (1): 138-45; Sutter et al., Am J Vet Res (2004) 65 (7): 924-30). Multiple FDA-cleared devices have been used in horses and dogs to produce autologous PRP for point of care use by clinicians in the treatment of musculoskeletal injuries and wound management (Brossi, 2015). FDA 510 (k) clearances have been issued for centrifuge-type therapeutic apheresis devices, including the Cryosan, the Spectra Optia® Apheresis System by Terumo BCT, the COBE Spectra® Blood Cell Separation System by COBE Laboratories, the Automated 8400 Blood Cell Separator (IBM), the Fresenius® AS 104 Cell Separator by Fresenius Hemocare Inc., the MCS® and MCS®+ Systems by Haemonetics® Corp., and the Amicus Separator System by Fenwal Inc.

Commercially available apheresis devices include the NexSys PCS® with persona technology by the Haemonetics® Corporation, the Rheocarna® membrane based apheresis modules and MA-03 Apheresis Machine by the Kaneka Corporation, the PLASAUTOE PlasutoE™ by the Asahi Kasei Corporation, the H.E.L.P.R. Plasmat® Futura apheresis system by B. Braun Avitum AG, the INTERCEPT™ Blood System by the Cerus Corporation, the COBE Spectra® Apheresis System and the COBE Trima® Apheresis System by Gambro BCT, the Spectra Optia Apheresis System with the Depuro D2000 Adsorption Cartridge by Cambro BCT, the Therapeutic Apheresis System by the Millipore Corporation, the AFERSMART™ and AFERSMART PLUS™ therapeutic apheresis machines by Medica, the AMICUS™ Thrombapheresis Machine by Fresenius Kabi, the PMC Plasmapheresis Machine by Zheng Yuan Technology Co Ltd., the ADAsorb® Therapeutic Apheresis System by Medicap, the TWIN6920 Benchtop Blood Component Extractor by Bristol Myers Squibb (BMS), the MAGELLAN® thomapheresis machine by Arteriocyte, the Luxomatic V2 automatic blood component extractor by LMB, the BIO45 automatic blood component extractor by Bioelettronica, the ALYZ™ erythropheresis machine by Fresenius Kabi, the TRIMA ACCEL® transfusion apheresis machine by TERUMO, the DX21 apheresis machine by Kaneka, the GIOTTO MONZA blood component extractor by Delcon, the LA25 leukapheresis machine by Medica, the Aurora Plasma Collection System and Alyx Apheresis Collection System from Fresenius Kabi, and the Fractiomatic® Plus 2 Automated Blood Component Separator from GRIFOLS.

Commercially available centrifuges can be used in the apheresis procedures and for otherwise isolating platelets, such as after collection from whole blood, such as any of the procedures described below (i.e., the buffy coat method). Commercially available centrifuges include, but are not limited to, centrifuges from Thermo Scientific® (e.g., Sorvall® centrifuges (e.g., Sorvall® RC-3 using an HG4L rotor, Sorvall® RC3BP plus low-speed centrifuge, Sorvall® BP 8, Sorvall® BP 16, Sorvall® RC4, Cryofuge™ 5500i, Cryofuge™ 16, Cryofuge™ 8), and centrifuges from Cardinal Health™ (e.g., Blood Bank benchtop Centrifuge SERO 12, Cardinal Health™ Benchtop STAT Centrifuge DASH Coag). The particular machine does not dictate the quality of the separation. Any centrifuge for use for separating blood components can be used herein. For example, a centrifuge that can separate platelets from other blood components such as white or red blood cells, can be used.

iv. Additional Apheresis Techniques

### Membrane Separation Apheresis

In membrane-based apheresis, membranes of varying pore sizes are used to collect the platelets. Membrane pore sizes range from nanometers to micrometers and are selected based on the blood component, protein, or toxin of interest for capture and/or filtering out. Membrane materials also vary based on the intended use and biocompatibility. Materials used in membranes include, but not limited to, polyester fibers, microporous polyurethane, polypropylene, polybutylene terephthalate, cellulose, cellulose acetate, cellulose triacetate, polysulfone, polyether sulfone, polylactic acid, polyamide, polyacrylonitrile, polymethylmethacrylate, polyurethane, and nanoporous silicon. Membrane-based apheresis can be used to remove uremic toxics and excess plasma proteins in patients with chronic kidney disease and autoimmune disorders. Disadvantages with membrane filtration devices include the need to use heparin as an anticoagulant and the requirement of higher blood flow rates necessitating central vascular access.

### Selective Adsorption

Apheresis devices can also hold columns varying in pore size and/or materials that selectively adsorb or remove substances of interest, including but not limited to lipoproteins, monocytes, granulocytes, and immunoglobulins. The method uses ligands or electrostatic interactions specific for toxins or pathogenic substances, thereby allowing non-pathogenic components to be returned to the subject. Adsorption columns are costly and not widely available, and thus are not widely available for clinical use in certain countries (e.g., Canada).

### Apheresis Procedures for Separating Particular Cellular Components

Apheresis procedures can be used for isolating specific blood components; leukapheresis or leukopheresis removes leukocytes or white blood cells, plasmapheresis removes disease-causing proteins or replaces missing protein in a subject/patient, plateletpheresis isolates platelets from blood, and erythropheresis isolates erythrocytes or red blood cells. Other apheresis procedures such as extracorporeal photopheresis, thrombocytapheresis, and lymphopheresis, are used for treating illnesses, diseases, or disorders. Depending on the procedure or treatment, the mechanism of collecting product of interest also will vary. For instance, isolation of healthy blood components can be achieved by centrifugal apheresis, whereas removal of proteins or toxin can require a membrane-based apheresis.

b. Whole Blood-Derived (WBD) Platelets

Platelets also can be isolated from collected whole blood. Whole blood is obtained from a single whole blood collection and centrifuged softly to collect platelets in the supernatant. For example, venous blood can be drawn using sterile techniques from a vein of the donor. The appropriate gauge needle is used in order to avoid irritation and/or trauma to the platelets in the whole blood. The collected whole blood is then placed in a centrifuge and subject to centrifugation at speeds and for time that effect platelet collection without disrupting platelet integrity (e.g., for 15 min at 3,200 rpm or for a similar time and/or speed). Centrifugation separates the blood into red blood cells, plasma lacking or with low levels of platelets, and platelet-rich plasma (PRP). At this point, the PRP is extracted from the total specimen of partitioned blood. The PRP from the first centrifuge cycle can be subjected to another, more aggressive or harder, centrifuge cycle to sediment and isolate platelets, which are then resuspended in a reduced volume of plasma and stored as platelet concentrates. For example, the PRP can be agitated to resuspend the platelets uniformly in the plasma. The final volume of PRP depends on the initial blood drawn.

WBD platelets from a single whole blood collection generally do not provide the adequate therapeutic dose for adults as defined by the United States Food and Drug Administration (U.S. FDA) of 5.5×10¹⁰ platelets per unit; multiple units of platelets (generally 3-10 units) from multiple donors are pooled to form a therapeutic dose.

The anti-coagulant used or included during platelet collection can depend on the platelet collection method. For example, WBD platelets and plasma can include citrate-phosphate-dextrose (CPD) as an anti-coagulant during the preparation process, whereas methods of apheresis generally include acid-citrate-dextrose as the anti-coagulant. Phosphate was previously shown to increase glycolysis in platelets, and thus, can affect storage conditions of platelets when isolating platelets and plasma using the manual whole blood collection and isolation method (Gulliksson (2000) Vox Sang 78:176-184).

In some examples, whole blood is collected from a donor and the collected whole blood is centrifuged to separate the platelet-rich plasma from the other components. In a second stage of the procedure, platelets are separated from plasma to form a platelet concentrate. The platelet concentrate can be resuspended in a liquid, such as plasma. The resulting composition, from the whole blood, contains concentrated platelets compared to whole blood and lacks other blood components, such as erythrocytes. Where the platelet concentrate is resuspended in plasma the resulting composition is a platelet-rich plasma (PRP) composition.

In other examples, platelets are sequestered and collected, for example from whole blood, using other devices, such as commercially available devices and/or equipment. For example, platelets can be isolated using the Autologous Fluid Concentrator (from Circle Biologics™, Minneapolis, MN) or the Gravitational Platelet Separation System (GPS, Biomet, Warsaw, IN, USA). The Autologous Fluid Concentrator (from Circle Biologics™, Minneapolis, MN) recently was approved by the U.S. Food and Drug Administration for use for platelet isolation in the United States. In other examples, a GPS® Platelet Concentration System, such as the GPS® III Platelet Concentration System from Biomet (Warsaw, IN, USA) can be used to separate and concentrate platelets from whole blood.

Any equipment or devices known in the art, and commercially available equipment or devices for platelet isolation can be used for isolating or collecting platelets from whole blood. Such equipment or devices known in the art, such as commercially available equipment or devices for platelet isolation can be used for isolating or collecting platelets from whole blood for inclusion in the PRP compositions provided herein. In the PRP compositions herein, platelets collected from whole blood using the platelet isolation devices and/or equipment can be held at refrigerated temperatures, such as at or about 4°, for several hours after collection and prior to further treatment. For example, in the PRP compositions herein, platelets collected from whole blood using the platelet isolation devices and/or equipment can be held at refrigerated temperatures, such as at or about 4°, for several hours after collection and prior to freeze drying (lyophilization).

c. Buffy Coat (BC) Method of Platelet Collection

Buffy Coat (BC) methods of platelet collection use whole blood as the source material for collecting platelets. Compared to the WBD technique, the BC technique centrifuges with a higher relative centrifugal force to form a buffy coat layer of leukocytes and platelets atop erythrocytes. The BC layer is then isolated, and subjected to a lower Relative Centrifugal Force centrifugation cycle. Next, the platelet-rich supernatant is collected and prepared for transfusion. In examples, platelets from multiple donors can be collected using the buffy coat method and pooled to generate a composition of pooled, concentrated platelets. The platelets isolated from the buffy coat can be incubated at cold temperatures and later subjected to further treatment, such as freeze drying.

For example, platelets are collected from the buffy coat by a series of centrifugation and blood separation steps (see, e.g., Hagen et al., (2021) Front. Bioeng. Biotechnol. 8:613621). For example, blood can be collected from donor animal and subjected to centrifugation. For example, a commercial centrifuge designed for blood separation can be used (e.g., a Hettich Rotanta 460R, Andreas Hettich GmbH & Co. KG, Tuttlingen, Germany) using acceleration and deceleration settings designed for blood component separation. In examples, the centrifugation procedure is conducted at temperatures that are below ambient temperatures but are above freezing. After centrifugation, the buffy coat can be recovered by separating the plasma and the erythrocytes using a commercially available blood-separating device (such as, for example, the Optipress® II blood separation device from Baxter S. A., Maurepas, France). The resulting composition contains concentrated platelets that can be subjected to further treatment such as freeze drying. In examples herein, the composition containing concentrated platelets is incubated at refrigerated temperatures prior to further treatment, such as freeze drying.

### E. COLD INCUBATION OF PLATELETS

Cold-stored platelets were previously characterized as moderately activated as compared to room temperature-stored platelets. In vitro characterization of cold-stored platelets showed that the activation phenotype of the cold-stored platelets was more consistent with the desired function of PRP for therapeutic administration compared to room-temperature-stored platelets (Zhao, et al. Journal of Proteome Research 20, 2251-2265 (2021)).

Platelet cells in the quiescent state that are delivered for therapeutic purposes generally require external activation for therapeutic activity and do not activate normally upon administration, such as administration to a joint. Platelets stored in refrigerated temperatures immediately after collection can show characteristics of a partially activated or primed state. Cold storage slows metabolism and increases the activation status of the platelets resulting in activated and primed platelets with properties (as shown herein) that mimic the healing properties of naturally-occurring platelets delivered by hemorrhage to an area of injury. Platelets in the PRP compositions provided herein are stored at refrigerated temperatures immediately after collection and show characteristics of a partially activated or primed state. Primed platelets can provide advantages for therapeutic uses. Other benefits of refrigerating platelets immediately after collection include decreased risk of contamination (i.e., bacterial, mycoplasma).

The PRP compositions provided herein do not include cryopreserves. Cold storage of platelets following collection and until further use or processing, such as lyophilization, allows intact platelets to survive the process without any cryopreservative. Platelets in the PRP compositions herein are incubated at refrigerated temperatures prior to combination with plasma. The majority of intact platelets in the PRP compositions incubated at refrigerated temperatures and prepared in the absence of cryopreservatives herein are in the primed, partially activated state.

After collection, platelets are incubated as detailed herein, for at least 4 hours up to 14 days, such as 48 hours to 5 days, at refrigerated temperatures, such as, for example, platelets at or about 0° C.-10° C., such as at 4° C. For example, after collection, platelets are incubated at refrigerated temperatures for at least 18 hours, 24 hours, 36 hours, 48 hours, 60 hours, 72 hours, 3 days, 3.5 days, 4 days, 4.5 days, 5 days, 5.5 days, 6 days, 6.5 days, 7 days, 7.5 days, 8 days, 8.5 days, 9 days, 9.5 days, 10 days, 10.5 days, 11 days, 11.5 days, 12 days, 12.5 days, 13 days, 13.5 days, or 14 days. For example, after collection, platelets are incubated at or about 0° C., 1° C.°, 2° C., 3° C.°, 4° C.°, 5° ° C.°, 6° C.°, 7° C.°, 8° C.°, 9° C.°, or 10° C.° for at least 18 hours, 24 hours, 36 hours, 48 hours, 60 hours, 72 hours, 3 days, 3.5 days, 4 days, 4.5 days, 5 days, 5.5 days, 6 days, 6.5 days, 7 days, 7.5 days, 8 days, 8.5 days, 9 days, 9.5 days, days, 10.5 days, 11 days, 11.5 days, 12 days, 12.5 days, 13 days, 13.5 days, or 14 days. In some examples herein, platelets are collected by apheresis and stored at refrigerated temperatures following collection (e.g., 0° C.-10° C., such as 4° C.-8° C.). No cryopreservatives are added to the platelets following collection.

In exemplary embodiments the PRP composition is prepared to avoid platelet activation and storage injury. In some examples, one or more of the following is incorporated into the preparation and handling practices of the PRP compositions provided herein to decrease and/or prevent platelet activation and/or storage injury. For example, the platelets in plasma in the platelet concentrates are refrigerated immediately after collection, such as, for examples, at temperatures between 2-8° C. and are maintained at refrigerated temperatures until further pooling and/or combination with additional plasma. In order to avoid platelet activation and/or storage lesion, in examples, platelets are collected in plasma and retained at platelet counts of at or less than 1,000,000 platelets/μL. In these examples, platelet concentrates of platelets in plasma containing platelets counts above at or about 1,000,000 platelets/μL are diluted with additional plasma at the end of the collection to, for example, avoid aggregation due to physical contact between platelets. For example, platelet concentrates (platelets and plasma) are diluted to concentrations of, for example, 500,000 platelets/μL by pooling with thawed plasma to a target of 500,000 platelets/μL+/−100,000 platelets/μL. In examples, during all stages of platelet and PRP preparation, including reconstitution of a lyophilized product, the compositions are not allowed to foam.

### F. LYOPHILIZATION

The PRP composition provided herein can be freeze-dried in the absence of any cryopreservative. Of interest herein are lyophilized powders containing PRP comprising platelets in the primed state. The platelets retain the partially activated or primed state during freeze-drying and also retain the primed state upon reconstitution. The lyophilized powders can be reconstituted for administration as solutions, emulsions, and other forms. The lyophilized powders also can be reconstituted and formulated as solids or gels. The method of making freeze-dried PRP compositions comprises lyophilizing, or freeze-drying, the PRP compositions containing primed platelets. Numerous protocols for lyophilization of eukaryotic cells, including platelets, are known in the art, and any suitable protocol can be used for producing the lyophilized PRP compositions comprising primed platelets provided herein. The PRP compositions provided herein do not contain a cryopreservative; no cryopreservative is added to the PRP composition prior to lyophilization.

Lyophilization or freeze-drying is a method of drying a substance using a combination of cold temperature and vacuum. Lyophilization or freeze-drying uses freezing of the substance followed by desiccation by sublimation and/or desorption of water and other liquids through the use of a vacuum. The method of making freeze-dried platelets comprises lyophilizing, or freeze-drying the PRP composition comprising pooled platelets and plasma. Protocols for lyophilization of eukaryotic cells and platelets, are known in the art, and any suitable protocol (see, e.g., U.S. Pat. Nos. 7,811,558, 8,486,617, and 8,097,403), with the exception that no cryopreservative is added, can be used for preparing a lyophilized PRP composition comprising primed platelets that are provided herein. No cryopreservative is added prior to lyophilization of the PRP composition comprising primed platelets as described herein.

Lyophilization results in compositions comprising platelets having a water content of less than 10%, such as, for example, with a water content of less than at or about 5%, such as 4%, 3%, 2%, 1%, or less. A lower water content can improve stability (e.g., extend the shelf-life) of the resulting freeze-dried platelets. Thus, in some examples, it is preferred to reduce the water content to as low of an amount as possible. The lyophilized PRP composition containing primed platelets provided herein contains little water and is in a solid (i.e., powder) form.

The PRP composition provided herein can be prepared using any protocols for lyophilization of eukaryotic cells, including platelets, known in the art. For example, the PRP composition can be lyophilized at a desired temperature, such as less than-55° C., less than-50° C., less than −45° C., less than −40° C., or less than −35° C.

For example, the PRP composition is lyophilized at −40° C. in accord with the following lyophilization protocol:
1. Freeze rate from ambient to −40° C. is approximately 1° C./minute for 60 minutes.
2 Freeze Soak time at −40° C. is approximately 3 hours.
3. Primary Dry ramps from −40° C. to 0° C. over 20 hours. Chamber Vacuum is 100 millitorr
4. Primary Dry Soak time at 0° C. is 8 hours.
5. Secondary Dry ramps from 0° C. to 30° C. over 12 hours. Chamber Vacuum is 100 millitorr
6. Primary Dry Soak time at 30° C. is 6 hours.
7. Total Cycle time including product freeze step: 50 hours, 35 minutes.

The particular times and temperatures listed above can be altered as required to produce a lyophilized PRP composition containing primed platelets. For example, the freeze rate from ambient to subzero temperatures, the freeze soak time, and the primary dry soak time can be modified to prepare a lyophilized PRP product containing primed platelets. For example, the freeze rate from ambient to below freezing temperatures (i.e., temperatures below at or about −55° C., −50° C., −45° C., −40° C., or −35° C.) can be 1° C./minute or 1.5° C./minute or 2° C./minute or 2.5° C./minute or less. In other examples, the freeze soak time at the subzero temperature (e.g., −40° C.) is approximately 3 hours, such as at least 1 hour, 1.5 hours, 2 hours, 2.5 hours, or 3 hours. It is known that any freeze drying procedure or technique can be used for preparing a PRP product. Exemplary freeze drying procedures or techniques are those where the lyophilized PRP composition is in a solid state (i.e., powder) and where the majority of platelets are primed platelets.

The sterile, lyophilized powder is prepared by subjecting the PRP composition containing pooled platelets, such as primed platelets, and pooled plasma to a freeze drying process. In some examples, the mixture of platelets and plasma can be sterile filtered or treated to remove particulates and to insure sterility, and apportioned into vials for lyophilization. The filtered platelet and plasma mixture (composition) can be lyophilized. The lyophilized PRP composition can be prepared as provided herein by combining pooled platelets and pooled plasma followed by lyophilization under standard conditions known to those of skill in the art to prepare the desired lyophilized PRP composition.

The lyophilized powder containing primed platelets can be stored under appropriate conditions. In some examples a lyophilized powder containing primed platelets can be stored at ambient temperatures, or at refrigerated temperatures such as at about 4° C. A liquid PRP composition provided herein above can be prepared by reconstituting the lyophilized PRP composition. Reconstitution of a lyophilized powder containing primed platelets provides a liquid composition for use in parenteral administration. The reconstituted PRP compositions provided herein do not contain a cryopreservative; no cryopreservative is added to the PRP composition following lyophilization and/or after reconstitution.

The lyophilized powders can be reconstituted as solids or gels or other such form, including for topical application into a wound, or tissue, or a tooth, or ear, or eye. For example, the platelet powder can be reconstituted as a small volume, such as in a 2 ml to 4 ml volume, which inactivated, such as in a petri dish, to form a gel-clot to use for treatment. Because the amount of platelets in a vial of lyophilized product is known, the lyophilized product can be reconstituted to a concentration useful or a particular treatment or indication.

### G. RECONSTITUTION

Lyophilized platelets, such as primed platelets in a PRP composition provided herein, can be reconstituted in solutions where the platelets uniformly resuspend in the liquid and where platelet activity and activation status are maintained. Lyophilized platelets, such as primed platelets in a PRP composition provided herein can be reconstituted in a solution that is relatively nontoxic and/or innocuous and/or does not cause significant harm to a recipient at platelet concentrations for administration. In some examples, adverse side effects ascribable to the preparation do not impair the beneficial effects of the platelets in the PRP composition. Lyophilized PRP compositions provided herein can be reconstituted in saline or water.

### 1. Reconstitution in Water or Saline

In examples herein, a lyophilized platelet-rich plasma is reconstituted in saline solution. For example, a lyophilized PRP composition can be reconstituted in a solution such as, for example, 0.9% NaCl. In such examples, 0.9% NaCl can be prepared by dissolving 900 mg of solid NaCl in approximately 0.1 liter of water to obtain a 0.9% concentration salt solution. In other examples, a lyophilized platelet-rich plasma is reconstituted in water.

The liquid for reconstituting the lyophilized PRP composition can contain an excipient which improves the stability or other pharmacological component of the powder or reconstituted solution, prepared from the powder, and does not contain a cryopreservative. In examples where the PRP composition includes a carrier, diluent or excipient, the carrier, diluent, or excipient is compatible with the components of the PRP composition, including, for example, primed platelets. The carrier, diluent or excipient is not a cryopreservative. The excipient is not a cryopreservative and provides another function that is not cryopreservation. Selection of the carrier or excipient is within the skill of the administering profession and can depend upon a number of parameters. These include, for example, the mode of administration (i.e., intra-articular, intralesional oral, nasal, pulmonary, local, topical or any other mode of administration) and disorder treated.

Each vial contains a single dosage or multiple dosages of the PRP composition. The compositions provided herein can be formulated for single dosage (direct) administration or for dilution or other modification. The concentrations of the compounds in the formulations are effective for delivery of an amount, upon administration, that is effective for the intended treatment. Typically, the compositions are formulated for single dosage administration. To formulate a composition, the weight fraction of a compound or mixture thereof is dissolved, suspended, dispersed, or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the mode of administration.

The reconstituted solution can contain an anticoagulant, such as residual coagulant added during the platelet collection process. For example, the lyophilized powder can be prepared by dissolving in sterile water or saline and also can contain residual amounts of an anti-coagulant that is added during platelet and/or plasma collection. In other examples the lyophilized powders are dissolved in water or saline and are combined with another therapeutic known to those of skill in the art.

### 2. Other Solutions for Platelet Reconstitution and Resuspension

The lyophilized platelets provided herein generally are reconstituted and/or resuspended in saline or water. Other solutions that do not contain cryopreservatives also can be used for resuspending platelets. For example, solutions other than a saline solution or water can be used for reconstituting the lyophilized platelets that are stored at cold temperatures prior to lyophilization. For example, solutions that are used for platelet storage can be used for resuspending lyophilized platelets. For example, Platelet Additive Solutions (PAS) include components to support platelet storage and viability and can increase storage times for viable platelets. In some examples, PAS that are designed for long platelet term storage, can be used for platelet reconstitution. In addition to sodium chloride, PAS can include other components for platelet preservation, such as, for example, one or more of sodium citrate, mannitol, phosphate, potassium, potassium chloride, magnesium, magnesium chloride; and can also include sodium acetate as a nutrient for platelets, or adenine, lidocaine (see e.g., van der Meer and de Korte (2018) Transfus Med Hemother 45 (2): 98-102). Platelet concentrates can be resuspended in a solution to provide a suspension of platelets suitable for storage or can be readily available for transfusion to a patient.

Lyophilized platelets, such as primed platelets in a PRP composition provided herein, can be reconstituted in plasma. For example, lyophilized platelets and lyophilized PRP compositions can be reconstituted in plasma isolated from the subject to whom the platelets are to be administered (e.g., native patient specific plasma). In other examples, lyophilized platelets and lyophilized PRP compositions can be reconstituted in native allogeneic plasma solutions where the platelets uniformly resuspend in the liquid and where platelet activity and activation status is maintained. For example, a rehydrated PRP composition comprising primed platelets can be reconstituted in plasma that does not elicit an adverse reaction in the subject to whom the PRP composition is administered.

### H. PRP AND PLATELET CHARACTERIZATION—Characterization of Platelet Activation and/or Activity in a PRP Composition

Platelet phenotype can impact platelet function, circulation time, and membrane characteristics. Total platelet dose, growth factor content, and leukocyte count are among factors for in vitro characterization and evaluating effectiveness outcomes upon administration or therapeutic use of platelets (McCarrel & Fortier (2009) J Orthop Res 27 (8): 1033-42). Platelets in the PRP compositions provided herein exhibit differential activation compared to fresh platelets and compared to platelets prepared in the presence of cryopreservatives. Exemplary PRP compositions provided herein contain primed platelets. Exemplary freeze-dried PRP compositions prepared provided herein contain primed platelets after reconstitution or rehydration.

### 1. Assays and Markers for Assessing Platelet Activation State

Various in vitro and in vivo assays can be used to monitor or screen or assess PRP compositions for the cell types in the compositions and the properties of the cells, such as platelets, in the compositions. Such assays are well known to those of skill in the art. One of skill in the art can assess platelet properties and/or characteristics and/or assess any change in the platelet properties and/or characteristics compared to platelet properties and/or characteristics prior to treatment (i.e., incubation in the cold) or prior to another treatment, such as freeze drying. Some such assays are exemplified herein. Assays for platelet properties and/or characteristics include, but are not limited to commercial analysis kits, such as commercially available assays, or FDA-approved kits for assessing platelet properties. For example, in vitro assays can be performed on pooled platelet-rich plasma (e.g., pooled platelets combined with plasma), as exemplified in Example 1, or on reconstituted PRP as exemplified in Example 1. For example, in vitro assays such as flow cytometry can be performed as exemplified in Example 2, to assess platelet particle size. In another example, in vitro assays such as flow cytometry can be performed as exemplified in Example 2 or Examples 3 and 5, to assess the composition of various cell types in the PRP composition, such as the PRP composition prior to lyophilization or the reconstituted lyophilized PRP composition. In another example, in vitro assays such as flow cytometry can be performed as exemplified in Examples 3 and 5, to assess the activation status of platelets in the PRP composition, such as the PRP composition prior to lyophilization or the reconstituted lyophilized PRP composition.

Exemplary in vitro and in vivo assays can be used for characterization of platelet activity. Numerous assays, such as assays for measuring platelet activity or activation, are known to one of skill in the art. Assays for platelet activation include, for example, assessment of platelet activation markers, clot activators, and growth factor and cytokine production. For example, in vitro assays known in the art can be used, such as ELISA or flow cytometry. For example, in vitro assays can be performed on pooled platelet-rich plasma (e.g., pooled platelets combined with plasma), as exemplified in Example 1, or on reconstituted PRP as exemplified in Example 1. For example, in vitro assays can be used to assess the composition of the platelet granules or chemokine release from platelet granules or platelet response to agonist, such as exemplified in Example 7.

Assays to measure platelet activity also include functional assays that measure platelet functional activity or activation, such as, for example, coagulation assays. Platelet function assays include, but are not limited to, optical aggregometry, activated clotting time, whole blood aggregometry, platelet count ratio, Platelet Function Analyzer (e.g., the PFA-100 from Dade-Behring, Deerfield, IL, USA), and Rapid Platelet function Assay (e.g., the Rapid Platelet function Assay from Accumetrics).

The platelets prepared in accord with the methods provided herein are not quiescent and are not activated, rather they are partially activated or primed. By virtue of incubation of the platelets at refrigerated temperatures immediately following collection (i.e., by apheresis) the platelets remain in a partially activated or primed state. For example, the partially activated, primed platelets have externalized phosphatidylserine. In some examples of the rehydrated lyophilized PRP compositions described herein, the majority of the platelets in the composition survive the process of lyophilization intact with externalized phosphatidylserine. Administration of primed or activated PRP for intra-articular, intralesional, or topical use, allows for accelerated release of dense and alpha granules compared to administration of quiescent platelets. The PRP composition provided herein contains both platelet microvesicles, intact platelets and free growth factors/chemokines.

Previous PRP production shows that platelets in PRP cannot be prepared without cryopreservatives because they are not active or are much less active. For example, previous studies show that freeze drying platelets without cryopreservatives results in decreased viability; thus, it was understood in the art that addition of cryopreservatives prior to lyophilization is necessary to retain platelet function. The methods for preparing platelets and PRP provided herein, however, eliminate the need for cryopreservatives and result in primed platelets that, when used, are active, and they can be lyophilized and stored for long periods of time. The instant results demonstrate that when PRP is prepared in accord with the methods set forth in the examples and as described herein, including the handling methods and pre-lyophilization refrigeration, cryopreservatives are not needed to retain platelet viability in a PRP product. The methods show that when the platelets are handled as detailed herein, they retain a primed state prior to lyophilization. Flow cytometry (FC) data show that the majority of the platelets are not activated.

Platelet function can be assessed in vitro. In vitro assays to determine platelet activity, including activated platelet activity, are known in the art, and described herein. Exemplary assays include any of those listed herein and those known in the art. Activity of platelets in a PRP composition, such as a reconstituted lyophilized PRP composition, can be compared to fresh platelets isolated from a subject, or fresh pooled platelets prepared from multiple subjects, or compared to a PRP composition prior to lyophilization.

For example, cell culture assays can be used to assess platelet function. In some examples, the effect of platelets on cell viability is assessed in vitro, using cell culture systems and assays. For example, a commercially available platelet product produced for intravenous administration and developed for use as a hemostat and healing wounds showed similar effects on proliferation to fresh platelets in human umbilical vein endothelial and fibroblast cells as measured by the MTT assay (U.S. Pat. No. 7,811,558; Pietramaggiori et al., Wound Rep. Reg. 15:213-220 (2007)). Platelet activity on wound healing also can be assessed by collagen contraction and reorganization after incubation with platelets (U.S. Pat. No. 7,811,558). In addition to promoting cell proliferation and collagen reorganization, the various methods used to create StablePlates RX™ platelets resulted in about between 20 to 90% platelet microparticles, which affected clotting times such that the composition and methods of FDP with the highest amount of microparticles provided the shortest clotting times (U.S. Pat. No. 7,811,558). The methods of creating the freeze-dried platelets (FDPs) also had shorter clotting times compared to fresh platelets. The use of the StablePlates RX™ platelets in vivo showed that it was capable and better at arresting bleeding, including heavy arterial bleeds compared to other hemostatic agents (U.S. Pat. No. 7,811,558) and improved wound healing (U.S. Pat. No. 7,811,558; Pietramaggiori et al., (2007) Wound Rep. Reg. 15:213-220).

### 2. Antibody Labeling and Flow Cytometry

The cell types in the composition and the state of the cells can be assessed by antibody labeling and/or flow cytometry. Platelet activation also can be assessed by flow cytometry. Platelets can be labeled, for example antibody labeled, for particular markers. For example, platelets can be labeled for markers that indicate platelet activation.

Markers of platelet activation include P-selectin (CD62P) and CD40L on the platelet membrane, and PS exposure. The activation state of circulating platelets can be assessed by whole-blood flow cytometry of P-selectin (CD62P) and/or the activated form of integrin αIIbβ3 (CD41/CD61) expression on the surface of platelets. As is known in the art, the number of positive cells can be influenced by antibody affinity. For example, species differences between the sample and the antibody can lead to incomplete antibody binding due to species differences between the species of the sample and antibody.

Platelet activation triggers the release of their granular contents and other metabolites into plasma. Activation state also can be assessed by measuring levels of granule release markers, such as beta-thromboglobulin (BTG) and platelet factor 4 (PF4). In other examples, sCD40L and TxB2 are released upon platelet activation. CD40L is a protein expressed in platelets that translocates to the surface of agonist-activated platelets, and is shed from the platelet surface as the soluble form, sCD40L. CD40L plays a role in inflammation, thrombosis, and restenosis (Andre et al., (2002) Circulation 106 (8): 896-899). Labeling of granule release markers by flow cytometry can be used to assess platelet activation. Proteins on the platelet surface also can be assessed by flow cytometry. Proteins on the platelet cell surface can indicate activation status.

In some examples, phosphatidylserine externalization is assessed by flow cytometry assay and/or immunohistochemical staining. For example, in some examples of the PRP composition described herein, the ratio of platelets that are lactadherin positive by flow cytometry to platelets that are lactadherin negative in the composition by flow cytometry is at least or at least about 1:1, 1.1:1, 1.2:1, 1.3:1, 1.4:1, 1.5:1, 1.6:1, 1.7:1, 1.8:1, 1.9:1, 2.1:1, 2.2:1, 2.3:1, 2.4:1, 2.5:1, 2.6:1, 2.7:1, 2.8:1, 2.9:1, 3:1, 3.1:1, 3.2:1, 3.3:1, 3.4:1, 3.5:1, 3.6:1, 3.7:1, 3.8:1, 3.9:1, 4:1, 4.1:1, 4.2:1, 4.3:1, 4.4:1, 4.5:1, 4.6:1, 4.7:1, 4.8:1, 4.9:1, 5:1, 5.1:1, 5.2:1, 5.3:1, 5.4:1, 5.5:1, 5.6:1, 5.7:1, 5.8:1, 5.9:1, 6:1, 6.1:1, 6.2:1, 6.3:1, 6.4:1, 6.5:1, 6.6:1, 6.7:1, 6.8:1, 6.9:1, 7:1, 7.1:1, 7.2:1, 7.3:1, 7.4:1, 7.5:1, 7.6:1, 7.7:1, 7.8:1, 7.9:1, 8:1, 8.1:1, 8.2:1, 8.3:1, 8.4:1, 8.5:1, 8.6:1, 8.7:1, 8.8:1, 8.9:1, 9:1, 9.1:1, 9.2:1, 9.3:1, 9.4:1, 9.5:1, 9.6:1, 9.7:1, 9.8:1, 9.9:1, 10:1, 10.1:1, 10.2:1, 10.3:1, 10.4:1, 10.5:1, 10.6:1, 10.7:1, 10.8:1, 10.9:1, 11:1, 11.1:1, 11.2:1, 11.3:1, 11.4:1, 11.5:1, 11.6:1, 11.7:1, 11.8:1, 11.9:1, 12:1, 12.1:1, 12.2:1, 12.3:1, 12.4:1, 12.5:1, 12.6:1, 12.7:1, 12.8:1, 12.9:1, 13:1, 13.1:1, 13.2:1, 13.3:1, 13.4:1, 13.5:1, 13.6:1, 13.7:1, 13.8:1, 13.9:1, 14:1 or more.

In some examples of the PRP composition described herein, at least about or about 50%, 55%, 60%, 65%, 70%, 75%, 80%, 85%, 90%, 91%, 92%, 93%, 94%, 95%, 96%, 97%, 98%, 99% or 100% of the cells in the PRP composition are positive for phosphatidylserine externalization by flow cytometry. For example, at least or at least about 50%, 55%, 60%, 65%, 70%, 75%, 80%, 85%, 90%, 91%, 92%, 93%, 94%, 95%, 96%, 97%, 98%, 99% or 100% of the cells in the PRP composition are positive for lactadherin by flow cytometry.

In some examples of the PRP composition described herein, at least about or about 50%, 55%, 60%, 65%, 70%, 75%, 80%, 85%, 90%, 91%, 92%, 93%, 94%, 95%, 96%, 97%, 98%, 99% or 100% of the platelets in the PRP composition are positive for phosphatidylserine externalization by flow cytometry. For example, at least or at least about 50%, 55%, 60%, 65%, 70%, 75%, 80%, 85%, 90%, 91%, 92%, 93%, 94%, 95%, 96%, 97%, 98%, 99% or 100% of the platelets in the PRP composition are positive for lactadherin by flow cytometry.

In some examples of the PRP composition described herein, at least about or about 50%, 55%, 60%, 65%, 70%, 75%, 80%, 85%, 90%, 91%, 92%, 93%, 94%, 95%, 96%, 97%, 98%, 99% or 100% of the cells in the PRP composition are positive for platelet markers by flow cytometry. For example, at least or at least about 50%, 55%, 60%, 65%, 70%, 75%, 80%, 85%, 90%, 91%, 92%, 93%, 94%, 95%, 96%, 97%, 98%, 99% or 100% of the cells in the PRP composition are positive for CD61 or CD9 by flow cytometry.

In some examples, up to at or about 40%, 41%, 42%, 43%, 44%, 45%, 46%, 47%, 48%, 49%, or 50% of the platelets in the PRP composition described herein are activated platelets as assessed by the presence of activated platelet markers by flow cytometry. For example, at least or at least about 40%, 41%, 42%, 43%, 44%, 45%, 46%, 47%, 48%, 49%, or 50% of the platelets in the PRP composition are positive for CD62/p-selectin by flow cytometry.

In some examples, up to at or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10% of the cells in the PRP composition described herein are white blood cells as assessed by the presence of white blood cells markers by flow cytometry. For example, up to at or about 1%, 2%, 3%, 4%, 5%, 6%, 7%, 8%, 9%, or 10% of the cells in the PRP composition are positive for CD44 by flow cytometry.

### 3. Platelet Size and Shape

Platelet activation status can be assessed by examining platelet size and shape, alone or in combination with other markers. Upon activation, platelets undergo morphological changes from a discoid shape (discocyte) into a stellate-shape and form pseudopods or a balloon, spherical shape (spherocytes). Activated platelets have a spherical shape compared to the discoid shape of inactivated platelets. Upon activation, platelets flatten out and the mean platelet volume increases, this is shown as a larger platelet particle size. Membrane restructuring increases the surface area of the platelet membrane. Platelet shape change can be determined and measured by flow cytometry, microscopy (e.g., electron microscopy), or a combination of flow cytometry and microscopy (e.g., imaging flow cytometry). Using flow cytometry and/or microscopy to determine circulating activated platelets can be detected by quantifying shape change on the basis of different light scatter properties of discocytes and spherocytes using antibodies for CD61 (integrin β3) and CD42a (Ozpolat et al., Platelets 34 (1): 1-6 (2022)). The change in platelet shape also can be detected by a decrease in light transmission and disappearance of oscillations of stirred platelet suspensions in the platelet aggregometer although these methods have limitations and are not reliable compared to methods of imaging flow cytometry (Milton and Frojmovic, (1983) J Pharmacol Methods 9:101-15).

Platelet activation can increase both mean platelet volume (MPV) and platelet distribution width (PDW). Primed platelets have an MPV that is between the MPV of inactivated and activated platelets, up to the MPV of an activated platelet. Inactivated human platelets generally are 2-4 μm and activated platelets generally are 5-10 μm. Activated platelets are larger than inactivated platelets. MPV can be used to assess platelet activation.

In some examples herein, platelets in a PRP composition provided herein are not activated and do not have an increased size compared to inactive platelets. In examples herein, the equine lyophilized PRP composition, when rehydrated, provides a platelet-rich plasma (PRP) composition where the majority of particles in the composition are between 0.5 μm and 5 μm, such as between 0.5 μm and 2 μm, which is a size indicative of partial activation. In some examples, an equine PRP composition prepared in accord with the methods described herein contains more than 80% intact platelets sized between 0.5 μm and 5 μm.

### 4. Phosphatidylserine Externalization

Phosphatidylserine externalization can be assessed by assays known in the art. For example, using flow cytometry techniques, it is possible to analyze the biological activity of platelets after reconstitution after freeze drying. For example, phosphatidylserine externalization can be assessed by lactadherin binding. Lactadherin binds to phosphatidylserine in a stereospecific and calcium independent manner. Phosphatidylserine externalization also can be measured by annexin V staining. Annexin V binds negatively charged phospholipids like phosphatidylserine (PS) with high affinity in the presence of Ca²⁺ ions.

In examples of a PRP composition provided herein, at least about or about 50%, 55%, 60%, 65%, 70%, 75%, 80%, 85%, 90%, 91%, 92%, 93%, 94%, 95%, 96%, 97%, 98%, 99% or 100% of the platelets in the PRP composition have externalized phosphatidylserine (e.g., phosphatidylserine on the outer leaflet of the plasma membrane). In examples of a PRP composition provided herein, the ratio of platelets with externalized phosphatidylserine to platelets without externalized phosphatidylserine in the composition is at least or at least about 1:1, 1.1:1, 1.2:1, 1.3:1, 1.4:1, 1.5:1, 1.6:1, 1.7:1, 1.8:1, 1.9:1, 2.1:1, 2.2:1, 2.3:1, 2.4:1, 2.5:1, 2.6:1, 2.7:1, 2.8:1, 2.9:1, 3:1, 3.1:1, 3.2:1, 3.3:1, 3.4:1, 3.5:1, 3.6:1, 3.7:1, 3.8:1, 3.9:1, 4:1, 4.1:1, 4.2:1, 4.3:1, 4.4:1, 4.5:1, 4.6:1, 4.7:1, 4.8:1, 4.9:1, 5:1, 5.1:1, 5.2:1, 5.3:1, 5.4:1, 5.5:1, 5.6:1, 5.7:1, 5.8:1, 5.9:1, 6:1, 6.1:1, 6.2:1, 6.3:1, 6.4:1, 6.5:1, 6.6:1, 6.7:1, 6.8:1, 6.9:1, 7:1, 7.1:1, 7.2:1, 7.3:1, 7.4:1, 7.5:1, 7.6:1, 7.7:1, 7.8:1, 7.9:1, 8:1, 8.1:1, 8.2:1, 8.3:1, 8.4:1, 8.5:1, 8.6:1, 8.7:1, 8.8:1, 8.9:1, 9:1, 9.1:1, 9.2:1, 9.3:1, 9.4:1, 9.5:1, 9.6:1, 9.7:1, 9.8:1, 9.9:1, 10:1, 10.1:1, 10.2:1, 10.3:1, 10.4:1, 10.5:1, 10.6:1, 10.7:1, 10.8:1, 10.9:1, 11:1, 11.1:1, 11.2:1, 11.3:1, 11.4:1, 11.5:1, 11.6:1, 11.7:1, 11.8:1, 11.9:1, 12:1, 12.1:1, 12.2:1, 12.3:1, 12.4:1, 12.5:1, 12.6:1, 12.7:1, 12.8:1, 12.9:1, 13:1, 13.1:1, 13.2:1, 13.3:1, 13.4:1, 13.5:1, 13.6:1, 13.7:1, 13.8:1, 13.9:1, 14:1 or more.

For example, in examples of the PRP composition described herein, at least about or about 50%, 55%, 60%, 65%, 70%, 75%, 80%, 85%, 90%, 91%, 92%, 93%, 94%, 95%, 96%, 97%, 98%, 99% or 100% of the platelets in the PRP composition are lactadherin positive. For example, in some examples of the PRP composition described herein, the ratio of platelets that are lactadherin positive to platelets that are lactadherin negative in the composition is at least or at least about 1:1, 1.1:1, 1.2:1, 1.3:1, 1.4:1, 1.5:1, 1.6:1, 1.7:1, 1.8:1, 1.9:1, 2.1:1, 2.2:1, 2.3:1, 2.4:1, 2.5:1, 2.6:1, 2.7:1, 2.8:1, 2.9:1, 3:1, 3.1:1, 3.2:1, 3.3:1, 3.4:1, 3.5:1, 3.6:1, 3.7:1, 3.8:1, 3.9:1, 4:1, 4.1:1, 4.2:1, 4.3:1, 4.4:1, 4.5:1, 4.6:1, 4.7:1, 4.8:1, 4.9:1, 5:1, 5.1:1, 5.2:1, 5.3:1, 5.4:1, 5.5:1, 5.6:1, 5.7:1, 5.8:1, 5.9:1, 6:1, 6.1:1, 6.2:1, 6.3:1, 6.4:1, 6.5:1, 6.6:1, 6.7:1, 6.8:1, 6.9:1, 7:1, 7.1:1, 7.2:1, 7.3:1, 7.4:1, 7.5:1, 7.6:1, 7.7:1, 7.8:1, 7.9:1, 8:1, 8.1:1, 8.2:1, 8.3:1, 8.4:1, 8.5:1, 8.6:1, 8.7:1, 8.8:1, 8.9:1, 9:1, 9.1:1, 9.2:1, 9.3:1, 9.4:1, 9.5:1, 9.6:1, 9.7:1, 9.8:1, 9.9:1, 10:1, 10.1:1, 10.2:1, 10.3:1, 10.4:1, 10.5:1, 10.6:1, 10.7:1, 10.8:1, 10.9:1, 11:1, 11.1:1, 11.2:1, 11.3:1, 11.4:1, 11.5:1, 11.6:1, 11.7:1, 11.8:1, 11.9:1, 12:1, 12.1:1, 12.2:1, 12.3:1, 12.4:1, 12.5:1, 12.6:1, 12.7:1, 12.8:1, 12.9:1, 13:1, 13.1:1, 13.2:1, 13.3:1, 13.4:1, 13.5:1, 13.6:1, 13.7:1, 13.8:1, 13.9:1, 14:1 or more.

Up to 100% of the platelets in a PRP composition described herein have externalized phosphatidylserine; up to 100% of the reconstituted lyophilized platelets are in the partially activated or primed state.

### 5. Swirling

In some examples, methods such as swirling are used to characterize platelet shape. Platelets in the inactivated state have a discoid shape, which causes light to be scattered in multiple directions, whereas platelets in an activated state lose their discoid shape and do not scatter light. Swirling and platelet shape can be used as a visual cue for platelet activation. Swirling is a subjective assessment and it is unconfirmed whether swirling results correlate with other platelet characteristics, such as transfusion efficiency, or activation status. Swirling is not dispositive of activation. If swirling is used to characterize platelet activation status it should not be used in isolation, and should be used in conjunction with another assay for activation status (e.g., biochemical or immunohistological analysis). Swirling is used to indicate pH of the PRP composition.

### 6. Light Transmission Aggregometry (LTA)

Light transmission aggregometry (LTA) can be used to assess platelet activation. Agonists can be added to platelets, such as a PRP composition, and the change in light transmission caused by platelet aggregation is assessed. In other examples, LTA can be used to assess release of granule contents following platelet activation. Release of granule contents indicates activated platelets.

### 7. Granulation

Granules remain stored in circulating platelets until platelet activation triggers the exocytosis of their contents. Platelet granularity can be assessed to measure platelet activation and platelet activity. Platelet granulation of platelets in a reconstituted lyophilized PRP product can be compared to granularity of fresh platelets. Inactivated platelets are degranulated.

Proteins (e.g., P-selectin or CD62P, fibrinogen, von Willebrand factor (vWF), factor V/Va, X/Xa, VIII, platelet derived growth factor (PDGF), platelet factor 4, beta-thromboglobulin, albumin, fibronectin, cytokines, chemokines, ligand-induced binding sites in glycoprotein IIb/IIIa (αIIβ3a) and thrombospondin), nonproteins (e.g., serotonin, histamine, ADP, ATP, pyrophosphate, and calcium), and enzymes (e.g., glycosidases, acid proteases, and cationic proteins) are released from the platelet granules upon platelet activation platelets (Moritz et al., Vet Clin Pathol. 32:6-12 (2003)). Expression of alpha granule exocytosis can be measured by assays known in the art, such as, for example, ELISA, enzyme-linked immunospot assay, radioimmunoassay, flow cytometry, immunoprecipitation, Western blot, and/or immunocytochemistry using biomarkers or antibodies including, but not limited to, anti-CD62P (marker for P-selectin or CD62P), anti-CD40, anti-CD40L, anti-thrombospondin/GMP-33, and multimerin. Expression of alpha granule exocytosis can be measured by mepacrine staining, ELISA, enzyme-linked immunospot assay, radioimmunoassay, flow cytometry, immunoprecipitation, Western blot, and/or immunocytochemistry using biomarkers or antibodies including, but not limited to, CD63 and LAMP-2 (CD107b). Expression of lysosomal exocytosis can be measured by ELISA, enzyme-linked immunospot assay, radioimmunoassay, flow cytometry, immunoprecipitation, Western blot, and/or immunocytochemistry using biomarkers or antibodies including, but not limited to, LAMP-1, LAMP-2, and CD63. Other proteins and nonproteins secreted from platelet granules can be measured using markers for platelet factor 4 (PF4) or CXCL4, beta-thromboglobulin, MIP-1, RANTES, serotonin (5-HT), histamine, albumin, vWF, factor V, PDGF, fibronectin, and antibodies and assays for detecting a wide range of cytokines and chemokines that are released from platelet granules.

### 8. Calcium Levels

Calcium is a major chemical messenger in platelet activation, and intracellular calcium levels are increased in activated platelets. In quiescent platelets, intracellular levels of calcium are relatively low or absent compared to activated platelets.

Intraplatelet calcium dynamics can be measured by assays known in the art. For example, intraplatelet calcium dynamics can be measured by ratiometric flow cytometry using calcium indicators such as, for example, Quin-2, Fura-2, and Indo-1 (Assinger et al., PLOS ONE 10 (4): e0122527 (2015)). Calcium dyes also can be used. Other methods of measuring calcium levels include cuvette-based methods, radiometric measurements, fluorescence microscopy, and flow cytometry using fluorescent Ca²⁺ indicators such as, for example, Fura-2 AM, Fura-2 pentapotassium, Fura Red AM, Indo-1 AM, Fluo-3, Fluo-4, and/or Indo-1 pentapotassium.

### 9. Platelet Vesiculation or Microparticle Generation

Platelet microparticles (MPs) are small, membrane-bound vesicles of 0.1 to 1.0 μm diameter that are generated by budding or shedding from the plasma membrane responsive to platelet activation. Platelet microparticles contain proteins and negatively charged phospholipids, such as phosphatidylserine (PS), which impacts their procoagulant character and proinflammatory properties.

Microparticle generation can be used to measure platelet activity. Platelet microparticles participate (e.g., support) in thrombus formation and also are associated with various pathological processes. Platelet microparticles derived from activated platelets are heterogenous in size and differ in their contents of growth factors, chemokines, and membrane receptors. Platelet microparticle generation can be measured by flow cytometry using antibodies for platelet membrane markers and optionally size exclusion.

Platelet-derived MPs can be assessed by flow cytometry. For example, flow cytometry assessing CD41 labeling can be used to assess platelet-derived MPs (Nielson et al., Journal of Extracellular Vesicles 2:20795 (2014)). Anti-human CD235a can be used to detect erythrocyte-derived MPs. CD31, which is expressed on both platelets and endothelial cells, and CD42b (GPIba), which is expressed only on platelets can be used to discriminate between PMPs and EMPs, thereby detecting endothelial cell-derived MPs. CD142 can be used to detect tissue factor V-positive MPs.

### 10. Receptor Changes

Upon platelet activation, receptor expression on the platelet can increase or decrease depending on the particular receptor(s). Fibrinogen receptors, the most abundant integrin on the platelet surface, are formed from two subunits: glycoprotein IIb (GPIIb; integrin am and GPIIb/Illa activation. Its quantification can be detected using radiolabeled 7E3 (Wagner et al., Blood 88 (3): 907-913 (1996) and markers for PAC-1, LIBS/RIBS-specific antibodies, fluorescent fibrinogen, anti-fibrinogen antibodies, CD41/61 antibodies. Collagen receptor GPIa/IIa activation can be detected using antibodies detecting conformational changes such as GPla/Ila (a2β1, CD49/CD29).

### 11. Platelet Aggregation

Platelet aggregation occurs upon activation. Platelet aggregation can be measured using methods known in the art. For example, aggregation can be assessed by flow cytometry, bleeding times, platelet aggregometry, light transmission aggregometry, lumi-aggregometric assay, light transmission platelet aggregation, impedance aggregometry, Plateletworks, PFA-100, Innovance PFA-200, Impact or Cone and Platelet Analyzer, global thrombosis test, TEG or platelet mapping system, ROTEM platelet, radio- or enzyme-linked immune assays, coagulation time to form a platelet plug (Paniccia et al., Vasc Health Risk Manag. 11:133-148 (2015); Duke, JAMA 55:1185-1192 (1910)). For example, light transmission aggregometry can be used to assess platelet aggregation after exposure to external aggregating agents (e.g., agonists such as ADP, arachidonic acid, collagen, and epinephrine) in vitro (Zhou et al., Am J Clin Pathol 123:172-183 (2005)). Light transmission aggregometry can determine the percent of platelet aggregation in platelet-rich plasma by measuring the increase in light transmission responsive to the addition of a platelet agonists to the platelet suspension.

Activated platelets also bind to integrin receptors, facilitated by P-selectin via P-selectin glycoprotein ligand (PSGL-1), GP IIb/Illa, and CD40L, and aggregate with monocytes, neutrophils, eosinophils, and leukocytes. Platelet aggregation is commonly measured by flow cytometry and FACS by using forward- and side-angle light scatter plots, and also can be detected by other methods including, for example, microscopy, ELISA, IHC, IP, WB, and ICC using markers/antibodies for CD31 and CD9 (Cuyper et al., Blood 121 (10): e70-e80 (2013)), and P-selectin (Moritz et al., Vet Clin Pathol. 32:6-12 (2003)).

### 12. Other Assays

Other assays that indicate platelet activity or activation can be used to assess platelet status. For example, thrombin peak height can be assessed. Other events that occur in response to platelet activation include intracellular phosphorylation events and changes in the mitochondrial membrane potential. Intracellular phosphorylation events can be measured by flow cytometry using VASP (Schwarz et al., Thromb Haemost 82 (3): 1145-1152). Changes to the platelet's mitochondrial membrane potential can be measured using a variety of probes based on carbocyanine (e.g., JC-1) and rosamine (e.g., CMSRos) (Johnson et al., Life technologies 2010:924-936; Gyulkhandanyan et al., J Thromb Thrombolysis 33 (4): 397-411 (2012)).

### I. THERAPEUTIC USES AND METHODS OF TREATMENT

Provided herein are uses of PRP compositions that contain primed platelets. Provided herein are uses of PRP compositions containing primed platelets such as PRP compositions prepared in accord with the methods described herein. The methods described herein includes methods for therapeutic uses of primed platelets in a PRP composition. The therapeutic uses described below are exemplary and do not limit the applications of the methods described herein.

The compositions and uses provided herein employ platelet rich plasma and platelets that are prepared without cryopreservatives. The platelets in the PRP compositions provided herein are maintained in a primed state and have not released the contents of their alpha granules. Platelets in the primed state are advantageous for intravenous, intralesional and intraarticular administration. Incubation of the platelets at refrigerated temperatures for a limited time, of at least about 48 hours up to 14 days, immediately post-donation preserves a high number of platelets, maintain the functional integrity of platelets, provide platelets in a primed but not fully activated state, and provide an increased in vivo circulatory life span compared to previously described lyophilized-reconstituted platelet products. By controlling activation of platelets, by refrigerating them prior to lyophilization and/or administration, the metabolic process of the platelets is slowed, making them less reactive to activators (i.e., compounds or conditions) that result in platelet activation prior to administration. Such primed platelets that are not in the fully activated state, are useful for therapeutic uses or treatment(s). For some therapeutic uses, clinical benefits of platelets rely on maintenance of the platelet granules, such as alpha granules, in the intact state prior to administration.

Injection of concentrated platelets can initiate tissue repair at a site of injury or site affected by a disease or disorder, such as, for example, by initiating tissue repair via the release of biological factors, such as growth factors, cytokines, and/or lysosomes. Platelets and platelet contents, such as growth factors produced by the platelets can be used to treat diseases and conditions that would benefit from platelet administration. For example, growth factors released from platelets can be used to treat diseases, disorders, or conditions that would benefit from growth factor treatment.

Compared to platelets administered in the activated state, primed platelets administered intra-articularly can remain in the space for an extended period of time for extended action. Platelets in a PRP composition described herein are in the primed state compared to other commercially available products, such as freeze dried products that are prepared in the presence of cryopreservatives, which are in a fully activated state. For example, commercially available products containing fully activated platelets are designed for cytokine release at the injection point. In examples herein, the PRP composition as described herein can deliver chemokines and cytokines, such as chemokines and/or cytokines for hemostasis, wound repair, and pro-inflammatory and anti-inflammatory processes, to remote locations. In examples where the PRP composition is administered at a distinct location from the platelet activation site the PRP compositions can be administered to deliver platelets to the site of injury and/or the area in need to therapeutic intervention.

Platelets in a PRP composition, upon administration, can recruit other beneficial cells to the site of injury and/or cellular or tissue dysfunction. Platelet release of a diverse set of adhesion molecules and chemokines, allows platelets to adhere to other cell types, such as leukocytes and facilitate recruitment to sites of tissue damage and/or infection. In other examples, alpha granule-derived factors such as platelet factor 4 (PF4) and β-thromboglobulin-F (NAP2) can recruit cells to site of inflammation. Platelets also can increase neutrophil recruitment signals and/or recruit neutrophils (Jenne et al., (2013) International J of Laboratory Hematology 35 (3): 254-261).

Treatment of diseases and conditions with PRP compositions provided herein can be effected by any suitable route of administration using suitable formulations as described herein including, but not limited to, subcutaneous injection, topical, intra-articular, intralesional, oral, intravitreal, intravenous, periocular, intracavitary, and transdermal administration. The PRP compositions described herein provides a species-specific source of concentrated platelets in plasma for, for example, topical, intra-articular, or intralesional administration. If necessary, a particular dosage and duration and treatment protocol can be empirically determined or extrapolated. For example, exemplary doses of fresh platelets can be used as a starting point to determine appropriate dosages. In some examples, PRP compositions provided herein containing platelets in the primed state can be effective at reduced dosage amounts and/or frequencies compared to platelets in the quiescent state or platelets that were fully activated prior to administration. Dosage levels can be determined based on a variety of factors, such as the condition to be treated, the severity of the condition to be treated, the mode or route of administration, the platelet concentration, the percent of platelets in the primed state or activated state, and others.

Upon improvement of a subject's condition, a maintenance dose of a PRP composition can be administered, if necessary; and the dosage, the dosage form, or frequency of administration, or a combination thereof can be modified. In some cases, a subject can require intermittent treatment on a long-term basis upon any recurrence of disease symptoms.

The therapeutic uses include administration of the compositions, alone or in combination with other treatments or agents, for treatment of diseases, disorders or conditions that would benefit from platelet administration and/or delivery of platelet contents to the administration site. The methods of treatment provided herein include combination therapies with a therapeutic agent for the treatment of a disease or disorder for which the therapeutic agent treats. PRP compositions can be administered prior to, subsequent to, intermittently with or simultaneously with the therapeutic agent preparation. Generally, the PRP composition is administered prior to or simultaneously with administration of the therapeutic agent preparation. The PRP compositions can be administered at a site different from the site of administration of the therapeutic molecule or the PRP compositions can be administered at a site the same as the site of administration of the therapeutic molecule.

In some examples, primed platelets in a PRP composition herein are activated immediately before administration for therapeutic use. For example, the platelets in a PRP composition herein can be treated and/or contacted with CaCl, thrombin and/or collagen, and/or exposed to electrical treatment, freezing, sonication or light to effect platelet activation. Dense granule components such as ADP, serotonin, polyphosphates, histamine, and epinephrine act as modifiers of platelet activation and thrombus formation. The activated platelets are immediately administered to the site of injury or site for therapeutic intervention. PRP compositions containing concentrated platelets allow for the delivery of biomolecules released by the platelets activated at the target tissue sites.

The provided methods include methods for use of platelets in platelet rich plasma to treat diseases, disorders or conditions that would benefit from platelet administration, such as for delivery of platelet contents to the administration site. Other methods and uses of a PRP composition include any that are known to one of skill in the art. For example, the PRP compositions are for use in any of the fields of orthopedics, sports medicine, dentistry, ENT, dermatology, neurosurgery, spinal disorders, ophthalmology, urology, wound healing, pain management, analgesic effects, pain relief (e.g., neuropathic, surgical, or wound pain; and/or pain due to sports injuries, orthopedic pathologies, spinal disorders), as well as regenerative therapeutic uses, cosmetic uses, cardiac uses, cardiothoracic uses, and for maxillofacial surgery. In other examples, PRP can be used for treating inflammation, post-operative blood loss, infection, narcotic requirements, osteogenesis, wound, and soft tissue healing and/or regeneration. For example, PRP can be used at sites of vascular injury. In some examples, the reconstituted lyophilized PRP composition can be used to treat diseases, disorders or conditions, such as, for example, inflammatory diseases, disorders or conditions; cardiovascular diseases, disorders or conditions; nervous system diseases, disorders or conditions; tumors; demyelinating diseases, disorders or conditions; digestive system diseases, disorders or conditions; endocrine system diseases, disorders or conditions; reproductive system diseases, disorders or conditions; hemic and lymphatic diseases; disorders or conditions; immunological diseases, disorders or conditions; mental disorders; musculoskeletal diseases, disorders or conditions; neuromuscular diseases, disorders or conditions; metabolic diseases, disorders or conditions; skin and connective tissue diseases, disorders or conditions or injuries; and urological diseases, disorders or conditions; or is for treating osteoarthritis (e.g., stifle joint osteoarthritis (OA)); for treating inflamed joints; for treating synovitis; for orthopedic tissue reconstruction; for spinal fusion; for treating adhesive capsulitis; for treating bone injury or bone dysfunction or bone degradation or lack of bone regrowth or regeneration or a bone disorder; for treating musculoskeletal pathologies; for treating tendon injury or tendon dysfunction or tendon degradation or lack of tendon regrowth or regeneration or a tendon disorder; for treating ligament injury or ligament dysfunction or ligament degradation or lack of ligament regrowth or regeneration or a ligament disorder; for treating a muscle injury or muscle dysfunction or muscle degradation or lack of muscle regrowth or regeneration or a muscle disorder; for tissue regeneration, such as tendon, bone or ligament regeneration; for wound healing; wound healing; or for treating contaminated wounds, tissue lesions or tears. It is understood that PRP compositions provided herein, including reconstituted lyophilized PRP compositions, can be used in any method of treatment or combination therapy for which platelets are used. Combination administration of a PRP composition can support a quicker return to normal clinical function compared to a secondary therapy conducted in the absence of PRP administration. The PRP compositions are effective for treating otitis externa in canines.

The presence and activity of platelets after administration can be monitored. For example, detection of functional platelets can be determined by testing the fluid after administration for platelet activity under appropriate conditions. Gene expression patterns, protein (e.g., growth factor) production, and collagen content at the administration site or site for therapeutic intervention can be assessed. Platelet function also can be assessed. For example, platelet function can be assessed by assessing platelet aggregation; time for platelets to form a plug; by viscoelastometry, to measure clot strength; by lumiaggregometry; by flow cytometry; and by assessing bleeding time, such as the amount of time for bleeding to stop after a wound is administered. Exemplary assays to assess the activity and/or activation of platelets are provided herein.

Exemplary, non-limiting, methods and uses are described in the following subsections.

### 1. Regenerative Therapies

As noted above, PRP compositions can be used as a therapeutic for regenerative therapies. A diverse set of molecular mechanisms have been implicated in the clinical benefits of PRP administration for tissue regeneration. PRP was shown histologically to increase cellular regeneration and angiogenesis. Platelets degranulate upon activation. Platelet activation leads to degranulation and release (e.g., secretion) of alpha granules' contents into the plasma. Chemokines and newly synthesized active metabolites are also released. Platelet α-granule cargo expulsion is fundamental for both hemostasis and thrombosis. Components secreted from granules are important for hemostatic plug formation. PRP injection can initiate tissue regeneration and repair by releasing from platelets biologically active factors and adhesion proteins involved in initiating the hemostatic cascade, connective tissue synthesis, and revascularization. Other bioactive molecules released from alpha-granules act as cytokines or growth factors. Platelet granule constituents also recruit and activate immune cells.

Platelets in PRP can act as therapeutics. For example, PRP as a source of growth factors can be used as a therapeutic. In some examples, platelets in PRP produce (e.g., secrete) growth factors and cytokines that can increase osteoblast and fibroblast proliferation, such as cell proliferation adjacent to a damaged site. PRP administration also can increase production of collagen, glycosaminoglycans and cellularity (Bosch et al., (2012) J Orthop Res. 28:211-7). In some examples, the therapeutic effect of platelet administration is due to anti-oxidant effects of platelets.

Platelets in PRP composition provided herein can be used to promote or enhance the delivery of growth factors contained in the platelets to any of a variety of mammalian tissues in vivo. Platelets contain storage organelles, including alpha granules, containing growth factors such as PDGF, transforming growth factor β (TGF-β) and VEGF, and cytokines and chemokines such as platelet factor 4 (PF4), interleukin 8 (IL-8) and CD40L. Other platelet derived growth factors include cytokines such as pro-platelet basic protein, P-selectin (activator of integrin), and chemokine RANTES (Regulated upon Activation, Normal T Cell Expressed and Presumably Secreted). Growth factors and active metabolites released from platelets can be used as therapeutics, such as therapeutics for disorders or conditions requiring rapid healing and/or tissue regeneration. Growth factors and other bioactive molecules released from platelets in PRP can be delivered to an affected joint can decrease inflammation and promote tissue regeneration and healing. PRP treatment is minimally invasive, does not require surgery or general anesthesia, and has a low risk of side effects.

Primed platelets have intact platelet α-granules. Platelets described herein can be used to deliver cytokines to a location, as once the platelets reach the desired location they can be activated, and, for example, initiate a chemokine cascade and/or recruit stem cells. Primed platelets in a PRP composition provided herein, upon activation, degranulate and secrete an array of mitogenic and chemotactic growth factors, including, for example, platelet derived growth factor (PDGF-αα, PDGF-αβ, PDGF-ββ), transforming growth factor (TGF-β1, TGF-β2), vascular endothelial growth factor (VEGF), fibroblast growth factor (FGF), interleukin-1 (IL-1), and epidermal growth factor (EGF). PRP compositions provided herein contain platelet-derived growth factors, platelet proteins, cytokines, and chemokines that can contribute to tissue repair mechanisms involving mitogenesis, angiogenesis, chemotaxis, and extracellular matrix formation.

For example, PRP based growth factors and cytokines set forth below can be isolated from the sources, and to produce the functions/effects set forth in the table below:

| Platelet Growth | | |
|---|---|---|
| Factor and | | |
| Cytokines | Cell Sources | Function and Effects |
| PDGF | Platelets, endothelial cells, | Mitogenic for mesenchymal cells and |
| (AA-BB-AB) | macrophages, smooth | osteoblasts; stimulates chemotaxis and |
| | muscle cells | mitogenesis in fibroblast/ glial/smooth |
| | | muscle cells; regulates collagenase secretion |
| | | and collagen synthesis; stimulates |
| | | macrophage and neutrophil chemotaxis |
| TGF (α, β) | Macrophages, T | Stimulates undifferentiated mesenchymal cell |
| | lymphocytes, keratinocytes | proliferation; regulates endothelial, |
| | | fibroblastic, and osteoblastic mitogenesis; |
| | | regulates collagen synthesis and collagenase |
| | | secretion; regulates mitogenic effects of other |
| | | growth factors; stimulates endothelial |
| | | chemotaxis and angiogenesis; inhibits |
| | | macrophage and lymphocyte proliferation |
| VEGF | Platelets, macrophages, | Increases angiogenesis and vessel |
| | keratinocytes, endothelial | permeability; stimulates mitogenesis for |
| | cells | endothelial cells |
| EGF | Platelets, macrophages, | Proliferation of keratinocytes, fibroblasts, |
| | monocytes | stimulates mitogenesis for endothelial cells |
| (a-b)-FGF | Platelets, macrophages, | Promotes growth and differentiation of |
| | mesenchymal cells, | chondrocytes and osteoblasts; mitogenic for |
| | chondrocytes, osteoblasts | mesenchymal cells, chondrocytes, and |
| | | osteoblasts |
| CTGF | Platelets, fibroblasts | Promotes angiogenesis, cartilage |
| | | regeneration, fibrosis, and platelet adhesion |
| IGF-1 | Platelets, plasma, epithelial | Chemotactic for fibroblasts and stimulates |
| | and endothelial cells, | protein synthesis. Enhances bone formation |
| | fibroblasts, osteoblasts, bone | by proliferation and differentiation of |
| | matrix | osteoblasts |
| HGF | Platelets, mesenchymal cells | Regulates cell growth and motility in |
| | | epithelial/endothelial cells, supporting |
| | | epithelial repair and neovascularization |
| | | during wound healing |
| KGF | Fibroblasts, mesenchymal | Regulates epithelial migration and |
| | cells | proliferation |
| Ang-1 | Platelets, neutrophils | Induces angiogenesis stimulating migration |
| | | and proliferation of endothelial cells. |
| | | Supports and stabilizes blood vessel |
| | | development via the recruitment of pericyte |
| PF4 | Platelets | Recruits leukocytes and regulates their |
| | | activation. Provides microbiocidal activities |
| SDF-1α | Platelets, endothelial cells, | Calls CD34+ cells, induces their homing, |
| | fibroblasts | proliferation and differentiation into |
| | | endothelial progenitor cells stimulating |
| | | angiogenesis. Recruits mesenchymal stem |
| | | cells and leukocytes |
| TNF | Macrophages, mast cells, | Regulates monocyte migration, fibroblast |
| | T lymphocytes | proliferation, macrophage activation, |
| | | angiogenesis |

Adapted from Everts et al., (2020) Int. J. Mold Sci. 21: 7794.
Abbreviations: PDGF: platelet-derived growth factors; TGF: transforming growth factor; VEGF: vascular endothelial growth factor; EGF: epidermal growth factor; FGF: fibroblast growth factor; CTCG: connective tissue growth factor; IGF: insulin-like growth factor; HGF: hepatocyte growth factor; KGF: keratinocyte growth factor; Ang-1: angiopoietin-1; PF4: platelet factor 4; SDF: stromal cell derived factor; TNF: tumor necrosis factor.

Platelet-rich plasma (PRP) and platelets therein can be used as a source of growth factors. For example, PRP-derived growth factors can be used for soft tissue and bone formation, such as bone healing. In some examples, PRP-derived growth factors can be used on nervous, bone, and epithelial tissue; for facial and cosmetic surgery, such as oral and maxillofacial surgery to increase the rate and degree of bone formation; and for treatment of burns or other injury. In some examples, PRP-derived growth factors are used for wound healing.

PRP can be used to accelerate soft tissue and bone healing, including regeneration of bone, cartilage, and ligaments (see Schnabel et al., (2007) J Orthopaedic Res DOI 10/.1002/jor). The PRP compositions provided herein can be used for the support of regenerative activities in the control of osteoarthritis (joint), tendinopathy, ligament damage or tear(s) or injury, topical wound or burn management or healing, conjunctivitis, corneal ulceration, reconstruction of alveolar-mandibular defects, hoof injuries including sole abscess in equine subjects, septic inflammatory response syndrome (SIRS), laminitis, inflammatory myopathies, endotheliopathy (traumatic), endotheliopathy (septic), asthma and inflammatory bronchitis.

The lyophilized pooled allogeneic platelet rich plasma product also can be used in vitro or ex vivo to support the action of MSCs through the release of growth factors and chemokines. In some examples, the product can be utilized as a media additive to replace bovine serum albumin (BSA). In some examples, the product can support growth and maturation of ex vivo explants for transfusion or transplant. In other examples, the PRP compositions herein can support the formulation of conditioned media to provide growth factors and anti-inflammatory chemokines.

The PRP compositions containing primed platelets can be used for regenerative therapies in any animal species, such as those described herein. For example, PRP compositions containing primed platelets can be used for regenerative therapies in humans, dogs, or horses. For example, primed platelets in a PRP composition herein can be used for treating osteoarthritis in horses and dogs.

PRP compositions provided herein can be used as a therapeutic for cellular and/or tissue regeneration, such as, for example, for use for treating any of the diseases, disorders, and/or conditions listed in, but not limited to, the following sections.

a. Musculoskeletal Disorders, Tissue, Tendon and Ligament Regeneration

Musculoskeletal disorders generally include injuries to or disorders of bone, muscle, tendons, and ligaments. Biologics have demonstrated promising healing benefits for the treatment of various musculoskeletal injuries. The combination of cellular and molecular components in PRP, including platelets, leukocytes, growth factors, fibrinogen, erythrocytes, hormones, and proteases can reduce musculoskeletal pain, accelerate ligament repair, and decrease recovery time (see Kunze et al., (2012) The Orthopaedic Journal of Sports Medicine 10 (2): 23259671211066504). PRP administrations confers benefits at the cellular and molecular levels including increased collagen content, improved cell viability, and decreased cellular regeneration time. Upon activation, platelets release growth factors and adhesion molecules to modulate cellular interactions, such as chemotaxis, cell adhesion, cell migration, cell differentiation, and for immunomodulatory activities. Modulation of these cellular activities can contribute to angiogenesis and inflammatory activities to simulate tissue repair (see e.g., Everts et al., (2020) Int. J. Mold Sci. 21:7794; Cole et al., Sports Health 2 (3): 203-210 (2010).

PRP injections can be more beneficial compared to other non-surgical management options for treating tendon and ligament injury. In inflammatory conditions, platelet compositions containing or in the absence of leukocytes presented anti-inflammatory effects (Castillo-Franz et al., (2019) Muscle Ligament Tendon J. 9:11). In examples, PRP compositions can be administered to treat and/or abrogate symptoms associated with tendon damage, such as, for example, calcaneal tendon rupture. PRP compositions provided herein can be used for treating diseases, disorders, and conditions selected from medial collateral ligament (MCL) injury, osteoarthritis, meniscal injuries, ACL reconstruction, total knee arthroplasty (TKA), and high tibial osteotomy. In examples, PRP can be administered by intra-tendonous administration for treatment of tendon injury or dysfunction. In other examples, PRP can be administered to treat synovitis (or synovial inflammation) or other conditions where the synovium of a joint becomes inflamed (swollen).

Benefits of PRP treatment of ligament injuries were previously associated with PRP-induced increases in an array of genes implicated in promoting extracellular matrix synthesis and tissue remodeling including, for example TGF-b1, biglycan, MMP-1, MMP-13, TIMP-1; angiogenesis such as CD31, VEGF, TSP-1, and markers of neurotrophic support and axonal regeneration such as NT-3, GAP-43, NGF (see Xie et al., (2013) J Surg Res 18-(1): 80-88; Xie et al., (2013) J Surg Res 183 (1): 214-222). Previous research supports a diverse set of mechanisms by which PRP may promote efficacious ligament healing. PRP can be administered to increase growth factor production. A PRP composition provided herein can be administered to provide growth factors, such as any of the growth factors described herein and known in the art to be stored in platelets. In vitro and preclinical studies show that growth factors such as PDGF, TGF-β1, and bFGF are involved in ligament development and healing. Primed platelets in a PRP composition can be administered to promote ligament healing. Primed platelets in a PRP composition can be administered to deliver growth factors such as, for example, PDGF, TGF-β1, and bFGF to a region of ligament injury or a disordered ligament for therapeutic purposes.

For example, PRP compositions provided herein can be administered to a subject with a knee or other joint injury. In some examples of uses for PRP knee injuries include injury to the medial collateral ligament (MCL), patellar tendon, anterior cruciate ligament (ACL), meniscus, lateral collateral ligament (LCL), and posterior cruciate ligament (PCL).

In examples herein, PRP compositions containing primed platelets provided herein can be used to treat tendinopathy or tendon injury. Tendons are slow to heal and rarely self-repair. IL-1β, TNF-α, and IL-6 have previously been identified as cytokines for initiating tendon healing (see Ellis et al., (2022). J Immunol and Regen Med 16:100059). PRP compositions containing primed platelets provided herein can be used to treat tendinopathy or tendon injury, such as, for example tendinopathy or tendon injury of the shoulder, wrist, knee, shin, or heel. PRP can be injected and/or administered at or near the site of tendinopathy. For example, a gel or solid containing the PRP composition provided herein can be added to the site of tendinopathy, for example at or in a joint, such as the shoulder, wrist, or knee. In other examples, a liquid PRP composition provided herein, such as, for example, a reconstituted lyophilized PRP composition can be injected directly into the site of tendinopathy, for example at or in a joint, such as the shoulder, wrist, or knee. In other examples, PRP can be injected and/or administered to the tendon by intra-tendonous administration. For example, a gel or solid containing the PRP composition provided herein can be by intra-tendonous administration. In other examples, a liquid PRP composition provided herein, such as, for example, a reconstituted lyophilized PRP composition can be injected directly into the tendon.

PRP compositions containing primed platelets provided herein can be used to treat muscle strain injuries. Local delivery of PRP was previously shown to increase the IGF-I, hepatocyte GF (HGF), fibroblast GF-2, and TGF-β1 growth factors and previous research indicates that PRP delivery can decrease muscle injury recovery time in animal models (Hammond et al. Am J Sports Med. 37 (6): 1135-1142 (2009)). Therapeutic uses of PRP compositions provided herein can utilize growth factors released from platelet α-granules (see S Schnabel et al., (2007) J Orthopaedic Res DOI 10/.1002/jor).

In examples, reconstituted lyophilized PRP composition containing primed platelets is administered to treat tendon, ligament or bone diseases, disorders, or conditions in any species of animal in need thereof. For example, a reconstituted lyophilized PRP composition containing primed platelets provided herein is administered to horses or elephants for treating orthopedic diseases, disorders, or conditions.

b. Osteoarthritis

Osteoarthritis (OA) is a degenerative joint disease affecting millions of human and veterinary patients worldwide. Non-human animals also are impacted by osteoarthritis-related joint dysfunction. Osteoarthritis is characterized by the loss of articular cartilage, which can lead to pain, stiffness, and loss of motility. Current treatments for osteoarthritis include physical therapy, joint replacement surgery, and treatment with medications. Current treatments have limitations, including adverse side effects and high costs. There is no effective therapeutic protocol for resolution of OA; treatments generally focus on resolving pain.

PRP administration can be used for treating osteoarthritis. Growth factors and other bioactive molecules released from platelets in PRP can promote tissue regeneration and healing, leading to long-lasting improvements in joint function and motility. PRP administered for treatment of arthritis showed sustained clinical improvement compared to other treatments (i.e., hyaluronan). PRP treatment is minimally invasive, does not require surgery or general anesthesia, and has a low risk of side effects. Administration of a PRP composition containing primed platelets provided herein can accelerate healing and reduce pain compared to treatment without primed platelets. In some examples, administration of a PRP composition containing primed platelets can accelerate healing such that other treatments can be administered more readily. For example, administration of a PRP composition containing primed platelets can accelerate healing such that physical therapy can be administered. Administration of a PRP composition can support a quicker return to normal clinical function compared to TPLO conducted in the absence of PRP administration.

PRP compositions containing primed platelets provided herein can be administered for treatment of osteoarthritis. For example, the PRP compositions containing primed platelets provided herein can be administered directly into the joint(s) affected by osteoarthritis. The concentration and volume of the PRP composition can be varied based on several factors, such as, for example, the severity and location of the osteoarthritis. In some examples, PRP compositions containing primed platelets provided herein are administered for treatment of stifle joint osteoarthritis. For example, PRP compositions containing primed platelets provided herein are administered at the time of tibial leveling osteotomy (TPLO) for treating stifle joint osteoarthritis. For example, a PRP composition provided herein is administered to dogs at the time of tibial leveling osteotomy, such as by intra-articular administration during or after surgery. In some examples, the PRP composition for administration for treating osteoarthritis is leukoreduced.

In some examples, the PRP compositions containing primed platelets provided herein can be combined with other therapeutic agents for treating osteoarthritis and/or agents that enhance the regenerative properties of the PRP and/or platelet, such as, for example, hyaluronic acid.

c. Adhesive Capsulitis

Adhesive capsulitis is a painful and debilitating condition characterized by stiffness and limited range of motion in the shoulder joint. The exact cause of adhesive capsulitis is unknown, and may involve inflammation and/or scarring of the capsule surrounding the shoulder joint. Current treatments for adhesive capsulitis are often ineffective or have limited success rates, and include physical therapy, medication, and surgery.

PRP administration can be used for treating adhesive capsulitis. In some examples, the PRP composition comprising primed platelets provided herein is injected directly into the shoulder joint. For example, the PRP can be injected using imaging, such as, for example, ultrasound, to injection and placement for administration of the PRP composition. The concentration and volume of the PRP composition can be varied based on several factors, such as, for example, the severity and location of the adhesive capsulitis. In some examples, a larger volume is injected into the larger shoulder joint compared to an injection volume into a smaller region (e.g., joint) such as a wrist. In some examples, the PRP composition for administration for treating adhesive capsulitis is leukoreduced.

d. Wound Healing

The PRP compositions described herein can be for use as a hemostat and for wound healing. Previous in vivo and in vitro research shows that platelet products can be beneficial for modulating inflammatory processes and for tissue and wound healing. PRP can be administered to wound that was previously not healing. PRP compositions provided herein can be used for treating wounds on the external surface of the subject (e.g., skin or epithelium) or internal wounds. Internal injuries or wounds include intrauterine injuries, for ulcer management, and other internal conditions or injuries. External injuries include injury to the skin and include ophthalmic and ocular injuries and conditions.

PRP compositions provided herein can be beneficial for decreasing inflammation and decreasing the time to heal from tissue damage or injury. For example, administration or topical application of the PRP composition described herein can facilitate endothelial and fibroblast proliferation at the wound site. PRP administration confers therapeutic benefits at the cellular and molecular levels including increased collagen content, improved cell viability, and decreased cellular regeneration time. PRP administration can enhance healing by increasing collagen synthesis, such as by generating dense collagen bundles. In other examples, PRP can increase expression of anti-inflammatory cytokines and matrix enzymes (see Lee et al., 2021). In some examples, the reconstituted lyophilized PRP composition described herein improves wound healing compared to fresh platelets.

Other proteins that promote wound healing and other therapeutic benefits also are PRP-derived. Plasma proteins in PRP can promote healing and other therapeutic benefits, such as, for example, fibrinogen, prothrombin, and fibronectin. For example, fibrinogen in PRP can promote wound healing. In other examples, platelet growth factors such as hepatic growth factor and insulin-like growth factor 1 are localized in the plasma portion of PRP and are involved in tissue repair mechanisms. PRP administration can be used for wound healing. Growth factors, other bioactive molecules released from platelets, and platelet growth factors involved in tissue repair in PRP can promote tissue regeneration and healing, leading to shorter duration of time to heal. PRP can be administered directly to the wound. For example, PRP can be injected, such as injected as a liquid form, into the wound, or can be administered topically, such as in the form of a liquid, gel or solid (e.g., powder). In examples, a PRP compositions can be administered topically or intradermally at the wound edge. In examples, a PRP composition herein can be used to accelerate topical wound healing and to reduce scar formation.

In examples, a PRP composition herein can be used to stop bleeding, such as by forming a clot at the site of bleeding (e.g., a wound), or can be used to promote wound healing by reducing the amount of bleeding at a site, in some instances acting as an adjunct or aid in the clot forming process provided by the patient's blood system. Thus, among many uses of the platelets in the PRP compositions herein, the compositions and primed platelets in the compositions can be used both as hemostatic agents and to accelerate the process of wound healing.

Wound healing can be measured by methods and assays known in the art. For example, wound healing can be measured using the MTT, by collagen contraction and reorganization after PRP administration (i.e., time to or extent of collagen contraction and/or reorganization), cell proliferation at the wound site, clotting times (i.e., shorter clotting times), extent of bleeding arrest, type of bleed the PRP was capable of arresting (i.e., heavy arterial bleeds), and other indices of improved wound healing. PRP containing primed platelets herein can stimulate the supraphysiological release of growth factors to initiate and accelerate acute injury (e.g., wound) repair.

e. Rheumatoid Arthritis

The PRP compositions described herein can be used to treat Rheumatoid arthritis (RA). Rheumatoid arthritis (RA) is a chronic inflammatory illness. It is an autoimmune disease in which the immune system attacks normal tissue components as if they were invading pathogens. The inflammation associated with rheumatoid arthritis primarily attacks the linings of the joints. The membranes lining the blood vessels, heart, and lungs also can become inflamed. RA is characterized by activated B cells and plasma cells that are present in inflamed synovium, and in established disease lymphoid follicles and germinal centers. This results in high levels of local immunoglobulin production and the deposition of immune complexes, which can include IgG and IgM rheumatoid factors, in the synovium and in association with articular cartilage which can serve as initiators of the complement pathway. These actions induce a variety of proinflammatory activities such as for example, alterations in vascular permeability, leukocyte chemotaxis, and the activation and lysis of multiple cell types, and resulting inflammation in the lining of the joints. PRP compositions provided herein can be beneficial for decreasing inflammation associated with rheumatoid arthritis.

### 2. Other Diseases, Disorders and Conditions

PRP compositions provided herein can be used to treat diseases or conditions that would benefit from administration of platelets and/or the growth factors released from platelets. For example, PRP compositions provided herein can be used to provide increased numbers or concentrations of platelets to a site of injury or tissue or cellular dysfunction.

Platelets play an essential role in hemostatic clot formation at the site of vascular injury or hemorrhage (Le Vine et al., (2019) Platelets 30 (1): 88-97). Platelets also can support vascular integrity. In some examples, PRP compositions comprising primed platelets can be used for treating blood clotting disorders. For example, the PRP compositions herein can be used for treating blood clotting disorders selected from among Von Willebrand Disease, a hemophilia, thrombasthenia, thrombocytopenia, thrombocytopenia purpura, trauma, Glanzmann's thrombasthenia, Bernard-Soulier syndrome, or Storage pool disease, or a combination thereof. For example, the PRP composition provided herein is for providing platelets for treating thrombocytopenia. In other examples, PRP compositions provided herein are administered to prevent excess surgical bleeding (e.g., bleeding during or after a surgery). In other examples, platelets in a PRP composition herein can be administered for treating an injury or other trauma. In examples, a PRP composition herein can be used to completely stop bleeding, such as, for example, by forming a clot at the site of bleeding.

PRP compositions provided herein can be used to as a hemostatic agent to treat inherited or acquired bleeding and/or clotting disorders. PRP compositions provided herein also can be used for preventing or treating expected or active excessive bleeding associated with anticoagulant therapy or other therapies or environmental effects that result in inhibition of the clotting cascade. For example, PRP compositions provided herein can be administered to provide rapid control of bleeding to prevent unacceptable blood loss due to injury or surgery, and to minimize concomitant injury to other sites of the body, due to invasion of microbes or activation of blood proteins that negatively affect other body tissues, including joints. For example, the PRP compositions provided herein are for treating diseases, disorders, and conditions that can be treated by PRP and/or platelet administration, including, but not limited to hemophilias, such as Hemophilia A, Hemophilia B, Hemophilia C, and Acquired Hemophilia with Inhibitors. Any level of hemophilia (total, severe, or moderate) can be treated with the PRP compositions herein.

The PRP compositions provided herein can be used for treating subjects with insufficient clotting, such as, for example, subjected with insufficient clotting due to treatment with anticoagulant therapy. The PRP compositions provided herein can be used for treating patients undergoing treatment with anticoagulant agents or other agents or therapies that cause clotting systems to be compromised. In some examples, PRP compositions provided herein can be used in subjects who have normal platelet numbers and normal platelet clotting activity and function, but who would benefit from administration of increased numbers of platelets or platelets in the primed state. In some examples herein, the PRP compositions provided herein are administered in combination with another therapeutic. For example, for treatment of a bleeding or clotting disorder, the PRP compositions provided herein can be added to a second therapeutic such as a procoagulant drug, for accelerated efficacy of the procoagulant drug(s). In other examples, primed platelets in a PRP compositions provided herein are activated prior to administration and clotting is accelerated upon immediate administration of the activated platelets.

In some examples, the PRP compositions containing primed platelets described herein can be for use for treating or preventing diseases, disorders, and conditions with impaired or inappropriate angiogenesis, and diseases involving the vasculature or endothelial cells. These can be, but are not limited to, age-related macular degeneration, coronary artery disease, peripheral vascular disease, islet cell transplantation, fracture and tendon repair, reconstructive surgery, tissue engineering, restenosis, cancer, diabetic retinopathy, rheumatoid arthritis, psoriasis, hemangioma/AEDS-related Kaposi's sarcoma, atherosclerotic plaque rupture, and others.

In some examples, the PRP compositions containing primed platelets described herein can be for use to contribute to healing of injured or diseased tissues and organs, such as, for example, heart, kidney, liver, immune and neurological conditions, and wound healing. In some examples where PRP is used to contribute to healing of diseased tissues, the PRP composition is combined or co-administered with ECM- or MSC-derived exosomes and/or mitochondria. MSC-derived exosomes can be added to the composition or co-administered with the PRP composition.

In some examples, the PRP compositions comprising primed platelets described herein can be for use for treating or preventing viral infections. For example, platelets can capture and sequester pathogens within the vasculature. Interactions between platelets and neutrophils can induce the release of neutrophil extracellular traps (NETs) responsive to bacterial or viral infection. Platelets also were previously shown to internalize pathogens by engulfing them and sequestering them into vacuoles (Jenne et al., (2013) International J of Laboratory Hematology (3): 254-261). In some examples, the PRP compositions provided herein can be used for preventing and/or treating any suitable infection by administering the PRP composition containing primed platelets to the infection site. In the methods, the PRP compositions provided herein are administered to the subject prior to infection or are administered after infection or are administered after the subject has shown symptoms of infection. In some examples the platelets can be for use alone or in combination with another therapeutic for treating a virus such as, for example, human immunodeficiency virus (HIV), Beta coronavirus, a member of the coronavirdae family, SARS-COV, MERS-COV, SARS-COV-2, Ebola virus, Marburg Virus, Lassa virus, Dengue virus, denoviridae, Herpes virus, Papillomaviridae, Polyoma viridae, Poxviridae, Hepadtia viridae, Parvoviridae, Astroviridae, Caliciviridae, Picornaviridae, Coronaviridae, Flaviviridae, Togaviridae, Retroviridae, Orthomyxoviridae, Arenaviridae, Bunyaviridae, Filoviridae, Paramyxoviridae, Rhabdoviridae, or Reoviridae.

The PRP compositions provided herein, lyophilized and/or reconstituted or other forms thereof, can be used for any application for which platelets or their contents are used. Such uses include, but are not limited to, treatment of acute wound/bleeding problems, treatment of ocular diseases, disorders, and conditions, treatment of otic diseases, disorders, and conditions involving the ear, in any subject, including dogs, humans, and other animals. The compositions can be injected into the scalp or applied topically to treat hair loss, such as hair loss related to or resulting from dermatitis or autoimmune diseases, disorders, and conditions. The compositions can be formulated in an ear as an otic flush for otitis externa or any infection like Hot Spots, or for application to the eye for infections, inflammation, or wounds. As described herein the PRP compositions can be rehydrated with an appropriate agent into a form for a particular application, such as formed into a gel or clot for topical application, and used for treatment of any use for which platelets and/or their contents can be therapeutic or prophylactic. The subjects for treatment include animals, such as dogs and horses, and also include humans.

### J. COMBINATION THERAPIES

The PRP compositions provided herein can be combined with another therapeutic or therapeutics. Combination therapies, such as by co-administration or as co-formulations containing a PRP composition containing primed platelets can be employed. Co-formulations containing a PRP composition containing primed platelets are prepared with an additional therapeutic or therapeutics. For example, a PRP composition described herein can be prepared or combined with an additional therapeutic or therapeutics prior to lyophilization and the PRP and the additional therapeutic or therapeutics are lyophilized together. In another example, a PRP composition herein can be prepared or combined with an additional therapeutic or therapeutics after lyophilization and reconstitution of the PRP composition. In such examples, the additional therapeutic or therapeutics is combined with the reconstituted, liquid PRP composition. In some examples the liquid PRP composition (e.g., reconstituted lyophilized PRP composition) is combined with the additional therapeutic or therapeutics immediately prior to administration. In such examples, the liquid PRP composition (e.g., reconstituted lyophilized PRP composition) is combined with the additional therapeutic or therapeutics prior to administration by syringe, such as a two-column syringe. A PRP composition can be co-administered with another therapeutic agent where the PRP composition and other agent are added to a syringe where the PRP composition and other agent are mixed immediately before administration.

In some examples, the PRP composition containing primed platelets provided herein is not physically combined with the additional therapeutic or therapeutics. In such examples, a PRP composition containing primed platelets described herein can be administered concomitantly with or before or after administration of the additional therapeutic or therapeutics.

The PRP compositions provided herein can be prepared or used in combination with other existing drugs and therapeutic agents to treat diseases or disorders or conditions. Such treatments can be performed in conjunction with other anti-inflammatory drugs and/or therapeutic agents. Examples of anti-inflammatory drugs and agents useful for combination therapies include non-steroidal anti-inflammatory drugs (NSAIDs) including salicylates, such as aspirin, traditional NSAIDs such as ibuprofen, naproxen, ketoprofen, nabumetone, piroxicam, diclofenac, or indomethacin, and Cox-2 selective inhibitors such as celecoxib (sold under the trademark CelebrexR) or Rotecoxin (sold under the trademark VioxxR). Other compounds useful in combination therapies include antimetabolites such as methotrexate and leflunomide, corticosteroids or other steroids such as cortisone, dexamethasone, or prednisone, analgesics such as acetaminophen, aminosalicylates such as mesalamine, and cytotoxic agents such as azathioprine (sold under the trademark ImuranR), cyclophosphamide (sold under the trademark CytoxanR), and cyclosporine A. Additional agents that can be used in combination therapies include biological response modifiers. Biological response modifiers can include pro-inflammatory cytokine inhibitors including inhibitors of TNF-alpha such as etanercept (sold under the trademark EnbrelR), infliximab (sold under the trademark RemicadeR), or adalimumab (sold under the trademark HumiraR), and inhibitors of IL-1 such as anakinra (sold under the trademark KineretR). Biological response modifiers also can include anti-inflammatory cytokines such as IL-10, B cell targeting agents such as anti-CD20 antibodies (sold under the trademark RituximabR), compounds targeting T antigens, adhesion molecule blockers, chemokines receptor antagonists, kinase inhibitors such as inhibitors to mitogen-activated protein (MAP) Kinase c-Jun N-terminal Kinase (JNK), or nuclear factor (NF) KB, and peroxisome proliferator-activated receptor-gamma (PPAR-γ) ligands. Additional agents that can be used in combination therapies include immunosuppressants. Immunosuppressants can include tacrolimus or FK-506; mycophenolic acid; calcineurin inhibitors (CNIs); CsA; sirolimus or other agents known to suppress the immune system.

PRP compositions provided herein also can be prepared and/or used in combination with immunomodulatory agents, such as immunomodulatory agents that increase or decrease production of one or more cytokines, up- or down-regulate self-antigen presentation, mask MHC antigens, or promote the proliferation, differentiation, migration, or activation state of one or more types of immune cells. Examples of immunomodulatory agents include but are not limited to non-steroidal anti-inflammatory drugs (NSAIDs) such as aspirin, ibuprofen, celecoxib, diclofenac, etodolac, fenoprofen, indomethacin, ketorolac, oxaprozin, nabumetone, sulindac, tolmetin, rofecoxib, naproxen, ketoprofen, and nabumetone; steroids (e.g., glucocorticoids, dexamethasone, cortisone, hydroxycortisone, methylprednisolone, prednisone, prednisolone, triamcinolone, azulfidine eicosanoids such as prostaglandins, thromboxanes, and leukotrienes; as well as topical steroids such as anthralin, calcipotriene, clobetasol, and tazarotene); cytokines such as TGFβ, IFNα, IFNβ, IFNγ, IL-2, IL-4, IL-10; cytokine, chemokine, or receptor antagonists including antibodies, soluble receptors, and receptor-Fc fusions, such as those against BAFF, B7, CCR2, CCR5, CD2, CD3, CD4, CD6, CD7, CD8, CD11, CD14, CD15, CD17, CD18, CD20, CD23, CD28, CD40, CD40L, CD44, CD45, CD52, CD64, CD80, CD86, CD147, CD152, complement factors (C5, D), CTLA4, eotaxin, Fas, ICAM, IFNα, IFNβ, IFNγ, IFNAR, IgE, IL-1, IL-2, IL-2R, IL-4, IL-5R, IL-6, IL-8, IL-9 IL-12, IL-13, IL-13R1, IL-15, IL-18R, IL-23, integrins, LFA-1, LFA-3, MHC, selectins, TGFβ, TNFα, TNFβ, TNF-R1, T-cell receptor, including Enbrel® (etanercept), Humira® (adalimumab), and Remicade® (infliximab) medications; heterologous anti-lymphocyte globulin; other immunomodulatory molecules such as 2-amino-6-aryl-5 substituted pyrimidines, anti-idiotypic antibodies for MHC binding peptides and MHC fragments, azathioprine, brequinar, Bromocriptine, cyclophosphamide, cyclosporine A, D-penicillamine, deoxyspergualin, FK506, glutaraldehyde, gold, hydroxychloroquine, leflunomide, malononitriloamides (e.g., leflunomide), methotrexate, minocycline, mizoribine, mycophenolate mofetil, rapamycin, and sulfasalazine.

PRP compositions provided herein also can be prepared and/or used in combination with cytokines which include, but are not limited to lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; Müllerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, -beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL).

Other exemplary therapeutics that can be prepared and/or used with a PRP composition containing primed platelets as detailed herein include cytokines and other agents that stimulate cells of the immune system and enhance desired effector function. For example, agents that stimulate NK cells include IL-2; agents that stimulate macrophages include but are not limited to C5a, formyl peptides such as N-formyl-methionyl-leucyl-phenylalanine. Cargo include agents that stimulate neutrophils, such as, for example, G-CSF and GM-CSF. Additional agents include, but are not limited to, interferon gamma, IL-3, and IL-7.

PRP compositions provided herein also can be prepared and/or used in combination with other agents that are administered to treat wounds or to increase wound healing, such as decrease the time for the wound to heal. For example, PRP compositions provided herein can be prepared or used in combination with antibiotics. Antibiotics that can be included in a PRP composition or combination include but are not limited to: aminoglycoside antibiotics (e.g., apramycin, arbekacin, bambermycins, butirosin, dibekacin, gentamicin, kanamycin, neomycin, netilmicin, paromomycin, ribostamycin, sisomicin, and spectinomycin), aminocyclitols (e.g., spectinomycin), amphenicol antibiotics (e.g., azidamfenicol, chloramphenicol, florfenicol, and thiamphenicol), ansamycin antibiotics (e.g., rifamide and rifampin), carbapenems (e.g., imipenem, meropenem, and panipenem); cephalosporins (e.g., cefaclor, cefadroxil, cefamandole, cefatrizine, cefazedone, cefozopran, cefpimizole, cefpiramide, cefpirome, cefprozil, cefuroxime, cefixime, cephalexin, and cephradine), cephamycins (cefbuperazone, cefoxitin, cefminox, cefmetazole, and cefotetan); lincosamides (e.g., clindamycin, and lincomycin); macrolide (e.g., azithromycin, brefeldin A, clarithromycin, erythromycin, roxithromycin, and tobramycin), monobactams (e.g., aztreonam, carumonam, and tigemonam); mupirocin; Oxacephems (e.g., flomoxef, latamoxef, and moxalactam); penicillins (e.g., amdinocillin, amdinocillin pivoxil, amoxicillin, bacampicillin, benzylpenicillinic acid, benzylpenicillin sodium, epicillin, fenbenicillin, floxacillin, penamecillin, penethamate hydriodide, penicillin o-benethamine, penicillin O, penicillin V, penicillin V benzoate, penicillin V hydrabamine, penimepicycline, and phenethicillin potassium); polypeptides (e.g., bacitracin, colistin, polymyxin B, teicoplanin, and vancomycin); quinolones (such as for example, amifloxacin, cinoxacin, ciprofloxacin, enoxacin, enrofloxacin, fleroxacin, flumequine, gatifloxacin, gemifloxacin, grepafloxacin, lomefloxacin, moxifloxacin, nalidixic acid, norfloxacin, ofloxacin, oxolinic acid, pefloxacin, pipemidic acid, rosoxacin, rufloxacin, sparfloxacin, temafloxacin, tosufloxacin, and trovafloxacin); rifampin; streptogramins (e.g., quinupristin, and dalfopristin); sulfonamides (sulfanilamide, and sulfamethoxazole); and tetracyclines (chlortetracycline, demeclocycline hydrochloride, demethylchlortetracycline, doxycycline, Duramycin, minocycline, neomycin, oxytetracycline, streptomycin, tetracycline, and vancomycin).

In another example, PRP compositions provided herein can be prepared with or used in combination with anti-fungal agents, which include, but are not limited to, amphotericin B, ciclopirox, clotrimazole, econazole, fluconazole, flucytosine, itraconazole, ketoconazole, miconazole, nystatin, terbinafine, terconazole, and tioconazole. In some examples, PRP compositions described herein are administered or prepared in combination with one or more antiviral agents, including but not limited to protease inhibitors, reverse transcriptase inhibitors, and others, including type I interferons, viral fusion inhibitors, neuraminidase inhibitors, acyclovir, adefovir, amantadine, amprenavir, clevudine, enfuvirtide, entecavir, foscarnet, ganciclovir, idoxuridine, indinavir, lopinavir, pleconaril, ribavirin, rimantadine, ritonavir, saquinavir, trifluridine, vidarabine, and zidovudine.

Pharmaceutical compositions containing a PRP composition, such as a PRP composition containing primed platelets described herein, can be used to treat any disease or condition treated by platelet administration or treated by platelets in combination with plasma or treated by platelets or platelet-rich plasma. Also provided are combinations of a PRP composition and another treatment or compound for treatment of a disease, disorder, or condition in which platelets play a role or are involved in the etiology or can effect treatment. The PRP composition and additional therapeutic agent(s) can be packaged as separate compositions for administration together or sequentially or intermittently. Alternatively, they can be provided as a single composition for administration or as two compositions for administration as a single composition. The combinations can be packaged as kits, optionally with additional reagents, instructions for use, vials and other containers, syringes, and other items for use of the PRP composition.

PRP compositions can be formulated or used in combination with an additional therapeutic agent(s) where the additional therapeutic agent is for treating a disease, disorder, or condition, such as, for example, any of the therapeutic agents listed and described herein. The PRP composition comprising primed platelets can be formulated with chemokines to support a particular indication/use. The PRP composition comprising primed platelets can be combined with MSC conditioned media, MSCs and/or targeted macrophages. The PRP composition comprising primed platelets can be co-administered with autologous stem cells or allogeneic stem cells. In such examples, where PRP is used or co-administered with autologous or allogeneic stem cells the cells and platelets work in a synergistic manner. In some examples PRP is added to autologous or allogeneic stem cell cultures ex vivo and cells are cultured in the presence of PRP and the cells are added as a therapy. In other examples, PRP is used or co-administered with autologous or allogeneic stem cells or cell culture where the presence of the PRP prolongs the effect of stem cells upon administration to a subject. In some examples, PRP compositions provided herein can be used in combination with other agents (i.e., therapeutic agent(s)) that are administered to treat tendon and ligament injury or degeneration or tendinopathy. For example, PRP compositions containing primed platelets herein for treating tendon and/or ligament injury and/or degeneration and/or tendinopathy can be prepared or formulated or used in combination with one or more of inert PSSAG, hyaluronic acid, chondroitin, triamcinolone and/or small molecules such as IRAP-1. For example, PRP compositions can be used in combination with other agents that are administered to treat medial collateral ligament (MCL) injury; osteoarthritis, meniscal injuries, ACL reconstruction, total knee arthroplasty (TKA), and/or high tibial osteotomy. PRP compositions provided herein also can be used in combination with other agents that are administered to treat musculoskeletal disorders, and for soft tissue repair.

PRP compositions provided herein also can be prepared or used in combination with other agents that are administered to treat muscle strain injuries. In examples, PRP compositions provided can be prepared with or administered in combination with the IGF-I, hepatocyte GF (HGF), fibroblast GF-2, and TGF-β1 growth factors. In some embodiments, the combination of PRP with other agents can decrease muscle injury recovery time.

In some examples, PRP compositions provided herein can be prepared or used in combination with other agents (i.e., therapeutic agent(s)) that are administered to treat ocular diseases, disorders, or conditions. For example, a PRP composition containing primed platelets, such as a reconstituted lyophilized PRP composition can be prepared or administered with mesenchymal stem cells (MSCs) for repairing ocular tissue. PRP compositions administered with MSCs can increase the efficacy of PRP administration for treating ocular conditions, such as ocular lesions. In other examples, a PRP composition containing primed platelets, such as a reconstituted lyophilized PRP composition can be prepared or administered with antibiotics, antimycotics, and/or other anti-inflammatory medications as needed. PRP compositions provided herein also can be used in combination with other agents that are administered to treat ligament injuries.

A PRP composition described herein can be filtered through a filter to exclude cell membranes to produce a platelet lysate. For example, a PRP composition can be passed through a 0.2 micron filter to exclude cells and membranes to become platelet lysate. A platelet lysate from which cell membranes are removed can be used to deliver platelet components, such as contents of platelet granules, to a subject.

In some examples, platelets for inclusion in a PRP composition herein are cultured with other cells prior to administration. For example, platelets can be isolated from a subject or multiple subjects and pooled and then cultured with other cells. Platelets can be cultured with neutrophils prior to administration. Platelets can modulate neutrophil function; platelets can act as immunomodulators to attenuate some or augment different neutrophil functions (see Hally et al., (2019) PLOS One 14 (10): e0223444). Co-culture of platelets with other cell types can affect the neurotrophic factors released from the cells and/or platelets. In some examples PRP is added to autologous or allogeneic stem cell cultures ex vivo and cells are cultured in the presence of PRP. PRP co-culture with autologous or allogeneic stem cells or cell culture can prolong the effect of stem cells that are administered in vivo or that are prepared in vitro.

### K. FORMS OF PRP COMPOSITIONS

The PRP compositions containing primed platelets described herein can be formulated as the sole therapeutic agent in the composition, or can be combined with other active ingredients and/or agents. The PRP composition contains primed platelets from any species, such as, for example any mammalian species human or nonhuman. In examples, the PRP compositions containing primed platelets described herein can be in a solid or liquid form. Liquid PRP compositions that are lyophilized are in a solid form such as a powder or a wafer. The PRP compositions described herein can be used as a topical powder after lyophilization. Liquid PRP compositions comprising primed platelets can be administered as a liquid after rehydration of the lyophilized powder. PRP compositions comprising primed platelets can be administered as a gel form after rehydration of the lyophilized powder.

### Liquid PRP Composition

The normal human range of platelet concentration is about or at 150,000 to 450,000 platelets per μL of whole venous blood (see e.g., Ehrenfest et al., Trends Biotechnol. 27 (3): 158-167 (2009); de Mos, M., et al. Am J Sports Med. 36 (6): 1171-1178 (2008)). Normal dogs and cats have platelet counts at or about or greater than 200,000/μL; normal horses have platelet counts at or about or greater than 100,000/μL, younger foals can have higher platelet counts than adults, of at or about or greater than 200,000/μL; normal ruminants have higher platelet counts than horses that generally is at or about or greater than 400,000/μL, particularly in younger animals (see Cornell University College of Veterinary Medicine EClinPath (URL://eclinpath.com/hemostasis/tests/platelet-number/)). A PRP composition containing primed platelets provided herein has the same concentration of platelets as donor blood or is more concentrated with platelets than normal, physiological whole blood and/or plasma. In examples, a PRP composition prepared herein has a higher concentration of platelets than whole blood. In examples, a PRP composition prepared herein from one animal species (e.g., horse) has a higher concentration of platelets than whole blood from the corresponding species (e.g., horse). In such examples of PRP compositions prepared herein, platelet concentrations can range from 1 to 14, such as 2.5 to 8 times the platelet concentration in whole blood or plasma. For example, the platelet concentration in a PRP composition provided herein contains at least or at least about 1, 1.1, 1.2, 1.3, 1.4, 1.5, 1.6, 1.7, 1.8, 1.9, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, 14 times the concentration of platelets in whole blood, such as whole blood from the same species of animal from which the platelets are isolated.

The number of platelets in a PRP composition provided herein can vary depending on various factors such as the number of donors, manufacturing batch size, collection time, collection efficiency, collection method, and other parameters. The concentration of platelets in a reconstituted lyophilized PRP composition will vary depending on the volume of the reconstitution solution (e.g., volume of water or saline). Platelets at too high of a concentration can self-activate and/or aggregate due to, for example, physical contact between platelets; the PRP compositions provided herein contain at or less than 1,000,000 platelets/μL, such as the concentrations listed below. The concentrations of platelets in PRP compositions are below levels where platelets self-activate and/or aggregate, such as at or below 1,000,000 platelets/μL. A PRP composition described herein contains 200,000 to 1,000,000 platelets per microliter, such as, for example between 200,000 platelets/μL and 1,000,000 platelets/μL, 200,000 platelets/μL and 800,000 platelets/μL, 200,000 platelets/μL and 600,000 platelets/μL, 200,000 platelets/μL and 500,000 platelets/μL, 200,000 platelets/μL and 400,000 platelets/μL, 200,000 platelets/μL and 300,000 platelets/μL, 200,000 platelets/μL and 250,000 platelets/μL, 250,000 platelets/μL and 1,000,000 platelets/μL, 250,000 platelets/μL and 800,000 platelets/μL, 250,000 platelets/μL and 600,000 platelets/μL, 250,000 platelets/μL and 500,000 platelets/μL, 250,000 platelets/μL and 400,000 platelets/μL, 250,000 platelets/μL and 300,000 platelets/μL, 400,000 platelets/μL and 1,000,000 platelets/μL, 400,000 platelets/μL and 800,000 platelets/μL, 400,000 platelets/μL and 600,000 platelets/μL, or 400,000 platelets/μL and 500,000 platelets/μL, or has at least 100,000, 200,000, 250,000, 300,000, 350,000, 400,000, 500,000, 600,000, 700,000, 800,000, 900,000, or 1,000,000, platelets/μL. For example, each microliter of a liquid PRP composition contains at least 300,000 platelets. For example, each microliter of a liquid PRP composition contains up to 1,000,000 platelets.

### Lyophilized PRP Composition

Of interest herein are lyophilized powders, which can be reconstituted for administration as solutions, emulsions, and other mixtures. Lyophilized powders containing primed platelets also can be reconstituted and formulated as liquids, solids, or gels. The lyophilized PRP composition contains primed platelets and is prepared without a cryopreservative.

The lyophilized PRP composition is provided as a freeze-dried powder or solid. The lyophilized PRP composition containing primed platelets can be used as a topical powder. In examples herein, a lyophilized PRP composition contains at least 2×10⁸ platelets, such as at least 2×10⁸, 5×10⁸, 10⁹, 5×10⁹, 10¹⁰, 5×10¹⁰, 10¹¹, 5×10¹¹, 10¹², or 5×10¹² platelets. For example, a vial of a lyophilized PRP composition provided herein contains between 2×10⁸ and 5×10¹² platelets, such as between 5×10⁸ and 5×10¹² platelets, between 10⁹ and 5×10¹² platelets, between 5×10⁹ and 5×10¹² platelets, between 10¹⁰ and 5×10¹² platelets, between 5×10¹⁰ and 5×10¹² platelets, between 10¹¹ and 5×10¹² platelets, between 5×10¹¹ and 5×10¹² platelets, between 10¹² and 5×10¹² platelets, between 5×10⁸ and 10¹² platelets, between 10⁹ and 10¹² platelets, between 5×10⁹ and 10¹² platelets, between 10¹⁰ and 10¹² platelets, between 5×10¹⁰ and 10¹² platelets, between 10¹¹ and 10¹² platelets, between 5×10¹¹ and 10¹² platelets, between 5×10⁸ and 5×10¹¹ platelets, between 10⁹ and 5×10¹¹ platelets, between 5×10⁹ and 5×10¹¹ platelets, between 10¹⁰ and 5×10¹¹ platelets, between 5×10¹⁰ and 5×10¹¹ platelets, between 10¹¹ and 5×10¹¹ platelets, between 5×10⁸ and 10¹¹ platelets, between 10⁹ and 10¹¹ platelets, between 5×10⁹ and 10¹¹ platelets, between 10¹⁰ and 10¹¹ platelets, between 5×10¹⁰ and 10¹¹ platelets, between 5×10⁸ and 5×10¹⁰ platelets, between 10⁹ and 5×10¹⁰ platelets, between 5×10⁹ and 5×10¹⁰ platelets, between 10¹⁰ and 5×10¹⁰ platelets, between 5×10⁸ and 10¹⁰ platelets, between 10⁹ and 10¹⁰ platelets, between 5×10⁹ and 10¹⁰ platelets, between 5×10⁸ and 5×10⁹ platelets, between 10⁹ and 5×10⁹ platelets, and between 5×10⁸ and 10⁹ platelets. In some examples herein, a lyophilized PRP composition, such as a vial of a lyophilized PRP composition for administration, contains at least or at least about or about 1×10⁹ platelets.

In some examples, the lyophilized PRP composition described herein contains at or about at least 5×10⁸, 10⁹, 5×10⁹, 10¹⁰, 5×10¹⁰ platelets per milligram of lyophilized powder, such as, for example between 5×10⁸ and 5×10¹⁰ platelets, 8×10⁸ and 5×10¹⁰ platelets, between 10⁹ and 5×10¹⁰ platelets, between 3×10⁹ and 5×10¹⁰ platelets, between 5×10⁹ and 5×10¹⁰ platelets, between 10¹⁰ and 5×10¹⁰ platelets, between 5×10⁸ and 10¹⁰ platelets, between 10⁹ and 10¹⁰ platelets, between 5×10⁹ and 10¹⁰ platelets, between 5×10⁸ and 5×10⁹ platelets, between 10⁹ and 5×10⁹ platelets, and between 5×10⁸ and 10⁹ platelets/mg power. In a particular example, a lyophilized PRP composition produced from a liquid composition containing 1× 10⁹ to 4×10⁹ platelets, contains at or about 1×10⁹ to 4×10⁹ platelets in a solid form after lyophilization. In such examples, if the solid (e.g., powder) PRP composition is at or about, for example, 1.2 mg then the total platelets of at or about 1×10⁹ to 4×10⁹ platelets are in the 1.2 mg. For example, if 4×10⁹ platelets are in the 1.2 mg of lyophilized (powder) PRP composition, then the lyophilized PRP composition contains about 3×10⁹ platelets/mg.

In other examples, the concentration of platelets in a freeze-dried PRP composition is at or about 300,00 to 4,000,000 platelets/μg, or is 300,00 to 3,000,000 platelets/μg, or is 300,00 to 2,000,000 platelets/μg, or is 500,00 to 4,000,000 platelets/μg, or is 500,00 to 3,000,000 platelets/μg, or is 500,00 to 2,000,000 platelets/μg, or is 700,00 to 4,000,000 platelets/μg, or is 700,00 to 3,000,000 platelets/μg, or is 700,00 to 2,000,000 platelets/μg.

A freeze-dried PRP composition of any of claims 1-32, wherein the concentration of platelets is at least or at least about 500,000 platelets/μg, 550,000 platelets/μg, 600,000 platelets/μg, 650,000 platelets/μg, 700,000 platelets/μg, 750,000 platelets/μg, 800,000 platelets/μg, 950,000 platelets/μg, 1,000,000 platelets/μg, 1,100,000 platelets/μg, 1,200,000 platelets/μg, 1,300,000 platelets/μg, 1,400,000 platelets/μg, 1,500,000 platelets/μg, 1,600,000 platelets/μg, 1,700,000 platelets/μg, 1,800,000 platelets/μg, 1,900,000 platelets/μg, 2,000,000 platelets/μg, 2,100,000 platelets/μg, 2,200,000 platelets/μg, 2,300,000 platelets/μg, 2,400,000 platelets/μg, 2,500,000 platelets/μg, 2,600,000 platelets/μg, 2,700,000 platelets/μg, 2,800,000 platelets/μg, 2,900,000 platelets/μg, 3,000,000 platelets/μg, 3,100,000 platelets/μg, 3,200,000 platelets/μg, 3,300,000 platelets/μg, 3,400,000 platelets/μg, 3,500,000 platelets/μg, 3,600,000 platelets/μg, 3,700,000 platelets/μg, 3,800,000 platelets/μg, 3,900,000 platelets/μg, 4,000,000 platelets/μg, 4,100,000 platelets/μg, 4,200,000 platelets/μg, 4,300,000 platelets/μg, 4,400,000 platelets/μg, 4,500,000 platelets/μg, 4,600,000 platelets/μg, 4,700,000 platelets/μg, 4,800,000 platelets/μg, 4,900,000 platelets/μg, or 5,000,000 platelets/μg.

Reconstitution of this lyophilized powder with a solution provides a formulation for use for administration. The precise amount depends upon the indication treated and platelet concentration. Such amount can be empirically determined.

### Reconstituted Lyophilized PRP Composition

The sterile, lyophilized powder is prepared by freeze drying of a liquid PRP solution. The lyophilized PRP composition can be reconstituted in a liquid wherein the primed state of the platelets in maintained. In examples herein, the lyophilized PRP composition is reconstituted in sterile water (e.g., sterile water for injection) or saline. The reconstituted PRP composition can be used, for example, for intralesional injection or intra-articular infusion or injection.

The lyophilized PRP composition is rehydrated in a volume sufficient to prepare a liquid PRP composition comprising primed platelets for treatment. For example, the lyophilized PRP composition is rehydrated to prepare a volume of liquid sufficient to prepare a liquid composition containing 200,000 to 1,000,000 platelets per microliter of the composition. In some examples, the lyophilized PRP composition is rehydrated in at least or at least about 1 mL, such as 1.5 mL, 2 mL, 2.5 mL, 3 mL, 3.5 mL, 4 mL, 4.5 mL, 5 mL, 5.5 mL, 6 mL, 6.5 mL, 7 mL, 7.5 mL, 8 mL, 8.5 mL, 9 mL, or more.

The reconstituted lyophilized PRP composition described herein contains 200,000 to 1,000,000 platelets per microliter, such as, for example between 200,000 platelets/μL and 1,000,000 platelets/μL, 200,000 platelets/μL and 800,000 platelets/μL, 200,000 platelets/μL and 600,000 platelets/μL, 200,000 platelets/μL and 500,000 platelets/μL, 200,000 platelets/μL and 400,000 platelets/μL, 200,000 platelets/μL and 300,000 platelets/μL, 200,000 platelets/μL and 250,000 platelets/μL, 250,000 platelets/μL and 1,000,000 platelets/μL, 250,000 platelets/μL and 800,000 platelets/μL, 250,000 platelets/μL and 600,000 platelets/μL, 250,000 platelets/μL and 500,000 platelets/μL, 250,000 platelets/μL and 400,000 platelets/μL, 250,000 platelets/μL and 300,000 platelets/μL, 400,000 platelets/μL and 1,000,000 platelets/μL, 400,000 platelets/μL and 800,000 platelets/μL, 400,000 platelets/μL and 600,000 platelets platelets/μL, or 400,000 platelets/μL and 500,000 platelets/μL, or has at least 100,000, 200,000, 250,000, 300,000, 350,000, 400,000, 500,000, 600,000, 700,000, 800,000, 900,000, or 1,000,000 platelets/μL. For example, each microliter of a reconstituted lyophilized PRP composition contains an average of at least 300,000 platelets. For example, each microliter of a reconstituted lyophilized PRP composition contains an average of up to 1,000,000 platelets.

A reconstituted lyophilized PRP composition containing primed platelets provided herein has the same concentration of platelets as donor blood or is more concentrated with platelets than normal, physiological whole blood and/or plasma. In examples, a reconstituted lyophilized PRP composition prepared herein has a higher concentration of platelets than whole blood. In examples, a reconstituted lyophilized PRP composition prepared herein from one animal species (e.g., horse) has a higher concentration of platelets than whole blood from the corresponding species (e.g., horse). In such examples of reconstituted lyophilized PRP compositions prepared herein, platelet concentrations in can range from 1 to 14, such as 2.5 to 8, times the platelet concentration in whole blood or plasma. For example, the platelet concentration in a PRP composition herein, such as a reconstituted lyophilized PRP composition provided herein contains at least or at least about 1, 1.1, 1.2, 1.3, 1.4, 1.5, 1.6, 1.7, 1.8, 1.9, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, 14 times the concentration of platelets in whole blood, such as whole blood from the same species of animal from which the platelets are isolated.

A reconstituted lyophilized PRP composition containing primed platelets provided herein and/or prepared by the methods described herein has the same or about the same number of platelets after lyophilization and reconstitution as the number of platelets in the composition prior to lyophilization. In examples herein, a reconstituted lyophilized PRP composition, such as a vial of a reconstituted lyophilized PRP composition for administration, contains at least 2×10⁸ platelets, such as at least 2×10⁸, 5×10⁸, 1×10⁹, 5×10⁹, 1×10¹⁰, 5×10¹⁰, 1×10¹¹, 5×10¹¹, 1×10¹², or 5×10¹² platelets. For example, a vial of a reconstituted lyophilized PRP composition contains between 2×10⁸ and 5×10¹² platelets, such as between 5×10⁸ and 5×10¹² platelets, between 10⁹ and 5×10¹² platelets, between 5×10⁹ and 5×10¹² platelets, between 10¹⁰ and 5×10¹² platelets, between 5×10¹⁰ and 5×10¹² platelets, between 10¹¹ and 5×10¹² platelets, between 5×10¹¹ and 5×10¹² platelets, between 10¹² and 5×10¹² platelets, between 5×10⁸ and 10¹² platelets, between 10⁹ and 10¹² platelets, between 5×10⁹ and 10¹² platelets, between 10¹⁰ and 10¹² platelets, between 5×10¹⁰ and 10¹² platelets, between 10¹¹ and 10¹² platelets, between 5×10¹¹ and 10¹² platelets, between 5×10⁸ and 5×10¹¹ platelets, between 10⁹ and 5×10¹¹ platelets, between 5×10⁹ and 5×10¹¹ platelets, between 10¹⁰ and 5×10¹¹ platelets, between 5×10¹⁰ and 5×10¹¹ platelets, between 10¹¹ and 5×10¹¹ platelets, between 5×10⁸ and 10¹¹ platelets, between 10⁹ and 10¹¹ platelets, between 5×10⁹ and 10¹¹ platelets, between 10¹⁰ and 10¹¹ platelets, between 5×10¹⁰ and 10¹¹ platelets, between 5×10⁸ and 5×10¹⁰ platelets, between 10⁹ and 5×10¹⁰ platelets, between 5×10⁹ and 5×10¹⁰ platelets, between 10¹⁰ and 5×10¹⁰ platelets, between 5×10⁸ and 10¹⁰ platelets, between 10⁹ and 10¹⁰ platelets, between 5×10⁹ and 10¹⁰ platelets, between 5×10⁸ and 5×10⁹ platelets, between 10⁹ and 5×10⁹ platelets, and between 5×10⁸ and 10⁹ platelets. In some examples herein, a reconstituted lyophilized PRP composition, such as a vial of a reconstituted lyophilized PRP composition for administration, contains at least or at least about or about 1×10⁹ platelets. For example, if the PRP composition in a vial prior to lyophilization contains at least about or about 1×10⁹ platelets, the PRP composition in a vial prior after lyophilization, including after lyophilization and reconstitution, contains at least about or about 1×10⁹ platelets or can contain 1%, 2%, 3%, 4%, 5%, 6%, 7%, 8%, 9%, 10%, 11%, 12%, 13%, 14%, 15%, 16%, 17%, 18%, 19%, 20% less platelets than the PRP composition prior to lyophilization. For example, the number of platelets in a PRP composition after lyophilization, including after lyophilization and reconstitution, contains at least about or about 1×10⁹ platelets or can contain at least or at least about 80%, 81%, 82%, 83%, 84%, 85%, 86%, 87%, 88%, 89% 90%, 91%, 92%, 93%, 94%, 95%, 96%, 97%, 98%, 99% or 100% of the platelets in the composition prior to lyophilization.

### Other Blood Cells in the Composition

PRP compositions provided herein contain primed platelets. PRP compositions containing primed platelets provided herein also can contain other blood cells in the composition. PRP compositions, such as the PRP compositions containing primed platelets as described herein, can be prepared as leukoreduced, containing leukocytes, or leukorich (e.g., enriched for leukocytes compared to whole blood).

In some examples, the PRP composition, which is freeze-dried in the absence of any cryopreservative yields a composition where the majority of the cells in the composition are platelets. In these examples, the composition is substantially free of red and white blood cells. By virtue of the collection and preparation protocol described herein, in some examples the platelet-rich plasma (PRP) composition is leukoreduced, with uniform functional cell types.

Leukoreduced PRP compositions, such as reconstituted lyophilized PRP compositions containing primed platelets, have a lower concentration of WBCs compared or fewer WBCs compared to the same volume of whole blood. Platelets isolated from subjects can be subjected to treatment to remove WBCs. For example, platelets isolated from subjected can be filtered to exclude WBCs by size. In some examples, during apheresis to collect platelets (i.e., plateletpheresis) white blood cells also are isolated with the platelets. To produce a leukoreduced composition containing platelets, following apheresis the platelet composition is filtered to remove the WBCs. For example, the WBCs filtered out by size; the WBCs are removed by a filter which allows platelets to pass through but wherein the WBCs remain embedded in the filter or are excluded from passing through the filter. Leukoreduced platelet compositions contain fewer WBCs or contain a lower concentration of WBCs compared to whole blood.

A leukoreduced PRP composition described herein contains 200,000 to 1,000,000 platelets per microliter, and less than 2000 white blood cells per microliter. For example, the ratio of the number of platelets per microliter to the number of white blood cells is at least or at least about 400:1, 380:1, 360:1, 340:1, 320:1, 300:1, 280:1, 260:1, 240:1, 220:1, 200:1, 180:1, 160:1, 140:1, 120:1, 100:1, 80:1, 60:1, 40:1, 20:1, or 10:1. In a particular example of a leukoreduced PRP composition described herein, the reconstituted lyophilized PRP containing primed platelets is provided as a single dose of 400,000 platelets/μl+/−100,000 platelets/μl of and less than 1500 white blood cells/μl in the PRP composition.

PRP compositions also can be prepared as leukorich. A leukorich PRP composition contains primed platelets and contain white blood cells. In some examples, leukorich PRP compositions have the same concentration of WBCs compared to whole blood. In other examples, leukorich PRP compositions have a higher concentration of WBCs compared to whole blood. Platelets isolated from subjects can be subjected to treatment to collect WBCs along with platelets or can be subjected to treatment to enrich for WBCs. Leukorich platelet compositions contain the same or higher concentration of WBCs compared to whole blood.

The PRP composition contains platelets and can contain red blood cells and/or white blood cells. Platelets can be distinguished from red blood cells and/or white blood cells. For example, platelets can be distinguished from red blood cells and/or white blood cells by size. The diameter of healthy RBCs is species-dependent. The diameter of healthy RBCs in humans is at or about 6-8 μm and the thickness of healthy RBCs in humans is at or about 1.7 to 2.2 μm. RBCs in cattle and horses have diameters of at or about 5-6 μm; sheep have RBC diameters of at or about 4-5 μm; goats have RBC diameters of at or about 2.5-3.9 μm; and rodents have RBC diameters of at or about 4-7 μm (see e.g., Adili et al., “Species determination using the red blood cells morphometry in domestic animals,” Veterinary World 9 (9): 960-963; Ward et al., “Chapter 19-Hematopoietic and Lymphoid Tissues,” Comparative Anatomy and Histology 2^{nd} edition, p. 365-401 (2019)).

White blood cells have variability in sizes and diameters depending on the type and activation status. Among the granulocytes, healthy, normal neutrophils in adult humans are at or about 12-15 μm in diameter; eosinophils are at or about 12-17 μm in diameter; and basophils are at or about 12-16 μm in diameter. Healthy monocytes in humans measure at or about 12-20 μm in diameters. Maturation of monocytes into macrophages result in larger particles or diameters of at or about 21 μm. Among the lymphocytes, most are at or about 6-9 μm and others are at or about 9 to 19 μm in their activated state. For example, T- and B-lymphocytes are typically at or about 7-8 μm in diameter, and natural killer cells typically measure at or about 12-15 μm in diameter.

A leukoreduced or leukorich PRP composition can be used for treating diseases or conditions for which platelets are administered for therapeutic purposes. The disease or condition to be treated can influence whether a leukoreduced or leukorich PRP composition is administered. For example, for treatment of an infected wound, a leukorich preparation can be desirable to attack and destroy the bacteria, virus, or other organism causing the infection. Monocytes are responsible for control and clearance of viral, bacterial, fungal, and protozoal infections. WBCs (e.g., monocytes) in the PRP composition can be administered to treat injury or prevent infection (e.g., viral, bacterial, fungal, and protozoal infections). Macrophages detect, phagocytose, and eliminate bacteria and other harmful microorganisms. WBCs (e.g., macrophages) in the PRP composition can be administered to treat injury or prevent infection (e.g., bacteria and other harmful microorganism infections) or decrease bacterial contamination. In other examples, a leukoreduced PRP composition is desirable. For example, a leukoreduced PRP composition can be used for administration to joints, or for treatment of musculoskeletal therapies.

In some examples, the PRP composition can contain erythrocytes, or can be treated (e.g., filtered) to remove erythrocytes. In examples, a PRP composition provided herein is treated to remove erythrocytes. For example, a PRP composition herein contains less than 2000 erythrocytes per microliter. For example, the ratio of the number of platelets per microliter to the number of erythrocytes is at least or at least about 400:1, 380:1, 360:1, 340:1, 320:1, 300:1, 280:1, 260:1, 240:1, 220:1, 200:1, 180:1, 160:1, 140:1, 120:1, 100:1, 80:1, 60:1, 40:1, 20:1, or 10:1.

### Stability

The platelets and compositions described herein exhibit long term stability. Long term stability is an advantage in providing platelet products to those in need thereof, such as when transport of the platelet product is required to reach the patients. Lyophilization enables longer stability at ambient temperatures. Freeze-dried platelets can be stored at room temperature, obviating the need for bulky and expensive storage containers/systems (e.g., refrigerators). The PRP composition prepared in the absence of cryopreservative and containing primed platelets can be stored in the freeze-dried state to extend the time for cell viability. The lyophilized PRP composition has a shelf life of at least six months at room temperature or below. In some examples, the lyophilized PRP composition is shelf stable at ambient temperature and humidity for several years. For example, the lyophilized PRP composition can be stored at ambient temperatures or lower than ambient temperatures for at least about 6 months, 9 months, 1 year, 1.5 years, 2 years, 2.5 years, 3 years, 4 years, 5 years, 6 years, 7 years, 8 years, 9 years, 10 years, 15 years, 20 years or more. The PRP composition prepared without cryopreservatives as described herein is lyophilized for long term storage. In some examples, the lyophilized PRP composition is shelf stable at ambient temperature and humidity. In some examples, the lyophilized PRP composition is stored at about 18° C.-25° C., such as about 18° C.-25° C., 18° C.-25° C., 18° C.-25° C., 18° C.-25° C., 18° C.-25° C., 18° C.-25° C., or 18° C.-25° C., or at least or at least about 18° C., 19° C., 20° C., 21° C., 22° C., 23° C., 24° C. or 25° C.

Upon reconstitution, the PRP composition is stored at room temperature, such as ambient temperatures of at or about 18° C.-25° C. until use (e.g., administration to a subject (i.e., patient)). For example, the reconstituted lyophilized PRP composition is stored at ambient temperatures for not more than about six, five, four and a half, or four (4) hours before use. In examples herein, the reconstituted lyophilized PRP composition is stored at ambient temperatures for not more than about four (4) hours before use, such as administration.

Stability of platelets in the rehydrated lyophilized PRP composition is determined by comparing the platelets in the rehydrated lyophilized PRP composition to platelets in the PRP composition prior to lyophilization. The platelets in the rehydrated lyophilized PRP composition are stable if they retain at least one property of platelets in the PRP composition prior to lyophilization. For example, the platelets in the rehydrated lyophilized PRP composition are stable if they retain at least one phenotypic characteristic of platelets in the PRP composition prior to lyophilization. For example, stable platelets in the rehydrated lyophilized PRP composition retain the primed status of the platelets in the PRP composition prior to lyophilization (e.g., platelets subjected to treatment at refrigerated temperatures immediately after collection). For example, in examples of the stable platelets reconstituted lyophilized PRP composition described herein, at least about or about 50%, 55%, 60%, 65%, 70%, 75%, 80%, 85%, 90%, 91%, 92%, 93%, 94%, 95%, 96%, 97%, 98%, 99% or 100% of the platelets retain the primed state. In examples herein, stable platelets in the rehydrated lyophilized PRP composition maintain phosphatidylserine on the outer leaflet of the platelet membrane. For example, in examples of the stable platelets reconstituted lyophilized PRP composition described herein, at least about or about 50%, 55%, 60%, 65%, 70%, 75%, 80%, 85%, 90%, 91%, 92%, 93%, 94%, 95%, 96%, 97%, 98%, 99% or 100% of the platelets retain phosphatidylserine on the outer leaflet of the platelet membrane. As detailed herein, phosphatidylserine externalization can be assessed by lactadherin staining or any other methods known in the art. Platelet stability also refers to the ability of platelets, when rehydrated, to function within normal parameters for fresh platelets, such as providing blood clotting functions, providing growth factors, increasing regenerative properties in surrounding tissues, or other uses described herein (see e.g., section entitled “USES”).

### L. ADMINISTRATION

The PRP composition provided herein can provide a species-specific source of concentrated platelets in plasma for administration. The PRP contains primed platelets for administration. PRPs can be administered by methods known to the skilled artisan, such as a physician or licensed veterinarian. In some examples, the PRP composition is for topical, intra-articular, intravenous, intra-tendinous or intralesional administration, such as intra-articular or intralesional injection. Depending upon the condition treated other routes of administration, such as transdermal patches, oral and rectal administration are also contemplated herein. In some examples, PRP compositions are administered by intralesional injection or administration, intra-articular infusion, intra-tendinous injection, or administration and/or are used as a topical powder under the order of a licensed veterinarian.

In examples herein, for administration, the lyophilized PRP composition is rehydrated in a liquid (e.g., sterile water or sterile saline) prior to administration. For example, a reconstituted lyophilized PRP composition prepared in the absence of cryopreservative and containing primed platelets is loaded and administered using a sterile syringe and needle. Once the lyophilized PRPs are resuspended in the fluid, the liquid PRP mixture is gently mixed by swirling the vial. The reconstituted PRP composition generally is used within 4 hours of rehydration. In examples where the platelets in the PRP composition are activated, such as by addition of an agonist or Ca++, the platelets are activated and the PRP composition containing the activated platelets are immediately administered.

In some examples the lyophilized PRP can be resuspended in sterile water or sterile saline solution. The resuspension volume can vary depending on various factors, including, but not limited to, the mode of administration, desired platelet concentration, indication for use, and other factors. In embodiments, the PRP is reconstituted in a volume of at least or at least about 1 mL, such as 1.5 mL, 2 mL, 2.5 mL, 3 mL, 3.5 mL, 4 mL, 4.5 mL, 5 mL, 5.5 mL, 6 mL, 6.5 mL, 7 mL, 7.5 mL, 8 mL, 8.5 mL, 9 mL, or more. In embodiments, the lyophilized PRP composition can be reconstituted in a volume of 8 mL (e.g., 8 mL of sterile water or sterile saline) and drawn into a sterile syringe containing an attached needle. For example, for administration, the fluid can be drawn into a syringe using the bidirectional clave, and a needle greater than at or about or equal to 22 gauge can be used for administration.

The appropriate volume for reconstitution and/or administration can depend on various factors and it is within the skill of the skilled physician or scientist or veterinarian to determine the volume for administration, such as, for example, the volume to effect a therapeutic effect.

The volume for administration of a PRP composition can depend on the subject, the indication for treatment, and the area of administration. Different uses employ or can employ different concentrations of platelets. Platelet concentration can be varied by varying the amount of liquid, such as sterile water or PBS, added to the lyophilized composition. The dose, for example, can be modified depending upon the subject and size of the subject, and the indication treated. It is within the skill of the doctor to select an appropriate dose and concentration. For example, a small joint will require a smaller volume for administration than a large joint. For example, a PRP composition for intraarticular administration to a knee joint can be prepared and administered in a volume of at or about 2 mL to 5 mL.

In examples, the lyophilized PRP composition is reconstituted in a volume to prepare the rehydrated PRP composition for administration. The rehydrated PRP composition generally is used (e.g., administered) within 12 hours of rehydration, such as within at or about 11.5 hours, 11 hours, 10.5 hours, 10 hours, 9.5 hours, 9 hours, 8.5 hours, 8 hours, 7.5 hours, 7 hours, 6.5 hours, 6 hours, 5.5 hours, 5 hours, 4.5 hours, 4 hours, 3.5 hours, 3 hours, 2.5 hours, 2 hours, 1.5 hours, 1 hour, or less of rehydration. In some examples herein, the rehydrated PRP composition is used (e.g., administered) within 4 hours of rehydration, or less.

Treatment of a subject with a PRP composition provided herein can comprise a single treatment or multiple treatments. In some examples, an animal is provided with two or more treatments. In some examples, PRP containing primed platelets is administered multiple times to the same subject by the same mode of administration (i.e., intra-articular or topical administration). In some examples, PRP containing primed platelets is administered multiple times to the same subject by the different modes of administration (i.e., intra-articular or topical administration).

In examples where the PRP composition provided herein is administered multiple times, such as in a cycle of administration, the length of time of the cycle of administration can be empirically determined, and is dependent on the disease or condition or injury to be treated, the severity of the disease or condition or injury, the subject, and other considerations within the level of skill of the treating physician or veterinarian. In examples where a PRP composition is administered multiple times to the same subject, individual PRP administrations are spaced apart by at least or at least about 2 hours, 3 hours, 6 hours, 12 hours, one day, two days, three days, four days, five days, six days, one week, two weeks, three weeks, one month, 6 weeks, two months, three months, four months, six months, eight months, ten months, one year, fourteen months, sixteen months, eighteen months, two years, or more. The length of time of treatment with a PRP composition containing primed platelets can be one week, two weeks, one month, several months, one year, several years or more. For example, a PRP composition containing primed platelets can be administered twice weekly over a period of a year or more. If disease symptoms persist in the absence of discontinued treatment, treatment can be continued for an additional length of time. Over the course of treatment, evidence of disease and/or treatment-related toxicity or side effects can be monitored.

In addition, the cycle of administration can be tailored to add periods of discontinued treatment to provide a rest period from exposure to the PRP composition. The length of time for the discontinuation of treatment can be for a predetermined time or can be empirically determined depending on how the subject is responding or depending on observed side effects. For example, the treatment can be discontinued for one week, two weeks, one month or several months. As noted above, the cycle of administration can be for any desired length of time. It is within the level of skill of the treating physician or veterinarian to adopt a cycle of administration and dosing regimen that meets the needs of the patient depending on personal considerations specific to the patient and disease or condition or injury to be treated.

Many factors can influence administration of a PRP composition comprising primed platelets, including but not limited to, the route of administration, the volume administered, the platelet concentration, the animal to whom the composition is to be administered (i.e., species, size), the severity of the injury, disease, disorder or adverse indication or condition, and other factors.

### Administration Route

Platelets (e.g., primed platelets) in a PRP composition described herein can be administered in vitro, ex vivo, or in vivo by contacting a mixture, such as a body fluid or a tissue sample, with platelets in a PRP composition provided herein, including primed platelets described herein. For example, when administering a compound ex vivo, a body fluid or tissue sample from a subject can be contacted with the primed platelets in a PRP composition and later administered to a subject. When administering in vivo, the primed platelets in a PRP composition can be administered by any appropriate route, for example, orally, nasally, pulmonary, parenterally, intradermally, intravitreally, periocularly, subcutaneously, intracavitary, or topically, in liquid, semi-liquid or solid form and are formulated in a manner suitable for each route of administration. The PRP composition can be administered for soft tissue use. Determination of dosage is within the skill of the physician or veterinarian, and can be a function of the disorder, route of administration and subject. In some examples the primed platelets in a PRP composition are injected into a subject. In examples, where the PRP is injected, the PRP composition containing primed platelets can be administered through a volume appropriate, standard blood filter.

In some embodiments, the lyophilized powder can be administered, such as by uncapping the vial and pouring the powder on the wound, as a topical wet wound treatment or, for example, packing into a tooth socket. The powder can be activated prior to use, such as activation with calcium, and concentrate the mixture into a gel for clotting.

A PRP composition described herein can be formulated for intra-tendonous administration. For delivery to the tendons, the primed platelets in a PRP composition can be delivered by intra-tendonous administration to, for example, affect (i.e., improve) properties of collagen microarchitecture, collagen and/or tendon morphology, and subsequent mechanical properties of the injected tendon. Intra-tendinous injection of primed platelets in PRP can alter tendon morphology and mechanics. For example, intra-tendonous administration can be used to directly deliver high concentrations of therapeutics such as a PRP composition containing primed platelets to a dysfunctional tendon. In some examples, intra-articular injections of primed platelets in PRP compositions are delivered in amounts effective for treating subjects with tendinopathies. For example, PRP injected into the tendon can be retained within the tendon, and distributes longitudinally along the tendon.

A PRP composition described herein can be formulated for intra-articular administration. For delivery to the joints, the primed platelets in a PRP composition can be delivered by intra-articular (IA) administration. For example, IA administration can be used to directly deliver high concentrations of therapeutics such as a PRP composition to the joint space. In some examples, intra-articular injections of primed platelets in PRP compositions are delivered in amounts effective for treating subjects with moderate osteoarthritis. In some examples, a leukoreduced PRP composition can be used for administration to joints.

A PRP composition described herein can be formulated for topical administration. In some examples, the PRP composition can be applied topically to the skin, such as to wounds in the skin. For example, if the PRP composition is administered topically if the PRP composition is administered for wound healing. In such examples, the PRP composition is applied to or administered at or around the wound. In some examples the PRP composition is applied topically to gaping wounds to effect clotting with urgency, for example at wounds that are bleeding. In other examples the PRP composition is applied topically prior to a medical intervention which will effect bleeding, such as significant bleeding. For example, the PRP composition can be administered prior to a dental surgery or upon a tooth removal to prevent excessive bleeding.

Intra-lesional administration can be used to deliver the PRP compositions. A PRP composition is delivered by intralesional injection to deliver a high concentration of the PRP composition (e.g., primed platelets in the PRP composition) to the site of the injury, pathology, or adverse indication. The primed platelets are delivered intralesionally to maximize efficacy while minimizing systemic adverse effects of administration. In some examples, because the PRP administered by intra-lesional administration directly to the site a lower dose of platelets can be administered.

Implantation of a slow-release or sustained-release system, such that a constant level of dosage is maintained (see, e.g., U.S. Pat. No. 3,710,795) also is contemplated herein. The percentage of active compound contained in such parenteral compositions is highly dependent on the specific nature thereof, as well as the activity of the compound and the needs of the subject.

A PRP composition described herein can be formulated for ocular or ophthalmic delivery. Ocular platelet delivery includes, for example, topical, oral, or systemic, and/or injection. For example, a PRP composition containing primed platelets described herein can be administered topically, such as in the form of eye drops. For example, a reconstituted lyophilized PRP composition comprising primed platelets can be administered to the topically, such as in the form of eye drops, to the eye. In another example, a PRP composition containing primed platelets described herein can be administered by periocular and/or intravitreal administration, such as, for example, by periocular or intravitreal injection(s). For example, a reconstituted lyophilized PRP composition containing primed platelets is administered by subconjunctival administration to the eye. In some examples, a PRP composition administered with MSCs for treating ocular conditions, such as ocular lesion.

In some examples, such as where the PRP composition is delivered by intra-articular (IA) administration, topical administration, or intralesional administration, or other modes as appropriate, the platelets can be activated, such as by addition of an agonist or Ca++, prior to administration to the subject. In such examples, the platelets are activated and the PRP composition containing the activated platelets are immediately administered. For example, if the subject for administration will be treated under anesthesia, the subject is anesthetized before platelets in the PRP composition are activated ex vivo. The primed status of the platelets in the PRP composition herein provides platelets that are available for intralesional activation upon administration.

### Subject for Administration

In examples where the PRP composition is for administration to non-human animals, the PRP composition can be administered under the order of a licensed veterinarian. The skilled veterinarian can determine the location, administration techniques, administration volumes, etc. based on the knowledge in the field of veterinary sciences, and the animal subject for administration. In examples herein, a PRP composition containing primed platelets is administered to a dog, or a horse.

The subject and indication for treatment can influence the volume and dose of platelets administered. For example, for a small animal or a small volume or area of the subject, a small volume of platelets should be administered. The volume administered should not exceed the volume of the intra-articular space. The injury size, size of the animal, and chronicity of administration can impact the volume and dose of platelets in a PRP composition administered.

### M. DOSAGES

The PRP compositions containing primed platelets provided herein can be formulated as compositions for single dosage or multiple dosage administration. The dosage of the PRP composition depends upon the concentration of platelets in the compositions and the indication for treatment and the subject to whom the compositions is administered. The PRP compositions, as described above, are lyophilized for long term storage, and then can be reconstituted for administration. Generally, reconstitution is effected with saline or other suitable vehicle. Additional active agents or other components can be added as appropriate for the treatment.

The PRP composition containing primed platelets is administered in an amount sufficient to exert a therapeutically useful effect in the absence of undesirable side effects on the subject treated. The compositions are formulated accordingly. The amount of a PRP composition and/or the platelet concentration to be administered for the treatment of a disease, disorder, or condition also can be determined by standard clinical techniques. The therapeutically effective concentration of platelets can be determined empirically by testing the platelets in known in vitro or in vivo systems, such as by assays known in the art and then extrapolated therefrom for dosage in the subject or species to be treated. In addition, in vitro assays and animal models can be employed to help identify optimal dosage ranges. The precise dosage, which can be determined empirically, can depend on the form of the PRP composition (i.e., solid, liquid), the route of administration, the type of disease to be treated and the seriousness of the disease.

The severity of the disorder or condition can impact the platelet dose for administration. A higher dose can be administered for a severe indication or injury or disorder compared to a less severe indication or injury or disorder. In some examples a higher platelet count is associated with better therapeutic outcomes. For example, administration of more platelets can release more cytokines than fewer platelets, which can exert an improved therapeutic effect. In some examples, a temporary or minor disorder, condition, or injury can be treated with a lower platelet dose compared to a chronic or severe disorder, condition, or injury. In some examples an intralesional administration may require a lower dose or lower concentration of platelets administered. For example, a subject with a small intralesional tear can require a lower platelet dose. Chronic conditions, such as osteoarthritis can require a higher platelet dose.

The dosage of the PRP composition can vary depending on the administration location and/or route (see Everts et al., Int J Mol 21 (20): 7794 (2020); Garbin et al., Front Vet Sci 8:641818 (2021)). For example, the dosage for intralesional injection can be different than the dosage administered by topical administration. In another example, the dosage for intralesional injection can be different than the dosage administered by intravenous administration. A higher platelet count, within the ranges listed herein, is associated with better clinical outcomes.

Dosing also can vary depending on the indication relating and whether the use is in vitro, in vivo, or ex vivo. For example, for use in regenerative medicine, the dosage of the PRP composition depends on the lesion and site of injection. More severe or worse diseases, disorders, conditions, or indications can require a higher dose of platelets for administration. Hence, it is understood that the precise dosage and duration of treatment is a function of the disease being treated and can be determined empirically using known testing protocols or by extrapolation from in vivo or in vitro test data. The concentrations and dosage values also can vary with the severity of the condition to be alleviated. For any subject, specific dosage regimens should be adjusted over time according to the individual need and the professional judgment of the person administering or supervising the administration of the compositions, and that the concentration ranges set forth herein are exemplary only and are not intended to limit the scope or use of compositions and combinations containing them. The PRP compositions can be administered hourly, daily, weekly, monthly, yearly, or once. The regimen depends upon the indication treated.

Generally, dosage regimens are designed to maximize therapeutic effectiveness and to limit toxicity. It is within the skill of physician or other treating professional, such as a veterinarian for treating animals, to select dosages and treatment protocols, and know when to terminate, interrupt or adjust therapy to lower dosage due to toxicity, or bone marrow, liver or kidney or other tissue dysfunctions. Conversely, the skilled physician or veterinarian also would know how to and when to adjust treatment to higher levels if the clinical response is not adequate (precluding toxic side effects).

The dosage of the PRP composition also depends on the subject for administration. For example, the dosage of the PRP composition depends upon the species to whom it is administered and the locus of administration, and the disease, disorder, or condition treated. In some examples, different species can have differences, such as, for example, differences in clotting activities, species differences (i.e., in platelet numbers), or metabolic differences that can impact the efficacy (i.e., clotting efficacy) of the platelets in the PRP composition. In other examples, differences among animals can impact dosage. For example, body weight, age, and sex of the subject for administration can impact the dosage. In other example, the status or number of endogenous platelets in the animal can impact the dosage.

The exact dose depends on the particulars of, such as age, weight, and condition, of the patient or animal as is known in the art. The unit-dose PRP preparations are packaged in an ampoule, a vial, or a syringe with a needle. The volume of liquid solution or reconstituted powder preparation, containing the PRP composition, is a function of the disease to be treated and the article of manufacture chosen for package. All preparations for administration should be sterile, as is known and practiced in the art.

A platelet-rich plasma composition provided herein, such as, for example, a reconstituted lyophilized PRP composition, can be used in an amount effective for treating a subject. Such PRP compositions, for example, can be administered to effect a platelet level that exceeds the platelet level in a subject who is not administered a PRP composition. In some examples, an effective amount of a PRP composition is an amount that results in reduced bleeding or reduced potential for bleeding of a subject. In some examples, reduced bleeding results in normal hemostasis. In some examples, the bleeding or bleeding potential is reduced for at least 10, 20, 30, 40, 50 or 60 minutes after PRP administration. In some examples, administration of a second, third, fourth, fifth, sixth or more dose(s) of a PRP composition is required to produce an effective dose.

In some examples, an effective amount of a PRP composition containing primed platelets is an activity-based number of platelets. Activity of platelets in a reconstituted lyophilized PRP composition herein is compared to platelet activity prior to lyophilization. For example, platelet activity following lyophilization and reconstitution is similar to or less than platelet activity prior to lyophilization. For example, platelet activity following lyophilization and reconstitution is at least about or about 50%, 55%, 60%, 65%, 70%, 75%, 80%, 85%, 90%, 91%, 92%, 93%, 94%, 95%, 96%, 97%, 98%, 99% or 100% of the platelet activity prior to lyophilization. In examples, activity can be determined based on thrombin generation potency as assessed in thrombin generation potency units (TGPU) (see e.g., International Pat. Pub. No. WO 2020/227149). Such activity-based amount used to determine the dosage for administration within the platelet ranges/kg listed herein.

In some examples any of the dose ranges provided above can be administered in multiple treatments. For example, the dosages listed above can be administered one time and then administered one or more additional times. As detailed, the dosage administered will depend on the subject, such as the species, weight, and/or health, the administration site (e.g., joint), the intended use or disease, disorder, or condition to be treated, and other factors. In some examples, up to 1×10⁹ platelets/kg of the subject is administered more than 1 time to a subject. For example, a dose range of between 1.0×10⁷ platelets to about 1.0×10¹⁰ platelets, are administered between 2 and times, or between 2 and 8 times, or between 2 and 6 times, or between 3 and 8 times, or between 3 and 6 times, or between 4 and 6 times wherein the dose(s) after the initial dose are administered between within 1, 2, 3, 4, 5, or 7 days from the first dose.

In some examples, the number of platelets, such as primed platelets in a PRP composition, such as, for example, a reconstituted lyophilized PRP composition, includes any appropriate dosage of a composition comprising the primed platelets as described herein that can be administered to the subject, in some examples that results in a therapeutic benefit in a subject. For example, a dose of a composition comprising primed platelets can include between

Treatment can comprise a single treatment or multiple treatments. In some examples, an animal is provided with one or two treatments, wherein each treatment comprises administering to the animal about 500 million to about 5 billion platelets, about 500 million to about 1.5 billion platelets, about 500 million to about 2.5 billion platelets, about 500 million to about 3.5 billion platelets, about 500 million to about 4 billion platelets, about 500 million to about 4.5 billion platelets, or at least or at least about 5 billion platelets, 4.5 billion platelets, 4.0 billion platelets, 3.75 billion platelets, 3.5 billion platelets, 3.25 billion platelets, 3 billion platelets, 2.75 billion platelets, 2.5 billion platelets, 2.0 billion platelets, at least 1.75 billion platelets, at least 1.5 billion platelets, at least 1.25 billion platelets, at least 1 billion platelets, or at least 500 million platelets, per treatment. As detailed herein, at least 50% of the platelets in the reconstituted PRP compositions prepared without cryopreservatives are in the primed state, thus, at least 50% of the numbers of platelets listed above are in the primed state. For example, at least or at least about 250 million to about 2.5 billion platelets, about 250 million to about 750 million platelets, about 250 million to about 1.25 billion platelets, about 250 million to about 1.75 billion platelets, about 250 million to about 2 billion platelets, about 250 million to about 2.75 billion platelets, or at least or at least about 2.5 billion platelets, 2.25 billion platelets, 2.0 billion platelets, 1.9 billion platelets, 1.75 billion platelets, 1.6 billion platelets, 1.5 billion platelets, 1.375 billion platelets, 1.25 billion platelets, 1.0 billion platelets, at least 875 million platelets, at least 750 million platelets, at least 600 million platelets, at least 500 million platelets, or at least 250 million platelets, are in the primed state. In examples herein, a dose of platelets is at least or at least about or about 1×10⁹ platelets per lesion.

When the PRP treatment is used in conjunction with an additional therapeutic or treatment, the dosage of reconstituted lyophilized platelet-rich plasma (PRP) composition is administered before, after, or concomitantly with the additional therapeutic treatment. In some examples, the number of platelets or concentration of platelets in the PRP composition is decreased when the PRP composition is administered with an additional therapeutic. In other examples, the concentration of platelets, or number of platelets, or the dosage of the PRP composition is not modified when the PRP composition is administered with an additional therapeutic compared to the concentration of platelets, or number of platelets, or the dosage of the PRP composition administered in the absence of an additional therapeutic. In these examples, the concentration of platelets, or number of platelets, or the dosage of the PRP composition is the same when the PRP composition is administered with the additional therapeutic as the concentration administered without the additional therapeutic. In examples herein, a dose of platelets that is administered or used with an additional therapeutic is at least or at least about or about 1×10⁹ platelets per lesion.

### N. EXAMPLES

The following examples are included for illustrative purposes only and are not intended to limit the scope of any invention herein.

### Example 1

### Production and Characterization of the Lyophilized Pooled Platelet-Rich Plasma Composition

### Donor Screening and Selection

Equine donor selection, evaluation, and quarantine prior to use was performed by a USDA licensed equine facility according to their quality management system protocol, which is compliant with the guidance set forth by the U.S. Food and Drug Administration (FDA) Center for Veterinary Medicine (CVM) (see, Government Furnished Information (GFI) #254 Donor Eligibility for Animal Cells, Tissues, and Cell- and Tissue-Based Products, updated October 2022). All donors were from a closed-colony herd selected and maintained with standard operating procedures for donor screening, quarantine, and evaluation, and routine ongoing surveillance. Horses are qualified donors through the USDA licensed equine donor program. All donors are part of a closed colony for the production of USDA-licensed biologics.

Equine donors for use in raw material collection were screened by PCR and/or ELISA for the presence of infectious disease, including: equine infectious anemia (EIA), equine viral arteritis (EVA), Dourine (Trypanosoma equiperdum), Brucellosis, Piroplasmosis (Babesia equi and Babesia caballi), Glanders (Burkholderia mallei), and Equine Parvovirus-hepatitis Virus (EqPV-H). Infectious disease evaluation also was in compliance with FDA CVM Guidance #254 (see, Government Furnished Information (GFI) #254 Donor Eligibility for Animal Cells, Tissues, and Cell- and Tissue-Based Products, updated October 2022).

Horses in the closed colony from which the samples were collected are vaccinated for equine influenza, tetanus, rhinopneumonitis (equine herpes virus), Clostridium botulinum Serotype B toxin, rabies, Streptococcus equi, Potomac Horse fever, Escherichia coli, West Nile virus, equine rotavirus, Salmonella, Rhodococcus equi, Eastern and Western Encephalomyelitis, and Clostridium perfringens Type C&D. Horses were screened biannually for Equine Infectious Anemia (EIA), Equine Viral Arteritis (EVA), Dourine, Glanders, Brucellosis, Piroplasmosis, EHV-1, and Equine Parvo-Hepatitis. Donors that were negative by PCR and/or ELISA for the tested infectious diseases were selected. All donors were also blood typed by tube agglutination, and positive for Aa and Ca by standard serologic evaluation. Donors also were negative for plasma antibody to erythrocyte antigens to support universal blood type compatibility.

Equine donors also were pre-screened by standard complete blood count (CBC) for hematocrit and platelet count. Donors with a hematocrit percentage greater than 32% and a platelet count greater than 130,000/μL were selected for automated plateletpheresis.

### Platelet Collection

Recovery of platelets by apheresis (plateletpheresis) was conducted using a Haemonetics® mobile collection system (MCSR) automated apheresis device to separate platelets from the whole blood and recover platelet concentrate of platelets in plasma. Anticoagulant Citrate Dextrose Solution-Formula A (ACD-A) was added at a ratio of 1:12 and used as an anticoagulant for plateletpheresis. The apheresis protocol was adapted to accommodate the size of the horses (e.g., at or about 1000 pounds and 60 inches or taller). The total number of platelet units in plasma varied based on the manufacturing batch size. Recovered platelet concentrates generally were 400-500 mL of platelets in plasma with a total platelet count of about 4.00×10¹¹. Platelet concentrates (PCs) were passed through a leukoreduction filter (PALL® Corporation, Port Washington, New York) to reduce and limit the number of white blood cells (WBCs) in the final composition. The final platelet concentrate (PC) was leukoreduced using filtration (e.g., the Pall filter) or centrifugation, decreasing the amount of white blood cells from about 9000/μL to less than 1500/μL (e.g., <1200 WBCs/μL), or, on average, 0.15% of the cells in the composition are white blood cells assessed by flow cytometry analysis.

After apheresis and platelet collection, platelet concentrates initially were divided into two sets of samples. The first sample, approximately 2 mL of platelets resuspended in plasma, was used for evaluation of platelet count, red blood cell count and white blood cell count by impedance device.

The second set of platelets resuspended in plasma were immediately refrigerated at 4-8° C. after collection and until they were sent for further manufacturing. These samples were transported in containers validated for refrigerated shipping of the platelet concentrate to a qualified GMP manufacturing facility at 1-10° C. Upon arrival at the facility, samples were refrigerated at 4-8° C. until use. The units of platelets in plasma can be held at 0-10° C. for up to 14 days, such as up to 13, 12, 11, 10, 9, 8, 7, 6, or 5 days post-collection before further processing, manufacturing, sterile pooling, vialing, and/or lyophilization.

### Plasma Collection

Equine donors screened and selected as above were plasmapheresed using a modified AutoC apheresis device programmed for plasma collection. Units were produced in 1 L volumes and frozen at −20° C. or lower until the time of pooling. Per Association for the Advancement of Blood and Biotherapies (AABB) guidelines for frozen plasma, the plasma is assigned a five-year expiration from the date of collection. The plasma units were transported by an overnight courier in a validated standard shipping container at room temperature to allow uncontrolled thaw during transport to the manufacturing center for further treatment, plasma also can remain frozen during transport.

### Pooling

Once apheresis-derived equine platelet concentrates (EQADPC) and apheresis derived equine frozen plasma (EQADFP) were received at the GMP manufacturing center; the units were qualified for use by the Quality Assurance department and refrigerated until use. Thawed plasma and platelet concentrates can be stored for up to 5 days at 1° C.-10° C. before processing. Units were brought into a clean room in accord with vendor standard operating procedures (SOPs), and given unique unit identification numbers. Pooling is effected in an International Standards Organisation (ISO) 6 environment using closed blood banking systems, or as an open process within a biosafety cabinet environment. Cleanrooms are standardly classified by the cleanliness of the air, and cleanroom standards are based on the size of the particles (i.e., in μm), to be filtered and the subsequent particle count, including the quantity and size of particles per volume of air.

Individual platelet donors vary widely in count, growth factor concentration, including, PDGF-BB, VEGF, and activation potential (Alhumaidan, 2011 #71). As shown and described herein, pooling platelets yields a more uniform function profile. First, platelets and plasma from up to 18 equine donors were pooled (pools of apheresis-derived equine platelet concentrates and separate pools of apheresis-derived equine plasma concentrates) based on batch size and/or platelet counts provided by analysis by IDEXX BioAnalytics. Leukoreduced allogeneic freeze-dried PRP is produced in small batch lots (<1500 vials) with a target platelet count of 500,000 platelets/μL+/−100,000/μL.

### Pooling of Apheresis Derived Equine Platelet Concentrates

For pooling of apheresis derived equine platelet concentrates (EQADPC), each unit was first documented in the batch record with a donor ID. Plasma units (FP) for pooling were removed from refrigerated temperatures and maintained at ambient temperatures for not more than 4 hours before mixing the platelet concentrate units. Two-lead harnesses were brought into the work area, and the plastic overwrap was removed within the sterile cabinet workplace. A plastic hemostat from Dravon Medical Inc. (Clackamas, Oregon) was placed on the central outflow tube, and isopropyl alcohol was used to clean the port region of each PC bag and allowed to air dry 2 minutes. The piercing pin was uncapped and inserted firmly into the first EQADPC; this was repeated until all units were attached to a piercing pin. The piercing pin was attached to a 3000 mL transfer bag (Product No. T3109; by Charter Medical; Winston-Salem, NC), and a plastic hemostat from Dravon Medical Inc. (Clackamas, Oregon) was placed on a tube below the pin to obstruct flow. The hemostat from the central outflow tube of the 10-lead harness (Product No. T8010 by Charter Medical; Winston-Salem, NC) and the hemostat from the tube below the transfer bag pin was removed; the clamp on each tube was released, allowing the PC to flow into the transfer bag. The PC was gently swirled throughout the transfer to mix while filling the transfer bag. Once all the PC was transferred to the 10 L transfer bag, a labeled 3 mL sample was removed and placed in a 15 mL Falcon tube for cell counting by a veterinary laboratory. The inlet tube of the transfer bag was heat sealed in two places using a tube welder or clamped with an aluminum sealing clamp. The transfer bag, containing about 4.0×10¹² platelets/4600 mL, was left flat in a safety cabinet pending vialing. Once pooled the product is handled at ambient temperature and further processed within 4 hours.

### Pooling of Apheresis Derived Equine Frozen Plasma

For pooling of apheresis derived equine frozen plasma (EQADFP) each unit was first documented in the batch record with a donor ID. Frozen plasma as defined under the Association for the Advancement of Blood and Biotherapies (AABB) guidelines as set forth in the 2022 Technical Manual for Blood Products and collected by apheresis or whole blood, single-unit centrifugation, was used. All units were thawed and stored at 0-8° C. until the time of pooling. Per AABB Guidelines the refrigerated plasma is utilized within 5 days of thawing to refrigerated temperature.

For plasma that was held frozen at −20° C. until the time of pooling then a plastic overwrap was placed over the unit and overwrap/unit were placed in a 35° C.+5° C. water bath, any plasma samples that remained frozen were thawed in the plastic overwrap, to avoid contact of the samples with the water in the water bath, for 30-45 minutes. Once thawed, plasma samples were stored at ambient temperatures for no more than 4 hours before mixing the plasma samples together. A piercing pin was attached to a 3000 mL transfer bag (Product No. T3109 by Charter Medical; Winston-Salem, NC), and a plastic hemostat from Dravon Medical Inc. (Clackamas, Oregon) was placed on the tube below the pin to obstruct flow. Next, the hemostat from the central outflow tube of a 10-lead harness (Product No. T8010 by Charter Medical; Winston-Salem, NC) and the hemostat from the tube below the transfer bag pin were removed. Isopropyl alcohol was used to clean the port region of each frozen plasma (FP) bag and the port region was allowed to air dry for 2 minutes. The piercing pin was uncapped and inserted into the first unit of EQADFP; this was repeated until all units were attached to a piercing pin. After the last plasma unit was transferred, the inlet line was sealed using aluminum sealing clamps to close the inlet line. An inlet blood filter was inserted into one of the ports of the 3000 mL transfer bag (Product No. T3109 by Charter Medical; Winston-Salem, NC), ensuring that the clamp was closed during the insertion process. Next, and, using aseptic techniques, plasma was transferred to the 10 L custom transfer bag holding the combined platelet concentrate. Once the EQADFP was transferred, the 10 L transfer bag is placed on a 3 D Rotator, and the sealed 10 L transfer bag was gently rotated during pooling of the EQADPC and EQADFP (described below).

Pooling of Apheresis Derived Equine Platelet Concentrate Pool with Apheresis Derived Equine Frozen Plasma Pool

Apheresis derived platelet concentrate and frozen plasma (thawed prior to pooling) were pooled to control the final platelet count and provide uniformity in the platelet rich plasma (PRP) composition. No excipients were added. During the transfer of the thawed plasma into the 10 L custom transfer bag containing the pooled platelet concentrate (PC) units, it was ensured that the platelets did not foam and that no pressure was placed on the bag during the transfer. If foaming occurred, the fluid was rested for 2-5 minutes before reinitiating flow. Once pooled, the mixture containing PC and FP was placed on the 3 D Rotator and gently stirred for 30-60 minutes at room temperature before progressing to vial filling. The combined PC and FP mixture yielded platelet rich plasma (PRP). The PRP was visually inspected for platelet swirling and aggregation. If there are no visual abnormalities, the product was prepared via the standard port for transfer of the product through sterile plastic tubing to vials using an automated filling device. Using the internal vialing and filing protocol, 8 mL+/−0.2 mL of the platelet rich plasma (PRP) was added to 50 mL glass vials (obtained from Schott Pharma; Mainz, Germany).

A platelet-rich plasma composition with approximately 400,000/μL+/−100,00 platelets and less than 1500/μL of white blood cells was prepared. The final PRP composition includes apheresis-derived platelet concentrate, apheresis-derived frozen plasma (thawed), and anticoagulant (ACD-A). The amount of ACD-A in the end product is based on the following calculation:

(Total amount of mLs in apheresis event)/12=Total mLs of ACD-A per event

(ACD-A in mLs per event)×(No. of events)=Total mLs of ACD-A

(Total mLs of ACD-A)×0.60=Total mLs of ACD-A in the plasma portion

In the standard blood product, most of the ACD-A is located in the plasma component. For the pooled PRP composition, approximately ¼ of the product is plasma.

(Total Amount of ACD-A in plasma)×0.25=ACD-A in pooled PRP

(ACD-A in pooled PRP)/(Total amount of PRP in mLs)=Total ACD-A in

final product per ml

For Example:

Total milliliters of ACD-A in Horse A plateletpheresis=(4250 mLs)/12=354 mLs

Total milliliters of ACD-A for pooled product=(354 mLs)×12 donors=4250 mLs

Total milliliters of ACD-A for plasma portion=4250 mLs×(0.60)=2550 mLs

Total milliliters of ACD-A in pooled PRP=2550 mLs×(0.25)=637.50 mLs

Total ACD-A in final product per milliliter=637.50 mLs/7200 mLs=0.08 mLs

The final PRP composition including apheresis-derived platelet concentrate, apheresis derived frozen plasma, and anticoagulant (ACD-A) was subjected to a controlled freeing process from ambient temperature to −40° C. prior to lyophilization.

### Lyophilization

Vials containing the platelet rich plasma (PRP) were loosely stoppered, placed in lyophilization trays and transferred under sterile cover to a lyophilizer. A lyophilization cycle was developed using three pilot batches and one engineering batch for the lyophilization process. Lyophilization was continuously monitored throughout the cycle. The PRP composition was lyophilized at −40° C. in accord with the following lyophilization protocol:
1. Freeze rate from ambient to −40° C. is approximately 1° C./minute for 60 minutes.
2. Freeze Soak time at −40° C. is approximately 3 hours.
3. Primary Dry ramps from −40° C. to 0° C. over 20 hours. Chamber Vacuum is 100 millitorr
4. Primary Dry Soak time at 0° C. is 8 hours.
5. Secondary Dry ramps from 0° C. to 30° C. over 12 hours. Chamber Vacuum is 100 millitorr
6. Primary Dry Soak time at 30° C. is 6 hours.
7. Total Cycle time including product freeze step: 50 hours, 35 minutes.

Following lyophilization, and after the chamber was backfilled with nitrogen under a vacuum, the vials were stoppered, removed from the lyophilizer, and crimped using a 20 mm aluminum sealing camp. The sterile, nonpyrogenic, lyophilized PRP composition was stored in 50 ml vials, and was a white to tan powder. The vials containing the lyophilized PRP were placed in single layer of 50-60 vials in cardboard trays, labelled with a unique lot and vial identification, overwrapped with plastic, and stored at room temperature.

### Reconstitution of the Lyophilized PRP in Sterile Water or Sterile Saline

The lyophilized PRP is rehydrated with 8 mL of sterile saline (e.g., 0.9% saline) or sterile water. 8 mL of sterile water or sterile saline is drawn into a sterile needle and syringe, and the vented clave was applied to the PRP vial and firmly set onto the crimped seal. Then, using sterile techniques, the needle is disengaged, the syringe is attached to the clave hub, and the fluid is slowly added down the side of the vial, to avoid foaming, and the lyophilized PRP cake is immersed in the rehydrating fluid. The fluid and the lyophilized powder are gently mixed by swirling the vial to rehydrate for two minutes. The reconstituted PRP presents as a gold translucent fluid of 8mLs with 300,000 to 500,000 platelets per microliter and less than 1500 white blood cells per microliter.

The reconstituted PRP can be stored at room temperature until administration, but generally not for more than 4 hours.

### Characterization of the Reconstituted Lyophilized PRP Composition

Random vials of the lyophilized PRP composition were selected for characterization. The lyophilized PRP was evaluated for residual moisture using a standard validated technique (Karl Fischer moisture titration). Once the percentage of residual moisture was verified to be within characterization standard of less than 2.5%, two vials were rehydrated using sterile water for injection. The pH was of the reconstituted PRP was recorded using the rehydrated material and verified to be within characterization standard. (e.g., pH 7.0-9.0) An interim certificate of conformance was prepared describing the residual moisture, pH, and visual inspection.

Chemokines (PDGF-BB, VEGF) were identified by validated ELISA, as detailed in the Examples below. Cluster of differentiation (CD) marker analysis and sizing was performed using a standard operating procedure (SOP).

Species-appropriate individual (non-pooled, fresh) PRP was used as a positive control. USP standard techniques were used to evaluate for anerobic and aerobic organisms as well as Mycoplasma sp. Table 1, below, details the number of vials and characterization performed. Table 2, below, details the characteristic of the assessment, assay performed, acceptance criteria, and comments on the assessment.

TABLE 1
Characterization Detail

| No. of | |
|---|---|
| vials | Characterization description |
| 3 | Chemokine ELISA: PDGF-BB, VEGF, TNF-a, IL1B |
| 3 | CD Marker Analysis: CD61, CD9, CD44, CD62P, |
| | surface Lactadherin |
| 3 | Aerobic/anaerobic sterility (14 day)USP71 |
| 1 | Mycoplasma USP63 |
| 3 | Endotoxin USP85 |

TABLE 2
Characterization Detail

| | | Acceptance | |
|---|---|---|---|
| Characteristic | Assay | Criteria | Comments |
| Identity | Flow | CD 61 >/= 40% | Presence of CD 61 |
| | cytometric | | supports the presence |
| | analysis | | of platelet particles |
| Identity | Flow | CD 9 >/= 40% | Presence of CD 9 |
| | cytometric | | supports the presence |
| | analysis | | of platelet particles |
| Identity | Flow | CD 44 >/= 2% | Panleukocyte |
| | cytometric | | marker indicates |
| | analysis | | leukoreduction |
| Identity | Flow | CD 61 and/or 9 | Particle size with |
| | cytometric | positive; >/=75% | platelet markers |
| | analysis | 0.2 μm to 5 μm | supports the presence |
| | | particle size | of platelet particles |
| Purity | Residual | </=2% | Karl Fischer Method |
| | Moisture | | |
| Strength | Impedance | 400,000/μl +/− | Performed prior to |
| | count | 100,000/μl | lyophilization |
| Safety | USP 71 | Negative | Sterility test |
| Safety | USP 63 | Negative | Sterility test |
| Safety | USP 85 | <2 EU/mL | LAL method |
| Potency | ELISA | PDGF-BB | Acceptance criteria |
| | | VEGF | established by evaluation |
| | | | of 10 vials from 3 |
| | | | different batch/lots |

Experiments and results are set forth in Examples 2 and 3, below.

### Example 2

### Characterization of Cell Size in Lyophilized Equine Platelet Rich Plasma Composition by Flow Cytometry

A. Assessing the Particle Size of Reconstituted Lyophilized Equine Platelet-Rich Composition Compared to Equine Platelet and Leukocyte Rich Plasma Isolated from Fresh EDTA Blood-Pilot Study

Flow cytometric assays were performed to characterize the lyophilized equine platelet-rich plasma composition. Two vials of lyophilized PRP from each of three pilot manufacturing processes were reconstituted in 8 mL sterile saline (0.9% saline) were assessed. Antibody-conjugated beads and aliquots of frozen/thawed equine platelet-rich plasma were used as controls, and isotype antibodies were used to set positive/negative threshold boundaries. The particle size was assessed.

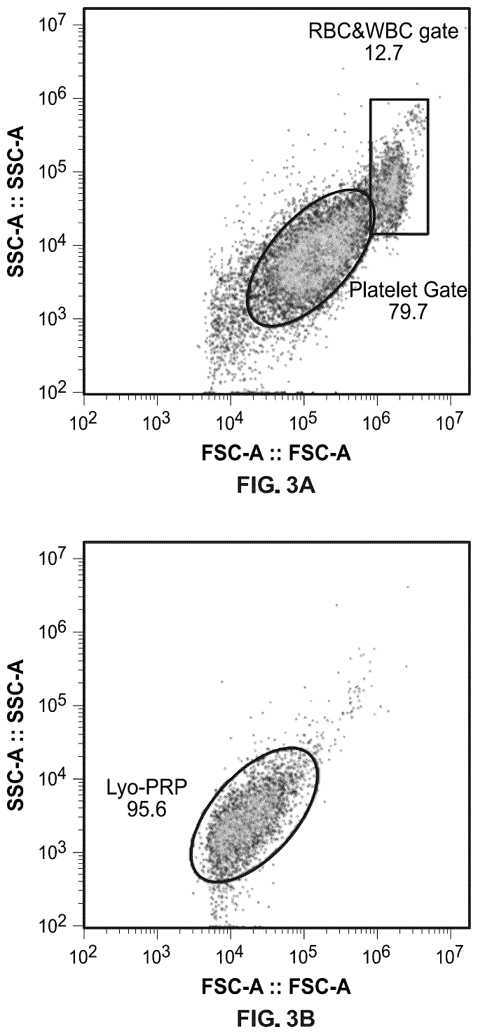

Flow cytometric assay sample plots are set forth in FIGS. 3A and 3B. FIGS. 3A and 3B set forth the side scatter/forward scatter (SSC/FSC) properties of equine platelet and leukocyte rich plasma (FIG. 3A) isolated from fresh EDTA blood or the reconstituted PRP composition (FIG. 3B). The results show that the reconstituted PRP composition particles are shifted left on the FSC axis (see FIG. 3B), denoting a smaller particle size compared to the fresh equine platelets (see FIG. 3A), which indicates that the process excludes larger platelets from the end product. Large platelets previously have been associated with an activated state, as platelets change shape, flattening to a larger mean platelet volume when activated. The results also show the relative absence of events corresponding to the presence of red blood cells or leukocytes in size and complexity in the reconstituted PRP composition (FIG. 3B). Red blood cells and leukocytes were present in the samples isolated from fresh blood. These results indicate that the PRP contains platelets with the majority in the size range of 0.5 to 2 microns, but does not contain red blood cells or leukocytes.

### B. Assessing the Particle Size of Reconstituted Lyophilized Equine Platelet-Rich Composition

Flow cytometric assays were performed as detailed above to characterize the lyophilized equine platelet rich plasma composition. Six vials of lyophilized platelet rich plasma (PRP) reconstituted in 8 mL sterile saline (0.9% saline) were assessed.

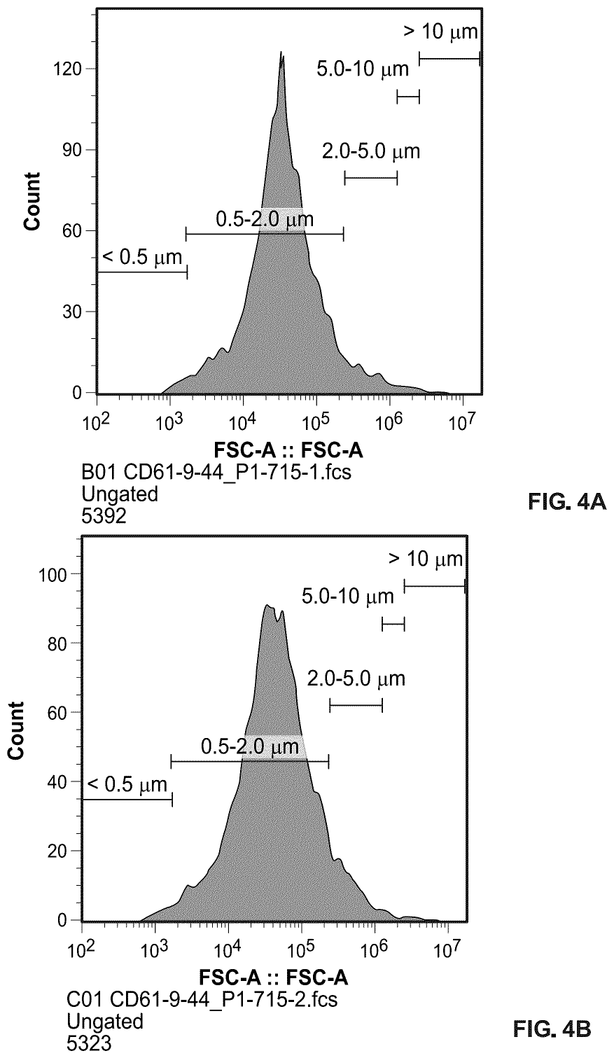

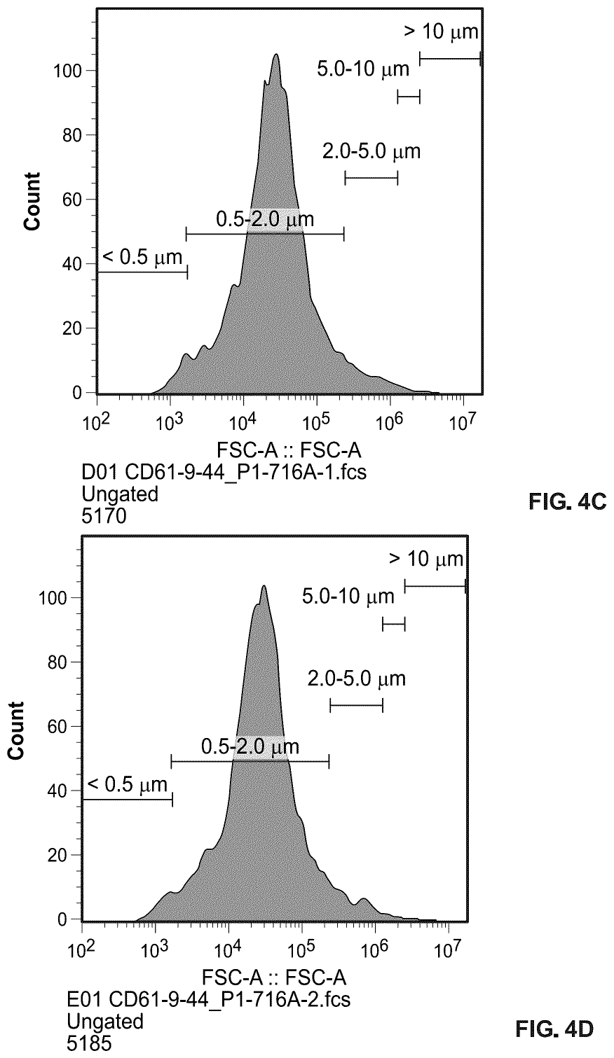

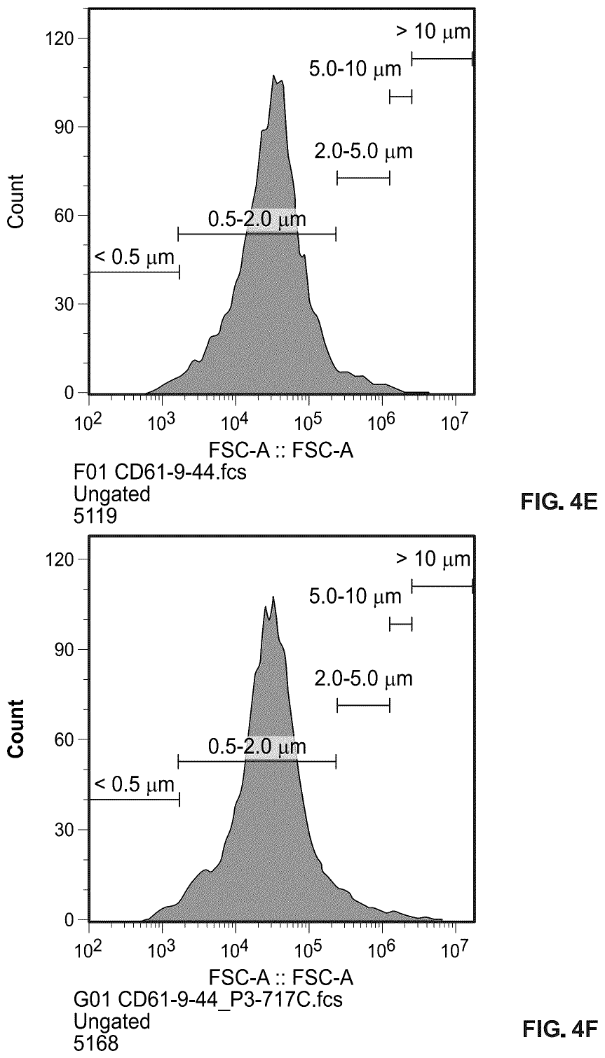

The samples were diluted 1:4,000 and the average particle size in the samples was assessed. The results show that over 90% of the particles from the reconstituted PRP composition fell within a 0.5 to 2 micron gate. Material listed in the particle sizing <0.5 μm are considered microvesicles. These results indicate that the majority of particles in the PRP composition are platelets, and that the composition is substantially free of red and white blood cells. The results also demonstrate that the platelets are not ‘broken,’ and are intact. The results from the six vials are set forth in FIGS. 4A-4F and Table 3, below:

TABLE 3
Summary of Particle Size and Percent of Each Particle Size

| | Number of Events or Percentage within Gate | | | | | | | | | |
|---|---|---|---|---|---|---|---|---|---|---|
| Particle | | | 0.5-2.0 | %0.5-2.0 | 2-5 | %2-5 | 5-10 | %5-10 | >10 | %>10 |
| Count | <0.5 | %<0.5 | μM | μM | μM | μM | μM | μM | μM | μM |
| 5392 | 82 | 1.5% | 4928 | 91.4% | 289 | 5.4% | 56 | 1.0% | 30 | 0.6% |
| 5323 | 66 | 1.2% | 4795 | 90.1% | 382 | 7.2% | 33 | 0.6% | 30 | 7.9% |
| 5170 | 152 | 2.9% | 4748 | 91.8% | 225 | 4.4% | 33 | 0.6% | 13 | 13.3% |
| 5185 | 132 | 2.5% | 4749 | 91.6% | 243 | 4.7% | 30 | 0.6% | 26 | 12.3% |
| 5119 | 79 | 1.5% | 4789 | 93.6% | 197 | 3.8% | 26 | 0.5% | 17 | 15.2% |
| 5168 | 97 | 1.9% | 4751 | 91.9% | 238 | 4.6% | 50 | 1.0% | 29 | 12.6% |

### Example 3

### Additional Characterization of Lyophilized Equine Platelet-Rich Plasma Composition by Flow Cytometry

### A. Assessing the Cell Types and Activation Status in the Reconstituted Lyophilized Equine Platelet-Rich Composition

Aliquots of the reconstituted composition were antibody-labeled to detect a pan-leukocyte marker, platelet membrane markers, and/or to identify the activation status of the platelets. The leukocyte and membrane markers used for characterization are set forth in Table 4, below.

TABLE 4
Leukocyte and platelet membrane markers

| Marker/clone | Target | Supplier |
|---|---|---|
| CD44-FITC /CVS18 | pan-leukocyte | Invitrogen |
| CD61-PE /SZ21 | platelet- GpIIIa | Beckman Coulter |
| CD9-APC/H19A | platelet -tetraspanin | Biolegend |
| CD62P-DY488/Psel.KO.2.7 | platelet-P selectin | Novus Biologicals |
| Lactadherin-FITC | outer membrane | Haematologic |
| | phosphatidylserine | Technologies |

Antibody-conjugated beads (Isotype (non-specific) conjugated antibodies fluorochromes: FITC, PE, APC, DY488) or control beads (CST from BD Biosciences or compensation control beads from Thermo Fisher) and aliquots of frozen/thawed canine platelet rich plasma were used as controls, and isotype antibodies were used to set positive/negative threshold boundaries. Reconstituted lyophilized PRP was diluted 1:20 in PBS (137 mM NaCl, 2.7 mM KCl, 8 mM Na₂HPO₄, and 2 mM KH₂PO₄), pH 7.4, and 5 μl of the diluted reconstituted lyophilized PRP was combined with a working labeled dilution (10 μL) in PBS to a total volume of 100 μL. The working dilutions are as set forth below:
a. CD44 & isotype FITC (1:10)
b. CD61 & isotype PE (1:2)
c. CD9 APC & isotype APC (neat)
d. CD62P & isotype DY488 (1:20)
e. Lact-FITC (1:20)

The reconstituted lyophilized PRP was incubated to label particles for 30 minutes in the dark at room temperature. After a thirty minute incubation, the reaction was quenched and diluted by the addition of 300 μL of PBS to each tube. At least 5,000 events were collected for membrane marker analysis. FSC/SSC gated events were analyzed to define the percent positive cells for each marker. All events were analyzed in size-calibrated templates to define the number of particles within defined micron-sized gates.

The particle size and the percentage of particles that expressed each cell and/or membrane marker were assessed. Three labeling reactions were performed for each sample as detailed above. The presence of three constitutive platelet markers (antibody-labeled with anti-CD61-PE, CD9-APC, and CD44-FITC); constitutive platelet membrane marker and phosphatidylserine (PS) marker (antibody-labeled with anti-CD61-PE & lactadherin-FITC, respectively); and constitutive platelet membrane marker and P-selectin marker (antibody-labeled with anti-CD9-APC & CD62PDY488, respectively) were assessed. An acquisition forward scatter threshold of 8,000 was used to allow detection of small (<0.5 μM) events and minimize machine noise and debris.

The results show that there was an 80% survival after reconstitution as assessed by total cell number. The results also show that approximately 40-50% of reconstituted PRP composition particles were positive by FACS for the constitutive platelet membrane markers CD61 and CD9, with relatively few (<1%) particles expressing leukocyte marker CD44. The anti-CD61 and anti-CD9 antibodies, however, were generated against human antigen; the fact that about 45% of the particles in the composition are CD61 and CD9 positive is indicative of incomplete binding due to species differences between human and equine CD61 and CD9. The experiments in Example 2 indicate that greater than 90% of the particles in the composition are the size of platelets. The CD44 results show that about 0.1% of the particles in the sample are leukocytes. Thus, up to 100% of the particles in the sample are platelets. The discrepancy in the particle size results and the CD61/CD9 results likely is due to incomplete binding by the CD61/CD9 antibodies.

The results also show that 40-50% of the cells in the sample are positive for lactadherin, indicating externalized phosphatidylserine. The percentage of cells that are positive for platelet membrane markers (e.g., CD61 and CD9) and those that are positive for lactadherin overlap (average about 45.7% platelet positive and 44.4% lactadherin positive), indicating that the majority (up to 100%) of the platelets in the sample are positive for lactadherin and have externalized phosphatidylserine. The results indicate that all the platelets that survive the process of lyophilization do so intact with externalized phosphatidylserine. An average of only about 6% of the cells were CD62P-positive (indicative of activation), indicating that only this low percentage of the partially activated platelets continued to full activation during or after freeze-drying.

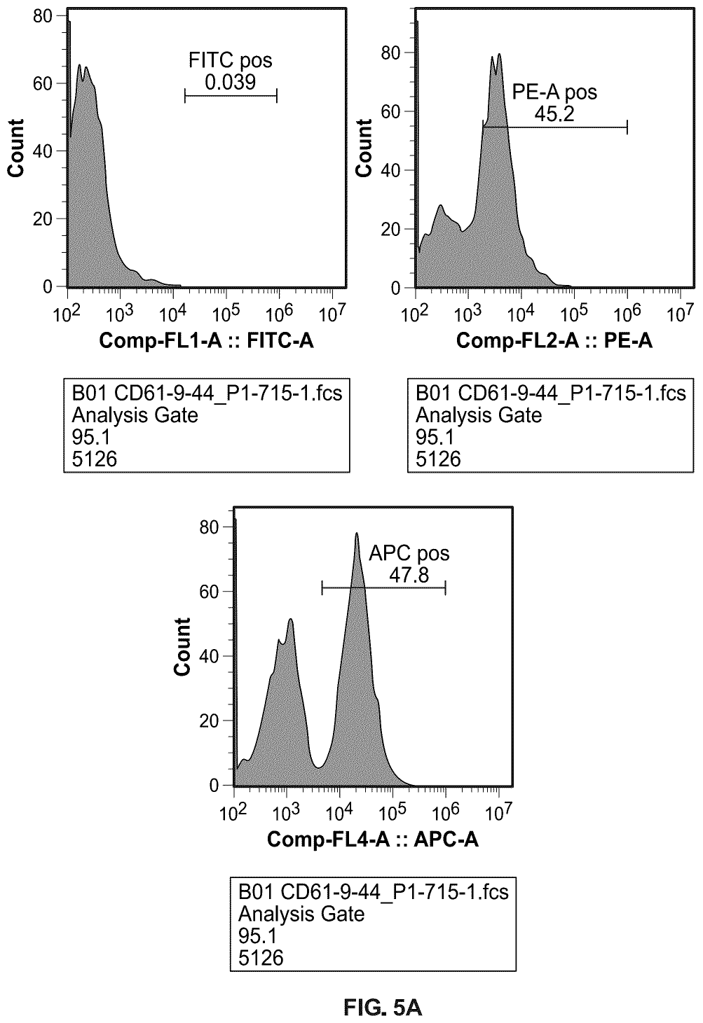

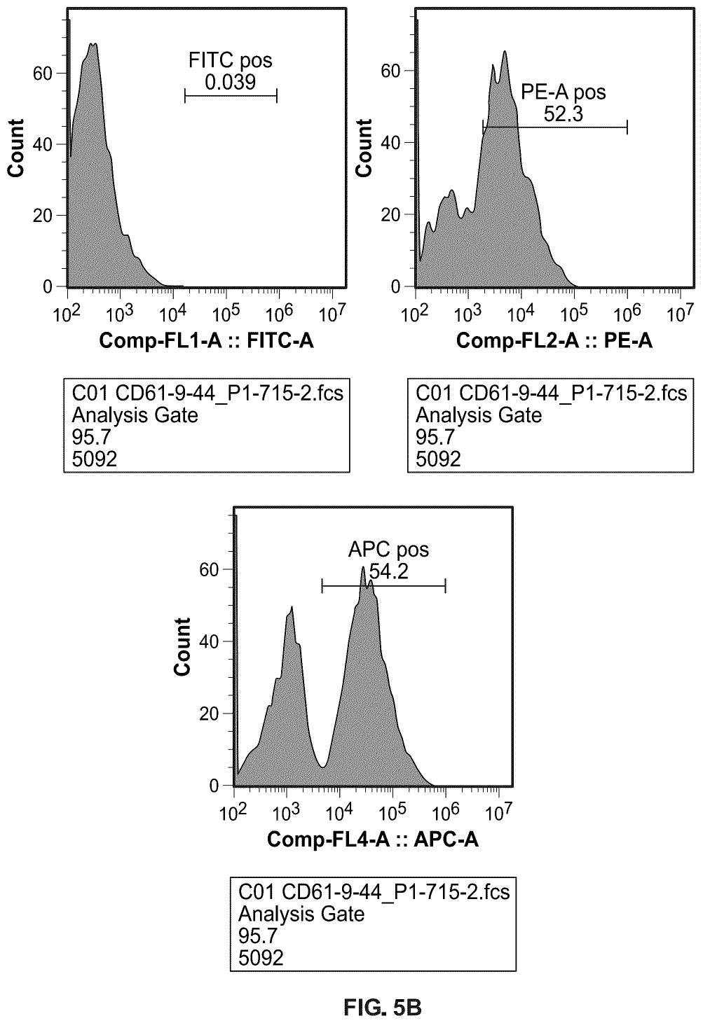

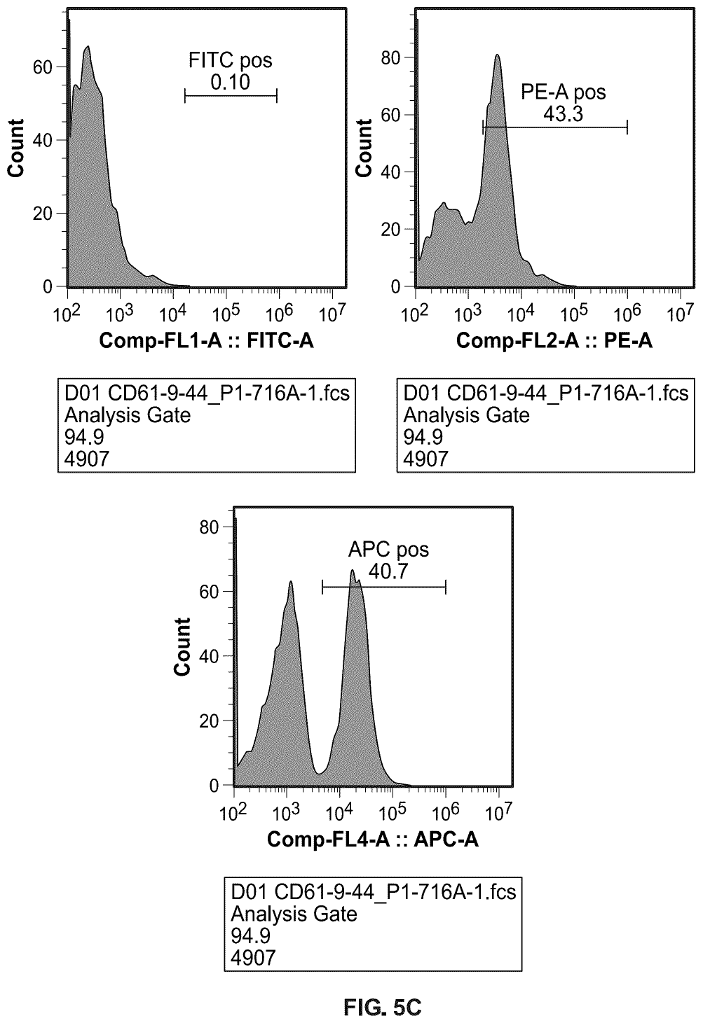

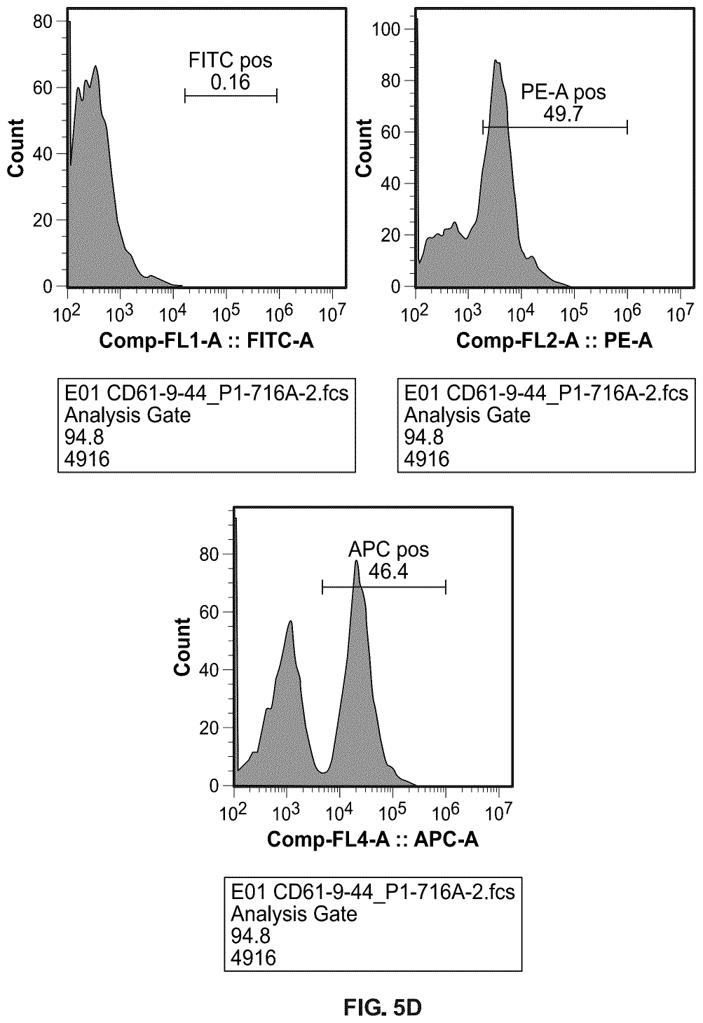

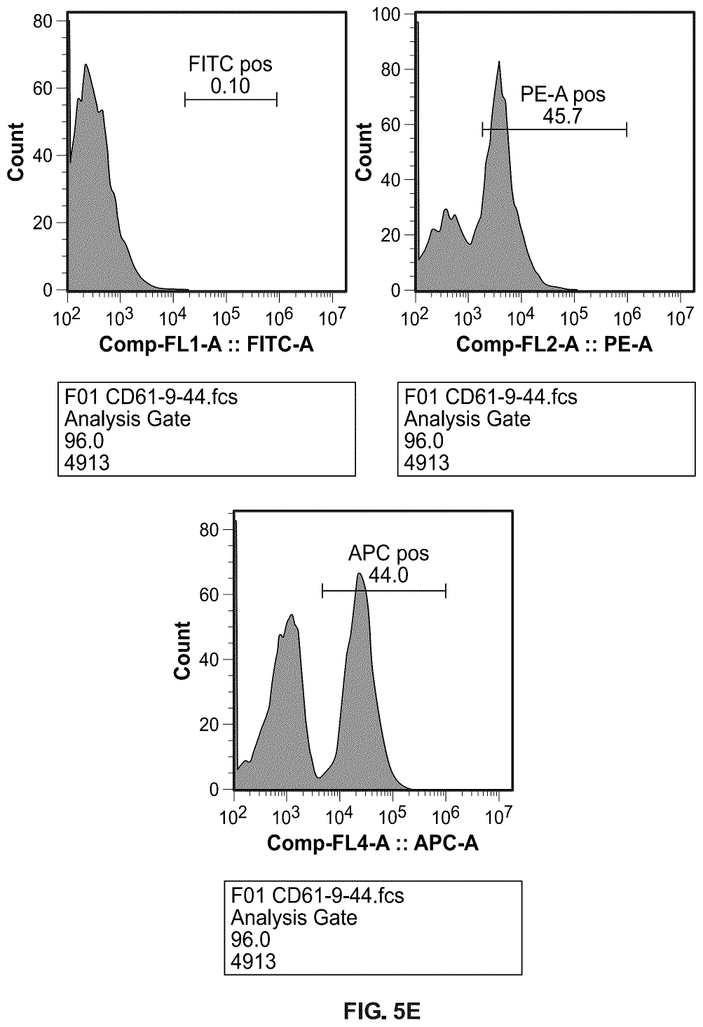

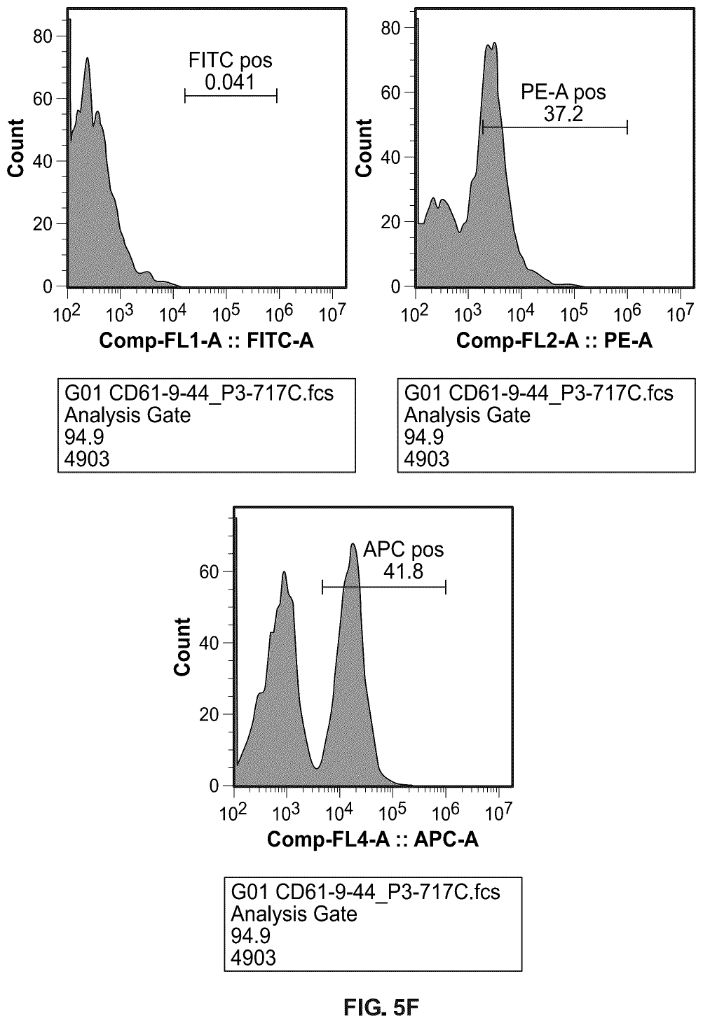

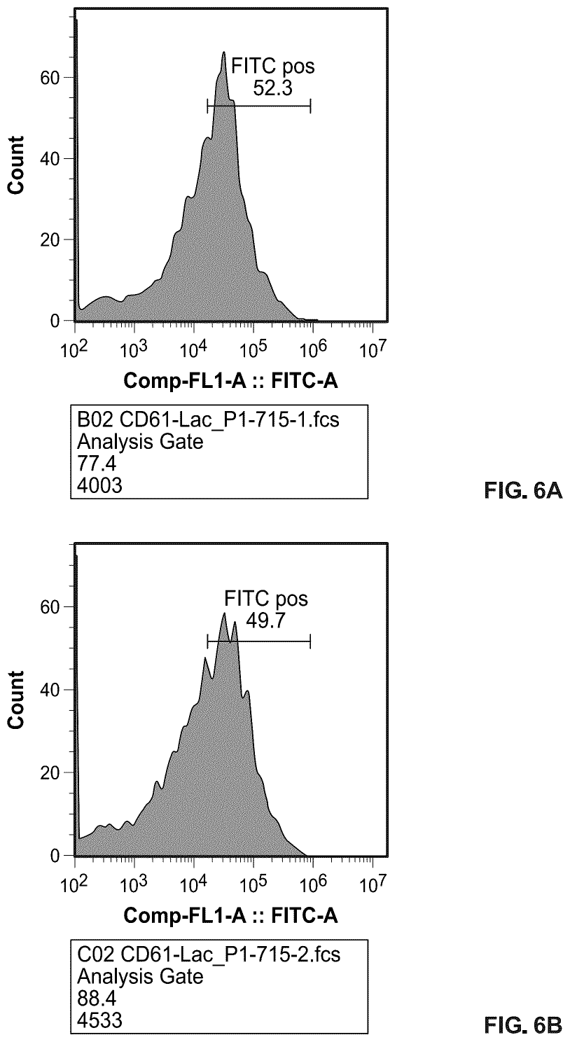

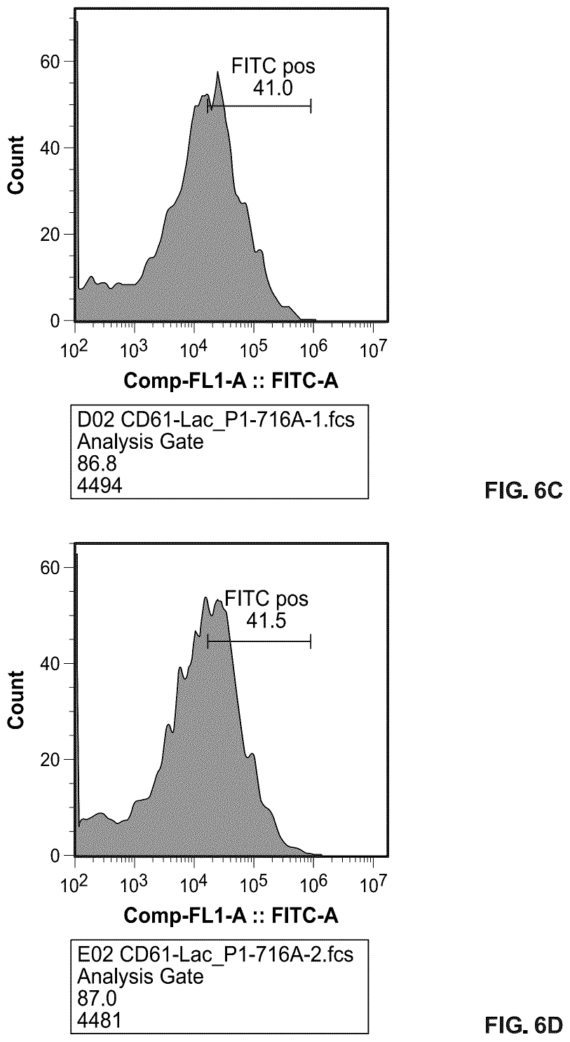

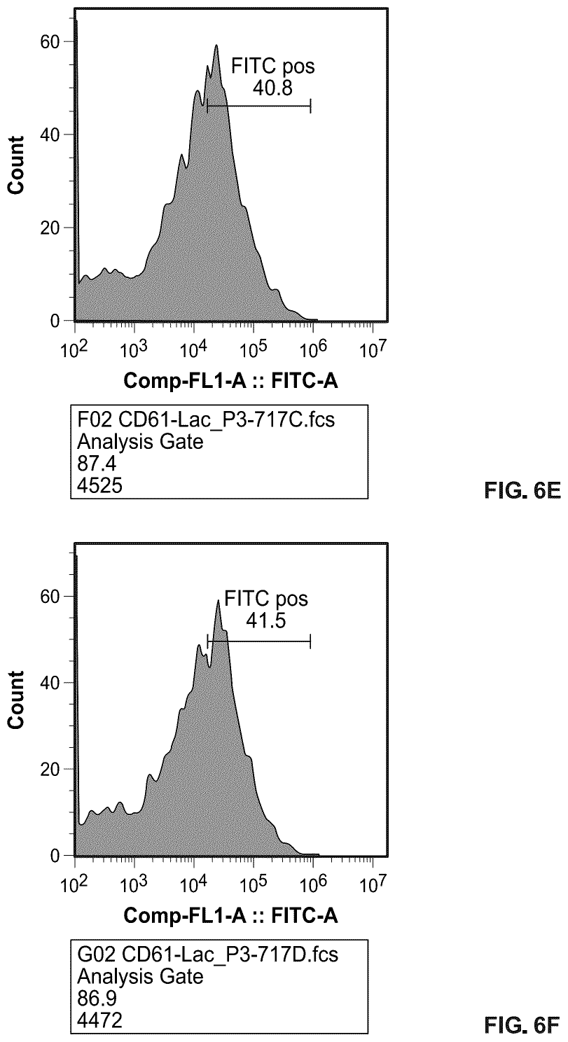

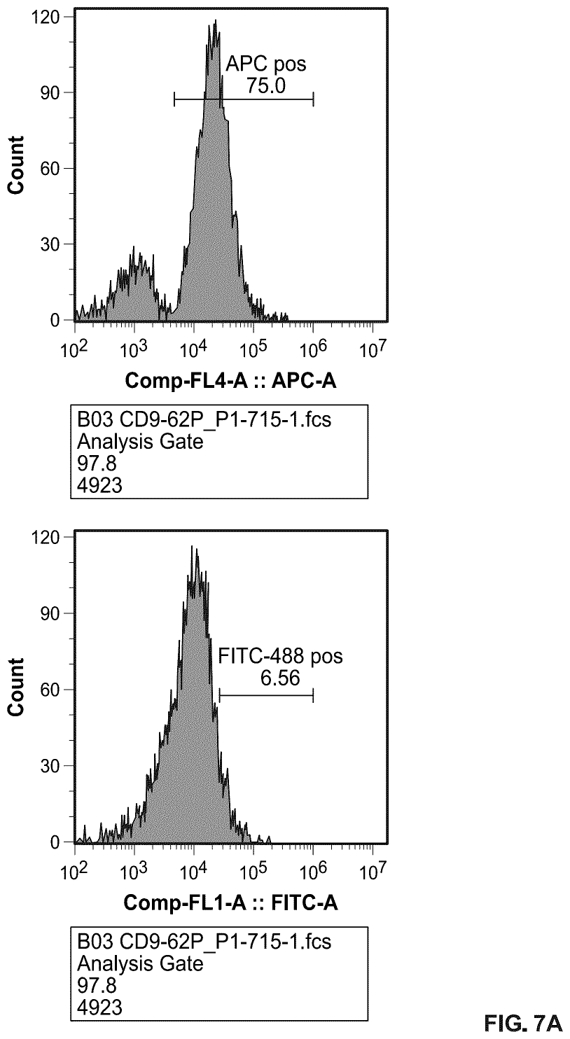

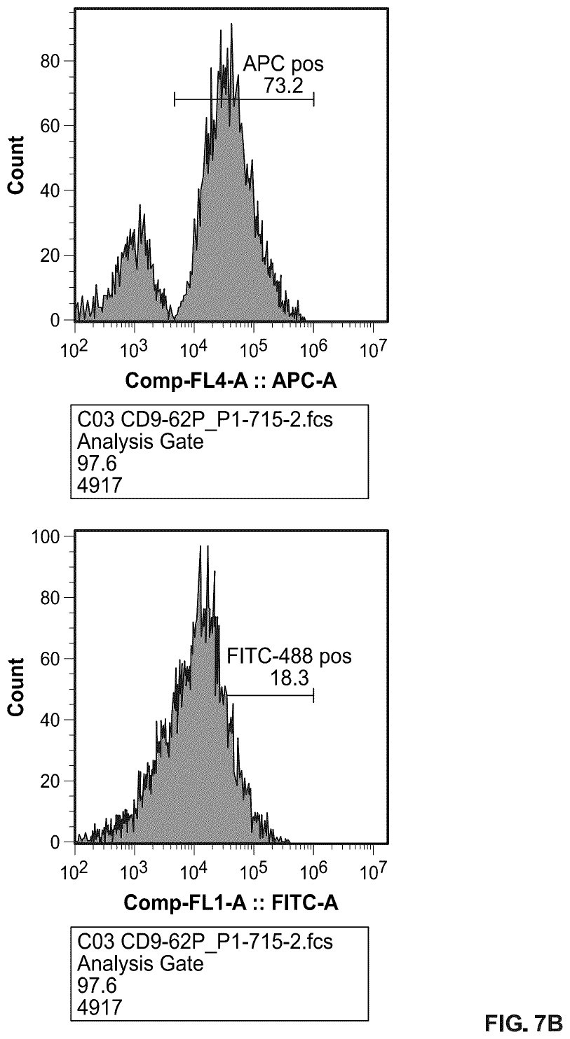

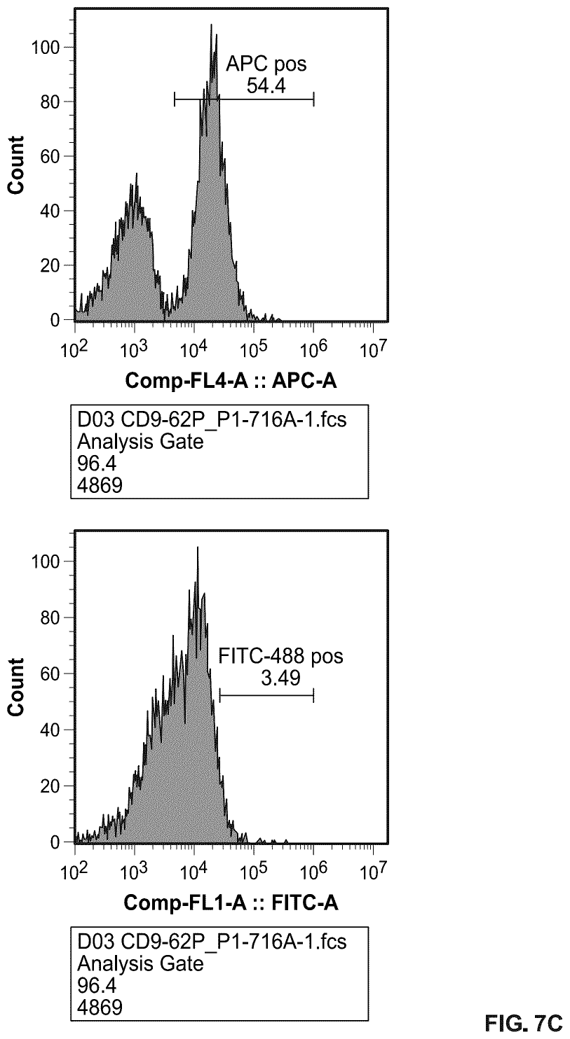

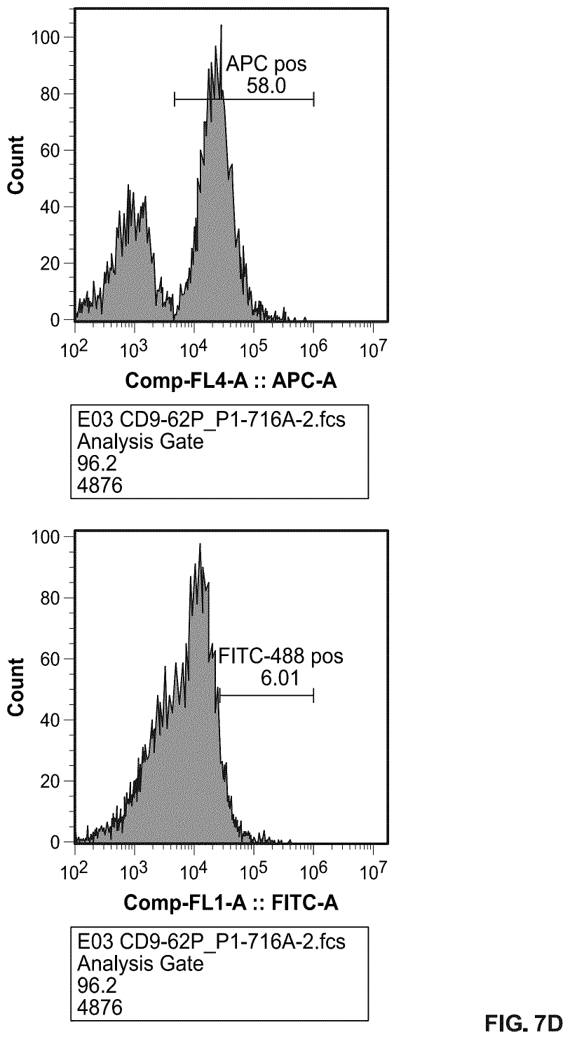

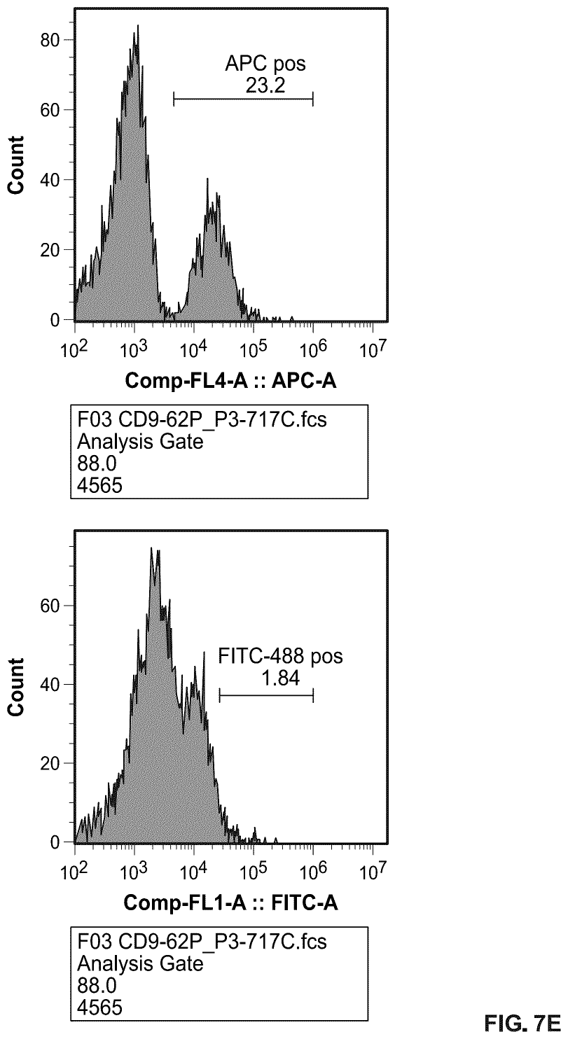

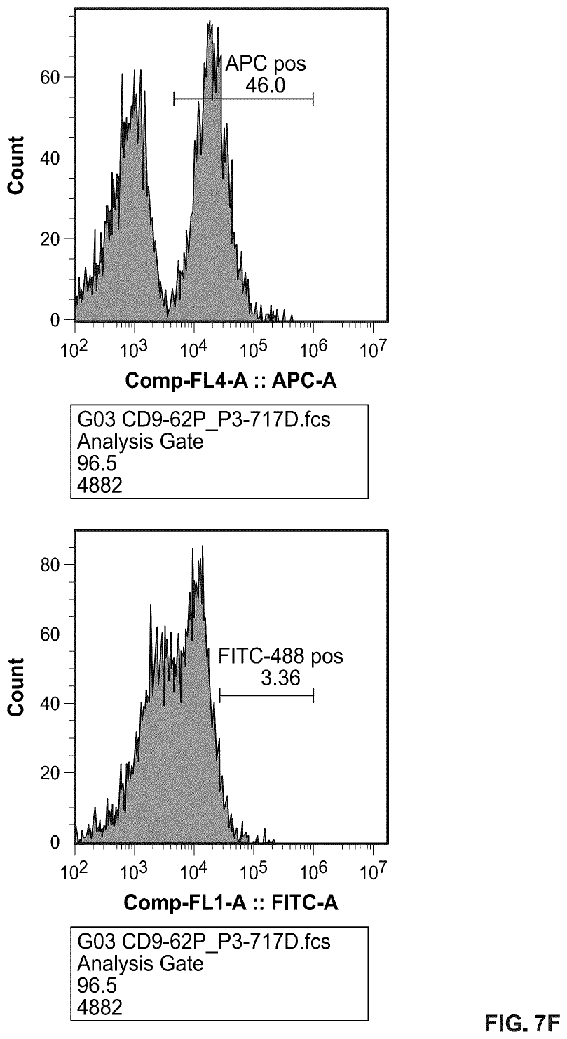

All the samples exhibited uniform particle size with the majority of all samples falling within the 0.5 to 2 micron gate. The combination of flow cytometry cell type, activation status and size data indicate about 90% to 99% of the cells are platelets. The CD61 and CD9 positivity values of less than 90% result from detection limits or binding affinities of the monoclonal antibodies, which are not equine specific, used the flow cytometry analysis. The overlap in platelet and lactadherin results, in combination with the size data provided above, indicate that 90 to 99% of the cells in the reconstituted lyophilized PRP composition are platelets in the partially activated state, containing phosphatidylserine on the outer leaflet of the plasma membrane, but are not fully activated, as the majority are not CD62P positive. The pan-leukocyte marker CD44 represented fewer than 1% of particles for all samples tested, indicating that less than 1% of the cells in the samples were white blood cells. The results are set forth in FIGS. 5A-5F which depict the results for the platelet (CD61 and CD9) and white blood cell (CD44) markers for samples 1-6, respectively; FIGS. 6A-6F which depict the results for outer membrane phosphatidylserine (lactadherin) for samples 1-6, respectively; FIGS. 7A-7F which depict the flow cytometry results for platelets (CD9) and activated platelets (P-selectin) for samples 1-6, respectively; and in Table 5, below:

TABLE 5
Summary of Membrane CD61, CD9,
CD44 and lactadherin labeling

| | | | | % | |
|---|---|---|---|---|---|
| | % CD61 | % CD9 | % CD44 | Lactadherin | % CD62P |
| Sample | positive | positive | positive | positive | positive |
| Number | (1) | (1) | (1) | (2) | (3) |
| 1 | 45.2 | 47.8 | 0.04 | 52.0 | 6.5 |
| 2 | 52.3 | 54.2 | 0.04 | 49.7 | 18.3 |
| 3 | 43.3 | 40.7 | 0.1 | 41.0 | 3.5 |
| 4 | 49.7 | 46.4 | 0.16 | 41.5 | 6.0 |
| 5 | 45.7 | 44.0 | 0.1 | 40.8 | 1.8 |
| 6 | 37.2 | 41.8 | 0.04 | 41.5 | 3.4 |
| Average | 45.6 | 45.8 | 0.1 | 44.4 | 6.6 |

(1) % positive displayed for triple label reaction: CD61, CD9, CD44
(2) % positive displayed for double label reaction: CD61 & Lactadherin
(3) % positive displayed for double label reaction (CD9 & CD62P)

Extrapolating these numbers, if an average of 45.6% of particles are positive for platelet markers, but, based on the size data and the CD44 data up to about 99% of the particles are platelets, it is predicted that about 14.5% of the cells are activated (if 45.6% positive for platelet markers indicates 100% (a factor of 2.2) then 6.6% CD62P positive indicates that the total percent of CD62P positive cells in the sample is 14.5% (6.6% times a factor of 2.2).

### Example 4

### Lyophilized Equine Platelet-Rich Plasma Composition Stability

Lyophilized PRP is stable for at least 2 years at ambient conditions (room temperature, 20° C. to 25° C. and at or about 62% relative humidity (RH)). Stability is assessed based on the low residual moisture in lyophilized products. Lyophilized PRP compositions, which contain cryopreservatives, have a shelf life of approximately 20 years. Residual moisture in shelf stable lyophilized PRP compositions is less than 2.5%, which is similar to the commercially available products with shelf life of multiple years. StablePlate RX® platelets are highly stable, having a shelf-life of at least 18 months at room temperature or below (U.S. Pat. No. 8,486,617). Thrombosomes® platelets have a shelf-life of up to 3 years at ambient temperatures. Other lyophilized platelet products in the art suggest a shelf-life of at least 2 years. Based on the results herein, the lyophilized PRP compositions provided herein exhibit a similar shelf-life, but possess the advantageous properties demonstrated herein, such as, for example, the higher percentage of primed platelets, and the absence of a cryopreservative.

### Composition Stability at Ambient Temperatures

PRP, as detailed in Example 1 was prepared. The PRP was tested for stability at ambient temperatures for six months. The results show that after six months the platelets in the PRP composition retained their primed state as defined by lactadherin and p-selectin staining and were not fully activated, as the amount of CD62 positivity did not increase. These results indicate that PRP is stable for at least six months and maintains the properties of the PRP after lyophilization. Increased stability at ambient temperatures is advantageous, such as in areas of the world with limited refrigeration.

### Pooled Platelet-Rich Plasma Composition Stability Compared to Fresh and Frozen PRP

Lyophilized PRP described as detailed in Example 1 is prepared. The lyophilized PRP is reconstituted in sterile saline and tested for stability at ambient temperatures compared to pooled PRP prepared as in example 1 prior to lyophilization (“fresh PRP”), and compared to “fresh PRP” frozen without freeze drying. Platelets in the PRP composition described herein are primed as indicated by the phosphatidylserine on the outer membrane. They are more primed than platelets previously characterized from fresh PRP and less primed than previously characterized frozen cryopreserved platelets and reconstituted lyophilized platelets (see Johnson et al., (2015) Transfusion 55 (4): 838-847; Goggs et al., (2020) J Vet Intern Med. 34 (6): 2384-2397). doi: 10.1111/jvim.15922).

### Example 5

### Characterization of Cell Size in Lyophilized Canine Platelet-Rich Plasma Composition by Flow Cytometry

### A. Assessing the Particle Size of Reconstituted Lyophilized Canine Platelet-Rich Composition

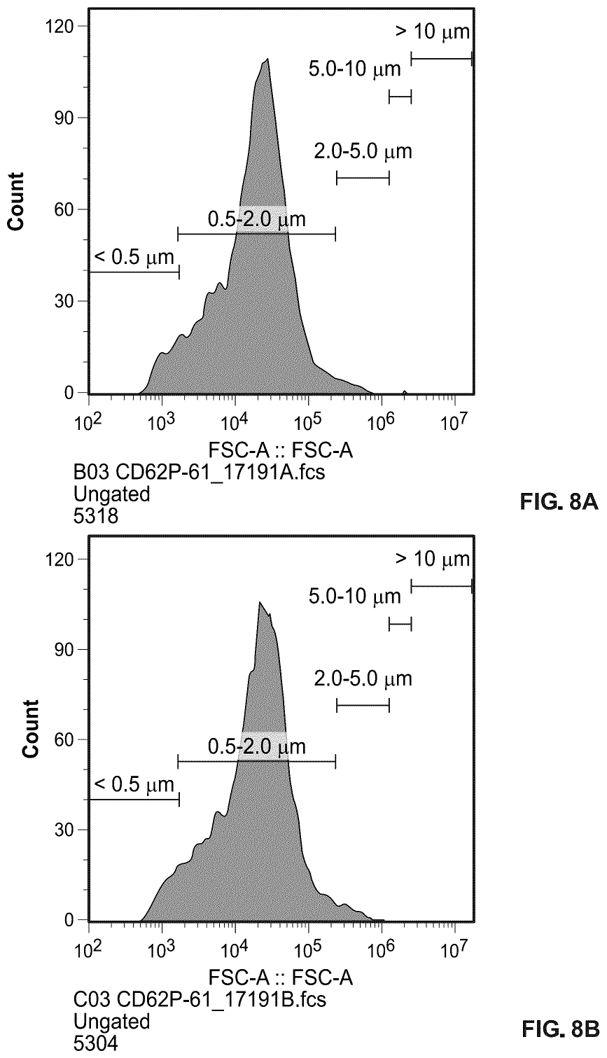

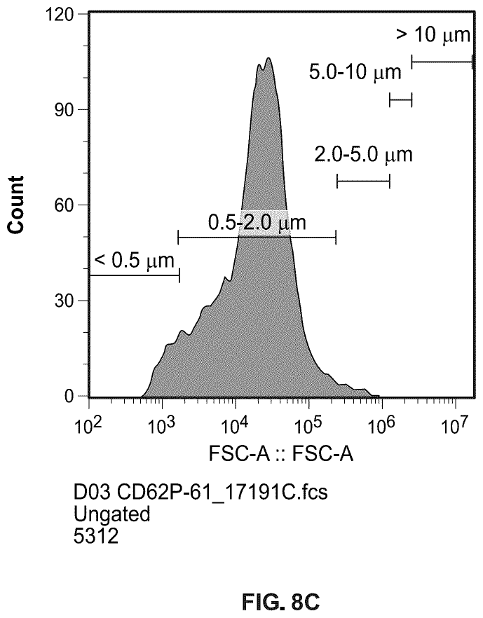

Flow cytometric assays were performed as detailed above in Example 2 to characterize the lyophilized canine platelet rich plasma composition. Three vials of lyophilized platelet rich plasma (PRP) reconstituted in 8 mL sterile water (0.9% saline) were assessed. The samples were diluted 1:800 and the average particle size in the samples was assessed. The results show that the three vials demonstrated uniform particle size, over 90% of the particles from the reconstituted canine PRP composition fell within a 0.5 to 2 micron gate. These results indicate that the majority of particles in the PRP composition are platelets, and that the composition is substantially free of red and white blood cells. The results from the three vials are set forth in FIGS. 8A-8C and the Table 6, below:

TABLE 6
Summary of Particle Size and Percent of Each Particle Size

| | Number of Events or Percentage within Gate | | | | | | | | | |
|---|---|---|---|---|---|---|---|---|---|---|
| Particle | | | 0.5-2.0 | %0.5-2.0 | 2-5 | %2-5 | 5-10 | %5-10 | >10 | %>10 |
| Count | <0.5 | %<0.5 | μM | μM | μM | μM | μM | μM | μM | μM |
| 5318 | 312 | 5.9% | 4937 | 92.8% | 81 | 1.5% | 6 | 0.1% | 0 | 0 |
| 5304 | 295 | 5.6% | 4916 | 92.7% | 102 | 1.9% | 4 | 0.1% | 0 | 0 |
| 5312 | 323 | 6.1% | 4921 | 92.6% | 84 | 1.6% | 1 | 0.0% | 0 | 0 |

### B. Assessing the Cell Types and Activation Status in the Reconstituted Lyophilized Canine Platelet-Rich Composition

Aliquots of each vial were labeled with a panel of markers summarized in Table 7 below. Analyses include percentage of positive events within a gate defined by size/scatter properties, and particle size within gates defined by calibrated microspheres. Antibody-conjugated beads and aliquots of frozen/thawed canine platelet rich plasma were used for compensation and labeling controls, and isotype antibodies were used to set positive/negative fluorescence boundaries.

TABLE 7
Leukocyte and platelet membrane markers

| Marker/clone | Target | Supplier |
|---|---|---|
| CD18-PE/7E4 | pan-leukocyte | Beckman Coulter |
| CD61-PE /SZ21 | platelet- GpIIIa | Beckman Coulter |
| CD61-FITC/Y251 | platelet- GpIIIa | Dako |
| CD9-FITC/H19A | platelet -tetraspanin | Biolegend |
| CD62P-PE/AC1.2 | platelet-P selectin | BD Biosciences |
| Lactadherin-FITC | outer membrane | Haematologic |
| | phosphatidylserine | Technologies |

Antibody-conjugated beads and aliquots of frozen/thawed canine platelet rich plasma were used as controls, and isotype antibodies were used to set positive/negative threshold boundaries. The particle size and the percentage of particles that expressed each cell and/or membrane marker were assessed. Three labeling reactions were performed for each sample: constitutive platelet marker and pan-leukocyte marker (CD9-FITC, CD18-PE); constitutive platelet membrane marker & phosphatidylserine (PS) marker (CD61-PE & lactadherin-FITC); constitutive platelet membrane marker & P selectin marker (CD61-FITC & CD62P-PE.) An acquisition forward scatter threshold of 2,500 was used to allow detection of small (<0.5 μM) events.

The labeling reaction results show that 50-60% of reconstituted canine PRP composition particles were positive by FACS for the platelet membrane markers CD61 and CD9. The anti-CD61 and anti-CD9 antibodies, however, were generated against human antigen; the fact that about 50-60% of the particles in the composition are CD61 and CD9 positive is indicative of incomplete binding due to species differences between human and canine CD61 and CD9. The experiments in part A, above, indicate that greater than 90% of the particles in the composition are the size of platelets. Thus, up to 100% of the particles in the sample are platelets. The discrepancy in the particle size results and the CD61/CD9 results likely is due to incomplete binding by the CD61/CD9 antibodies.

The results also show that about 60% of the cells in the sample are positive for lactadherin, indicating externalized phosphatidylserine. The percentage of cells that are positive for platelet membrane markers (e.g., CD61 and CD9) and those that are positive for lactadherin overlap (average about 57% platelet positive and 60% lactadherin positive), indicating that the majority (up to 100%) of the platelets in the sample are positive for lactadherin and have externalized phosphatidylserine. The results indicate that all of the platelets that survive the process of lyophilization do so with intact with externalized phosphatidylserine. An average of only about 31% of the cells were CD62P-positive (indicative of activation).

All of the samples exhibited uniform particle size with the majority of all samples falling within the 0.5 to 2 micron gate. The combination of flow cytometry cell type, activation status and size data indicate about 90% to 99% of the cells are platelets. The CD61 and CD9 positivity values of less than 90% result from detection limits or binding affinities of the monoclonal antibodies, which are not canine specific, used the flow cytometry analysis. The overlap in platelet and lactadherin results, in combination with the size data provided above, indicate that 90 to 99% of the cells in the reconstituted lyophilized PRP composition are platelets in the partially activated state, containing phosphatidylserine on the outer leaflet of the plasma membrane, but are not fully activated, as the majority are not CD62P positive. The results are set forth in in Table 8, below, which depicts the results for the platelet (CD9-FITC) and white blood cell (CD18-PE) markers for samples 1-3; the results for platelet (CD61-PE) and outer membrane phosphatidylserine (lactadherin-FITC) for samples 1-3; and the flow cytometry results for platelet (CD61-FITC) and activated platelets (CD62P-PE, P-selectin) markers for samples 1-3.

TABLE 8
Summary of Membrane CD61, CD9, CD18,
CD62P and lactadherin labeling

| | | | | % | |
|---|---|---|---|---|---|
| | % CD9 | % CD61 | % CD18 | Lactadherin | % CD62P |
| Sample | positive | positive | positive | positive | positive |
| Number | (1) | (2) | (1) | (2) | (3) |
| 1 | 58.3 | 59.6 | 4.3 | 63.2 | 30.9 |
| 2 | 56 | 57.7 | 4.3 | 59.8 | 30.6 |
| 3 | 56.2 | 55 | 4.4 | 56.2 | 31.5 |
| Average | 56.8 | 57.4 | 4.3 | 59.7 | 31.0 |

(1) % positive displayed for triple label reaction: CD9, CD18
(2) % positive displayed for double label reaction: CD61 & Lactadherin
(3) % positive displayed for double label reaction: CD61 & CD62P

### Example 6

### Evaluation of Growth Factor and Chemokine Concentration in Equine and Canine Platelet-Rich Plasma (PRP)

The concentration of species-specific growth factors (TGF-β, PDGF-BB, VEGF) and cytokines (TNF-α and IL1B) are evaluated for all subjects using ELISA. Platelets are activated by the addition of CaCl₂). Platelet poor plasma, prepared by subjecting the PRP composition prepared as detailed herein to centrifugation to concentrate the platelets at the bottom; the supernatant is removed and is used as a negative control (platelet poor plasma (PPP)).

### Evaluation of Anti-Inflammatory Properties of an Equine and Canine Platelet-Rich Plasma Composition

The anti-inflammatory effect of a PRP composition and reconstituted lyophilized PRP composition is evaluated. Platelet poor plasma is used as negative control. Equine and canine monocytes are isolated and stimulated with Lipopolysaccharide (LPS) to induce an acute inflammatory response. Next, 80% or 20% v/v of PRP is added to the stimulated monocytes. In response to the addition of endotoxin LPS, monocytes are triggered to produce large quantities of proinflammatory cytokines such as tumor necrosis factor alpha (TNF-α). After a 24 hour incubation of the PRP with the stimulated monocytes, cell culture supernatants are obtained and TNF-α expression is assessed. A PRP composition with anti-inflammatory properties induces decreased TNF-α expression compared to platelet poor plasma.

Evaluation of Neutrophil Reactive Oxygen Species (ROS) Release After Treatment with an Equine and Canine Platelet-Rich Plasma Composition

The effect of a PRP composition and reconstituted lyophilized PRP composition on release of reactive oxygen species in neutrophils is evaluated. Platelet Canine and equine neutrophils isolated from peripheral blood and 2×10\hat{ }5 cells are stimulated with Phorbol-12-Myristate-13-Acetate (PMA) in the presence or absence of PRP. PMA activation of NADPH oxidase results in a consequent increase in ROS production in neutrophils. ROS following PMA stimulation is measured using an assay utilizing nonfluorescent reactive oxygen species (ROS) indicator dihydrorhodamine 123 (DHR-123). DHR-123 passively diffuses across cell membranes where it is oxidized to cationic rhodamine 123, which localizes to the mitochondria and exhibits green fluorescence. 5 μM dihydrorhodamine 123 (DHR-123) is added to the neutrophils and incubated for 2 hours at 37° C., 5% CO₂. Subsequently, fluorescence intensity is measured at 485 nm excitation and 538 nm emission on a fluorescence plate reader (from Biotek, Synergy). Neutrophils that release lower levels of ROS will show decreased fluorescence compared to neutrophils that release higher levels of ROS; fluorescence level corresponds with ROS level. PRP that inhibits ROS formation in neutrophils exhibit decreased fluorescence compared to neutrophils that are stimulated with PMA in the absence of PRP.

### Example 7

### Intact Granules Containing Growth Factors in Lyophilized Equine and Canine Platelet-Rich Plasma Composition

### Chemokine Results in Equine PRP

The freeze-dried equine PRP composition was prepared as detailed above and reconstituted in 0.9% saline. Three lots of pooled equine PRP were tested. Chemokine (PDGF, VEGF and TGF-B) levels were assessed by validated ELISA.

| | PDGF | | VEGF | | TGFβ1 | |
|---|---|---|---|---|---|---|
| | (pg/ml) | | (pg/ml) | | (pg/ml) | |
| Lot tested | mean | SD | mean | SD | mean | SD |
| equine PRP | 751.90 | 8.79 | 1829.18 | 4.84 | 50882.76 | 963.18 |
| Lot A | | | | | | |
| equine PRP | 722.96 | 11.30 | 1062.18 | 50.28 | 26557.14 | 1249.62 |
| Lot B | | | | | | |
| equine PRP | 939.98 | 54.59 | 971.12 | 14.56 | 25995.96 | 1019.76 |
| Lot C | | | | | | |
| Average PRP | 804.95 | 117.83 | 1287.49 | 23.23 | 34478.62 | 1077.52 |

The results show that PDGF, VEGF, and TGF-B remain in the platelet granules throughout platelet collection, platelet pooling, platelet, and plasma pooling, and PRP preparation and freeze-drying processes, and remain in the platelet granules in the granules after reconstitution.

### Chemokine Results in Canine PRP

The lyophilized canine PRP composition was prepared as detailed in Example 1, above, with platelet rich plasma collected from dogs. The lyophilized canine PRP was reconstituted in 0.9% saline, and chemokine (PDGF) levels were assessed by validated ELISA.

PDGF (pg/ml)

| Lot tested | mean | SD |
|---|---|---|
| canine PRP Lot A | 362.06 | 48.1 |
| canine PRP Lot B | 538.1 | 106.6 |
| Average PRP | 450.08 | 77.35 |

The results show that PDGF remains in the platelet granules throughout platelet collection, platelet pooling, platelet, and plasma pooling, and PRP preparation and freeze-drying processes, and remain in the platelet granules in the granules after reconstitution.

### Pooled Platelet-Rich Plasma Composition Response to Agonist

Response of platelets in the reconstituted lyophilized pooled PRP composition to agonists is assessed. The PRP composition is treated with thrombin or CaCl₂) to induce platelet activation, and chemokine release is assessed by ELISA. Growth factors are released after the addition of agonist or calcium (e.g., CaCl₂)). The platelets containing the chemokines (growth factors) in the granules are not activated, and can be in a primed state, where the granules have not released their contents. Platelets in the unactivated or primed state can be activated, such as by the addition of CaCl₂) or by subjecting the PRP to freeze-thaw cycle(s), where upon activation growth factors, such as PDGF, VEGF, and TGFB1, are released from the granules. Platelets in the PRP composition described herein are in the primed state, and can be activated by an external stimulus, such as, for example, thrombin or calcium.

### Example 8

### Therapeutic Use of the Reconstituted Lyophilized Platelet-Rich Plasma Composition in Horses

The reconstituted lyophilized platelet-rich plasma (PRP) composition is administered to horses for treating osteoarthritis (OA) associated with a joint. The reconstituted lyophilized PRP composition is administered intra-articularly to an animal demonstrating reduced mobility or lameness. The dosage for administration is calculated by the size of the animal in kg and amount of joint space available for PRP infusion. Final volume administered in horses and megavertebrates is 4 mL-8 mL PRP in larger joints (stifle, knee) and 2 mL to 4 mL in smaller joints (elbow, carpus). The PRP composition administered is leukoreduced or is not leukoenriched.

The reconstituted lyophilized PRP composition is administered to elephants to improve wound healing where the wound previously was not healing. The elephant is treated with 10-15 mL of the reconstituted lyophilized PRP composition administered topically or intradermally at the wound edge. The PRP can be activated with calcium prior to administration to form a white platelet clot or gel. The reconstituted lyophilized PRP composition can be administered with inert PSSAG, hyaluronic acid, triamcilone and/or small molecules such as IRAP-1.

The reconstituted lyophilized PRP composition is administered to horses or elephants for treating orthopedic diseases, disorders, or conditions. The reconstituted lyophilized PRP composition is administered to treat tendon, ligament or bone diseases, disorders, or conditions.

### Example 9

### Therapeutic Use of the Reconstituted Lyophilized Platelet-Rich Plasma Composition in Dogs

### Osteoarthritis

The effectiveness of canine reconstituted lyophilized platelet-rich plasma (PRP) composition after intra-articular injection in joint or joints affected by osteoarthritis is assessed. For example, the reconstituted lyophilized platelet-rich plasma (PRP) composition is administered to dogs for treating osteoarthritis (OA) or for ameliorating or lessening the symptoms of OA or preventing worsening of OA or the symptoms of OA. For example, the reconstituted lyophilized platelet-rich plasma (PRP) composition is administered to dogs for treating stifle joint osteoarthritis (OA) or for ameliorating or lessening the symptoms of stifle joint OA or preventing worsening of stifle joint OA or the symptoms of stifle joint OA.

Domestic canines of any breed, either male or female, are included in the study if, prior to the study start: 1) there is identified lameness upon veterinary examination, where the affected joints demonstrate identifiable lameness; 2) a radiograph of the selected joints demonstrate osteoarthritis; 3) the canine is ≥six months of age and in good general health; 4) animals demonstrated general behavior to allow for handling and not interfere with outcome assessment; and 5) a generally normal CBC/blood chemistry result that do not show any impact assessment of variables assessed. Animals are excluded from the study if, prior to the study start: 1) animal had surgery or received anesthesia within 30 days prior to enrollment; 2) animal had joint surgery within 90 days prior to enrollment; 3) animal received pain or anti-inflammatory drugs allowed 14 days prior to the study (includes NSAIDS, HA, PSGAG, steroids, antibiotics with anti-inflammatory properties), with the exception if the dogs were on a stable dosing profile for more than 14 days; 4) animal received autologous or allogeneic biologic therapies in the 90 days prior to enrollment or during the study (including platelet rich plasma, IL2ra products, exosomes, A2M or other serum, blood, or tissue derived therapies including stem cells); 5) animal received chiropractic, acupuncture, shockwave, or laser treatment within thirty days of enrollment or during study; and/or 6) animals had other known health problems or condition that would interfere with outcome measurements. Animals that were receiving joint or other health supplements for at least thirty days prior to enrollment and who maintained at same level during the study, were not excluded.

Two cohorts of animals are selected for treatment and evaluation. Each cohort is administered a different dose of canine reconstituted lyophilized PRP composition. For example, cohort 1 (C1) is administered 1 mL, and cohort 2 (C2) is administered 2 mL canine reconstituted lyophilized platelet-rich plasma (PRP) composition intra-articularly. A randomized dose is injected into one or two joints. Up to two joints may be injected if clinician deems it feasible to independently determine lameness assessment in each joint. If an animal is injected in two joints, the dosage is the same for both joints.

The reconstituted lyophilized platelet-rich plasma (PRP) composition is administered by intra-articular injection. When required, the reconstituted lyophilized platelet-rich plasma (PRP) composition is administered multiple times. For example, a second or third treatment may be necessary depending on the severity of the pathology.

Primary outcomes are assessed. A baseline assessment is conducted on Day 7, including assessing the following: 1) CBC/serum chemistry; 2) by physical examination; 3) by lameness examination; 4) for passive pain upon manipulation; and 5) by imaging, such as radiographs of selected joints compared to imaging (e.g., radiographs) prior to treatment. Post-treatment outcomes also are assessed. For example, the following are assessed on the days set forth in Table 9, below:

TABLE 9
Primary outcomes assessed

| Post-treatment Outcome Assessment | Day(s) assessed |
|---|---|
| Physical exam | 0, 7, 21 |
| Injection site observations | 0, 7, 21 |
| Daily health journal of | 0, 1, 2, 3, 4, 5, 6, 7, |
| observing veterinarian | 16, 17, 18, 19, 20, 21 |
| CBC/ Chem | 0 |
| Lameness (veterinary observation) | 0, 7, 21 |
| Passive pain on manipulation | 0, 7, 21 |
| Client Specific Outcome Measures (CSOM) | 0, 7, 21 |

When the PRP treatment is used in conjunction with an additional therapeutic treatment, the dosage of reconstituted lyophilized platelet-rich plasma (PRP) composition is administered before, after or with the additional therapeutic treatment. The dosage of the PRP administered can be modified compared to when the PRP composition is administered in the absence of an additional therapeutic; the dosage of the reconstituted lyophilized PRP composition is less or more when administered with an additional therapeutic(s). The therapeutics can be a steroid, hyaluronic acid, an antibiotic if infection is suspected, or other joint inflammation modifying treatments.

Treatment of Canines with Stifle Joint Osteoarthritis (OA) at the Time of Tibial Leveling Osteotomy (TPLO)

The reconstituted lyophilized PRP composition is administered to dogs for treating stifle joint osteoarthritis (OA) at the time of tibial leveling osteotomy (TPLO). The reconstituted lyophilized PRP composition is administered to dogs for treating stifle joint osteoarthritis (OA) at the time of tibial leveling osteotomy (TPLO) to enable rapid recovery after surgery. When the PRP composition is administered to dogs for treating stifle joint osteoarthritis (OA) at the time of tibial leveling osteotomy (TPLO), the PRP is administered by intra-articular injection during or after surgery. Administration of the PRP composition at the time of TPLO will accelerate healing and reduce pain, allowing physical therapy to proceed. Postoperative evaluation of TPLO combined with PRP administration supports quicker return to normal clinical function compared to TPLO conducted without PRP administration.

### Calcaneal Tendon Damage

The reconstituted lyophilized platelet-rich plasma (PRP) composition is administered to dogs for treating damage to the calcaneal tendon. The reconstituted lyophilized platelet-rich plasma (PRP) composition is administered intralesionally. The reconstituted lyophilized platelet-rich plasma (PRP) composition is administered by intra-tendinous injection as a single or repeated injection, depending on the healing rate. The reconstituted lyophilized platelet-rich plasma (PRP) composition is administered at dosages of 1-2 mL, depending on the size of the lesion. The dosage for administration is calculated considering the injury size, size of the animal, and chronicity. When the PRP treatment is used in conjunction with an additional therapeutic treatment, the dosage of reconstituted lyophilized platelet-rich plasma (PRP) composition is administered before or after the additional therapeutic treatment and the dosage can be reduced or increased; the dosage of the reconstituted lyophilized PRP composition is dependent on the injury severity and chronicity when administered with an additional therapeutic(s). Hyaluronic acid or other matrix materials may be administered with, or before or after administration of the PRP composition.

### Example 10

### Therapeutic Use of the Reconstituted Lyophilized Platelet-Rich Plasma Composition in Humans

### Osteoarthritis

The reconstituted lyophilized platelet-rich plasma (PRP) composition is administered to human subjects for treating osteoarthritis (OA) or ameliorating or lessening the symptoms of OA or preventing worsening of OA or the symptoms of OA. The reconstituted lyophilized PRP composition is administered to human subjects directly into the joint(s) affected by osteoarthritis or administered intra-articularly. The dosage for administration can depend on various factors and is calculated in view of these factors including, but not limited to the particular human subject, such as the age or size of the subject, and also on the particular condition, such as the severity or stage or location of the osteoarthritis.

### Stifle Joint Osteoarthritis

The reconstituted lyophilized platelet-rich plasma (PRP) composition is administered to human subjects for treating stifle joint osteoarthritis (OA) or ameliorating or lessening the symptoms of stifle joint OA or preventing worsening of stifle joint OA or the symptoms of stifle joint OA. The reconstituted lyophilized platelet-rich plasma (PRP) composition is administered to human subjects directly into the joint(s) affected by osteoarthritis or administered intra-articularly. The reconstituted lyophilized platelet-rich plasma (PRP) composition is administered multiple times, including on a regular schedule, such as monthly. Administration continues until symptoms are ameliorated or lessened. The dosages of a PRP composition for treating stifle joint OA can vary as detailed above. When the PRP treatment is used in conjunction with an additional therapeutic treatment, the dosage of reconstituted lyophilized platelet-rich plasma (PRP) composition is administered with or before or after administration of the additional therapeutic treatment and the dosage can be modified compared to when PRP is administered as the single therapeutic.

The reconstituted lyophilized PRP composition is administered to human subjects for treating stifle joint osteoarthritis (OA) at the time of tibial leveling osteotomy (TPLO). The reconstituted lyophilized PRP composition is administered to human subjects for treating stifle joint osteoarthritis (OA) at the time of tibial leveling osteotomy (TPLO) to accelerate recovery after surgery. When the PRP composition is administered to human subjects for treating stifle joint osteoarthritis (OA) at the time of tibial leveling osteotomy (TPLO), the PRP is administered before or during or after TPLO surgery. PRP is administered at the time of TPLO to increase efficacy of other therapeutic treatments, accelerate recovery, decrease pain during recovery, and/or speed return to normal clinical function. Postoperative evaluation of TPLO combined with PRP administration supports a quicker return to normal clinical function and/or other therapeutic benefits compared to TPLO conducted without PRP administration.

### Wound Healing

The reconstituted lyophilized PRP composition also is administered to human subjects for wound healing. The reconstituted lyophilized PRP composition is administered to human subjects to improve wound healing where the wound previously was not healing naturally or with the assistance of other therapeutics or is not healing quickly. The human subject is treated with the reconstituted lyophilized PRP composition administered topically to an exposed wound, and is injected to treat internal wounds, such as, for example, tissue lesions or tears. The amount of reconstituted lyophilized PRP composition administered to the wound is calculated based on various factors, including the severity of the wound, wound location, type of wound, size of wound and others. The reconstituted lyophilized PRP composition is administered with therapeutics that act in concert with the PRP or augment the effects of the PRP or that otherwise act to improve or accelerate wound healing.

### Tendon Damage

### Calcaneal Tendon Damage

The reconstituted lyophilized platelet-rich plasma (PRP) composition is administered to human subjects for treating damage to the calcaneal tendon. The reconstituted lyophilized platelet-rich plasma (PRP) composition is administered intra-lesionally or via intra-tendinous administration (e.g., intra-tendinous injection) for treating damage to the calcaneal tendon. The reconstituted lyophilized platelet-rich plasma (PRP) composition is administered intratendinous injection as a single or repeated injection, depending on the healing rate. The repeated injection is administered if additional healing is desired or if the healing rate is slower than desired. The reconstituted lyophilized platelet-rich plasma (PRP) composition is administered in volumes of 1-2 mL. The dosage for administration is calculated considering the injury size, size of the animal, and chronicity. When the PRP treatment is used in conjunction with an additional therapeutic treatment, the dosage of reconstituted lyophilized platelet-rich plasma (PRP) composition is administered before or after the additional therapeutic treatment and the dosage can be reduced or increased; the dosage of the reconstituted lyophilized PRP composition is dependent on the injury severity and chronicity when administered with an additional therapeutic(s).

### Example 11

### Therapeutic Use Lyophilized Pooled Platelet-Rich Plasma Composition for Treating Ophthalmic or Ocular Diseases, Disorders and Conditions

### Ocular Lesions in Dolphins

The reconstituted lyophilized platelet-rich plasma (PRP) composition is administered to dolphins for treating ocular lesions, such as keratitis, corneal lesion, and ulcer. The reconstituted lyophilized PRP composition is administered to dolphins by subconjunctival administration to the eye. The dosage for administration is generally 1-2 mL to accommodate the location. The reconstituted lyophilized PRP composition is administered alone or is administered with other therapeutic agents. The reconstituted lyophilized PRP composition is administered with mesenchymal stem cells (MSCs) for repairing ocular tissue. The reconstituted lyophilized PRP composition also is administered with antibiotics, antimycotics, and/or other anti-inflammatory medications as needed.

Administration of a reconstituted lyophilized platelet-rich plasma (PRP) composition decreases sequelae of ocular lesions, such as visual impairment, corneal scarring, perforation, deepening of the ulcer, and/or others. Administration of a reconstituted lyophilized platelet-rich plasma (PRP) composition decreases the healing time of the ocular lesion.

The reconstituted lyophilized PRP composition is administered to dolphins for treating ophthalmic or ocular diseases, disorders, or conditions. The reconstituted lyophilized PRP composition administered with MSCs shows unexpected efficiency for treating ocular conditions, such as ocular lesion, in human subjects.

### Ocular Diseases, Disorders or Conditions in Dogs

The reconstituted lyophilized platelet-rich plasma (PRP) composition is administered to dogs for treating ocular diseases, disorders, or conditions. The reconstituted lyophilized PRP composition is administered to dogs for treating keratoconjunctivitis sicca (KCS), or dry eye. The reconstituted lyophilized PRP composition is administered to dogs by topical ocular administration. The dosage for administration is calculated by assessing the size of the patient and ability to place a subconjunctival volume. The volume administered to an average dog with KCS is 0.5-1 mL. The amount of reconstituted lyophilized PRP composition administered to the eye is calculated based on size of the animal. The reconstituted lyophilized PRP composition is administered to the eye alone or is administered with other therapeutic agents. The reconstituted lyophilized PRP composition is administered with other therapeutic agents for treating KCS or repairing ocular dysfunction related to KCS. The reconstituted lyophilized PRP composition also is administered with an antibiotic, antimycotic or other anti-inflammatory medication and/or eye lubricant as necessary.

Administration of a reconstituted lyophilized platelet-rich plasma (PRP) composition decreases sequelae of KCS, such as visual impairment, recurrent conjunctivitis, dull cornea, conjunctival thickening, corneal vascularization, laceration, and/or others. Administration of a reconstituted lyophilized platelet-rich plasma (PRP) composition decreases the healing time of the ocular lesion.

The reconstituted lyophilized PRP composition is administered human subjects for treating ophthalmic or ocular diseases, disorders, or conditions. The reconstituted lyophilized PRP composition administered with MSCs shows unexpected efficiency for treating ocular conditions, such as ocular lesion, in human subjects.

### Example 12

### Clinical Safety of the Lyophilized Pooled Platelet-Rich Plasma Composition in Horses and Dogs

The clinical safety of the reconstituted lyophilized pooled PRP composition is assessed in horses and dogs.

### Safety Assessment in Horses

Safety of the reconstituted lyophilized pooled PRP composition was assessed after intra-articular injection in two different joints in normal horses. Two cohorts of six animals each were selected for treatment and evaluation. Animals were included in the study if, prior to the study start: 1) there was no measurable lameness that would interfere with outcome measurements; 2) animals were ≥nine months of age and in good general health; and 3) animals demonstrated general behavior to allow for handling and not interfere with outcome assessment. Animals were excluded from the study if, prior to the study start: 1) animals had CBC/Chem abnormalities that could interfere with study outcomes; 2) animals showed radiographic abnormalities that could interfere with outcome measurements; 3) animals had previously received corticosteroids administered intra-arterially or systemically within the past sixty days; 4) animals exhibited clinically relevant joint pathology that would interfere with outcome measurements; or 5) animals had other known health problems that would interfere with outcome measurements. In each group of six animals, three animals received the reconstituted lyophilized pooled PRP composition and three animals received saline (negative controls), as set forth in the table below:

| | |
|---|---|
| Cohort | Condition (number of animals treated) |

| | | |
|---|---|---|
| Cohort 1 | PRP composition (3) | Saline negative control (3) |
| Cohort 2 | PRP composition (3) | Saline negative control (3) |

Evaluators were blinded to the treatment condition. Study duration was thirty days from start to last in-life time point.

Animals were administered 4 mL of the PRP composition or saline as a single joint injection, with no crossover. Condition was determined randomly using a random number generator, and accord with the following steps: 1) select first horse for cohort 1, 2) place horse patient identification (ID) number next to randomized animal number, 3) continue until cohort 1 is filled, 4) select first horse for cohort 2, 5) place horse patient identification (ID) number next to randomized animal number, and 6) continue until cohort 2 is filled. Results are set forth in the table below:

| | |
|---|---|
| COHORT 1 | COHORT 2 |

| Order Selected | Patient # | Order Selected | Patient # |
|---|---|---|---|
| 1 | 2 | 1 | 12 |
| 2 | 4 | 2 | 7 |
| 3 | 3 | 3 | 11 |
| 4 | 5 | 4 | 9 |
| 5 | 6 | 5 | 10 |
| 6 | 1 | 6 | 8 |

Animals were randomized to either stifle or radial carpal joint, and randomized as to left or right. A different joint (stifle or radial carpal) was used for the repeat injection (per CVM concurrence). Site was confirmed by ultrasound and assessment of joint fluid in the injection site. Results of randomization, and conditions for particular animals, is as set forth in the table below:

| | | | Rx 1 | | RX 2 | |
|---|---|---|---|---|---|---|
| Patient # | Group | Code | Side | Joint | Side | Joint |
| COHORT 1 | | | | | | |
| 1 | VP | D | R | ST | L | RC |
| 2 | CP | W | L | ST | R | RC |
| 3 | CP | W | L | RC | R | ST |
| 4 | VP | D | L | RC | L | ST |
| 5 | VP | D | R | RC | L | ST |
| 6 | CP | W | R | ST | R | RC |
| COHORT 2 | | | | | | |
| 7 | VP | D | R | ST | L | RC |
| 8 | CP | W | L | ST | R | RC |
| 9 | CP | W | L | RC | R | ST |
| 10 | VP | D | L | RC | L | ST |
| 11 | VP | D | R | RC | L | ST |
| 12 | CP | W | R | ST | R | RC |

ST = stifle joint
RC = radial carpal joint
L = left
R = right

Primary outcomes were assessed in accord with an American Association of Equine Practitioners (AAEP) examination to determine lameness, including alternation in the animal's gait, and attitude and performance; heat (warmth to touch by scale); and general health. Secondary outcomes include adverse events, joint effusion, flexion pain, and appetite. Each of the preceding outcomes were assessed on a scale, as set forth in the table below:

| Observation | Timepoints | | Scale |
|---|---|---|---|
| Heat | 1 | hr | 0 = none |
| | 6 | hr | 1 = minor |
| | 12 | hr | 2 = moderate |
| | 1 | day | 3 = severe |
| Effusion | 2 | days | 0 = none |
| | 3 | days | 1 = minimal |
| | 4 | days | 2 = mild |
| | 5 | days | 3 = moderate |
| | 6 | days | 4 = marked |
| Flexion Pain | 7 | days | 0 = none |
| | | | 1 = minor |
| | | | 2 = moderate |
| | | | 3 = severe |
| Average and | | | 0 = lameness not perceptible under any circumstances |
| Maximum | | | 1 = Lameness is difficult to observe and is not consistently |
| Lameness | | | apparent, regardless of circumstances (e.g., under saddle, |
| Score, and | | | circling, inclines, hard surface, etc. |
| average | | | 2 = Lameness is difficult to observe at a walk or when trotting |
| duration | | | in a straight line but consistently apparent under certain |
| | | | circumstances (e.g., weight-carrying, circling, inclines, hard |
| | | | surface, etc.) |
| | | | 3 = Lameness is consistently observable at a trot under all |
| | | | circumstances. |
| | | | 4 = Lameness is obvious at a walk |
| | | | 5 = Lameness produces minimal weight bearing in motion |
| | | | and/or at rest or a complete inability to move. |
| Temperature | | | ° C. |
| Pulse | | | Beats/minute |
| Respiration | | | Respirations/minute |
| CBC | | | |
| Chem | | | |
| Adverse | | | |
| Events | | | |

Dose was administered on days 0 and 16, identified below as RX1 and RX2, respectively. The timeline for PRP or saline administration, and assessment conditions are set forth in the table below:

| | | | | | | Inj | | | | |
|---|---|---|---|---|---|---|---|---|---|---|
| | | Study | | | CBC | Site | Daily | Lameness | | JF |
| Timepoints | Activity | Day | PE | TPR | Chem | Obs | Obs | Exam | Rads | Collect |
| Baseline | Cages | −7 | x | x | x | x | x | x | x | |
| | | −6 | | | | | | | | |
| | | −5 | | | | | | | | |
| | | −4 | | | | | | | | |
| | | −3 | | | | | | | | |
| | | −2 | | | | | | | | |
| | | −1 | | | | | | | | |
| RX PERIOD | RX 1 | 0 | x | x* | x | x* | x | x | | x |
| 1 | | 1 | | x | | x | x | | | |
| | | 2 | | x | | x | x | | | |
| | | 3 | | x | | x | x | | | |
| | | 4 | | x | | x | x | | | |
| | | 5 | | x | | x | x | | | |
| | | 6 | | x | | x | x | | | |
| | | 7 | x | x | x | x | x | x | | x |
| Rest | Cages | 8 | | | | | | | | |
| Period | | 9 | | | | | | | | |
| | | 10 | | | | | | | | |
| | | 11 | | | | | | | | |
| | | 12 | | | | | | | | |
| | | 13 | | | | | | | | |
| | | 14 | | | | | | | | |
| | | 15 | | | | | x | | | |
| RX PERIOD | Rx 2 | 16 | x | x* | x | x* | x | x | | x |
| 2 | | 17 | | x | | x | x | | | |
| | | 18 | | x | | x | x | | | |
| | | 19 | | x | | x | x | | | |
| | | 20 | | x | | x | x | | | |
| | | 21 | | x | | x | x | | | |
| | | 22 | | x | | x | x | | | |
| | End | 23 | x | x | x | x | x | x | | x |
| | InLife | | | | | | | | | |

*= additional Rx day timepoints of 1, 6, and 12 hours
PE = physical exam
TPR = temperature, pulse, and respiration
CBC Chem = complete blood count (CBC) and chemistry profile
Inj Site Obs = Injection Site Observations
Daily Obs = Daily Observations
Lameness Exam = Lameness Exam in accord with AAEP protocols
Rads = Radiograph examination for evaluation of joint pathology
JF Collect for future analysis

Outcomes are assessed as change from baseline for each injected animal and the clinical significance is assessed by the attending veterinarian as to the significance and safety profile. In general, a safe product will not produce a significant adverse event such as lameness, swelling or inflammation.

### Safety Assessment in Dogs

Safety of the reconstituted lyophilized pooled PRP composition was assessed after intra-articular injection in two different joints in normal dogs. Two cohorts of six animals each were selected for treatment and evaluation. In each group of six animals, three animals received the reconstituted lyophilized pooled PRP composition and three animals received saline (negative controls), as set forth in the table below:

| | |
|---|---|
| Cohort | Condition (number of animals treated) |

| | | |
|---|---|---|
| Cohort 1 | PRP composition (3) | Saline negative control (3) |
| Cohort 2 | PRP composition (3) | Saline negative control (3) |

Evaluators were blinded to the treatment condition. Study duration was thirty days from start to last in-life time point.

Animals were administered 2 mL of the PRP composition or saline as a single joint injection, with no crossover. Condition was determined randomly using a random number generator, and accord with the following steps: 1) select first dog for cohort 1, 2) place dog patient identification (ID) number next to randomized animal number, 3) continue until cohort 1 is filled, 4) select first dog for cohort 2, 5) place dog patient identification (ID) number next to randomized animal number, and 6) continue until cohort 2 is filled. Results are set forth in the table below:

| | |
|---|---|
| COHORT 1 | COHORT 2 |

| Order Selected | Patient # | Order Selected | Patient # |
|---|---|---|---|
| 1 | 2 | 1 | 12 |
| 2 | 4 | 2 | 7 |
| 3 | 3 | 3 | 11 |
| 4 | 5 | 4 | 9 |
| 5 | 6 | 5 | 10 |
| 6 | 1 | 6 | 8 |

Animals were randomized to either stifle or hip joint, and randomized as to left or right. A different joint (stifle or hip) was used for the repeat injection (per CVM concurrence). Site was confirmed by assessment of joint fluid in the injection site. Results of randomization, and conditions for animals, is as set forth in the table below:

| | | | Rx 1 | | RX 2 | |
|---|---|---|---|---|---|---|
| Patient # | Group | Code | Side | Joint | Side | Joint |
| COHORT 1 | | | | | | |
| 1 | VP | D | R | FT | L | CF |
| 2 | CP | W | L | FT | R | CF |
| 3 | CP | W | L | CF | R | FT |
| 4 | VP | D | L | CF | L | FT |
| 5 | VP | D | R | CF | L | FT |
| 6 | CP | W | R | FT | R | CF |
| COHORT 2 | | | | | | |
| 7 | VP | D | R | FT | L | CF |
| 8 | CP | W | L | FT | R | CF |
| 9 | CP | W | L | CF | R | FT |
| 10 | VP | D | L | CF | L | FT |
| 11 | VP | D | R | CF | L | FT |
| 12 | CP | W | R | FT | R | CF |

CF = coxofemoral
FT = femurotibial
L = left
R = right

Primary outcomes were based on assessment of lameness, based on veterinary examination or pain upon manipulation. Secondary outcomes include adverse events, such as heat by manual detection, joint effusion, flexion pain, general health, and appetite. Each of the preceding outcomes were assessed on a scale, as set forth in the table below:

| Observation | Timepoints | | Scale |
|---|---|---|---|
| Heat | 1 | hr | 0 = none |
| | 6 | hr | 1 = minor |
| | 12 | hr | 2 = moderate |
| | 1 | day | 3 = severe |
| Effusion | 2 | day | 0 = none |
| | 3 | day | 1 = minimal |
| | 4 | day | 2 = mild |
| | 5 | day | 3 = moderate |
| | 6 | day | 4 = marked |
| Flexion Pain | 7 | day | 0 = none |
| | | | 1 = minor |
| | | | 2 = moderate |
| | | | 3 = severe |
| Average and | | | 0 = lameness not perceptible under any circumstances |
| Maximum | | | 1 = Lameness is difficult to observe and is not consistently |
| Lameness | | | apparent, regardless of circumstances (e.g., under saddle, |
| Score, and | | | circling, inclines, hard surface, etc. |
| average | | | 2 = Lameness is difficult to observe at a walk or when trotting in |
| duration | | | a straight line but consistently apparent under certain |
| | | | circumstances (e.g., weight-carrying, circling, inclines, hard |
| | | | surface, etc.) |
| | | | 3 = Lameness is consistently observable at a trot under all |
| | | | circumstances. |
| | | | 4 = Lameness is obvious at a walk |
| | | | 5 = Lameness produces minimal weight bearing in motion |
| | | | and/or at rest or a complete inability to move. |
| Temperature | | | ° C. |
| Pulse | | | Beats/minute |
| Respiration | | | Respirations/minute |
| CBC | | | |
| Chem | | | |
| Adverse | | | |
| Events | | | |

Dose was administered on days 0 and 16, identified below as RX1 and RX2, respectively. The timeline for PRP or saline administration, and assessment conditions are set forth in the table below:

| | | | | | | Inj | | | | |
|---|---|---|---|---|---|---|---|---|---|---|
| | | Study | | | CBC | Site | Daily | Lameness | | JF |
| Timepoints | Activity | Day | PE | TPR | Chem | Obs | Obs | Exam | Rads | Collect |
| Baseline | Pasture | −7 | x | x | x | x | x | x | x | |
| | | −6 | | | | | | | | |
| | | −5 | | | | | | | | |
| | | −4 | | | | | | | | |
| | | −3 | | | | | | | | |
| | | −2 | | | | | | | | |
| | | −1 | | | | | | | | |
| RX PERIOD | RX 1 | 0 | x | x* | x | x* | x | x | | x |
| 1 | | 1 | | x | | x | x | | | |
| | | 2 | | x | | x | x | | | |
| | | 3 | | x | | x | x | | | |
| | | 4 | | x | | x | x | | | |
| | | 5 | | x | | x | x | | | |
| | | 6 | | x | | x | x | | | |
| | | 7 | x | x | x | x | x | x | | x |
| Rest | Pasture | 8 | | | | | | | | |
| Period | | 9 | | | | | | | | |
| | | 10 | | | | | | | | |
| | | 11 | | | | | | | | |
| | | 12 | | | | | | | | |
| | | 13 | | | | | | | | |
| | | 14 | | | | | | | | |
| | | 15 | | | | | x | | | |
| RX PERIOD | Rx 2 | 16 | x | x* | x | x* | x | x | | x |
| 2 | | 17 | | x | | x | x | | | |
| | | 18 | | x | | x | x | | | |
| | | 19 | | x | | x | x | | | |
| | | 20 | | x | | x | x | | | |
| | | 21 | | x | | x | x | | | |
| | | 22 | | x | | x | x | | | |
| | End | 23 | x | x | x | x | x | x | | x |
| | InLife | | | | | | | | | |

*= additional Rx day timepoints of 1, 6, and 12 hours
PE = physical exam
TPR = temperature, pulse, and respiration
CBC Chem = complete blood count (CBC) and chemistry profile
Inj Site Obs = Injection Site Observations
Daily Obs = Daily Observations
Lameness Exam = Lameness Exam in accord with AAEP protocols
Rads = Radiograph examination for evaluation of joint pathology
JF Collect for future analysis

Outcomes are assessed as change from baseline for each injected animal and the clinical significance assessed by the attending veterinarian as to the significance and safety profile. In general, a safe product does not produce a significant adverse event such as lameness, swelling or inflammation.

### Example 13

### Pooled Platelet-Rich Plasma Composition Compared to Platelet-Rich Plasma Isolated from an Individual Animal

Platelets and plasma were collected from individual horses by apheresis and apheresis, respectively, as detailed above in Example 1. For assessment of PRP from individual animals, the platelets and plasma were pooled together. PRP from ten individual animals were tested. The results show animal to animal variation in growth factor output, platelet count, and white blood cell count between animals and compared to pooled PRP (PRP with platelets and plasma pooled from multiple animals). The variability between individual animals likely is due to genetic and physiological variability between horses and also due to the variation in veterinary operator techniques; such differences are observed as diverse cellular and cytokine profiles in the PRP. These results indicate that pooled platelets and plasma from multiple animals provide greater consistency and uniformity between batches of PRP.

### Example 14

### Platelet Count in a Lyophilized Platelet-Rich Plasma Composition for Therapeutic Use

The lyophilized PRP composition as prepared in Example 1 was assessed for cell number. The lyophilized PRP composition contains 400,000 to 500,000 platelets per microliter, in an 8 mL reconstituted sample.

Platelet concentration and/or total platelet count in a preparation can be varied depending on the indication for treatment or use. For example, higher amounts/platelet counts and/or more concentrated PRP are administered directly to the joints, and lower amounts/platelet counts and/or less concentrated PRP can be administered intra-lesionally.

### Example 15

### Pooled Platelet-Rich Plasma Composition Use in Conjunction with Other Compounds

The reconstituted lyophilized PRP composition is combined or co-administered with a secondary compound, and/or therapeutic. The co-administered compound and/or therapeutic is selected based on the disease, disorder or condition for treatment or the use of the PRP composition and the secondary compound and/or therapeutic.

Pooled PRP Composition for Use in Combination with Antibiotics

The reconstituted lyophilized PRP composition is combined or co-administered with antibiotics to human subjects or animals for wound healing and for treatment of contaminated wounds. The reconstituted lyophilized PRP composition is administered to human or animal subjects to improve wound healing in a contaminated wound, where the wound is contaminated with bacteria. The human or animal subject is administered a standard dose of the reconstituted lyophilized PRP composition administered topically. The amount of reconstituted lyophilized PRP composition administered to the wound is calculated based on severity of the wound, wound location, type of wound, size of wound. The reconstituted lyophilized PRP composition is administered to a contaminated wound, such as a bacterially infected wound, concomitantly with or before or after administration of an antibiotic, for treating a contaminated wound.

Pooled PRP Composition for Use in Combination with Bone Matrix Materials

The reconstituted lyophilized PRP composition is combined or co-administered with bone matrix materials to human subjects or animals for bone healing and for treatment of bone injury. The human or animal subject is administered a standard dose of the reconstituted lyophilized PRP composition administered mixed with the bone matrix material and then placed into the bone defect area. The lyophilized PRP composition is co-administered with bone matrix materials such as bone autograft, bone allograft, and/or calcium mixtures. The reconstituted lyophilized PRP composition is administered to a human or animal subject with a bone injury or bone dysfunction or bone degradation or lack of bone regrowth or regeneration or a bone disorder concomitantly with or before or after administration of an antibiotic, for treating a contaminated wound.

Pooled PRP Composition for Use in Combination with ECM- and/or MSC-Derived Exosomes or Mitochondria

The reconstituted lyophilized PRP composition is combined or co-administered with ECM- or MSC-derived exosomes and/or mitochondria. MSC-derived exosomes can be added to the composition or co-administered with the PRP composition to contribute to healing of injured or diseased tissues and organs, such as, for example, heart, kidney, liver, immune and neurological conditions, and wound healing.

Pooled Platelet-Rich Plasma Composition Use in Conjunction with Stem Cells

The reconstituted lyophilized PRP composition is co-administered with autologous stem cells or allogeneic stem cells. In some examples PRP use or co-administration with autologous or allogeneic stem cells results in a synergist treatment method. In some examples PRP is added to autologous or allogeneic stem cell cultures ex vivo and cells are cultured in the presence of PRP. In some examples PRP use or co-administration or co-culture with autologous or allogeneic stem cells or cell culture prolongs the effect of stem cells that are administered in vivo or that are prepared in vitro.

### Example 16

### Leukocyte Count and Type in Pooled Platelet-Rich Plasma Composition

The lyophilized PRP composition is assessed for the presence, number, and type of leukocytes in the composition. In examples, the PRP composition is leukoreduced and does not contain leukocytes or does not contain a substantial amount (e.g., more than 10% or more than 20%) of leukocytes. In examples, leukocyte count is verified using flow cytometry, such as by using leukocyte specific markers, or by an impedance counter.

### Example 17

### Rehydration of a Lyophilized Pooled Platelet-Rich Plasma Composition

The lyophilized PRP composition prepared in accord with the method set forth in Example 1 is rehydrated in sterile saline (0.9% saline). Rehydration with alternatives to saline is based on the therapeutic use of the PRP composition. The lyophilized PRP composition is rehydrated in sterile water for injection, saline, native patient specific plasma, and/or allogeneic plasma.

### Example 18

### Antimicrobial Effect of a Pooled Platelet-Rich Plasma Composition

The antimicrobial effect of a PRP composition and reconstituted lyophilized PRP composition is evaluated. Platelet poor plasma is used as negative control, and previously characterized antibiotics are used as positive control(s). The antimicrobial effect of a PRP composition is evaluated against Staphylococcus aureus and Escherichia coli.

Bacterial stocks are obtained by picking one colony of Staphylococcus aureus or Escherichia coli from a collection and incubating on an agar plate at 37° C. overnight. Bacterial liquid stocks are prepared by picking several colonies with a loop and diluting them in 9 mL of PBS. Bacterial count is performed before initiation of the experiments via determination of optical density (OD) at 600 nm, where values close to 0.3 correspond to more than 107 colony forming units (CFUs)/ml. Bacterial liquid stocks are grown overnight in growth media. 1 mL aliquots from bacteria grown overnight in growth media are collected and centrifuged at 3,000 rpm for 10 minutes to pellet the bacteria. Bacteria pellets are washed with PBS and canine or equine PRP (20 and 80% v/v) or PBS in growth media are added to the liquid bacterial cultures. Tube contents are transferred in duplicate to a 48-well plate, which is placed into an automated plate reader (from Synergy HT, BioTek) and allowed to incubate for 16 hours at 37° C. with rotation at 200 rpm. The numbers of bacteria per well (OD600) is determined every 10 minutes.

A decrease in the number of bacteria in the PRP samples compared to the PBS control indicates an antimicrobial effect of the PRP composition.

### Example 19

### Pooled Platelet-Rich Plasma Composition Manufacturing Process Provides a Sterile Composition

PRP is prepared in a sterile environment and utilizing a sterile manufacturing process. Sterility of the composition prevents introduction of microorganisms into the recipient upon administration, prevents degradation of the PRP composition components, and decreases the amount of endotoxin. PRP samples that are not prepared under sterile conditions may contain microorganisms, such as bacteria, which can impact the efficacy of the PRP composition upon administration and introduce adverse effects in the subjects.

### Example 20

### Canine PRP Preparation and Dosage

The PRP product provided herein (available under the trademark PrecisePRP™ PRP product), as detailed herein, is a leukoreduced allogeneic, pooled, freeze-dried platelet-rich plasma product from up to 32 canine donors. The biological source material for this product is in-date leukoreduced, apheresis-derived canine platelet concentrates and apheresis-derived or whole blood-derived frozen plasma. Donors are blood typed for DEA 1, 4, and 7 as well as tested for plasma antibody negative to routine canine red blood cell antigens. Selection of donors and evaluation for infectious disease is compliant with FDA CVM Guidance #254. Supplied in a 50 mL glass vial, this product is a sterile, nonpyrogenic white-to-tan powder. Once rehydrated with 8 mL of sterile water for injection, this product is a light yellow translucent fluid with 4.0×10⁹ platelets per vial and less than 1500 white blood cells per μL.

This product is formulated as a species-specific source of concentrated platelets in plasma for intra-articular administration. It can be provided in vials for single use or multiple use. One vial, for example for use for canines, provides 8 mL of allogeneic pooled platelet-rich plasma with a total of approximately 4×10⁹ canine platelets per vial. An example of this product, referred to as PrecisePRP Canine PRP, is a leukoreduced, allogeneic, pooled, freeze-dried platelet-rich plasma (PRP). It has received FDA review clearance and is the first animal cell, tissue, and cell, and tissue-based product (ACTP) intended for intra-articular use to have undergone FDA review and received this determination. Each vial of the injectable contains a consistent dose of four billion platelets at a concentration of 500,000 platelets per microliter and is leukoreduced with fewer than 1,500 white blood cells per microliter. Dosage can be adjusted based on the size of the dog and the joint being treated.

The lyophilized product can be rehydrated. For example, rehydration instructions can include the following:
Draw 8 milliliters of sterile water into a sterile syringe and needle.
Apply the supplied vented administration clave to the vial and seat it on the crimped seal.
Disengage the needle and, using sterile technique, attach the syringe to the clave hub.
Slowly add the sterile water down the side of the vial lumen to avoid foaming and immerse the cake in the rehydrating fluid.
Gently mix the fluid and the lyophilized powder by swirling the vial to rehydrate.
Draw into a syringe using the bidirectional clave.
Engage a needle greater than or equal to 22 gauge for administration.
Store at room temperature (18-25° C.) until administration for not more than four (4) hours.
Discard the excess product.

### Example 21

### Canine Safety Data

To demonstrate safety of the PRP product provided herein and its use in multiple joints in a single animal, animals were treated and data were collected. Follow-up with veterinarians who had injected dogs with the product in more than one joint was conducted.

### Methods

All dogs in the Table below had been injected in two or more joints at the same time and were followed for at least 14 days after the injection date. None of these 12 injected dogs demonstrated an adverse event. The Table below presents the results for all dogs that met the criteria of a minimum two weeks post injection in two or more joints. One dog injected in two joints reported an incidental adverse event of a right bundle branch block at pre-injection ECG with no reported change post injection. The data demonstrate clinical injection in multiple joints in a single animal with very low adverse event rate.

### PRP Canine Post-Injection Multi-Joint Adverse Event Summary

| | | | Number | Adverse |
|---|---|---|---|---|
| Canine | Age | Days after | of sites | events |
| Patient | (years) | injection | treated | (Y/N) |
| 1 | 3.5 | 109 | 2 | N |
| 2 | 1.6 | 92 | 3 | N |
| 3 | 5 | 76 | 2 | N |
| 4 | 1.6 | 73 | 6 | N |
| 5 | 12 | 62 | 4 | N |
| 6 | 12.7 | 50 | 2 | N |
| 7 | 13 | 37 | 8 | N |
| 8 | 5 | 30 | 4 | N |
| 9 | 3 | 20 | 2 | N |
| 10 | 11 | 20 | 2 | N |
| 11 | 2 | 19 | 4 | N |
| 12 | 8.4 | 17 | 4 | N |
| Averages | | 50.4 | 3.6 | |

### Canine Orthopedic Treatments

The following table shows the outcomes of eight canine patients treated for joint disease and orthopedic soft tissue (tendon, ligament) injuries with the patient demographics, type of injury, improvement, and whether the dog owner would recommend this therapy. This clearly shows reduction to practice of the use of PrecisePRP Canine in canine joint disease and canine orthopedic soft tissue injuries.

| Patient | | Days after | Number of | | | | Improvement |
|---|---|---|---|---|---|---|---|
| # | Age | injection | sites treated | type | location | Injury* | level** |
| 1 | 1 | 106 | 1 | joint | Stifle R/L | CCL | 2 |
| 2 | 4 | 106 | 1 | joint | Stifle R | CCL | 1 |
| 3 | 7 | 78 | 1 | joint | Stifle L | CCL | 1 |
| 4 | 9 | 58 | 3 | joint | Elbow R/L | OA | 1 |
| 5 | 6 | 69 | 1 | tendon | Bicep R | Tenosyn | 1 |
| 6 | 6 | 56 | 1 | joint | Stifle L/Hip R | OA | 1 |
| 7 | 12 | 123 | 2 | tendon | Bicep R | Tenosyn | 1 |
| 8 | 5 | 36 | 1 | Ligament | Carpus | OA | 1 |

*CCl = cranial cruciate ligament; OA = osteoarthritis; tenosyn = tenosynovitis
**1 = significant improvement; 2 = mild improvement

### Example 22

### Exemplary Equine Dosage and Efficacy Data

The PRP preparation for equine use is the leukoreduced allogeneic, pooled freeze-dried, platelet-rich plasma product prepared from apheresis-derived equine platelet concentrates and apheresis-derived frozen plasma from up to 18 equine donors. The product, provided under the trademark PrecisePRP™ Equine PRP as detailed herein provides a species-specific source of concentrated platelets in plasma for intra-articular administration.

All horses are blood type AaCa positive and negative for plasma antibodies directed at blood types that are their blood type. Donors were from a closed herd compliant with USDA regulations for production of licensed biologics. Selection of donors and evaluation for infectious disease was compliant with FDA CVM Guidance #254.

Supplied in a 50 mL glass vial, the product is a sterile, nonpyrogenic white-to-tan powder. Once rehydrated with 8 mL of sterile water for injection, this product is a gold translucent fluid with 4.0×10⁹ platelets per vial and fewer than 1500 white blood cells per μL. One vial provides 8 mL of leukoreduced allogeneic pooled freeze-dried platelet-rich plasma with a total of approximately 4×10⁹ equine platelets per vial. The product is rehydrated as described in the example above for rehydration of the canine product.

### Dosage

For use in regenerative medicine, dosage is lesion dependent and determined by the practitioner at the time of use. Each microliter contains 500,000+/−100,000 platelets. The dose given is adjusted according to parameters, including the size of the joint to be injected. Generally, the dosage per joint should be 2-4 mL.

### Equine Osteoarthritis Treatments

An equine veterinarian treated five performance horses with a single dose of the PRP product PrecisePRP® Equine in response to owner reported lameness in these horses. The following Table shows the joints treated and the outcomes of each horse:

| Horse # | Joint(s) treated | # doses | Outcome |
|---|---|---|---|
| Horse #1 | Tarsal (hock) | 1 | Complete soundness |
| Horse #2 | Tarsal (hock) | 1 | Complete soundness |
| Horse #3 | Tarsal (hock) | 1 | Complete soundness |
| Horse #4 | Coffin joint | 1 | Complete soundness |
| Horse #5 | Coffin joint | 1 | Significantly improved |

### Equine Wound Healing Data

An equine veterinarian, Dr. H, treated a 15-month-old draft horse with PrecisePRP® equine in combination with autologous adipose stem cells for a serious inguinal wound that had resulted from an infected castration surgery. This patient was treated a minimum of 14 times over a several week period with topical and tissue injection of the PrecisePRP while also administering intravenous autologous adipose stem cells. The patient started treatment with wound dimensions of 9 cm×3 cm×11 cm. Expected healing by the veterinarian was four months. Upon successful treatment conclusion at week six, the wound was completed healed. This demonstrates both a wound healing application of PrecisePRP and the co-administration or adipose stem cells (a mesenchymal stem cell) in a more serious wound environment.

### Example 23

### Feline Corneal Ulcer Treatment

A small animal veterinarian had a feline patient with a serious corneal ulcer. With no options for further treatment, the veterinarian used the Canine PRP product (prepared as described herein) in a xenogeneic (cross-species) application with a topical route of administration. The patient was treated a minimum of twice daily for five days. The ulcer healed completely.

### Example 24

### Antimicrobial Effects of the PrP Product

### Antimicrobial Effects

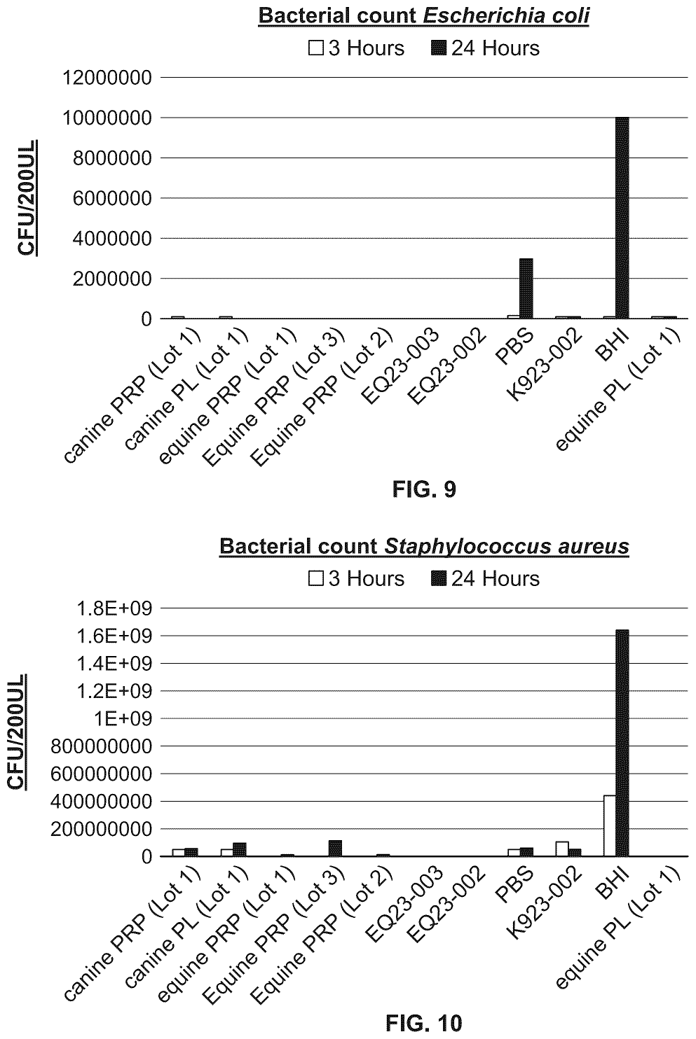

A laboratory evaluation of the antimicrobial properties of the PRP composition. The antimicrobial effects in equine (three batches) and canine (three batches) PRP preparations were evaluated. Platelet poor plasma and known antibiotics were used as controls. The antimicrobial effects were evaluated against Staphylococcus aureus and Escherichia coli. Bacterial stocks were obtained by picking one colony from the collection and incubating in agar plate at 37° C. overnight. Bacterial liquid stocks were prepared by picking several colonies with a loop and diluting them in 9 ml of PBS. Bacterial counts were performed before initiation of the experiments via determination of optical density (OD) at 600 nm (values close to 0.3 corresponds to more than 107 colony forming units (CFUs)/ml). 1 ml aliquots from bacteria grown overnight in growth media were collected and centrifuged at 3,000 rpm for 10 min. Bacteria pellets were washed with PBS and appropriate treatment containing canine and equine PRP (20 and 80% v/v) or PBS in growth medium were added. Tube contents were transferred in duplicate to a 48-well plate, placed into an automated plate reader (Synergy HT, BioTek), and incubated for 16 h at 37° C., 200 rpm. The numbers of bacteria per well (OD600) were determined every 10 min. The results in FIGS. 9 and 10, for, respectively, E. coli and S. aureus bacteria, summarize the data showing that all PRP lots exhibit zero or very low growth. FIGS. 9 and 10 show microbial growth in BHI growth medium (brain-heart infusion), and the growth in absence of the PRP, and growth in PBS (phosphate buffered saline). As shown in FIGS. 9 and 10, the various lots of the PRP preparations provided herein inhibit or prevent microbial growth.

### Example 25

### Stability Data

Three separate lots of the PRP formulated for equine administration were analyzed at approximately 8.8, 9.6 and 12.1 months after manufacturing and lyophilization. To demonstrate stability, these lots were analyzed for sterility, endotoxin, and growth factor (Platelet-derived Growth Factor BB). The resting and activated levels of PDGF-BB were compared to baseline to demonstrate maintenance of potency. The sterility and endotoxin were maintained at quality assurance specification levels for duration of the stability testing.

| | | | | PDGF-BB % | PDGF-BB % |
|---|---|---|---|---|---|
| Lot | | | | of Baseline | of Baseline |
| # | Months | Sterility | Endotoxin | (Resting) | (Activated) |
| 1 | 8.8 | Neg | <.15EU pass | 169% | 145% |
| 12 | 9.6 | Neg | <.15EU pass | 101% | 135% |
| 3 | 12.1 | Neg | <.15EU pass | 90% | 215% |

These data demonstrate stability after lyophilization and stability after storage at room temperature (unrefrigerated). These data demonstrate that the PRP formulation is effective in a plurality of indications and indicate that the PRP is effective for any indications in which primed and/or activated platelets have therapeutic effects.

### Example 26

### Treatment of Otitis

The PRP compositions provided herein are effective for treating otitis externa. Administration of the reconstituted composition to the ears of dogs effectively and quickly effect treatment. For example, a Newfoundland breed who had chronic ear issues since about one year of age. She had been treated with gentamicin based treatments for more than two years, requirement at least weekly cleanings and treatments to control the otitis itch. The ears, which had not been treated for about two weeks, were cleaned with a standard otic wash. A bottle of PrecisePRP Canine® PRP composition was reconstituted with 4 mLs of water and about 1 mL was introduced into each ear. The dog's head was held for about 3-5 minutes so she could not shake it out right away. A visual inspection two days later found little debris in her ears, just normal wax, and no smell. Within two days, the dog had stopped scratching her ears and shaking her head. Prior treatments did not work as quickly and required treatments for a week or so up to three times a day. Hence, the PRP compositions provided herein are effective for quickly effecting treatment of otitis externa.

Since modifications will be apparent to those of skill in the art, it is intended that the invention(s) provided herein is/are limited only by the scope of the appended claims.

1. A freeze-dried (lyophilized) PRP composition comprising plasma and platelets, wherein the freeze-dried PRP composition does not comprise a cryopreservative, which is an agent that protects platelets from cold-induced damage.

2. The freeze-dried platelet-rich plasma (PRP) composition of claim 1, wherein:
40% or more of the platelets are in a primed state; and
primed platelets exhibit at least two of the properties selected from among lack of aggregation upon swirling or light agitation, externalized phosphatidylserine, intact granules, negative for p-selectin on the platelet surface, and retention of discoid shape.

3. The freeze-dried PRP composition of claim 1, wherein the composition does not contain any of dimethyl sulfoxide (DMSO), trehalose, glucose, glycerol, maltodextrin, dextran, hydroxyethyl starch (HES), formaldehyde, paraformaldehyde, glutaraldehyde, and/or permanganate.

4. The freeze-dried PRP composition of claim 1, wherein the amount of platelets in the composition is between at or about 8×10⁹ platelets/mg and 3.3×10⁹ platelets/mg of freeze-dried composition.

5. A platelet-rich plasma (PRP) composition, comprising primed platelets and plasma, wherein:
at least 40%, of the platelets in the composition are primed, but not activated;
platelet-rich plasma (PRP) is a composition comprising plasma with a platelet count above that of peripheral blood; and
primed platelets exhibit two or more of the following properties or markers: externalized phosphatidylserine, intact granules, negative for p-selectin on the platelet surface, retain discoid shape, and do not aggregate upon swirling or light agitation, where if the property is lack of aggregation upon swirling or light agitation, a second property of externalized phosphatidylserine, intact granules, negative for p-selectin on the platelet surface, and retention of discoid shape.

6. The PRP composition of claim 5, wherein primed platelets have two or more properties selected from intact granules, externalized phosphatidylserine, and are negative for p-selectin.

7. The PRP composition of claim 5, wherein plasma comprises between at or about 30% up to 80%, or between at or about 20% up to 90%, by volume of the composition.

8. The PRP composition of claim 5 that is a reconstituted liquid composition comprising the freeze-dried composition and a liquid that comprises saline or sterile water, or plasma, or conditioned medium, or mixtures thereof.

9. The PRP composition of claim 5 that has been reconstituted by rehydration of a freeze-dried composition, wherein the PRP is reconstituted in a volume of at least or at least about 1 mL up to 9 mL.

10. The PRP composition of claim 5 that contains 1×10⁹ to 5×10⁹ platelets and up to 1×10⁸ to 1.5×10⁸ white blood cells.

11. The PRP composition of claim 5 that comprises about 4×10⁹±10% platelets.

12. The platelet-rich plasma (PRP) composition of claim 5, comprising platelets and plasma, wherein:
the concentration of platelets is the same as or greater than the concentration of platelets in blood;
the amount of plasma in the composition is 30% to 80% by volume; and
at least 40% of the platelets are in a primed state in which the platelets exhibit one or more of the following properties or markers: externalized phosphatidylserine, intact granules, negative for p-selectin on the platelet surface, retain discoid shape, and do not aggregate upon swirling or light agitation, where if the property is lack of aggregation upon swirling or light agitation, a second property of externalized phosphatidylserine, intact granules, negative for p-selectin on the platelet surface, or retention of discoid shape.

13. The PRP composition of claim 5, wherein the amount of plasma in the composition is 30% to 80% by volume.

14. The PRP composition of claim 5, wherein up to about or at 85% of the platelets are in a primed state.

15. A freeze dried PRP composition of claim 5 that does not contain cryopreservative.

16. The PRP composition of claim 5, wherein the concentration of platelets is at least 200,000 platelets/μl of composition, or, when freeze dried contains at least or at least about 300,000 platelets/μg.

17. The freeze-dried PRP composition of claim 1, wherein the concentration of platelets is at or about 300,00 to 4,000,000 platelets/μg.

18. The PRP composition of claim 5, wherein the concentration of platelets is 150,000 platelets/μl to 500,000 platelets/μl.

19. The PRP composition of claim 5, further comprising anti-coagulant.

20. The PRP composition of claim 19, wherein the anti-coagulant is selected from among one or more of acid citrate dextrose solution A (ACD-A), triple citrate, citrate-phosphate-dextrose solution with adenine (CPDA-1), heparin, ethylenediaminetetraacetic acid (EDTA) and analogs thereof, citrate, acid citrate dextrose solution B (ACD-B), oxalate, sodium fluoride.

21. The PRP composition of claim 5 that is leukoreduced, whereby the level of leukocytes in the PRP composition is lower than level of leukocytes in whole blood.

22. The freeze-dried PRP composition of claim 1, wherein fewer than about or 20% of the platelets in the composition are inactivated.

23. The PRP composition of claim 5, wherein fewer than about or 20% of the platelets in the composition are inactivated.

24. The PRP composition of claim 5, wherein the platelets and/or plasma were pooled from a plurality of donor animals of the same species.

25. The PRP composition of claim 24, wherein the number of donors is up to 40 donor animals.

26. The freeze-dried composition of claim 1, wherein the platelets and plasma are equine or canine.

27. The PRP composition of claim 5, wherein the platelets and plasma are equine or canine.

28. A container comprising the PRP composition of claim 5, wherein the container contains 1×10⁹ to 5×10⁹ platelets, or contains at least 1×10⁹ or 4×10⁹ platelets in a composition that is 1-20 mL or the platelets are in a resulting freeze-dried powder that does not contain cryopreservative.

29. The container of claim 28, wherein the platelets are canine or equine platelets.

30. A combination, comprising the PRP composition of claim 5 or a freeze-dried composition thereof, and an additional therapeutic agent, wherein the additional therapeutic agent is a coformulation with the composition, or is in a separate composition.

31. The combination of claim 30, wherein the additional therapeutic agent comprises one or more of a chemokine, an antibiotic, an anti-inflammatory, an immunotherapeutic, a growth factor, a hormone, a steroid, hyaluronic acid, small molecule(s), a cytokine, and stem cells.

32. A method of preparing a composition of claim 1, comprising refrigerating the platelets for at least 4 hours up to 14 days following collection without warming the platelets to room temperature after collection and during and after processing.

33. A method of making a platelet rich plasma (PRP) composition claim 5, comprising:
(a) collecting platelets from a donor or a plurality of donors, wherein platelets are refrigerated immediately after collection and prior to mixing with additional plasma, wherein platelets are refrigerated immediately after collection and prior to mixing with additional plasma;
(b) without warming the platelets to room temperature after collection and during and after processing, incubating the platelets at refrigeration temperatures, for about 4 hours to 14 days, inclusive, wherein refrigeration is effected at temperatures between at or about 0° C. and 10° C., inclusive, or 4° C. and 8° C., inclusive;
(c) without warming the platelets to room temperature, combining the platelets with plasma.

34. The method of claim 33, further comprising freeze-drying the composition of c) in the absence of a cryopreservative.

35. The method of claim 33, wherein platelets are refrigerated for 4 hours to 5 days, inclusive, or 4 hours to 7 days, inclusive, or for 3-5 days, inclusive.

36. A method of treatment of a disease, disorder, condition, or injury, comprising administering a PRP composition of claim 5 or a freeze-dried compositions thereof, wherein if the PRP composition is a freeze-dried composition the freeze-dried composition is reconstituted prior to administration, or if the PRP composition is a freeze-dried composition the freeze-dried PRP composition is administered as a solid.

37. The method of claim 36, wherein the disease, disorder, condition, or injury is selected from among inflammatory diseases, disorders or conditions; cardiovascular diseases, disorders or conditions; nervous system diseases, disorders or conditions; tumors; demyelinating diseases, disorders or conditions; dermatological system diseases, disorders or conditions; digestive system diseases, disorders or conditions; endocrine system diseases, disorders or conditions; reproductive system diseases, disorders or conditions; hemic and lymphatic diseases, disorders or conditions; immunological diseases, disorders or conditions; mental disorders; musculoskeletal diseases, disorders or conditions; neuromuscular diseases, disorders or conditions; metabolic diseases, disorders or conditions; skin and connective tissue diseases, disorders or conditions or injuries; diseases, disorders, or conditions of the ear; diseases, disorders, or conditions of the eye; spinal diseases, disorders or conditions or injuries; disorders or conditions or injuries of tendons, muscles, bone, and joints; acute bleeding, wounds, inflammation, hair loss, dermatitis, autoimmune disorders, and urological diseases, disorders or conditions.

38. The method of claim 36, comprising administering the composition into a joint in an animal via intra-articular injection.
